(19) United States
(12) Patent Application Publication
(10) Pub. No.: US 2026/0117248 A1
(43) Pub. Date: Apr. 30, 2026

(54) CORN ELITE EVENT MZIR098

(71) Applicant: Syngenta Crop Protection AG, Basel (CH)

(72) Inventors: Andrew Noe, Clinton, IL (US); Jared Conville, Research Triangle Park, NC (US); Weining Gu, Research Triangle Park, NC (US); Yaping Jiang, Research Triangle Park, NC (US); Ryan William Carlin, Research Triangle Park, NC (US); Wenjin Yu, Research Triangle Park, NC (US); Volker Mittendorf, Research Triangle Park, NC (US)

(73) Assignee: Syngenta Crop Protection AG, Basel (CH)

(21) Appl. No.: 19/411,694

(22) Filed: Dec. 8, 2025

Related U.S. Application Data
(62) Division of application No. 17/870,257, filed on Jul. 21, 2022, now Pat. No. 12,516,345.
Application No. 17/870,257 is a division of application No. 16/619,271, now Pat. No. 11,441,159.
(60) Provisional application No. 62/519,993, filed on Jun. 15, 2017.

(51) Int. Cl. C12N 15/82; C12Q 1/6895

(52) U.S. Cl. CPC C12N 15/8286; C12N 15/8274; C12Q 1/6895; C12Q 2600/13

(57) ABSTRACT

A novel transgenic corn elite event designated MZIR098 is disclosed. The invention relates to DNA sequences of the recombinant constructs inserted into the corn genome and of genomic sequences flanking the insertion site that resulted in elite event MZIR098. The invention further relates to assays for detecting the presence of the DNA sequences of corn elite event MZIR098, to corn plants and corn seeds comprising the genotype thereof, and to methods for producing a corn plant by crossing a corn plant comprising the elite event MZIR098 genotype with itself or another corn variety.

### RELATED APPLICATIONS

This application is a divisional of pending U.S. application Ser. No. 17/870,257 filed Jul. 21, 2022, which is a divisional of U.S. application Ser. No. 16/619,271 filed Dec. 5, 2019, now granted patent U.S. Pat. No. 11,441,159, which is a national stage entry under 35 U.S.C. § 371 of International Application No. PCT/US2018/037189, filed 13 Jun. 2018, which claims the benefit of U.S. Provisional Application No. 62/519,993, filed Jun. 15, 2017, the disclosures of all of which are incorporated by reference herein in their entirety.

### STATEMENT REGARDING ELECTRONIC SUBMISSION OF A SEQUENCE LISTING

A sequence listing in XML format, submitted under 37 C.F.R. § 1.831(a), entitled “81336_ST26_14Jan26”, 487,424 bytes in size, generated on Jan. 14, 2026 and filed via EFS-Web is provided in lieu of a paper copy. This sequence listing is hereby incorporated by reference into the specification for its disclosures.

### FIELD OF THE INVENTION

The invention relates generally to the field of plant molecular biology, plant transformation, and plant breeding. More specifically, the invention relates to insect resistant transgenic corn plants comprising a novel transgenic genotype and to methods of detecting the presence of the corn plant DNA in a sample and compositions thereof.

### BACKGROUND

Plant pests are a major factor in the loss of the world's important agricultural crops. About $8 billion are lost every year in the U.S. alone due to infestations of non-mammalian pests including insects. Species of corn rootworm are considered the most destructive corn pests. Important rootworm pest species include Diabrotica virgifera virgifera, the western corn rootworm; D. longicornis barberi, the northern corn rootworm, D. undecimpunctata howardi, the southern corn rootworm, and D. virgifera zeae, the Mexican corn rootworm.

Corn rootworm is mainly controlled by intensive applications of chemical pesticides. Good corn rootworm control can thus be reached, but these chemicals can sometimes also affect beneficial organisms. Another problem resulting from the wide use of chemical pesticides is the appearance of resistant insect varieties. This has been partially alleviated by various resistance management practices, but there is an increasing need for alternative pest control strategies. One such alternative includes the expression of foreign genes encoding insecticidal proteins in transgenic plants. This approach has provided an efficient means of protection against selected insect pests, and transgenic plants expressing insecticidal toxins have been commercialized, allowing farmers to reduce applications of chemical insecticides.

Bacillus thuringiensis (Bt) Cry proteins (also called 5-endotoxins) are proteins that form a crystalline matrix in Bacillus that are known to possess insecticidal activity when ingested by certain insects. Genes coding for Cry proteins have been isolated and their expression in crop plants have been shown to provide another tool for the control of economically important insect pests. Such transgenic plants expressing the Cry proteins have been commercialized, allowing farmers to reduce or augment applications of chemical insect control agents. Coleopteran-active Cry proteins useful in transgenic plants include, for example, Cry3A, Cry3B and the Cry34/Cry35 complex.

Although the usage of transgenic plants expressing Cry proteins is another tool in the insect control toolbox, it is still susceptible to resistance breakdown. Insect pests that now have resistance against the Cry proteins expressed in certain transgenic plants are known. A strategy to reduce the chances of resistance breakdown is to “stack” transgenic traits with different modes of action against the same insect pest species in a single plant. Currently, transgenic traits are frequently stacked through breeding and subsequent screening to get multiple transgenic traits in a single commercial germplasm. These breeding and screening steps are required for every variety of germplasm into which introduction of these two traits is desirable. For many agronomically important crops, such as corn, these two traits need to be maintained as hybrids for dozens of germplasm varieties. Additionally, factors such as the genetic linkage of undesirable traits or genetic recombination may complicate the introduction of two traits from two distinct loci into a single germplasm variety. Therefore, it would be advantageous to create a nucleic acid molecule which carries multiple insecticidal traits and can be introduced at a single locus in the genome of the transgenic plant. Such a molecule is described in publication WO2016209360 (herein incorporated by reference). However, the creation of such a molecule for transgenic insertion into a crop plant does not predictably create a desirable transgenic event.

The expression of foreign genes in plants can be influenced by their chromosomal position, for example due to chromatin structure and/or the proximity of transcriptional regulation elements close to the integration site (See for example, Weising et al., 1988, “Foreign Genes in Plants,” Ann. Rev. Genet. 22:421-477). A high-quality transgenic event is preferred to not be in a promoter or gene region of the genome. A high-quality transgenic event also must not have negative effects on the agronomic performance of the transgenic plant. Additionally, a high-quality transgenic event is the result of a single, intact, transgene insertion, with little or no transgene rearrangement, and without contamination by extraneous heterologous DNA, such as DNA from the backbone of a vector used during the transformation process. A high-quality transgenic event also is preferred to lack introduced ORFs, which potentially may be expressed in the transgenic plant.

Therefore, it is common to produce hundreds of different events and screen those events for a single event that has desired molecular qualities and transgene expression levels and patterns for commercial purposes. The identified event which satisfies all criteria required for a high-quality event which may be used for commercial purposes is considered an elite event. The elite event is characterized by its exact genomic location, as it is that location which is responsible for the molecular qualities, transgene expression levels, and agronomic performance of the event. This elite event is useful for introgressing its transgene into other genetic backgrounds by sexual outcrossing using conventional breeding methods. Progeny of such crosses maintain the transgene expression characteristics of the original transformant. This strategy may be used to generate an infinite number of hybrids and varieties comprising the elite event, and used to ensure reliable transgene expression in each variety and hybrid.

Because a particular elite event is characterized by its genomic location, it would be advantageous to be able to detect its presence in order to determine whether progeny of a sexual cross contain the elite event. In addition, a method for detecting a particular elite event would be helpful for complying with regulations requiring the pre-market approval and labeling of foods derived from recombinant crop plants, for example. It is possible to detect the presence of a transgene by any well-known nucleic acid detection method, including but not limited to thermal amplification (polymerase chain reaction (PCR)) using polynucleotide primers or DNA hybridization using nucleic acid probes. Typically, for the sake of simplicity and uniformity of reagents and methodologies for use in detecting a particular DNA construct that has been used for transforming various plant varieties, these detection methods generally focus on frequently used genetic elements, for example, promoters, terminators, and marker genes, because for many DNA constructs, the coding sequence region is interchangeable. As a result, such methods may not be useful for discriminating between constructs that differ only with reference to the coding sequence. In addition, such methods are not useful for discriminating between different events, particularly those produced using the same DNA construct. To solve this problem, the sequence of genomic DNA adjacent to the inserted heterologous DNA, the flanking sequence, needs to be known. In particular, the junction sequence between the flanking genomic sequence and the inserted transgene needs to be known.

### SUMMARY

The present invention includes insect resistant and herbicide tolerant transgenic elite event MZIR098 corn, which has incorporated into its genome a transgene comprising the insecticidal trait mCry3A, disclosed in U.S. Pat. No. 7,030,295 (incorporated herein by reference), the insecticidal trait eCry3.1Ab, disclosed in U.S. Pat. No. 8,309,516 (herein incorporated by reference), and the herbicide tolerance trait phosphinothricin acetyltransferase (PAT) (U.S. Pat. Nos. 5,531,236, 5,646,024, 5,648,477, and 5,276,268, herein incorporated by reference). Elite event MZIR098 corn comprises the elite event MZIR098 junction sequences in its genome. The invention further includes novel isolated nucleic acid sequences, namely the junction sequences, which are unique to elite event MZIR098 and are useful for identifying the transgenic corn comprising elite event MZIR098 and for detecting nucleic acids from transgenic elite event MZIR098 corn in a biological sample. The present invention also includes kits comprising the reagents necessary for use in detecting these nucleic acids in a biological sample.

The invention is drawn to an elite event, designated MZIR098, comprising a novel transgenic genotype that comprises coding sequences for insect resistance genes mCry3A and eCry3.1Ab, and for herbicide tolerance gene PAT. These genes are useful in controlling insect pests, particularly Diabrotica spp. insect pests, and also confer herbicide tolerance, particularly to glutamine synthetase (GS) inhibitor herbicides, to plants comprising this event and progeny thereof. The invention also provides transgenic MZIR098 corn plants comprising the genotype of the invention, seed, cells, and tissues from transgenic corn plants comprising the genotype of the invention, and methods for producing a transgenic MZIR098 corn plant comprising the genotype of the invention by crossing a corn inbred comprising the MZIR098 genotype of the invention with itself or another corn line of a different genotype. The transgenic MZIR098 corn plants of the invention may have essentially all of the morphological and physiological characteristics of the corresponding isogenic non-transgenic corn plant in addition to those conferred upon the corn plant by the novel MZIR098 genotype of the invention. The invention also provides compositions and methods for detecting the presence of nucleic acids from elite event MZIR098 based on the DNA sequence of the recombinant expression cassettes inserted into the corn genome that resulted in the elite event MZIR098, and of genomic sequences flanking the insertion site. The elite event MZIR098 can be further characterized by analyzing gene expression levels or protein levels of mCry3A, eCry3.1Ab, and PAT, as well as by testing efficacy against GS inhibitor herbicides, such as glufosinate or bialaphos, or by testing for Corn Rootworm resistance.

According to one aspect, the invention provides an optionally isolated nucleic acid molecule comprising at least 10 contiguous nucleotides of a heterologous DNA sequence inserted into the corn plant genome of the elite event MZIR098 initial corn transformant and at least 10 contiguous nucleotides of a corn plant genome DNA flanking the point of insertion of a heterologous DNA sequence inserted into the corn plant genome of the elite event MZIR098 initial corn transformant. The optionally isolated nucleic acid molecule according to this aspect may comprise at least 20 or at least 50 contiguous nucleotides of a heterologous DNA sequence inserted into the corn plant genome of the elite event MZIR098 initial corn transformant and at least 20 or at least 50 contiguous nucleotides of a corn plant genome DNA flanking the point of insertion of a heterologous DNA sequence inserted into the corn plant genome of the elite event MZIR098 initial corn transformant.

According to another aspect, the invention provides a optionally isolated nucleic acid molecule comprising at least one junction sequence of elite event MZIR098 selected from the group consisting of SEQ ID NO: 1 and SEQ ID NO: 2, and complements thereof. A junction sequence spans the junction between the heterologous DNA comprising the expression cassettes inserted into the corn genome and DNA from the corn genome flanking the insertion site and is diagnostic for the elite event MZIR098.

According to another aspect, the invention provides an optionally isolated nucleic acid linking a heterologous DNA molecule to the corn plant genome in corn elite event MZIR098 comprising a sequence of from about 11 to about 20 contiguous nucleotides selected from the group consisting of SEQ ID NO: 1, SEQ ID NO: 2, and complements thereof.

According to another aspect, the invention provides an optionally isolated nucleic acid molecule comprising a nucleotide sequence selected from the group consisting of SEQ ID NO: 1, SEQ ID NO: 2, SEQ ID NO: 3, SEQ ID NO: 4, and complements thereof.

According to another aspect of the invention, an amplicon comprising a nucleic acid molecule of the invention is provided.

According to still another aspect of the invention, flanking sequence primers for detecting elite event MZIR098 are provided. Such flanking sequence primers comprise a preferably isolated nucleic acid sequence comprising at least 10-15 contiguous nucleotides from nucleotides 1-1076 as set forth in SEQ ID NO: 8 (designated herein as the 5′ flanking sequence), or the complements thereof. In one embodiment of this aspect the flanking sequence primers are selected from the group consisting of SEQ ID NO: 10, 14, 15, 16, 38, 42, 65, and complements thereof.

In another aspect of the invention, the flanking sequences primers comprise a preferably isolated nucleic acid sequence comprising at least 10-15 contiguous nucleotides from nucleotides 1-1075 as set forth in SEQ ID NO: 9 (designated herein as the 3′ flanking sequence), or the complements thereof. In one embodiment of this aspect the flanking sequence primers are selected from the group consisting of SEQ ID NO: 19, 20, 21, 36, 41, 45, 67, and complements thereof.

According to another aspect of the invention, primer pairs that are useful for nucleic acid amplification, for example, are provided. Such primer pairs comprise a first primer comprising a nucleotide sequence of at least 10-15 contiguous nucleotides in length which is or is complementary to one of the above-described genomic flanking sequences (SEQ ID NO: 8 or SEQ ID NO: 9) and a second primer comprising a nucleotide sequence of at least 10-15 contiguous nucleotides of heterologous DNA inserted into the elite event MZIR098 genome. The second primer preferably comprises a nucleotide sequence which is or is complementary to the insert sequence adjacent to the plant genomic flanking DNA sequence as set forth in SEQ ID NO: 7. In one embodiment of this aspect the insert sequence primers are selected from the group consisting of SEQ ID NO: 11, 17, 18, 22, 23, 24, 29, 31, 33, 34, 35, 37, 43, 46, 47, 48, 49, 50, and complements thereof.

According to another aspect of the invention, methods of detecting the presence of DNA corresponding to elite event MZIR098 in a biological sample are provided. Such methods comprise: (a) contacting the sample comprising DNA with a pair of primers that, when used in a nucleic acid amplification reaction with genomic DNA from corn elite event MZIR098; produces an amplicon that is diagnostic for corn elite event MZIR098; (b) performing a nucleic acid amplification reaction, thereby producing the amplicon; and (c) detecting the amplicon. In one embodiment of this aspect, the amplicon comprises a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, and complements thereof.

According to another aspect, the invention provides methods of detecting the presence of a DNA corresponding to the elite event MZIR098 in a biological sample. Such methods comprise: (a) contacting the sample comprising DNA with a probe that hybridizes under high stringency conditions with genomic DNA from corn elite event MZIR098 and does not hybridize under high stringency conditions with DNA of a control corn plant; (b) subjecting the sample and probe to high stringency hybridization conditions; and (c) detecting hybridization of the probe to the DNA. The detected hybridized DNA sequence includes at least one polynucleotide sequence comprising SEQ ID NO: 1, SEQ ID NO: 2, SEQ ID NO: 3, SEQ ID NO: 4, SEQ ID NO: 5, SEQ ID NO: 6, and complements thereof.

According to another aspect of the invention, a kit is provided for the detection of elite event MZIR098 nucleic acids in a biological sample. The kit includes at least one DNA sequence comprising a sufficient length of polynucleotides which is or is complementary to SEQ ID NO: 1, SEQ ID NO: 2, SEQ ID NO: 3, SEQ ID NO: 4, SEQ ID NO: 5 or SEQ ID NO: 6, wherein the DNA sequences are useful as primers or probes that hybridize to isolated DNA from elite event MZIR098, and which, upon amplification of or hybridization to a nucleic acid sequence in a sample followed by detection of the amplicon or hybridization to the target sequence, are diagnostic for the presence of nucleic acid sequences from corn elite event MZIR098 in the sample. The kit further includes other materials necessary to enable nucleic acid hybridization or amplification methods.

In another aspect, the invention provides a method of detecting corn elite event MZIR098 protein in a biological sample comprising: (a) extracting protein from a sample of corn elite event MZIR098 tissue; (b) assaying the extracted protein using an immunological method comprising antibody specific for the mCry3A, eCry3.1Ab, or PAT protein produced by the corn elite event MZIR098 event; and (c) detecting the binding of said antibody to the mCry3A, eCry3.1Ab, or PAT protein.

In another aspect, the invention provides a biological sample derived from an elite event MZIR098 corn plant, tissue, or seed, wherein the sample comprises a nucleic acid comprising a nucleotide sequence which is or is complementary to a sequence selected from the group consisting of SEQ ID NO: 1 and SEQ ID NO: 2, and wherein the sequence is detectable in the sample using a nucleic acid amplification or nucleic acid hybridization method. In one embodiment of this aspect, the sample is selected from the group consisting of corn flour, corn meal, corn syrup, corn oil, cornstarch, and cereals manufactured in whole or in part to contain corn by-products.

In another aspect, the invention provides an extract derived from an elite event MZIR098 corn plant, tissue, or seed comprising a nucleotide sequence which is or is complementary to a nucleotide sequence selected from the group consisting of SEQ ID NO: 1 and SEQ ID NO: 2. In one embodiment of this aspect, the sequence is detectable in the extract using a nucleic acid amplification or nucleic acid hybridization method. In another embodiment of this aspect, the sample is selected from the group consisting of corn flour, corn meal, corn syrup, corn oil, cornstarch, and cereals manufactured in whole or in part to contain corn by-products.

According to another aspect of the invention, corn plants and seeds comprising the nucleic acid molecules of the invention are provided. In one embodiment of the invention, a deposit of elite event MZIR098 corn seed was made to the American Type Culture Collection (ATCC) in accordance with the Budapest Treaty on May 1, 2017. The seed was tested on May 12, 2017 and found to be viable. An example of said seed is deposited as ATCC Accession No: PTA-124143.

According to another aspect, the invention provides a method for producing a corn plant with insect resistance and herbicide tolerance to GS inhibitor herbicides comprising: (a) sexually crossing a first parent corn plant with a second parent corn plant, wherein first or second parent corn plant comprises corn elite event MZIR098 DNA, thereby producing a plurality of first generation progeny plants; (b) selecting a first generation progeny plant that is resistant to at least corn rootworm infestation; (c) selfing the first generation progeny plant, thereby producing a plurality of second generation progeny plants; (d) selecting from the second generation progeny plants, a plant that has insect resistance and herbicide tolerance to GS inhibitor herbicides; wherein the second generation progeny plants comprise a nucleotide sequence selected from the group consisting of SEQ ID NO: 1, SEQ ID NO: 2, SEQ ID NO: 3 and SEQ ID NO: 4.

According to yet another aspect, the invention provides a method for producing corn seed comprising crossing a first parent corn plant with a second parent corn plant and harvesting the resultant first generation corn seed, wherein the first or second parent corn plant is an inbred corn plant of the invention.

According to another aspect, the invention provides a method of producing hybrid corn seeds comprising the steps of: (a) planting seeds of a first inbred corn line according to the invention and seeds of a second inbred corn line having a different genotype; (b) cultivating corn plants resulting from said planting until time of flowering; (c) emasculating flowers of corn plants of one of the corn inbred lines; (d) allowing pollination of the other inbred line to occur, and (e) harvesting the hybrid seed produced thereby.

According to another aspect of the invention, the invention provides a method of selecting corn plants and seeds comprising the nucleic acid molecules of elite event MZIR098 on chromosome 10. In one embodiment of the invention, polymorphic markers are used to select or track the sequences specific to the elite event MZIR098. The invention provides a method of selecting sequences specific to the elite event MZIR098 comprising the steps of: (a) detecting a polymorphic marker sequence; (b) designing an assay for the purposes of detecting the marker; (c) running the assay on corn nucleic acid sequences from many corn lines, and (d) selecting corn lines based upon the sequences with nucleotides specific to elite event MZIR098.

According to another aspect of the invention, the invention provides a method of determining the zygosity of a corn plant comprising elite event MZIR098 comprising (a) obtaining a DNA sample of genomic DNA from said corn plant; (b) producing a contacted sample by contacting said DNA sample with (i) a first event primer and a second event primer, wherein said first event primer specifically binds said transgene construct, said second event primer specifically binds said 5′ corn genomic flanking DNA or said 3′ com genomic flanking DNA, and wherein said first event primer and said second event primer produce an event amplicon which is unique to event MZIR098, when subjected to quantitative PCR conditions, (ii) at least one native insertion site first primer and at least one native insertion site second primer, wherein the first primer is a forward primer and the second primer is a reverse primer, wherein a first primer and second primer function together to produce an amplicon from the native MZIR098 insertion site when elite event MZIR098 is not present in the genome, when subjected to quantitative PCR conditions, (iii) a fluorescent event probe that hybridizes with said event amplicon, (iv) a fluorescent native insertion site probe that hybridizes with said native insertion site amplicon; (c) subjecting said contacted sample to fluorescence-based endpoint quantitative PCR conditions; (d) quantitating said fluorescent event probe that hybridized to said event amplicon and quantitating said fluorescent native insertion site probe that hybridized to said native insertion site amplicon; (e) comparing amounts of hybridized fluorescent event probe to hybridized fluorescent native insertion site probe; and (f) determining zygosity of said corn plant comprising corn elite event MZIR098 by comparing fluorescence ratios of hybridized fluorescent event probe and hybridized fluorescent native insertion site probe. The event primer set and probe and native insertion site primer set and probe may be mixed with the same DNA sample, or they may be separate with different DNA samples derived from the same corn plant. There may be more than one forward native insertion site primer and/or more than one reverse native insertion site primer. The quantification of the fluorescence from the event probe and the fluorescence from the native insertion site probe may be sequentially or simultaneously. Zygosity determination may be made using data analysis software, such as SDS software on the ABI 7900HT, as described in Example 9 and shown in FIG. 2. The results indicate if the corn plant is homozygous for elite event MZIR098 (ie, has positive results for the event endpoint quantitative PCR but not for the native insertion site endpoint quantitative PCR), is heterozygous for elite event MZIR098 (ie, has a positive result for both the event and for the native insertion site endpoint quantitative PCRs) or is wild type (ie, has positive results for the native insertion site endpoint quantitative PCR but not for the event endpoint quantitative PCR).

According to another aspect of the invention, the invention provides a site on chromosome 10 for targeted integration of a heterologous nucleic acid. The invention provides a method of selecting sequences specific to the elite event MZIR098 for targeted integration comprising the steps of: (a) designing homologous sequences based on the insertion site or vector sequence; (b) using these homologous sequences at a target locus; (c) using a targeted endonuclease, such as a zinc finger nuclease, a meganuclease, a TALEN, or a Cas9 nuclease, to create a break in the target locus, and (d) inserting a heterologous donor molecule within nucleotides specific to elite event MZIR098. An example of this technique is demonstrated in Shukla et al. (Nature, 2009, 459: 437-441, herein incorporated by reference).

### DESCRIPTION OF THE SEQUENCES IN THE SEQUENCE LISTING

SEQ ID NO: 1 is a nucleic acid sequence of the 5′ genome-insert junction, unique to event MZIR098. SEQ ID NO: 2 is a nucleic acid sequence of the 3′ genome-insert junction, unique to event MZIR098 SEQ ID NO: 3 is a nucleic acid sequence of the 5′ genome-insert junction, sequence, plus additional 5′ flanking genomic sequence.

SEQ ID NO: 4 is a nucleic acid sequence of the 3′ genome-insert junction, sequence, plus additional 3′ flanking genomic sequence.

SEQ ID NO: 5 is a nucleic acid sequence of the transgene insertion plus the 5′ and 3′ junction sequences, unique to event MZIR098.

SEQ ID NO: 6 is a nucleic acid sequence of the transgene insertion plus the 5′ and 3′ junction sequences, unique to event MZIR098, plus additional 5′ and 3′ flanking genomic sequence.

SEQ ID NO: 7 is a nucleic acid sequence of the full-length transgene insertion of event MZIR098.

SEQ ID NO: 8 is a nucleic acid sequence of the 5′ flanking genomic sequence of the MZIR098 transgene insertion

SEQ ID NO: 9 is a nucleic acid sequence of the 3′ flanking genomic sequence of the MZIR098 transgene insertion

SEQ ID NO: 10-12 are primer and probe sequences useful for detection of the MZIR098 event

SEQ ID NO: 13 is a nucleic acid sequence of an amplicon produced using primers represented by SEQ ID NO: 10-11.

SEQ ID NO: 14-52 are primer and probe sequences useful for elite event MZIR098 detection and transgene sequencing.

SEQ ID NO: 53 is a nucleic acid sequence of the eNOS-02 enhancer of the eCry3.1Ab expression cassette within the MZIR098 transgene.

SEQ ID NO: 54 is a nucleic acid sequence of the prCMP-04 promoter of the eCry3.1Ab expression cassette within the MZIR098 transgene.

SEQ ID NO: 55 is a nucleic acid sequence of the eCry3.1Ab coding sequence, which encodes for an engineered insecticidal protein, of the eCry3.1Ab expression cassette within the MZIR098 transgene.

SEQ ID NO: 56 is a nucleic acid sequence of the tNOS-05-01 terminator of the eCry3.1Ab expression cassette within the MZIR098 transgene.

SEQ ID NO: 57 is a nucleic acid sequence of the prUbi1-18 promoter of the mCry3A expression cassette within the MZIR098 transgene.

SEQ ID NO: 58 is a nucleic acid sequence of mCry3A coding sequence, which encodes for an engineered insecticidal protein, of the mCry3A expression cassette within the MZIR098 transgene.

SEQ ID NO: 59 is a nucleic acid sequence of the tNOS-20 terminator of the mCry3A expression cassette within the MZIR098 transgene.

SEQ ID NO: 60 is a nucleic acid sequence of the pr35S-04-01 promoter of the PAT expression cassette within the MZIR098 transgene.

SEQ ID NO: 61 is a nucleic acid sequence of the cPAT-08 coding sequence, which encodes for a PAT protein that confers tolerance to GS inhibitor herbicides, of the PAT expression cassette within the MZIR098 transgene.

SEQ ID NO: 62 is a nucleic acid sequence of the eCry3.1Ab expression cassette within the MZIR098 transgene.

SEQ ID NO: 63 is a nucleic acid sequence of the mCry3A expression cassette within the MZIR098 transgene.

SEQ ID NO: 64 is a nucleic acid sequence of the PAT expression cassette within the MZIR098 transgene.

SEQ ID NO: 65-69 are primer and probe sequences useful for the detection of the MZIR098 insertion site when MZIR098 is not present, also referred to as the native insertion site, in endpoint zygosity assays.

SEQ ID NO: 70 is AC204437.3 Chromosome 10 sequence, where N is any base “A”, “T”, “G” or “C”. This sequence includes the chromosomal location of the MZIR098 insertion site.

SEQ ID NO: 71 is the reverse complement of SEQ ID NO: 70, AC204437.3 Chromosome 10 sequence, where N is any base “A”, “T”, “G” or “C”.

### BRIEF DESCRIPTION OF DRAWINGS

FIG. 1 (FIG. 1) is a graphical map illustrating the organization of the elements comprising the heterologous nucleic acid sequences inserted into the genome of corn to create elite event MZIR098 and sets forth the relative positions at which the inserted nucleic acid sequences are linked to corn genomic DNA sequences which flank the ends of the inserted heterologous DNA sequences, and also the positions of the junction sequences which comprise nucleic acid sequence of both the transgene and the flanking genomic sequence. Numbers indicate the graphical representation of sequences according to their Sequence Identification Number (SEQ ID NO). Therefore, “1” is a graphical representation of SEQ ID NO: 1, “2” represents SEQ ID NO: 2, “3” represents SEQ ID NO: 3, “4” represents SEQ ID NO: 4, “5” represents SEQ ID NO: 5, “6” represents SEQ ID NO: 6, “7” represents SEQ ID NO: 7, “8” represents SEQ ID NO: 8, “9” represents SEQ ID NO: 9, and numbers “53” through “64” represent SEQ ID NO: 53 through SEQ ID NO: 64, respectively. The gray regions for “8” and “9” indicate the flanking genomic sequence. The white regions for “53”-“61” indicate the transgene insertion.

FIG. 2 (FIG. 2) is a plot of the intensities of the signals from FAM (Y axis) which is the signal from the Event Specific Allele (ESA), which is the transgene insertion, and TET (X axis) which is the signal from the native insertion allele (WT allele; genomic location of transgene, but no transgene present) assay. The points or dots are from the final read of each sample of about 90 samples, after 40 cycles of PCR. Clusters in the upper left quadrant are from homozygous plants (HOM) because no TET (WT allele) signal is present. Clusters in the lower right are from null plants (NULL) because no FAM (ESA) signal is present. The heterozygotes (HET) are located in between in the upper right, because they have signal from both assays. The quadrant position and the gaps in between the groupings, or clusters, that allow for zygosity calls to be made based on their position. FIG. 2 is representative of data produced in Example 9.

### DETAILED DESCRIPTION

The foregoing and other aspects of the invention will become more apparent from the following detailed description.

The following definitions and methods are provided to better define the invention and to guide those of ordinary skill in the art in the practice of the invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “biological sample” is a plant, plant material or products comprising plant material. The term “plant” is intended to encompass corn (Zea mays) plant tissues, at any stage of maturity, as well as cells, tissues, organs taken from or derived from any such plant, including without limitation, any seeds, leaves, stems, flowers, roots, single cells, gametes, cell cultures, tissue cultures or protoplasts. “Plant material”, as used herein refers to material which is obtained or derived from a plant. Products comprising plant material relate to food, feed or other products which are produced using plant material or can be contaminated by plant material. A biological sample may be crushed, non-viable material. A biological sample may be derived from a commodity product, such as a corn commodity product. Corn, also known as maize, is used as human food, livestock feed, and as raw material in industry. The food uses of maize, in addition to human consumption of maize kernels, include both products of dry- and wet-milling industries. The principal products of maize dry milling are grits, meal and flour. The maize wet-milling industry can provide maize starch, maize syrups and dextrose for food use. Maize oil is recovered from maize germ, which is a by-product of both dry- and wet-milling industries.

A corn commodity product is typically derived from the grain, from the ear of the corn. Corn commodity products may also be derived from non-grain parts of the corn plant. A number of different industrial processes can be employed to extract or utilize these plant products, as are well known in the art. Corn commodity products include corn flour, corn meal, corn syrup, corn oil, corn starch, and cereals manufactured in whole or in part to contain corn by-products. Corn commodity products may be crushed, non-viable material derived from corn seeds but which are no longer capable of germination. Corn, including both grain and non-grain portions of the plant, is also used extensively as livestock feed, primarily for beef cattle, dairy cattle, hogs, and poultry. Industrial uses of maize, include production of ethanol, maize starch in the wet-milling industry and maize flour in the dry-milling industry. The industrial applications of maize starch and flour are based on functional properties, such as viscosity, film formation, adhesive properties and ability to suspend particles. The maize starch and flour have application in the paper and textile industries. Other industrial uses include applications in adhesives, building materials, foundry binders, laundry starches, explosives, oil-well muds, and other mining applications. Plant parts other than the grain of maize are also used in industry: for example, stalks and husks are made into paper and wallboard and cobs are used for fuel and to make charcoal.

A biological extract or “extract” may be derived from a biological sample or from a corn commodity product. A biological extract is from crushed biological material, and is no longer viable or capable of germination. It is understood that, in the context of the invention, such biological samples or extracts are tested for the presence of nucleic acids specific to corn elite event MZIR098, implying the presence of nucleic acids in the samples. Thus, the methods referred to herein for identifying elite event MZIR098 in biological samples or extracts relate to the identification in biological samples or extracts of nucleic acids which are from an elite event MZIR098 corn plant and are diagnostic for elite event MZIR098.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

“Detection kit” as used herein refers to a kit used to detect the presence or absence of DNA from corn elite event MZIR098 plants in a sample comprising nucleic acid probes and primers of the invention, which hybridize specifically under high stringency conditions to a target DNA sequence, and other materials necessary to enable nucleic acid hybridization or amplification methods.

“Expression cassette” as used herein means a nucleic acid molecule capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

“Genotype” as used herein is the genetic material inherited from parent corn plants not all of which is necessarily expressed in the descendant corn plants. The MZIR098 genotype refers to the heterologous genetic material transformed into the genome of a plant as well as the genetic material flanking the inserted sequence.

“MZIR098-specific” refers to a nucleotide sequence which is suitable for discriminatively identifying elite event MZIR098 in plants, plant material, or in products such as, but not limited to, food or feed products (fresh or processed) comprising or derived from plant material.

“Insert DNA” or “insert sequence” refers to the heterologous DNA within the expression cassettes used to transform the plant material. Insert DNA is derived from a T-DNA, which is contained within a binary vector used in Agrobacterium-mediated transformation of a plant. “Flanking DNA” or “flanking sequence” can exist of either genomic DNA naturally present in an organism such as a plant, or foreign (heterologous) DNA introduced via the transformation process which is extraneous to the original insert DNA molecule, e.g. fragments associated with the transformation event. Flanking sequence as used herein refers to a sequence of at least 10 bp, at least 20 bp, at least 50 bp, at least 100 bp, at least 150 bp, at least 200 bp, at least 250 bp, at least 300 bp, at least 400 bp, at least 500 bp, at least 1000 bp, at least 2000 bp, at least 3000 bp, at least 4000 bp, and at least 5000 bp, which is located either immediately upstream of and contiguous with or immediately downstream of and contiguous with the original foreign insert DNA molecule. Transformation procedures leading to random integration of the foreign DNA will result in transformants containing different flanking regions characteristic of and likely unique to each transformant. When recombinant DNA is introduced into a plant through traditional crossing, its flanking sequences will generally not be changed. Transformants will also contain unique junctions between a piece of heterologous insert DNA and genomic DNA, or two pieces of genomic DNA, or two pieces of heterologous DNA. A “junction” is a point where two specific DNA fragments join. For example, a junction exists where insert DNA joins flanking DNA. A junction point also exists in a transformed organism where two DNA fragments join together in a manner that is modified from that found in the native organism. “Junction DNA” or “junction sequence” refers to DNA that comprises a junction point. Two junction sequences set forth in this disclosure are the junction point between the maize genomic DNA and the 5′ end of the insert as set forth in SEQ ID NO: 1, and the junction point between the 3′ end of the insert and maize genomic DNA as set forth in SEQ ID NO: 2. An event may be defined by its junction sequences. These junction sequences can be transmitted to progeny and introgressed into other germplasms via traditional crossing.

The term “event” refers to the original transformant that includes the heterologous DNA and/or progeny of said event. More generally, the term “event” refers to an artificial genetic locus or genotype that, as a result of genetic engineering such as transformation, carries a foreign inserted DNA or transgene comprising at least one copy of at least one gene of interest and also comprises flanking genomic sequence immediately adjacent to the inserted DNA that would be expected to be transferred to a progeny that receives the inserted DNA, including the transgene of interest, as a result of a sexual cross of one parental line which comprises the inserted DNA (e.g., the original transformant and progeny resulting from selfing) and a parental line that does not contain the inserted DNA. The event comprises the junction sequences and the foreign inserted DNA, which may be referred to as the transgene or the T-DNA insertion. The presence of an event in a cell may be identified genotypically by its junction sequences. At the genetic level, an event is part of the genetic makeup of a plant.

The term, for example, “event MZIR098 plant”, “MZIR098 plant”, “elite event MZIR098 plant”, or “event MZIR098 corn” refers to a corn plant that comprises the MZIR098 event. An event MZIR098 plant may refer to progeny of the original transformant. The term “event MZIR098 plant” also refers to progeny produced by a sexual outcross between an event MZIR098 plant and another corn line. Even after repeated backcrossing to a recurrent parent, the inserted DNA, genomic flanking DNA, and junction sequences from the originally transformed plant are present in the progeny of the cross at the same chromosomal location. Similarly, an “MZIR098 seed” refers to a seed which comprises the MZIR098 event.

An “elite event” comprises all of the desirable characteristics of an event required for commercial utility. An elite event comprises one and only one complete copy of the transgene, with an absence of vector backbone sequence. The transgene is inserted at a desirable location in the genome, which, among other characteristics, allows easy introgression into desired commercial genetic backgrounds. The genomic location of the transgene of an elite event also allows for proper expression of the traits comprising the transgene. The expression of the traits of the transgene in an elite event is correct, appropriate, and stable spatially and temporally, both in heterozygous (or hemizygous) and homozygous conditions; is at a commercially acceptable level for a range of environmental conditions in which the plants carrying the event are likely to be exposed in normal agronomic use; and is stable through multiple generations of progeny. The transgene of the elite event also shows normal Mendelian segregation. An elite event has desirable agronomic characteristics, such as yield, vigor, fertility, and the like, which are not negatively impacted by the presence of the event in the genome of the plant. An elite event has a superior combination of efficacy, including herbicide tolerance and agronomic performance in broad genotype backgrounds and across multiple environmental locations. The status of an event as an elite event is confirmed by introgression of the elite event in different relevant genetic backgrounds and observing compliance with the criteria described above. An “elite event” may refer to a genetic locus comprising a foreign DNA as a transgene, which meets the above-described criteria. A plant, plant material or progeny such as seeds can comprise one or more elite events in its genome. The likelihood of having all of these characteristics in an event is small, such that an elite event is non-obvious and atypical of events recovered from the transformation process. An elite event may only be found by an extensive selection procedure.

Plants harboring elite event MZIR098 are characterized by their insect resistance, particularly to Diabrotica spp, as well as by their tolerance to GS inhibitors such as glufosinate or bialaphos. Corn plants comprising elite event MZIR098 are useful in controlling coleopteran insect pests including Diabrotica virgifera virgifera, the western corn rootworm, D. virgifera zeae, the Mexican corn rootworm, and D. longicornis barberi, the northern corn rootworm. Plants harboring elite event MZIR098 are also characterized by having agronomical characteristics that are comparable to commercially available varieties of corn, in the absence of herbicide application or insect pest pressure. Thus, plants comprising elite event MZIR098 can tolerate the application of GS inhibitor herbicides without negatively affecting the yield of said plants compared to isogenic lines lacking event MZIR098. Additionally, corn plants comprising elite event MZIR098 have no statistically significant difference in their disease susceptibility, or lodging compared to isogenic corn plants without the MZIR098 event. These characteristics make the elite event MZIR098 very useful for control Diabrotica spp. insect pests of corn, particularly western corn rootworm, as well as control of glyphosate-resistant weeds in corn fields. Because mCry3A and eCry3.1Ab provide two modes of action, elite event MZIR098 also can be used in IRM approaches to prevent or delay development of resistance to mCry3A or eCry3.1Ab in insect pests.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

The term “isolated” when used in relation to a nucleic acid refers to a nucleic acid sequence that is identified and separated from at least one contaminant nucleic acid with which it is ordinarily associated in its natural source. An isolated nucleic acid is present in a form or setting that is different from that in which it is found in nature. In contrast, a non-isolated nucleic acids such as DNA and RNA found in the state they exist in nature. An isolated nucleic acid may be in a transgenic plant or biological sample and still be considered “isolated”.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

The tools developed to identify an elite event or the plant or plant material comprising an elite event, or products which comprise plant material comprising the elite event, are based on the specific genomic characteristics of the elite event, such as, a specific restriction map of the genomic region comprising the inserted DNA, molecular markers, or the sequence of the flanking region(s) of the inserted DNA.

Once one or both of the flanking regions of the inserted DNA, or transgene, have been sequenced, primers and probes can be developed which specifically recognize this (these) sequence(s) in the nucleic acid (DNA or RNA) of a sample by way of a molecular biological technique. For instance, a PCR method can be developed to identify the elite event in biological samples (such as samples of plants, plant material or products comprising plant material). Such a PCR is based on at least two specific “primers”, one recognizing a sequence within the 5′ or 3′ flanking region of the elite event and the other recognizing a sequence within the foreign DNA. The primers preferably have a sequence of between 15 and 35 nucleotides which under optimized PCR conditions “specifically recognize” a sequence within the 5′ or 3′ flanking region of the elite event and the foreign DNA of the elite event respectively, so that a specific fragment (“integration fragment” or discriminating amplicon) is amplified from a nucleic acid sample comprising the elite event. This means that only the targeted integration fragment, and no other sequence in the plant genome or foreign DNA, is amplified under optimized PCR conditions.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. A primer pair comprises a “forward” primer and a “reverse” primer. Primer pairs or sets can be used for amplification of a nucleic acid molecule, for example, by the polymerase chain reaction (PCR) or other conventional nucleic-acid amplification methods.

PCR primers suitable for identification of elite corn event MZIR098 may be the following:
a) oligonucleotides ranging in length from 17 bp to about 200 bp, comprising a nucleotide sequence of at least 17 consecutive nucleotides, preferably 20 consecutive nucleotides, selected from DNA in the 5′ flanking sequence (SEQ ID NO: 8), such that the primer recognizes the 5′ flanking sequence; or
b) oligonucleotides ranging in length from 17 bp to about 200 bp, comprising a nucleotide sequence of at least 17 consecutive nucleotides, preferably 20 consecutive nucleotides, selected from DNA in the 3′ flanking sequence (SEQ ID NO: 9), such that the primer recognizes the 3′ flanking sequnce; or
c) oligonucleotides ranging in length from 17 bp to about 200 bp, comprising a nucleotide sequence of at least 17 consecutive nucleotides, preferably 20 consecutive nucleotides, selected from the DNA in the transgene (SEQ ID NO: 7), such that the primer recognizes the transgene.

The primers may of course be longer than the mentioned 17 consecutive nucleotides, and may, e.g., be 20, 21, 22, 23, 24, 25, 30, 35, 50, 75, 100, 150, 200 bp long or even longer. The primers may entirely consist of nucleotide sequence selected from the mentioned nucleotide sequences of flanking sequences and transgene DNA sequences. However, the nucleotide sequence of the primers at their 5′ end (i.e. outside of the 3′-located 17 consecutive nucleotides) is less critical. Thus, the 5′ sequence of the primers may comprise or consist of a nucleotide sequence selected from the flanking sequences or foreign DNA, as appropriate, but may contain several (e.g., 1, 2, 5, or 10) mismatches. The 5′ sequence of the primers may even entirely be a nucleotide sequence unrelated to the flanking sequences or foreign DNA, such as, e.g., a nucleotide sequence representing one or more restriction enzyme recognition sites. Such unrelated sequences or flanking DNA sequences with mismatches should preferably be not longer than 100, more preferably not longer than 50 or 25 nucleotides.

Moreover, suitable primers may comprise, consist or consist essentially of a nucleotide sequence spanning the junction region between the plant DNA derived sequences and the inserted DNA sequences (SEQ ID NO: 1 and 2). It will also be immediately clear to the skilled artisan that properly selected PCR primer pairs should also not comprise sequences complementary to each other.

Examples of suitable primers for detection or identification of elite event MZIR098 include SEQ ID NOs: 10, 11, 14 through 52, and complements thereof. Other examples of suitable oligonucleotide primers for the detection or identification of elite event MZIR098 comprise at least 10 contiguous nucleotides of SEQ ID NO: 7, 8, and 9, and complements thereof. A person of ordinary skill in the art will appreciate that for a primer set to be diagnostic for corn elite event MZIR098, the resulting amplicon must comprise SEQ ID NO: 1 or SEQ ID NO: 2.

A “probe” is an isolated nucleic acid to which is attached a conventional detectable label or reporter molecule, such as a radioactive isotope, ligand, chemiluminescent agent, or enzyme. Such a probe is complimentary to a strand of a target nucleic acid, in the case of the invention, to a strand of genomic DNA from corn elite event, MZIR098. The genomic DNA of elite event MZIR098 can be from a corn plant or from a sample that includes DNA from the event. Probes according to the invention include not only deoxyribonucleic or ribonucleic acids but also polyamides and other probe materials that bind specifically to a target DNA sequence and can be used to detect the presence of that target DNA sequence.

Probes and primers are generally 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 polynucleotides or more in length. Such primers and probes hybridize specifically to a target sequence under high stringency hybridization conditions. Primers and probes according to the invention may have complete sequence complementarity with the target sequence, although probes differing from the target sequence and which retain the ability to hybridize to target sequences may be designed by conventional methods.

“Stringent conditions” or “stringent hybridization conditions” include reference to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or wash conditions, target sequences can be identified which are 100% complementary to the probe (homologous probing). Alternatively, stringency conditions can be adjusted to allow some mismatching in sequences so that lower degrees of similarity are detected (heterologous probing). Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, Part I, Chapter 2 “Overview of principles of hybridization and the strategy of nucleic acid probe assays”, Elsevier: New York; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5th Ed. Cols Spring Harbor Laboratory, Cold Spring Harbor, NY).

Specificity is typically the function of post-hybridization washes, the critical factors being the ionic strength and temperature of the final wash solution. Generally, high stringency hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, under high stringency conditions a probe will hybridize to its target subsequence, but to no other sequences.

An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook, infra, for a description of SSC buffer).

Exemplary hybridization conditions for the invention include hybridization in 7% SDS, 0.25 M NaPO₄ pH 7.2 at 67° C. overnight, followed by two washings in 5% SDS, 0.20 M NaPO₄ pH7.2 at 65° C. for 30 minutes each wash, and two washings in 1% SDS, 0.20 M NaPO₄pH7.2 at 65° C. for 30 minutes each wash. An exemplary medium stringency wash for a duplex of, e.g., more than 100 nucleotides, is 1×SSC at 45° C. for 15 minutes. An exemplary low stringency wash for a duplex of, e.g., more than 100 nucleotides, is 4-6×SSC at 40° C. for 15 minutes.

For probes of about 10 to 50 nucleotides, high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, e.g., when a copy of a nucleic acid is created using the maximum codon degeneracy permitted by the genetic code.

The following are exemplary sets of hybridization/wash conditions that may be used to hybridize nucleotide sequences that are substantially identical to reference nucleotide sequences of the invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. The sequences of the invention may be detected using all the above conditions. For the purposes of defining the invention, the high stringency conditions are used.

As used herein, “amplified DNA” or “amplicon” refers to the product of nucleic acid amplification of a target nucleic acid sequence that is part of a nucleic acid template. For example, to determine whether a com plant resulting from a sexual cross contains transgenic event genomic DNA from the corn plant of the invention, DNA extracted from the com plant tissue sample may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol, and/or the combined length of the primer pairs plus about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500, 750, 1000, 1250, 1500, 1750, 2000, or more polynucleotides, plus or minus any of the increments listed above. Alternatively, primer pairs can be derived from flanking sequence on both sides of the inserted DNA so as to produce an amplicon that includes the entire insert nucleotide sequence of the transgene as well as the sequence flanking the transgenic insert. A member of a primer pair derived from the flanking sequence may be located a distance from the inserted DNA sequence, this distance can range from one nucleotide base pair up to the limits of the amplification reaction, or about 20,000 bp. The use of the term “amplicon” specifically excludes primer dimers.

TaqMan (ThermoFisher Scientific, Waltham, MA, USA.) is described as a method of detecting and quantifying the presence of a DNA sequence and is fully understood in the instructions provided by the manufacturer. Briefly, a FRET oligonucleotide probe is designed which overlaps the flanking and insert DNA junction. The FRET probe and PCR primers (one primer in the insert DNA sequence and one in the flanking genomic sequence) are cycled in the presence of a thermostable polymerase and dNTPs. Flybridization of the FRET probe results in cleavage and release of the fluorescent moiety away from the quenching moiety on the FRET probe. A fluorescent signal indicates the presence of the flanking/transgene insert sequence due to successful amplification and hybridization. This fluorescent signal can be quantified.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule. As used herein, “transgenic” refers to a plant, plant cell, or multitude of structured or unstructured plant cells having integrated, via well known techniques of genetic manipulation and gene insertion, a nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, Agrobacterium-mediated transformation, and ballistic transformation.

The nomenclature for DNA bases and amino acids as set forth in 37 C.F.R. § 1.822 is used herein.

This invention relates to a genetically improved line of corn that provides dual modes of action for control of corn rootworm (Diabrotica spp.) and tolerates herbicides containing a GS inhibitor, such as glufosinate-ammonium. The dual modes of action are provided by the presence of two insecticidal trait genes, namely mCry3A and eCry3.1Ab. Herbicide tolerance is provided by the presence of the herbicide tolerance trait gene PAT, which encodes a phosphinothricin acetyltransferase and is derived from Streptomyces viridochromogenes. The invention is particularly drawn to an elite transgenic corn event designated elite event MZIR098 comprising a novel genotype, as well as to compositions and methods for detecting nucleic acids from this event in a biological sample. The invention is further drawn to corn plants comprising the elite event MZIR098 genotype, to transgenic seed from the corn plants, and to methods for producing a corn plant comprising the elite event MZIR098 genotype by crossing a corn inbred comprising the elite event MZIR098 genotype with itself or another corn line. Corn plants comprising the elite event MZIR098 genotype of the invention are useful as part of insect pest management and as part of a weed control program because they possess herbicide tolerance to GS inhibitors.

The present invention embodies a novel genotype of corn, which is a result of the random insertion of a transgene in the genome of a corn plant by Agrobacterium-mediates transformation. It is recognized in the art that the genomic location of such an insertion cannot be predicted. The present invention encompasses the particular novel genotype and novel sequences, namely SEQ ID NO: 1, SEQ ID NO: 2, SEQ ID NO: 3, SEQ ID NO: 4, SEQ ID NO: 5, or SEQ ID NO: 6, and complements thereof, which are novel and diagnostic for elite event MZIR098. Furthermore, the present invention discloses a corn plant comprising elite event MZIR098, an example of which is deposited as ATCC Accession No: PTA-124143. A corn plant comprising elite event MZIR098 contains within its genome SEQ ID NO: 1, SEQ ID NO: 2, and SEQ ID NO: 5. Further, the present invention encompasses specific tools in the form of specific polynucleotide molecules that are capable of identifying the transgene insertion at its specific insertion site in the corn genome.

The present invention comprises a molecular stack comprising expression cassettes with mCry3A, eCry3.1Ab, and PAT coding sequences. The present invention possesses many advantages over a breeding stack which may comprise the same three traits. A plant comprising a breeding stack would comprise the three traits present as two or possibly single events, such that the genome of the plant has two or possibly three separate transgene insertions. The creation of such a plant technically can be complicated, as two parents each containing at least one copy of an event would need to be crossed, and the progeny would need to be screened for the presence of each trait. Additionally, because each event is present at a different genomic location, the resultant plant would need to be evaluated for agronomic and trait performance, to ensure that the presence of two or possibly three insertions in the genome had no deleterious effects. Additionally, the progeny of a plant comprising a breeding stack would need to be screened for the presence of each trait. Finally, such a procedure would need to be performed for every corn variety, or germplasm, into which the breeding stack was desired. The present invention solves these problems by having all traits as a molecular stack, so that they are singly inserted into the genome together. The molecular stack also provides greater stability in the expression of the traits in multiple germplasms, as the traits are inserted together at a single genomic locus and therefore have the same position effects regardless of the germplasm into which they have been introduced.

In one embodiment, the invention encompasses a transgenic corn seed of an elite event MZIR098 corn plant. An example of said seed being deposited as ATCC Accession No: PTA-124143. The transgenic seed, a transgenic plant, transgenic cell, and transgenic tissue of elite event MZIR098 comprises a nucleic acid molecule with at least 90%, 91%, 92%, 93%, 94%, 95%, 96%, 97%, 98%, 99%, or 100% identity to SEQ ID NO: 1, SEQ ID NO: 2, SEQ ID NO: 3, SEQ ID NO: 4, SEQ ID NO: 5, or SEQ ID NO: 6, and complements thereof. These sequences define a point of insertion of a heterologous DNA sequence inserted into the corn plant genome of corn elite event MZIR098. The invention further comprises a transgenic corn plant, transgenic seed, transgenic cell, and transgenic tissue of elite event MZIR098 capable of producing an elite event MZIR098 diagnostic amplicon, wherein said diagnostic amplicon hybridizes under stringent conditions to SEQ ID NO: 1, SEQ ID NO: 2, SEQ ID NO: 3, or SEQ ID NO: 4. The invention further comprises a transgenic insect resistant and herbicide tolerant plant, cell, seed, tissue, or progeny thereof, comprising: a DNA construct comprising three expression cassettes, wherein said first expression cassette in operable linkage comprises: (i) a NOS enhancer represented by SEQ ID NO: 53; (ii) a CMP promoter represented by SEQ ID NO: 54; (iv) an engineered insecticidal protein eCry3.1Ab coding sequence represented by SEQ ID NO: 55; and (v) a NOS transcriptional terminator represented by SEQ ID NO: 56; and wherein said second expression cassette in operable linkage comprises (i) a maize Ubi1 promoter represented by SEQ ID NO: 57; (ii) an engineered insecticidal protein mCry3A coding sequence represented by SEQ ID NO: 58; and (iii) a transcriptional terminator represented by SEQ ID NO: 59; and wherein said third expression cassette in operable linkage comprises (i) a CaMV 35S promoter represented by SEQ ID NO: 60; (ii) a PAT coding sequence represented by SEQ ID NO: 61; and (iii) a NOS transcriptional terminator represented by SEQ ID NO: 56, wherein the sequence overlapping the junction between the corn genomic DNA and the 5′ flank of the construct comprises SEQ ID NO: 1 and the overlapping junction between the corn genomic DNA and the 3′ flank of the construct comprises SEQ ID NO: 2, and wherein the DNA construct is present in the corn elite event MZIR098 deposited with American Type Culture Collection (ATCC) Accession No. PTA-124143.

In another embodiment, the invention encompasses a preferably isolated nucleic acid molecule comprising SEQ ID NO: 1, SEQ ID NO: 2, SEQ ID NO: 3, SEQ ID NO: 4, SEQ ID NO: 5, or SEQ ID NO: 6, and complements thereof. In another embodiment, the invention encompasses a optionally isolated nucleic acid molecule, wherein the nucleic acid molecule is comprised in a corn seed deposited as ATCC Accession No. PTA-124143. In another embodiment, the invention comprises an isolated nucleic acid molecule comprising: a) SEQ ID NO: 1; b) operably linked at the 3′ end of said sequence of step (a) an expression cassette comprising SEQ ID NO: 62 (eNOS-02:prCMP-04:eCry3.1Ab:tNOS-05-01); c) operably linked at the 3′ end of said cassette of step (b) and in the same orientation, an expression cassette comprising SEQ ID NO: 63 (prUbi1-18:mCry3A:tNOS-20); d) operably linked at the 3′ end of said cassette of step (c) and in the same orientation, an expression cassette comprising SEQ ID NO: 64 (pr35S-04-01:cPAT-08:tNOS-05-01) and e) operably linked at the 3′ end of said cassette of step (d) SEQ ID NO: 2.

In one embodiment, the invention encompasses a nucleic acid molecule, optionally isolated, comprising at least 10 or more (for example 15, 20, 25, or 50) contiguous nucleotides of a heterologous DNA sequence inserted into the corn plant genome of corn elite event MZIR098 and at least 10 or more (for example 15, 20, 25, or 50) contiguous nucleotides of a corn plant genome DNA flanking the point of insertion of a heterologous DNA sequence inserted into the corn plant genome of corn elite event MZIR098. Also included are nucleotide sequences that comprise 10 or more nucleotides of contiguous insert sequence from elite event MZIR098 and at least one nucleotide of flanking DNA from elite event MZIR098 adjacent to the insert sequence. Such nucleotide sequences are diagnostic for elite event MZIR098. Nucleic acid amplification of genomic DNA from the elite event MZIR098 may produce an amplicon comprising such diagnostic nucleotide sequences (namely, SEQ ID NO: 1, SEQ ID NO: 2, SEQ ID NO: 3, SEQ ID NO: 4, SEQ ID NO: 5, SEQ ID NO: 6 and the complements thereof).

In another embodiment, the invention encompasses a nucleic acid molecule, preferably isolated, comprising a nucleotide sequence which comprises at least one junction sequence of elite event MZIR098 selected from the group consisting of SEQ ID NO: 1, SEQ ID NO: 2, SEQ ID NO: 3, SEQ ID NO: 4, and complements thereof, wherein a junction sequence spans the junction between a heterologous expression cassette inserted into the corn genome and DNA from the corn genome flanking the insertion site and is diagnostic for the event. In a further embodiment, the invention comprises a nucleic acid molecule relating to the corn elite event MZIR098, characterized in that it consists of the sequence of SEQ ID NO: 1. In another embodiment, the invention comprises a nucleic acid molecule relating to the corn elite event MZIR098, characterized in that it consists of the sequence of SEQ ID NO: 2. In another embodiment, the invention comprises the use of SEQ ID NO: 1 or SEQ ID NO: 2 to identify corn elite event MZIR098 in a plant.

In another embodiment, the invention encompasses a nucleic acid molecule linking a heterologous DNA molecule to the corn plant genome in corn elite event MZIR098 comprising a sequence of from about 11 to about 20 contiguous nucleotides of a sequence selected from the group consisting of SEQ ID NO: 1, SEQ ID NO: 2, and the complements thereof.

In another embodiment, the invention encompasses a nucleic acid molecule, preferably isolated, comprising a nucleotide sequence selected from the group consisting of SEQ ID NO: 1, SEQ ID NO: 2, SEQ ID NO: 3, SEQ ID NO: 4, and complements thereof.

In another embodiment, the invention encompasses a nucleic acid molecule, comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and the complements thereof. In a further embodiment, the invention encompasses the nucleic acid molecule described above wherein the molecule is within the genome of a transgenic organism, for example a transgenic maize plant. The invention also includes a genome comprising said nucleic acid molecule.

In another embodiment, the invention encompasses flanking sequence primers for detecting elite event MZIR098. Such flanking sequence primers comprise an isolated nucleic acid sequence comprising at least 10-15 contiguous nucleotides from SEQ ID NO: 8 (designated herein as the 5′ flanking sequence), SEQ ID NO: 9 (designated herein as the 3′ flanking sequence) or the complements thereof. In one aspect of this embodiment the flanking sequence primers are selected from the group consisting of SEQ ID NO: 10, SEQ ID NO: 14, SEQ ID NO: 15, SEQ ID NO: 16, SEQ ID NO: 38, SEQ ID NO: 42, SEQ ID NO: 65, SEQ ID NO: 19, SEQ ID NO: 20, SEQ ID NO: 21, SEQ ID NO: 36, SEQ ID NO: 41, SEQ ID NO: 45, SEQ ID NO: 67, and complements thereof. The flanking sequences can be extended to include additional chromosome 10 sequence, with specific emphasis on nucleotides comprised within SEQ ID NO: 70 or SEQ ID NO: 71, useful in detecting sequences associated with the corn elite event MZIR098. In the context of SEQ ID NO: 70 and SEQ ID NO: 71, an “N” is defined as any base “A”, “T”, “G”, or “C”. SEQ ID NO: 71 is the reverse complement of SEQ ID NO: 70.

In still another embodiment, the invention encompasses a pair of polynucleotide primers comprising a first polynucleotide primer and a second polynucleotide primer which function together in the presence of a corn elite event MZIR098 DNA template in a sample to produce an amplicon diagnostic for the corn elite event MZIR098. In some aspects of this embodiment, the first primer sequence is or is complementary to a corn plant genomic sequence flanking the point of insertion of a heterologous DNA sequence inserted into the corn plant genome of corn elite event MZIR098, and the second polynucleotide primer sequence is or is complementary to the heterologous DNA sequence inserted into the corn plant genome of the corn elite event MZIR098. Another embodiment of the invention is the use of these polynucleotide primers to identify corn elite event MZIR098 in a plant.

In one aspect of this embodiment the first polynucleotide primer comprises at least 10 contiguous nucleotides from position 1-1076 of SEQ ID NO: 8, or at least 10 contiguous nucleotides from position 1-1075 of SEQ ID NO: 9, or complements thereof. In a further aspect of this embodiment, the first polynucleotide primer comprises the nucleotide sequence set forth in SEQ ID NO: 10, SEQ ID NO: 14, SEQ ID NO: 15, SEQ ID NO: 16, SEQ ID NO: 38, SEQ ID NO: 42, SEQ ID NO: 65, or complements thereof. In yet another aspect of this embodiment, the second polynucleotide primer comprises at least 10 contiguous nucleotides of SEQ ID NO: 7, or the complements thereof. In still a further aspect of this embodiment, the second polynucleotide primer comprises the nucleotide sequence set forth in SEQ ID NO: 11, SEQ ID NO: 17, SEQ ID NO: 22 through 24, SEQ ID NO: 29, SEQ ID NO: 43, SEQ ID NO: 46 through 49, or the complements thereof.

In another aspect of this embodiment, the first polynucleotide primer comprises the nucleotide sequence of SEQ ID NO: 19, SEQ ID NO: 20, SEQ ID NO: 21, SEQ ID NO: 36, SEQ ID NO: 41, SEQ ID NO: 45, SEQ ID NO: 67, or complements thereof, and the second polynucleotide primer comprises the nucleotide sequence of SEQ ID NO: 18, SEQ ID NO: 31, SEQ ID NO: 33 through 35, SEQ ID NO: 37, SEQ ID NO: 50, or the complement thereof, such that the pair function together in the presence of a corn elite event MZIR098 DNA template in a sample to produce an amplicon diagnostic for the corn elite event MZIR098. In another aspect of this embodiment, the first polynucleotide primer comprises at least 10 contiguous nucleotides of SEQ ID NO: 7, or the complements thereof and the second polynucleotide primer comprises at least 10 contiguous nucleotides from position 1-1075 of SEQ ID NO: 9, or complements thereof, such that the pair function together in the presence of a corn elite event MZIR098 DNA template in a sample to produce an amplicon diagnostic for the corn elite event MZIR098. Another embodiment of the invention is the use of these polynucleotide primers to identify corn elite event MZIR098 in a plant.

In another aspect of this embodiment, the first polynucleotide primer comprises SEQ ID NO: 10, and the second polynucleotide primer comprises SEQ ID NO: 11, and the pair function together in the presence of a corn elite event MZIR098 DNA template in a sample to produce an amplicon diagnostic for the corn event MZIR098 which can be detected by a probe comprising SEQ ID NO: 12, as described in Example 2.

It is well within the skill in the art to obtain additional sequence further out into the genome sequence flanking either end of the inserted heterologous DNA sequences for use as a primer sequence that can be used in such primer pairs for amplifying sequences that are diagnostic for the elite event MZIR098. For the purposes ofthis disclosure, the phrase “further out into the genome sequence flanking either end of the inserted heterologous DNA sequences” refers specifically to a sequential movement away from the ends of the inserted heterologous DNA sequences, the points at which the inserted DNA sequences are adjacent to native genomic DNA sequence, and out into the genomic DNA of the particular chromosome into which the heterologous DNA sequences were inserted. Preferably, a primer sequence corresponding to or complementary to a part of the insert sequence should prime the transcriptional extension of a nascent strand of DNA or RNA toward the nearest flanking sequence junction. Consequently, a primer sequence corresponding to or complementary to a part of the genomic flanking sequence should prime the transcriptional extension of a nascent strand of DNA or RNA toward the nearest flanking sequence junction. A primer sequence can be, or can be complementary to, a heterologous DNA sequence inserted into the chromosome of the plant, or a genomic flanking sequence. One skilled in the art would readily recognize the benefit of whether a primer sequence would need to be, or would need to be complementary to, the sequence as set forth within the inserted heterologous DNA sequence or as set forth in SEQ ID NO: 1 or SEQ ID NO: 2 depending upon the nature of the product desired to be obtained through the use of the nested set of primers intended for use in amplifying a particular flanking sequence containing the junction between the genomic DNA sequence and the inserted heterologous DNA sequence. Further more, one skilled in the art would be able to design primers for a multitude of native corn genes for the purposes of designing a positive control. One such example is the corn Adhi gene, where examples of suitable primers for producing an amplicon by nucleic acid amplification are well known in the art (see, for example, U.S. Pat. No. 8,466,346, incorporated by reference herein).

In another embodiment, the invention encompasses a method of detecting the presence of a nucleic acid molecule that is unique to event MZIR098 in a sample comprising corn nucleic acids, the method comprising: a) isolating a nucleic acid molecule from corn; b) combining the nucleic acid molecule with a pair of polynucleotide primers of the invention; c) performing a nucleic acid amplification reaction which results in an amplicon diagnostic for the corn elite event MZIR098; and d) detecting the amplicon.

In another embodiment, the invention encompasses a method of confirming the absence of a nucleic acid molecule that is unique to event MZIR098 in a sample comprising corn nucleic acids, the method comprising: a) isolating genomic DNA from corn; b) combining the nucleic acid molecule with a pair of polynucleotide primers of the invention and with a pair of polynucleotide primers to a corn native gene, for example to the corn Adhi gene, as a positive control; c) performing a nucleic acid amplification reaction which results in no amplicon specific to elite event MZIR098 and results in an amplicon specific to the corn native gene positive control; and d) detecting an amplicon specific to the corn native gene positive control.

In another embodiment, the invention encompasses a method of detecting the presence of a nucleic acid molecule that is unique to event MZIR098 in a sample comprising corn nucleic acids, for example a biological sample, the method comprising: a) isolating a nucleic acid molecule from corn; b) combining the nucleic acid molecule with a pair of polynucleotide primers of the invention and with a polynucleotide probe comprising a nucleotide sequence of SEQ ID NO: 1, SEQ ID NO: 2, SEQ ID NO: 10 through 12, SEQ ID NO: 14 through SEQ ID NO: 52, or a complement thereof, c) performing a nucleic acid amplification reaction which results in an amplicon which can be detected by the probe; and d) detecting the probe. In a further embodiment, the invention encompasses a DNA molecule comprising the amplicon produced by the methods of the invention. In a preferred aspect of this embodiment, the amplicon comprises a nucleotide sequence selected from the group consisting of SEQ ID NO: 1, SEQ ID NO: 2, SEQ ID NO: 3, SEQ ID NO: 4, and complements thereof.

In another embodiment, the invention encompasses a method of detecting the presence of DNA corresponding to the corn elite event MZIR098 in a biological sample, wherein the method comprises: (a) contacting the sample comprising DNA with a probe that hybridizes under high stringency conditions with genomic DNA from corn elite event MZIR098 and does not hybridize under high stringency conditions with DNA of a control corn plant; (b) subjecting the sample and probe to high stringency hybridization conditions; and (c) detecting hybridization of the probe to the DNA. In one aspect of this embodiment the probe comprises a nucleotide sequence selected from the group consisting of SEQ ID NO: 1, SEQ ID NO: 2, SEQ ID NO: 3, SEQ ID NO: 4, and complements thereof.

In another embodiment, the invention encompasses a method of detecting the presence of a DNA corresponding to the corn elite event MZIR098 event in a biological sample, wherein the method comprises: (a) contacting the sample comprising DNA with a probe that hybridizes under high stringency conditions with genomic DNA from corn event corn elite event MZIR098 and does not hybridize under high stringency conditions with DNA of a control corn plant; (b) subjecting the sample and probe to high stringency hybridization conditions; and (c) detecting hybridization of the probe to the DNA. Detection can be by any means well known in the art including but not limited to fluorescent, chemiluminescent, radiological, immunological, or otherwise. In the case in which hybridization is intended to be used as a means for amplification of a particular sequence to produce an amplicon which is diagnostic for the corn elite event MZIR098 corn event, the production and detection by any means well known in the art of the amplicon is intended to be indicative of the intended hybridization to the target sequence where one probe or primer is utilized, or sequences where two or more probes or primers are utilized. The term “biological sample” is intended to comprise a sample that contains or is suspected of containing a nucleic acid comprising from between five and ten nucleotides either side of the point at which one or the other of the two terminal ends of the inserted heterologous DNA sequence contacts the genomic DNA sequence within the chromosome into which the heterologous DNA sequence was inserted, herein also known as the junction sequences. In addition, the junction sequence comprises as little as two nucleotides: those being the first nucleotide within the flanking genomic DNA adjacent to and covalently linked to the first nucleotide within the inserted heterologous DNA sequence.

In yet another embodiment, the invention encompasses a kit for detecting the presence of corn elite event MZIR098 nucleic acids in a biological sample, wherein the kit comprises at least one nucleic acid molecule of sufficient length of contiguous nucleotides homologous or complementary to a nucleotide sequence selected from the group consisting of SEQ ID NO: 1, SEQ ID NO: 2, SEQ ID NO: 3, and SEQ ID NO: 4, that functions as a DNA primer or probe specific for corn elite event MZIR098, and other materials necessary to enable nucleic acid hybridization or amplification. A variety of detection methods can be used including TaqMan (ThermoFisher Scientific), thermal amplification, ligase chain reaction, southern hybridization, ELISA methods, and colorimetric and fluorescent detection methods. In particular the invention provides for kits for detecting the presence of the target sequence, i.e., at least one of the junctions of the insert DNA with the genomic DNA of the corn plant in corn elite event MZIR098, in a sample containing genomic nucleic acid from event corn elite event MZIR098. The kit is comprised of at least one polynucleotide capable of binding to the target site or substantially adjacent to the target site and at least one means for detecting the binding of the polynucleotide to the target site. The detecting means can be fluorescent, chemiluminescent, colorimetric, or isotopic and can be coupled at least with immunological methods for detecting the binding. A kit is also envisioned which can detect the presence of the target site in a sample, i.e., at least one of the junctions of the insert DNA with the genomic DNA of the corn plant in corn elite event MZIR098, taking advantage of two or more polynucleotide sequences which together are capable of binding to nucleotide sequences adjacent to or within about 100 base pairs, or within about 200 base pairs, or within about 500 base pairs or within about 1000 base pairs of the target sequence and which can be extended toward each other to form an amplicon which contains at least the target site

In another embodiment, the invention encompasses a method for detecting corn elite event MZIR098 protein in a biological sample, the method comprising: (a) extracting protein from a sample of corn elite event MZIR098 tissue; (b) assaying the extracted protein using an immunological method comprising antibody specific for the mCry3A, eCry3.1Ab, and/or PAT protein produced by the corn elite event MZIR098 event; and (c) detecting the binding of said antibody to the mCry3A, eCry3.1Ab, and/or PAT protein.

Another embodiment of the invention encompasses a corn plant, or parts thereof, comprising the genotype of the transgenic corn elite event MZIR098, wherein the genotype comprises the nucleotide sequence set forth in SEQ ID NO: 1, SEQ ID NO: 2, SEQ ID NO: 3, SEQ ID NO: 4, SEQ ID NO: 5, SEQ ID NO: 7, or the complements thereof. Said genotype is within the genome of a corn plant. In one aspect of this embodiment, the corn plant is from the inbred corn lines CG5NA58, CG5NA58A, CG3ND97, CG5NA01, CG5NF22, CG4NU15, CG00685, CG00526, CG00716, NP904, NP948, NP934, NP982, NP991, NP993, NP2010, NP2013, NP2015, NP2017, NP2029, NP2031, NP2034, NP2045, NP2052, NP2138, NP2151, NP2166, NP2161, NP2171, NP2174, NP2208, NP2213, NP2222, NP2275, NP2276, NP2316, BCTT609, AF031, H8431, 894, BUTT201, R327H, 2044BT, and 2070BT. One skilled in the art will recognize however, that the corn elite event MZIR098 genotype can be introgressed into any plant variety that can be bred with corn, including wild maize species, and thus the preferred inbred lines of this embodiment are not meant to be limiting.

In another embodiment, the invention encompasses a corn plant comprising at least a first and a second DNA sequence linked together to form a contiguous nucleotide sequence, wherein the first DNA sequence is within a junction sequence and comprises at least about 10-15 contiguous nucleotides selected from the group consisting of nucleotides SEQ ID NO: 1, SEQ ID NO: 2, SEQ ID NO: 3, SEQ ID NO: 4, SEQ ID NO: 10, and complements thereof, wherein the second DNA sequence is within the heterologous insert DNA sequence selected from the group consisting of SEQ ID NO: 11, SEQ ID NO: 17, SEQ ID NO: 18, SEQ ID NO: 22 through SEQ ID NO: 35, SEQ ID NO: 37, SEQ ID NO: 39, SEQ ID NO: 40, SEQ ID NO: 43, SEQ ID NO: 44, SEQ ID NO: 46 through SEQ ID NO: 52, and complements thereof. In another embodiment, the first sequence is a genomic flanking sequence upstream of the junction sequence, for example SEQ ID NOs: 10, 14, 15, 16, 19, 20, 21, 38, 41, 42, 45, 65, 67, or the complements thereof, and the second DNA sequence is within the heterologous insert DNA sequence selected from the group consisting of SEQ ID NO: 11, SEQ ID NO: 17, SEQ ID NO: 18, SEQ ID NO: 22 through SEQ ID NO: 35, SEQ ID NO: 37, SEQ ID NO: 39, SEQ ID NO: 40, SEQ ID NO: 43, SEQ ID NO: 44, SEQ ID NO: 46 through SEQ ID NO: 52, and complements thereof, and wherein the first and the second DNA sequences are useful as nucleotide primers or probes for detecting the presence of corn elite event MZIR098 nucleic acid sequences in a biological sample. In one aspect of this embodiment, a pair of nucleotide primers, one of which comprises the first nucleic acid sequences described above, and the other which comprises the second nucleic acid sequences described above, are used in a DNA amplification method to amplify a target DNA sequence from template DNA extracted from the corn plant and the corn plant is identifiable from other corn plants by the production of an amplicon corresponding to a DNA sequence comprising SEQ ID NO: 1 or SEQ ID NO: 2.

In one embodiment, the invention provides a corn plant, wherein the event corn elite event MZIR098 genotype confers upon the corn plant insect resistance, particular to the Diabrotica spp. insect pests, and tolerance to GS inhibitor herbicides. In one aspect of this embodiment, the genotype conferring insect resistance upon the corn plant comprises a mCry3A gene or an eCry3.1Ab gene. In another aspect of this embodiment, the genotype conferring upon the corn plant tolerance to GS inhibitor herbicides comprises aPAT gene.

In one embodiment, the invention provides a biological sample derived from an elite event MZIR098 corn plant, tissue, or seed, wherein the sample comprises a nucleotide sequence which is or is complementary to a sequence selected from the group consisting of SEQ ID NO: 1, SEQ ID NO: 2, SEQ ID NO: 3, SEQ ID NO: 4, SEQ ID NO: 5 and SEQ ID NO: 6, and wherein the sequence is detectable in the sample using a nucleic acid amplification or nucleic acid hybridization method. Thus, the genetic sequence functions a means of detection. In one aspect of this embodiment, the sample is selected from a corn commodity product, for example and not limited to corn flour, corn meal, corn syrup, corn oil, corn starch, and cereals manufactured in whole or in part to contain corn products. It is known in the art that a biological sample or extract may comprise proteins with biological activity. Therefore, in a further embodiment, the invention provides a biological sample derived from an elite event MZIR098 corn plant, tissue, or seed, wherein said biological sample comprises insecticidal proteins mCry3A and/or eCry3.1Ab, which continue to have insecticidal activity.

In another embodiment, the invention provides an extract derived from an elite event MZIR098 corn plant, tissue, or seed comprising a nucleotide sequence which is or is complementary to a nucleotide sequence selected from the group consisting of SEQ ID NO: 1, SEQ ID NO: 2, SEQ ID NO: 3, SEQ ID NO: 4, SEQ ID NO: 5, and SEQ ID NO: 6. An example of such seed is deposited at the ATCC under Accession No. PTA-124143. In one aspect of this embodiment, the sequence is detected in the extract using a nucleic acid amplification or nucleic acid hybridization method. In another aspect of this embodiment, the sample is selected from a corn commodity product, such as corn flour, corn syrup, corn oil, cornstarch, and cereals manufactured in whole or in part to contain corn products.

In another embodiment, the invention provides a method of producing a corn commodity product, comprising the steps of: a) obtaining transgenic elite event MZIR098 corn plant, cells or tissues thereof; and b) producing a corn commodity product from the said transgenic corn plant, cells, or tissue thereof, wherein the commodity product comprises protein concentrate, protein isolate, starch, meal, flour or oil therefrom.

In another embodiment, the invention provides a corn commodity product comprising a detectable amount of a DNA molecule unique for corn elite event MZIR098, wherein said molecule comprises SEQ ID NO: 1, SEQ ID NO: 2, SEQ ID NO: 3, SEQ ID NO: 4, SEQ ID NO: 5 or SEQ ID NO: 6. In a further embodiment, the invention provides a non-living plant material comprising a detectable amount of a nucleic acid molecule comprising SEQ ID NO: 1, SEQ ID NO: 2, SEQ ID NO: 3, SEQ ID NO: 4, SEQ ID NO: 5 or SEQ ID NO: 6, wherein the nucleic acid molecule is comprised in a corn seed deposited at the ATCC under the Accession No. PTA-124143.

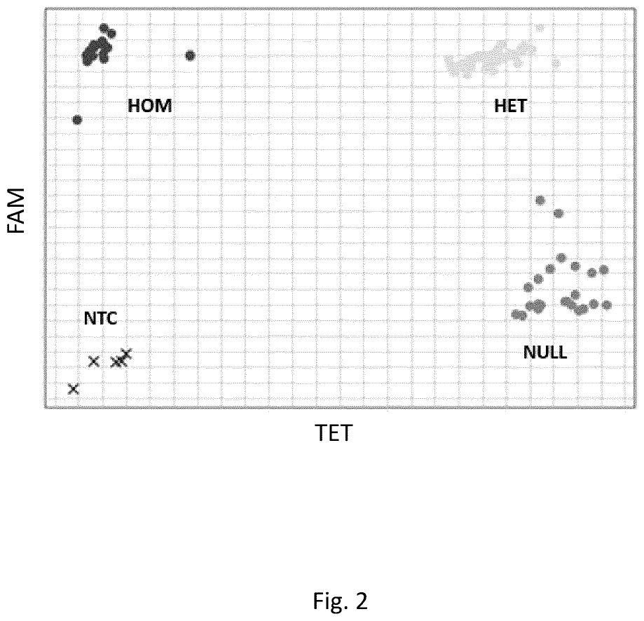

In another embodiment, the invention provides a method for determining zygosity of a corn plant comprising a corn elite event MZIR098 of the invention, said method comprising: (a) obtaining a DNA sample of genomic DNA from said corn plant; (b) producing a contacted sample by contacting said DNA sample with (i) a first event primer and a second event primer, wherein said first event primer specifically binds said transgene construct, said second event primer specifically binds said 5′ corn genomic flanking DNA or said 3′ com genomic flanking DNA, and wherein said first event primer and said second event primer produce an event amplicon which is unique to event MZIR098, when subjected to quantitative PCR conditions, (ii) at least one native insertion site first primer and at least one native insertion site second primer, wherein the first primer is a forward primer and the second primer is a reverse primer, wherein a first and second primer function together when subjected to quantitative PCR conditions to produce an amplicon from the native MZIR098 insertion site when elite event MZIR098 is not present in the genome, (iii) a fluorescent event probe that hybridizes with said event amplicon, (iv) a fluorescent native insertion site probe that hybridizes with said native insertion site amplicon; (c) subjecting said contacted sample to fluorescence-based endpoint quantitative PCR conditions; (d) quantitating said fluorescent event probe that hybridized to said event amplicon and quantitating said fluorescent native insertion site probe that hybridized to said native insertion site amplicon; (e) comparing amounts of hybridized fluorescent event probe to hybridized fluorescent native insertion site probe; and (f) determining zygosity of said corn plant comprising corn elite event MZIR098 by comparing fluorescence ratios of hybridized fluorescent event probe and hybridized fluorescent native insertion site probe. The event primer set and probe and native insertion site primer set and probe may be mixed with the same DNA sample, or they may be separate with different DNA samples derived from the same corn plant. The native insertion primer set may comprise more than one forward native insertion site primer and/or more than one reverse native insertion site primer. The quantification of the fluorescence from the event probe and the fluorescence from the native insertion site probe may be sequentially or simultaneously. Zygosity determination may be made using data analysis software, such as SDS software on the ABI 7900HT, as described in Example 9 and shown in FIG. 2. The results indicate if the corn plant is homozygous for elite event MZIR098 (ie, has positive results for the event endpoint quantitative PCR but not for the native insertion site endpoint quantitative PCR), is heterozygous for elite event MZIR098 (ie, has a positive result for both the event and for the native insertion site endpoint quantitative PCRs) or is wild type (ie, has positive results for the native insertion site endpoint quantitative PCR but not for the event endpoint quantitative PCR).

It will be recognized that some sequence diversity will be found for the native insertion site, based on genetic diversity of the various corn germplasms into which elite event MZIR098 is introduced. Therefore, for successful zygosity determination in novel corn germplasms, a native insertion site primer set may need to be identified so that an amplicon is produced when elite event MZIR098 is not present in the genome, when subjected to quantitative PCF conditions. Multiple native insertion site primer sets and probes may be needed to properly determine the zygosity of event MZIR098 in a variety of corn germplams. Multiple native insertion site primer sets and/or probes may be included in a single reaction to produce a native insertion site amplicon that hybridizes with a native insertion site probe. Similarly, event primers which specifically bind to the 5′ or 3′ flanking sequence of the MZIR098 may need to be identified for successful zygosity determination in novel corn germplasms, as the 5′ and/or 3′ flanking sequences may be diverse among a variety of germplams. Again, multiple event primer sets may be included in a single reaction to produce an event amplicon unique to event MZIR098.

In a further embodiment of the method for determining zygosity described above, the event and/or native insertion site amplicon may consists of 50-200 nucleotides in length. In a preferred embodiment, the amplicon for the event and for the native insertion site is 50-150 nucleotides in length. In another embodiment, the first event primer comprises at least 10 contiguous nucleotides from position 1-8476 as set forth in SEQ ID NO: 7, or a complement thereof, and the second event primer comprises at least 10 contiguous nucleotides from position 1-1076 as set forth in SEQ ID NO: 8 or from position 1-1075 as set forth in SEQ ID NO: 9, or a complement thereof. In a further embodiment, the first event primer is selected from SEQ ID NO: 11, 17, 18, 22, 23, 24, 25, 26, 27, 28, 29, 30, 31, 32, 33, 34, 35, 37, 39, 40, 43, 44, 46, 47, 48, 49, 50, 51, 52, or a complement thereof. In another embodiment, the second event primer is selected from SEQ ID NO: 10, 14, 15, 16, 19, 20, 21, 36, 38, 41, 42, 45, 65, 67, or a complement thereof. In another embodiment, the first native insertion site primer comprises at least 10 contiguous nucleotides from position 1-1076 as set forth in SEQ ID NO: 8, or a complement thereof, and the second native insertion site primer comprises at least 10 contiguous nucleotides from position 1-1075 as set forth in SEQ ID NO: 9, or a complement thereof. In another embodiment, the first native insertion site primer is selected from SEQ ID NO: 10, 14, 15, 16, 38, 42, 65, or a complement thereof. In another embodiment, the second native insertion site primer is selected from SEQ ID NO: 19, 20, 21, 36, 41, 45, 67, or a complement thereof. In another embodiment, the fluorescent native insertion site probe comprises SEQ ID NO: 68 or SEQ ID NO: 69. It is recognized that there may be more than one first primer or second primer for the native insertion site primers.

In a further embodiment, the results of the method for determining zygosity described above are read directly in a plate reader. The present invention also encompasses a kit for performing the method of determining zygosity described above. The kit comprises all primers and probes needed for performing the zygosity assay on a DNA sample, including a first event primer, a second event primer, at least one native insertion site primer, at least one native insertion site reverse primer, an event probe, and a native insertion site probe. The kit may include more than one primer set/probe for the event, for the native insertion site, or both. The kit may also include more than one pair of primers, for example two forward primers and a single reverse primer, as described in Example 9.

In a further embodiment, the present invention encompasses a method of breeding a corn plant comprising herbicide tolerant corn elite event MZIR098 wherein the zygosity of a corn plant comprising corn elite event MZIR098 is determined by the method described above. The zygosity determination method may be used in a breeding program to determine the zygosity of the event MZIR098 in a segregating progeny population. Corn plants may then be selected which are homozygous for event MZIR098 based on the results of the zygosity determination method described above. Corn plants may also be discarded if they are found to be heterozygous for event MZIR098 based on the results of the zygosity determination method.

In yet another embodiment, the invention provides a method for producing a corn plant useful for control of insects and also tolerant to herbicides comprising: (a) sexually crossing a first parent corn plant with a second parent corn plant, wherein said first or second parent corn plant comprises corn elite event MZIR098, thereby producing a plurality of first generation progeny plants; (b) selecting a first generation progeny plant that has herbicide tolerance to GS inhibitors and/or insect resistance to Diabrotica spp, such as western corn rootworm; (c) selfing the first generation progeny plant, thereby producing a plurality of second generation progeny plants; and (d) selecting from the second generation progeny plants a plant that has herbicide tolerance to GS inhibitors and/or insect resistance to corn rootworm; wherein the second generation progeny plants comprise a nucleotide sequence selected from the group consisting of SEQ ID NO: 1, SEQ ID NO: 2, SEQ ID NO:3, SEQ ID NO: 4, SEQ ID NO: 5, and SEQ ID NO: 6.

In yet another embodiment, the invention provides a use of corn elite event MZIR098 to confer insect resistances and/or herbicide tolerance to a plant lacking said event. This usage may comprise, for example, sexually crossing a parent corn plant comprising event MZIR098 with a second parent corn plant which does not comprise event MZIR098 and selecting for progeny which comprise event MZIR098. The progeny may further be backcrossed to the second parent, optionally multiple times, or crossed with additional corn plants as part of a breeding program to produce at least one variety of corn comprising elite event MZIR098 which previously did not comprise said event.

In another embodiment, the invention provides a method of asexually propagating corn elite event MZIR098. Asexual propagation of a corn plant may be performed using methods well-known in the art, for example by anther culture or by microspore-derived plant tissue culture. in vitro plant regeneration may be performed by micropropagation, which involves the suppression of apical dominance resulting in the activation and multiplication of auxillary buds, or by somatic embryogenesis, where for example cotyledon containing embryos are formed from somatic cells. Asexual propagation and in vitro plant regeneration are needed for asexual reproduction. In a further embodiment, the invention provides a corn elite event MZIR098 produced by asexual propagation. The invention also provides use of a corn elite event MZIR098 plant, cells, or tissues to produce a corn elite event MZIR098 plant. This plant may be produced by asexual propagation.

In another embodiment, the invention provides a method of producing hybrid corn seeds comprising: (a) planting seeds of a first inbred corn line, an example of said seed deposited as ATCC accession No. PTA-124143, wherein said seed comprises a nucleotide sequence selected from the group consisting of SEQ ID NO: 1, SEQ ID NO: 2, SEQ ID NO:3, SEQ ID NO: 4, SEQ ID NO: 5, and SEQ ID NO: 6, and seeds of a second inbred line having a different genotype than the first inbred corn plant; (b) cultivating corn plants resulting from said planting until time of flowering and the production of flowers; (c) emasculating said flowers of plants of either the first or the second corn inbred line; (d) sexually crossing the two different inbred lines with each other by pollinating the non-emasculated plant with pollen of the emasculated plant; and (e) allowing hybrid seed to be produced and harvesting the hybrid seed produced thereby. In one aspect of this embodiment, the first inbred corn line provides the female parents. In another aspect of this embodiment, the first inbred corn line provides the male parents. The invention also encompasses the hybrid seed produced by the embodied method and hybrid plants grown from the seed.

In another embodiment, the invention provides a method of selecting markers associated with corn elite event MZIR098 comprising: (a) screening corn elite event MZIR098 chromosome 10 sequences, (b) comparing these with a non-transgenic NP2222 sequences, (c) comparing the sequences for the purpose of detecting sequence variations, (d) using these sequence variations as a means to develop markers associated with corn elite event MZIR098, (e) using the markers to screen lines, and (f) detecting marker confirming the presence of corn elite event MZIR098 sequences on chromosome 10. In a further embodiment, the invention provides a method of breeding a corn plant comprising herbicide tolerant elite event MZIR098 which is genetically linked to or a complement of a nucleic acid marker, wherein said marker is identified using SEQ ID NO: 1, SEQ ID NO: 2, SEQ ID NO: 10 through SEQ ID NO: 12, SEQ ID NO: 14 through SEQ ID NO: 52, SEQ ID NO: 65, SEQ ID NO: 67, or their complements. In another embodiment, the invention provides a method of marker assisted selection for herbicide tolerant corn elite event MZIR098 comprising: (a) isolating nucleic acid molecule(s), or preparing a nucleic acid sample, from corn; (b) combining the nucleic acid molecule(s) with a pair of polynucleotide primers and probes, selected from the group comprising SEQ ID NO: 1, SEQ ID NO: 2, SEQ ID NO: 10 through SEQ ID NO: 12, SEQ ID NO: 14 through SEQ ID NO: 52, SEQ ID NO: 65, SEQ ID NO: 67, or their complements; (c) performing a nucleic acid amplification reaction which results in an amplicon; (d) detecting the amplicon; and (e) selecting the plant for the purposes of breeding herbicide tolerant corn comprising corn elite event MZIR098.

In another embodiment, the invention comprises a transgenic corn plant, cells, or tissues comprising elite event MZIR098, characterized by the SEQ ID NO: 1, SEQ ID NO: 2, SEQ ID NO: 3, SEQ ID NO: 4, SEQ ID NO: 5, or SEQ ID NO: 6, wherein the transgenic corn plant, cells, or tissues are further defined as a progeny or derived from a progeny of any generation of a corn plant comprising elite event MZIR098. In a further embodiment, the transgenic corn plant, cells, or tissues are or are derived from a hybrid bred from at least one parent comprising elite event MZIR098.

One skilled in the art will recognize that the transgenic genotype of the invention can be introgressed by breeding into other corn lines comprising different transgenic genotypes. For example, a corn inbred comprising the transgenic genotype of the invention can be crossed with a corn inbred comprising the transgenic genotype of the lepidopteran resistant Bt11 event, which is known in the art, thus producing corn seed that comprises both the transgenic genotype of the invention and the Bt11 transgenic genotype. Examples of other transgenic events which can be crossed with an inbred of the invention include: the Enogen event 3272, the glyphosate tolerant/lepidopteran insect resistant MON802 event, the lepidopteran insect resistant event DBT418, the lepidopteran insect resistant event DAS-06275-8, the lepidopteran insect resistant event MIR162, the male sterile event MS3, the lepidopteran insect resistant event MON 80100, the lepidopteran insect resistant event 176, and the coleopteran insect resistant event MON863, all of which are known in the art. It will be further recognized that other combinations can be made with the transgenic genotype of the invention and thus these examples should not be viewed as limiting.

In another embodiment, the invention encompasses a process for producing corn elite event MZIR098 seed. This process comprises crossing a corn elite event MZIR098 of the invention with a second corn plant. The second corn plant may or may not comprise the MZIR098 event. In preferred embodiments, the second corn plant does not comprise the MZIR098 event. Following the crossing, or pollination event, the seed is allowed to develop and set in the maternal plant. The invention further comprises the corn elite event MZIR098 seed produced by the process described above, as well as the corn elite event MZIR098 plant produced by germinating the seed.

In another embodiment, the invention provides a process of introducing an additional trait into a corn elite event MZIR098 plant, comprising: (a) crossing a corn elite event MZIR098 plant grown from corn elite event MZIR098 seed, representative seed deposited under ATCC Accession Number PTA-124143, with another maize plant that comprises an additional trait to produce hybrid progeny plants, (b) selecting hybrid progeny plants that have the additional trait to produce selected hybrid progeny plants; (c) crossing the selected progeny plants with the corn elite event MZIR098 parental plants to produce backcross progeny plants; (d) selecting for backcross progeny plants that have the additional trait to produce selected backcross progeny plants; and (e) repeating steps (c) and (d) at least three or more times to produce backcross progeny plants that comprise the additional trait and corn elite event MZIR098. The invention further comprises a plant produced by the process described above.

In another embodiment, the invention provides a method for developing a corn elite event MZIR098 plant germplasm in a corn plant breeding program, comprising applying plant breeding techniques wherein said techniques comprise recurrent selection, backcrossing, pedigree breeding, marker enhanced selection, haploid/double haploid production, or transformation of a corn elite event MZIR098 plant, or its parts, wherein application of said techniques results in development of a second corn germplasm comprising elite event MZIR098.

In another embodiment, the invention provides a method of producing a corn elite event MZIR098 plant with doubled haploid chromosomes, the method comprising: (a) crossing the plant of claim 2 with an inducer maize plant to produce a progeny with haploid chromosomes; and (b) doubling the haploid chromosomes in the progeny to produce a corn elite event MZIR098 plant with doubled haploid chromosomes.

### Breeding

Manipulations (such as mutation, further transfection, and further breeding) of plants or seeds, or parts thereof, may lead to the creation of what may be termed “essentially derived” varieties. The International Union for the Protection of New Varieties of Plants (UPOV) has provided the following guideline for determining if a variety has been essentially derived from a protected variety:
[A]variety shall be deemed to be essentially derived from another variety (“the initial variety”) when
(i) it is predominantly derived from the initial variety, or from a variety that is itself predominantly derived from the initial variety, while retaining the expression of the essential characteristics that result from the genotype or combination of genotypes of the initial variety;
(ii) it is clearly distinguishable from the initial variety; and
(iii) except for the differences which result from the act of derivation, it conforms to the initial variety in the expression of the essential characteristics that result from the genotype or combination of genotypes of the initial variety.

UPOV, Sixth Meeting with International Organizations, Geneva, Oct. 30, 1992; document prepared by the Office of the Union.

As used herein, a “line” is a group of plants that display little or no genetic variation between individuals for at least one trait. Such lines may be created by several generations of self-pollination and selection, or vegetative propagation from a single parent using tissue or cell culture techniques.

As used herein, the terms “cultivar” and “variety” are synonymous and refer to a line which is used for commercial production.

“Stability” or “stable” means that with respect to the given component, the component is maintained from generation to generation and, preferably, at least three generations at substantially the same level, e.g., preferably ±15%, more preferably ±10%, most preferably ±5%. The stability may be affected by temperature, location, stress and the time of planting. Comparison of subsequent generations under field conditions should produce the component in a similar manner.

“Commercial Utility” is defined as having good plant vigor and high fertility, such that the crop can be produced by farmers using conventional farming equipment.

The transgenic genotype of the invention can be introgressed in any corn inbred or hybrid using art recognized breeding techniques. The goal of plant breeding is to combine in a single variety or hybrid various desirable traits. For field crops, these traits may include resistance to insects and diseases, tolerance to herbicides, tolerance to heat and drought, reducing the time to crop maturity, greater yield, and better agronomic quality. With mechanical harvesting of many crops, uniformity of plant characteristics such as germination and stand establishment, growth rate, maturity, and plant and ear height, is important.

Field crops are bred through techniques that take advantage of the plant's method of pollination. A plant is self-pollinated if pollen from one flower is transferred to the same or another flower of the same plant. A plant is cross-pollinated if the pollen comes from a flower on a different plant.

Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and will not be uniform.

Corn can be bred by both self-pollination and cross-pollination techniques. Corn has separate male and female flowers on the same plant, located on the tassel and the ear, respectively. Natural pollination occurs in corn when wind blows pollen from the tassels to the silks that protrude from the tops of the ears.

A reliable method of controlling male fertility in plants offers the opportunity for improved plant breeding. This is especially true for development of corn hybrids, which relies upon some sort of male sterility system. There are several options for controlling male fertility available to breeders, such as: manual or mechanical emasculation (or detasseling), cytoplasmic male sterility, genetic male sterility, gametocides and the like.

Hybrid corn seed is typically produced by a male sterility system incorporating manual or mechanical detasseling. Alternate strips of two corn inbreds are planted in a field, and the pollen-bearing tassels are removed from one of the inbreds (female). Providing that there is sufficient isolation from sources of foreign corn pollen, the ears of the detasseled inbred will be fertilized only from the other inbred (male), and the resulting seed is therefore hybrid and will form hybrid plants.

The laborious, and occasionally unreliable, detasseling process can be avoided by using one of many methods of conferring genetic male sterility in the art, each with its own benefits and drawbacks. These methods use a variety of approaches such as delivering into the plant a gene encoding a cytotoxic substance associated with a male tissue specific promoter or an antisense system in which a gene critical to fertility is identified and an antisense to that gene is inserted in the plant (see: Fabinjanski, et al. EPO 89/3010153.8 publication no. 329,308 and PCT application PCT/CA90/00037 published as WO 90/08828).

### Development of Corn Inbred Lines

The use of male sterile inbreds is but one factor in the production of corn hybrids. Plant breeding techniques known in the art and used in a corn plant breeding program include, but are not limited to, recurrent selection, backcrossing, pedigree breeding, restriction length polymorphism enhanced selection, marker assisted selection and transformation. The development of corn hybrids in a corn plant breeding program requires, in general, the development of homozygous inbred lines, the crossing of these lines, and the evaluation of the crosses. Pedigree breeding and recurrent selection breeding methods are used to develop inbred lines from breeding populations. Corn plant breeding programs combine the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Plant breeding and hybrid development, as practiced in a corn plant-breeding program, are expensive and time-consuming processes.

Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to homogeneous lines as a result of self-pollination and selection. Typically in the pedigree method of breeding five or more generations of selfing and selection is practiced: F₁→F₂; F₂→F₃; F₃→F₄; F₄→F_{0.5}; etc.

Recurrent selection breeding, backcrossing for example, can be used to improve an inbred line and a hybrid that is made using those inbreds. Backcrossing can be used to transfer a specific desirable trait from one inbred or source to an inbred that lacks that trait. This can be accomplished, for example, by first crossing a superior inbred (recurrent parent) to a donor inbred (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

An inbred plant could also be produced by applying double haploid methods to the progeny of a cross between a corn plant comprising elite event MZIR098 and a different plant. Double haploid methods produce substantially homozygous plants without repeated backcrossing steps. The haploid/doubled haploid process of developing inbreds starts with the induction of a haploid by using, for example, KWS inducers lines, Krasnador inducers lines, stock six inducer lines (Coe, 1959, Am. Nat. 93:381-382). The haploid cell is then doubled, and the doubled haploid plant is produced. In some embodiments, the invention is a method of producing a corn plant with doubled haploid chromosomes derived from a corn elite event MZIR098, the method comprising: (a) crossing a plant, wherein said plant comprises elite event MZIR098, with an inducer maize plant to produce a progeny with haploid chromosomes; and (b) doubling the haploid chromosomes in the progeny to produce a maize plant with doubled haploid chromosomes. In some embodiments, the progeny may be for example a cell, seed, embryo or plant. In further embodiments, the maize plant with doubled haploid chromosomes produced by step (b) above is a maize inbred plant with the characteristics of corn elite event MZIR098. In other embodiments, the plant crossed with an inducer in step (a) is a hybrid maize plant produced by crossing a corn plant comprising elite event MZIR098 with a different plant.

For examples of the use of double hybrid methods, see Prasanna et al. (eds) Doubled Haploid Technology in Maize Breeding: Theory and Practice Mexico, D.F.: CIMMYT, Barnabus et al. “Colchicine, an efficient genome doubling agent for maize microsporescultured in anthero”, Plant Cell Reports, 1999, 18: 858-862 or US patent publication 2003/0005479. Sometimes this doubled haploid can be used as an inbred but sometimes it is further self pollinated to finish the inbred development. Another breeding process is pedigree selection which uses the selection in an F2 population produced from a cross of two genotypes (often elite inbred lines), or selection of progeny of synthetic varieties, open pollinated, composite, or backcrossed populations. Pedigree selection is effective for highly heritable traits, such as a transgenic event, but other traits, such as yield, require replicated test crosses at a variety of stages for accurate selection.

Elite inbred lines, that is, pure breeding, homozygous inbred lines, can also be used as starting materials for breeding or source populations from which to develop other inbred lines. These inbred lines derived from elite inbred lines can be developed using the pedigree breeding and recurrent selection breeding methods described earlier. As an example, when backcross breeding is used to create these derived lines in a corn plant-breeding program, elite inbreds can be used as a parental line or starting material or source population and can serve as either the donor or recurrent parent.

### Development of Corn Hybrids

A single cross corn hybrid results from the cross of two inbred lines, each of which has a genotype that complements the genotype of the other. The hybrid progeny of the first generation is designated F₁. In the development of commercial hybrids in a corn plant-breeding program, only the F₁ hybrid plants are sought. Preferred F₁ hybrids are more vigorous than their inbred parents. This hybrid vigor, or heterosis, can be manifested in many polygenic traits, including increased vegetative growth and increased yield.

The development of a corn hybrid in a corn plant breeding program involves three steps: (1) the selection of plants from various germplasm pools for initial breeding crosses; (2) the selfing of the selected plants from the breeding crosses for several generations to produce a series of inbred lines, which, although different from each other, breed true and are highly uniform; and (3) crossing the selected inbred lines with different inbred lines to produce the hybrid progeny (F₁). During the inbreeding process in corn, the vigor of the lines decreases. Vigor is restored when two different inbred lines are crossed to produce the hybrid progeny (F₁). An important consequence of the homozygosity and homogeneity of the inbred lines is that the hybrid between a defined pair of inbreds will always be the same. Once the inbreds that give a superior hybrid have been identified, the hybrid seed can be reproduced indefinitely as long as the homogeneity of the inbred parents is maintained. Much of the hybrid vigor exhibited by F₁ hybrids is lost in the next generation (F₂). Consequently, seed from hybrids is not used for planting stock.

Hybrid seed production requires elimination or inactivation of pollen produced by the female parent. Incomplete removal or inactivation of the pollen provides the potential for self-pollination. This inadvertently self-pollinated seed may be unintentionally harvested and packaged with hybrid seed.

Once the seed is planted, it is possible to identify and select these self-pollinated plants. These self-pollinated plants will be genetically equivalent to the female inbred line used to produce the hybrid.

As is readily apparent to one skilled in the art, the foregoing are only some of the various ways by which the inbred of the invention can be obtained by those looking to introgress the transgenic genotype of the invention into other corn lines. Other means are available, and the above examples are illustrative only.

One skilled in the art will also recognize that transgenic corn seed comprising the transgenic genotype of the invention can be treated with various seed-treatment chemicals, including insecticides. In one embodiment, the invention comprises a method for protecting a corn elite event MZIR098 plant against feeding damage by one or more pests, said method comprising (a) providing a MZIR098 seed of the corn elite event MZIR098 plant; and (b) treating the MZIR098 plant with an insecticide. In preferred embodiments, the insecticide may comprise an active ingredient selected from the group consisting of thiamethoxam, lambda-cyhalothrin, and tefluthrin. For example, the transgenic corn seed of the invention can be treated with the commercial insecticide Cruiser®. The present invention also encompasses a corn elite event MZIR098 seed treated with an insecticide. In a further embodiment, the invention comprises a corn elite event MZIR098 seed treated with an insecticide which comprises an active ingredient selected from the group consisting of thiamethoxam, lambda-cyhalothrin, and tefluthrin. In another embodiment, the invention encompasses a corn elite event MZIR098 plant treated with an insecticide. In a preferred embodiment, the invention encompasses a corn elite event MZIR098 plant treated with an insecticide which comprises an active ingredient selected from the group consisting of thiamethoxam, lambda-cyhalothrin, and tefluthrin.

In another embodiment, the invention provides a method of controlling weeds, where an herbicide is applied to a field comprising corn elite event MZIR098 plants. In preferred embodiments, the herbicide is a GS inhibitor, such as glufosinate or bialaphos. Examples of commercially available herbicides comprising a GS inhibitor include Herbiace, Meiji Herbiace, Liberty®, Ignite®, Rely®, Finale®, and Basta®.

In another embodiment, the invention provides a method of controlling glyphosate-resistant weeds in an area comprising at least corn elite event MZIR098 plant, wherein said method comprises applying a GS inhibitor herbicide, such as glufosinate or bialaphos, to at least a portion of said area. The glyphosate-resistant weeds may be unwanted volunteer Brassica ssp, millet, switchgrass, maize, sorghum, wheat, oat, turf grass, pasture grass, papaya, flax, peppers, potato, sunflower, tomato, crucifers, soybean, common bean, lotus, grape, peach, cacao, cotton, rice, soybean, sugarcane, sugar beet, tobacco, barley, cassava, cucumber, watermelon, melon, orange, clementine, castor bean, or grapevine. In another embodiment, the invention provides a method of controlling weeds in an area under cultivation, said area comprising a plurality of corn elite event MZIR098 plants, said method comprising applying a GS inhibitor herbicide, such as glufosinate or bialaphos, over the top of the plants.

In another embodiment, the present invention provides a corn plant comprising elite event MZIR098, wherein the corn plant is useful for control of Diabrotica spp. insect pests. In further embodiments, the corn plant is useful for control of corn rootworm. Examples of corn rootworm species include western corn rootworm and northern corn rootworm. Another embodiment of the invention is a method of controlling insect pests, comprising planting a corn plant comprising elite event MZIR098 in a field. The field may comprise at least one MZIR098 corn plant, at least 50% MZIR098 corn plants, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% MZIR098 corn plants. The present invention also provides a use of a corn plant comprising elite event MZIR098 for controlling insect pests in a field. The insect pests may be Diabrotica spp. The insect pests may further be corn rootworm, for example western corn rootworm and northern corn rootworm.

Another embodiment of the invention is a recombinant sequence, which comprises a maize chromosomal target site located on chromosome 10 between SNP markers SYN23814 and PZE-110102022 and a heterologous nucleic acid. The maize chromosomal target site on chromosome 10 is also located between SNP markers PZE-110101785, PZE-110101800, and SYN23814 (all at the 5′ flanking sequence) and SNP markers PZE-110102022, PZA02167.2, SYNGENTA16568, and SYN8530 (all at the 3′ flanking sequence). The heterologous nucleic acid may be introduced at the maize chromosomal target site by targeted insertion. A further embodiment is a recombinant nucleic acid molecule of chromosome 10 comprising a heterologous nucleic acid sequence inserted on chromosome 10 set forth as nucleotide 1 to nucleotide 172,841 of SEQ ID NO: 70. A preferred embodiment is a recombinant nucleic acid molecule comprising a heterologous nucleic acid sequence inserted on chromosome 10 set forth as nucleotide 18,206 to 18,246 of SEQ ID NO: 70. Another embodiment is a recombinant nucleic acid molecule comprising a heterologous nucleic acid proximal at its 5′ end to nucleotides 16,206 to 18,206 of SEQ ID NO: 70 and proximal at its 3′ end to nucleotides 18,246 to 20,246 of SEQ ID NO: 70. In another embodiment, the present invention provides a corn genome having a heterologous nucleic acid inserted on chromosome 10 set forth as nucleotide 18,206 to 18,246 of SEQ ID NO: 70, wherein the heterologous nucleic acid comprises SEQ ID NO: 7. In a further embodiment, the present invention provides a corn genome having a heterologous nucleic acid inserted on chromosome 10 set forth as nucleotide 18,206 to 18,246 of SEQ ID NO: 70, wherein the heterologous nucleic acid comprises SEQ ID NO: 62, 63, and/or SEQ ID NO: 64.

Another embodiment of the invention is a method of making a transgenic maize plant comprising inserting a heterologous nucleic acid at a position on chromosome 10 set forth as nucleotide 1 to nucleotide 172,841 of SEQ ID NO: 70. A preferred embodiment is a method of making a transgenic maize plant, wherein the heterologous nucleic acid is inserted on chromosome 10 set forth as between nucleotide 18,206 to nucleotide 18,246 of SEQ ID NO: 70. A further embodiment is a method of making a transgenic maize plant comprising inserting a heterologous nucleic acid at a position on chromosome 10, wherein the heterologous nucleic acid is proximal at its 5′ end to nucleotides 16,206 to 18,206 of SEQ ID NO: 70 and proximal at its 3′ end to nucleotides 18,246 to 20,246 of SEQ ID NO: 70.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); J. Sambrook, et al., Molecular Cloning: A Laboratory Manual, 3d Ed., Cold Spring Harbor, NY: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, NY (1984).

### Example 1. Transformation and Selection of the Elite Event MZIR098

The MZIR098 event was produced by Agrobacterium-mediated transformation of the inbred corn (Zea mays) line NP2222. Immature embryos were transformed essentially as described in Negrotto et al. (Plant Cell Reports 19: 798-803, 2000), incorporated herein by reference, using the T-DNA fragment from binary vector 17629 (PCT Application No. PCT/US16/29424, incorporated by reference herein). Vector 17629 contains a nucleotide sequence comprising eCry3.1Ab, mCry3A and PAT tandem expression cassettes as part of its T-DNA sequence. The first expression cassette comprises a NOS gene enhancer region, derived from the nopaline synthase (NOS) gene from A. tumefaciens (eNOS-02; SEQ ID NO: 38) operably linked to a Cestrum Yellow leaf curl virus promoter prCMP-04; SEQ ID NO: 39; US Patent Publication US20040086447), operably linked to an engineered Cry toxin eCry3.1Ab (SEQ ID NO: 40; International Publication No. WO 08/121633, published Oct. 9, 2008, herein incorporated by reference), operably linked at the 3′ end to a terminator sequence derived from the nopaline synthase (NOS) gene from A. tumefaciens (tNOS-05-01; SEQ ID NO: 41). The second expression cassette comprises a constitutive corn promoter based on a ubiquitin gene (prUbi1-18; SEQ ID NO: 42; Christensen et al., 1992, PMB 18: 675-689), operably linked to a modified Cry toxin mCry3A (SEQ ID NO: 43; U.S. Pat. No. 7,030,295), operably linked at its 3′ end to a terminator sequence derived from the NOS gene (tNOS-20; SEQ ID NO: 44). The third expression cassette comprises a CaMV 35S promoter (pr35S-04-01; SEQ ID NO: 45) operably linked to a PAT coding sequence (cPAT-08; SEQ ID NO: 46), operably linked at its 3′ end to a terminator sequence derived from the NOS gene (NOS-05-01; SEQ ID NO: 41). The T-DNA sequence comprising these three expression cassettes is SEQ ID NO: 7.

Immature embryos were excised from 8-12 day old ears and rinsed with fresh medium in preparation for transformation. Embryos were mixed with the suspension of Agrobacterium cells harboring the binary vector 17629, vortexed for 30 seconds, and allowed to incubate for an additional 5 minutes. Excess Agrobacterium solution was aspirated and embryos were then moved to plates containing a non-selective culture medium. Embryos were co-cultured with the remaining Agrobacterium at 22° C. for 2-3 days in the dark. The PAT gene was used as a selectable marker during the transformation process (Negrotto et al. 2000). The embryos producing embryogenic calli were transferred to a series of cell culture selection media containing bialaphos as selection agent and cultured for 10-11 weeks in total. The selection media contained 200 mg/ml timentin and/or 10 ml/1 PPM (Plant Preservative Mix) to ensure that the Agrobacterium was cleared from the transformed tissue.

Regenerated plantlets were tested by TaqMan PCR analysis (see Example 2) for the presence of the eCry3.1 Ab, mCry3A, and PAT genes, as well as for the absence of the antibiotic resistance spectinomycin (spec) gene, which is on the vector 17629 backbone. Plants positive for all transgenes, and negative for the spec gene, were transferred to the greenhouse for further propagation. Events then were analyzed a second time using TaqMan PCR analysis, this time to determine copy number of the transgenes. Single-copy events were then analyzed for transgenic proteins using ELISA (see Example 7). Event identified with good protein levels were then allowed to grow to maturity. The coding sequences of the transgenes in these transgenic events was verified, to identify events with mutations in the transgenic protein sequences. Finally, the remaining events were analyzed intactness of the T-DNA insert using the Fluidigm BioMark System (Fluidigm Corporation, San Francisco, CA, USA), to determine if a full-length single copy of the T-DNA was inserted into the genome of the event. Events with good seed set, both as the product of selfing and from out-crossing, were finally identified.

TABLE 1
T0 Event Selection

| T0 Event Selection | Events | % |
|---|---|---|
| Embryos used in corn transformation | 42698 | |
| Primary any-copy events evaluated | 2043 | 100 |
| Events that passed 1° TaqMan and sent to greenhouse | 610 | 30 |
| Events selected for further work based on ELISA | 196 | 10 |
| protein expression in leaves of one or both rootworm | | |
| genes | | |
| Events selected based on 2° TaqMan confirmation | 173 | 8.47 |
| Events that passed herbicide tolerance screen based on | 166 | 8.13 |
| leaf painting with glufosinate | | |
| Events that passed CDS coding sequence verification | 137 | 6.71 |
| to eliminate events with altered amino acid sequences | | |
| in three trait genes (mCry3A, eCry3.1Ab, PAT) | | |
| Events for which sufficient T1 and F1 seed was | 109 | 5.34 |
| obtained in greenhouse | | |
| Events planted in winter nursery, following additional | 92 | 4.50 |
| event attrition based on Southern blot data | | |

As shown in Table 1, a large-scale corn transformation effort was undertaken in which over 42,000 embryos were transformed to generate 2,043 putative any-copy events. An any-copy event may contain multiple T-DNA insertions at multiple locations in the genome and/or whole or partial duplications and/or T-DNA rearrangements at a given insertion site. 610 of these were putatively determined by primary TaqMan analysis to have a single insertion, with no backbone contamination. Following ELISA expression analysis, 196 events were determined to have desirable levels of expression of all trait genes and selected for additional extensive molecular characterization. This molecular characterization included verification of coding sequences to eliminate events with altered protein coding sequences. This molecular characterization data together with greenhouse seed yield was used to select a total of 109 events. Molecular analysis by Southern blot for each of these events was performed. To advance, an event needed to be free of the 17629 vector backbone, contain one and only copy of the intact T-DNA insertion, and have no genetic rearrangements. The mCry3A and eCry3.1Ab coding sequences contain a long stretch (1400 bp) of 100% identity, so the possibility of rearrangement within the transgene or during insertion into the genome was high. These events were each evaluated by Southern analysis. Based on those results, 92 backbone-free, intact (no genetic rearrangement) single-copy events were selected for field trials.

The field trials typically comprised a hybrid line from each event tested, with at least 6 plants of each hybrid per plot, three replicates per location, and at least two locations evaluated. For the field trial, Western Corn Rootworm damage was assessed using the ISU (Iowa State University) 0-3 scale. All field trials included positive controls of transgenic event MIR604 (which comprises mCry3A, but not eCry3.1Ab), transgenic event 5323 (which comprises eCry3.1Ab, but not mCry3A), and transgenic hybrids which result from a cross from MIR604 and 5323. All field trials also included a negative control which was non-transgenic corn. Events were also evaluated for herbicide tolerance; they were sprayed with a 2× or a 4× the maximum labeled rate of Ignite® at the V4 maize developmental stage. Events were also evaluated for agronomic performance, including yield. Events which performed the best compared to the positive and negative controls were selected for advancement.

Following two years of field trial data, 35 events were selected based on trait and agronomic performance, using assays similar to those described above. After two additional field trials, 18 events were selected for further evaluation, and finally 12 events were evaluated in extensive event selection trials (see Example 8). These field trials included trait expression studies with field grown root samples harvested at V6 and V8 developmental stages. These samples were obtained from roots of plants (from each of the 12 events) which were part of active CRW field efficacy trials. Briefly, the roots were obtained, evaluated for CRW damage, lyophilized, ground, and 100 mg of the lyophilized root tissue was used for mCry3A and eCry3.1Ab ELISA assays performed similar to as described in Example 7. Root samples from transgenic MIR604 plants (expressing mCry3A) and from transgenic 5323 (expressing eCry3.1Ab) were also analyzed by ELISA as positive controls.

The 12 events were also subjected to flanking sequence recovery, so that the genomic location of the transgene insertion was identified and characterized. There are a number of criteria for a high-quality genomic insertion. Firstly, the transgene insertion cannot be in a genic region of the genome. Genic regions are understood to include 5′-untranslated regions (UTRs), exons, introns and 3′-UTRs. Additionally, the transgene insertion cannot be in a promoter region. Promoter regions are estimated to be 2 kilobases (kb) upstream of a gene. Additionally, a high-quality junction sequence does not comprise a “junction ORF”, which is a putative Open Reading Frame (ORF) that crosses the T-DNA and genomic DNA junction. A junction ORF is initiated by an “ATG” nucleic acid sequence, which may be created as a result of the random insertion of the T-DNA into the genome, where genomic sequence breaks as well as small insertion, deletions, and/or rearrangements of nucleic acid sequence can occur. A junction ORF may comprise a sequence which could, if expressed, contain an amino acid sequence similar to amino acid sequences of known allergens. Therefore, junction ORFs are highly undesirable and cannot occur in a high quality genomic insertion, or in an elite transgenic event.

Of the 12 events selected for further study, 7 had the T-DNA insertion in a genic region or a promoter region. For another one, the RB could not be sequenced, suggesting a T-DNA rearrangement upon insertion. Of the remaining four candidate events (MZIR098, MZIR08G, MZIROAN, and MZIR0BC), event 08G had mCry3A expression in the roots lower at the V8 growth stage compared to the MIR604 positive control, and eCry3.1Ab expression in roots lower than the event 5323 positive control for both the V4 and V8 growth stages. The remaining three events expressed mCry3A and eCry3.1Ab at least to the levels of the positive controls. However, only event MZIR098 surprisingly and unexpectedly expressed mCry3A to a level significantly higher than MIR604. Event MIR604 is an elite event, which was selected because it had the best mCry3A expression amongst its sister events, in addition to a high-quality genomic insertion and junction sequences and excellent agronomic performance. The finding that event MZIR098 has an even higher level of mCry3A protein, especially in the roots, where the CRW insect pest feeds on the corn plant, was unpredictable and unexpected.

Based on the combination of all of the trait efficacy trials, agronomic trials, molecular and genomic characterization, and protein levels of mCry3A and eCry3.1Ab in the roots, only one event was cleanly identified to have the potential to be an elite event. This event was MZIR098. Event MZIR098 plants were further evaluated for CRW efficacy, CRW high pressure agronomic equivalence testing, and agronomic performance in a number of different hybrid genetic backgrounds. Trials were performed in 40 locations for three years. Agronomic traits evaluated include yield, lodging, flowering, plant height, and disease pressure. The trials showed that event MIZ098 has excellent agronomic performance across multiple locations, for multiple growing seasons, under a variety of environmental pressures, and in multiple genetic backgrounds.

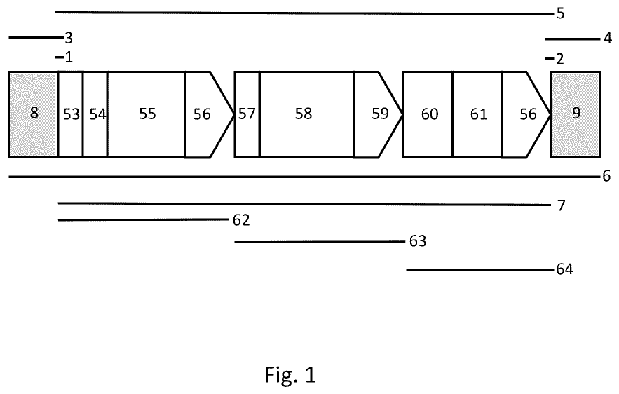

The superior performance of plants comprising the MZIR098 event is due to the intactness of the transgene itself and to the transgene insertion, at a specific location in the corn genome which supports high expression and thus high efficacy of the trait genes encoded on the transgene, and which further does not have any negative impact on plant performance. Additionally, the high agronomic performance and trait efficacy, with no negative effects, of all MZIR098 hybrids produced from multiple genetic backgrounds, further showcase the novelty of the MZIR098 event. Again, this is due to the exact genomic location of the transgene and characteristics of the transgene insertion, which are described by the nucleic acid sequence of the junction sequences (SEQ ID NO: 1 and SEQ ID NO: 2). Therefore, event MZIR098 was found to be an elite event, and it was selected as the lead event for commercial launch. FIG. 1 illustrates the transgene of MZIR098 inserted into the corn genome.

### Example 2. Elite Event MZIR098 Detection by TaqMan PCR

A real-time, MZIR098-specific polymerase chain reaction (PCR) method was developed to detect and quantify MZIR098 deoxyribonucleic acid (DNA) extracted from seed, grain and other plant material samples. The method consists of a maize-specific PCR method as a reference and an event-specific PCR method for detection and quantification of MZIR098 maize DNA. This method can be used to determine the relative content of event MZIR098 maize DNA in proportion to total maize DNA in samples.

For specific detection of MZIR098 maize genomic DNA, two specific primers (SEQ ID NO: 10 and SEQ ID NO: 11) were used to amplify a 73-bp fragment of the region that spans the 5′ insert-to-plant genome junction (SEQ ID NO: 13). A control primer set and probe, for example to an endogenous maize gene, may be used as a positive control. The amount of PCR product is determined during each cycle in real-time by measuring the fluorescence produced by an MZIR098-specific oligonucleotide probe labeled with 6-FAM™ as a reporter dye at its 5′ end and BHQ®-lplus as a quencher at its 3′ end (SEQ ID NO: 12). The primers and probe are shown in Table 2.

It is recognized that other primer combinations could be used to detect an MZIR098 junction sequences. For example, to detect the 5′ junction sequence (SEQ ID NO: 1), a first primer may be selected from SEQ ID NOs: 10, 14, 15, 16, 38, 42, 65, or the complement thereof, and a second primer may be selected from SEQ ID NOs: 11, 17, 22, 23, 24, 25, 29, 43, 46, 47, 48, 49, or the complement thereof. A skilled person of the art would recognize that a first primer comprising a sequence from the 5′ flanking sequence (SEQ ID NO: 8, or a complement thereof) and a second primer comprising a sequence from within the transgene (SEQ ID NO: 7, or a complement thereof) can function as a pair in a PCR reaction to produce an amplicon that comprises the 5′ junction sequence (SEQ ID NO: 1), and this amplicon may be detected by a probe, for example. Similarly, to detect the 3′ junction sequence (SEQ ID NO: 2), a first primer may be selected from SEQ ID NOs: 19, 20, 21, 36, 41, 45, 67, or the complement thereof, and a second primer may be selected from SEQ ID NOs: 18, 31, 33, 34, 35, 37, 50, 52 or a complement thereof. A skilled person of the art would recognize that a first primer comprising a sequence from the 3′ flanking sequence (SEQ ID NO: 9, or a complement thereof) and a second primer comprising a sequence from within the transgene (SEQ ID NO: 7, or a complement thereof) can function as a pair in a PCR reaction to produce an amplicon that comprises the 3′ junction sequence (SEQ ID NO: 2), and this amplicon may be detected by a probe, for example. It is important to note that these junction sequences are novel to the invention described herein. Therefore, it is only with the present disclosure that a skilled person would know the junction sequences which are novel and unique to event MZIR098, and also have motivation to detect them.

TaqMan analysis was essentially carried out as described in Ingham et al. (Biotechniques, 31:132-140, 2001) herein incorporated by reference. Briefly, genomic DNA was isolated from leaves of transgenic and non-transgenic corn plants using the Puregene® Genomic DNA Extraction kit (Gentra Systems, Minneapolis, MN) essentially according to the manufacturer's instruction, except all steps were conducted in 1.2 ml 96-well plates. The dried DNA pellet was resuspended in TE buffer (10 mM Tris-HCl, pH 8.0, 1 mM EDTA).

TaqMan PCR reactions were carried out in 96-well plates. For the endogenous corn gene control, primers and probes were designed specific to the Zea mays alcohol dehydrogenase (Adh) gene (Genbank accession no. AF044295). It will be recognized by the skilled person that other corn genes can be used as endogenous controls.

TABLE 2
Elite Event MZIR098 Detection by TaqMan PCR

| | | SEQ |
|---|---|---|
| Primer/ | | ID |
| probe name | Primer sequence 5′ to 3′ | NO: |
| Forward primer, | ATCTCAGACACCAAACCGAGATC | 10 |
| targeting | | |
| flanking | | |
| sequence | | |
| Reverse primer, | ACACCGTTAGGCTAGTGCCAGT | 11 |
| targeting | | |
| transgene | | |
| Probe, | 6-FAM ™- | 12 |
| targeting | CAAGTGACAGCGAACGGAGCTGGTTT- | |
| junction | | |
| sequence | BHQ ®-1plus | |

The master mix for the event MZIR098-specific TaqMan PCR is shown in the table below:

TABLE 3
Detection of MZIR098 by TaqMan PCR

| | Final | |
|---|---|---|
| Components | concentration | |
| DNA (5-10 ng/μL) | 30 | ng |
| JumpStart ™ Taq ReadyMix ™* | 1× | |
| MZIR098 forward primer ( | 300 | nM |
| MZIR098 reverse primer | 300 | nM |
| MZIR098 probe | 100 | nM |
| Adh forward primer | 300 | nM |
| Adh reverse primer | 300 | nM |
| Adh probe | 100 | nM |
| Nuclease-free water | as needed | |
| Total volumeª | 6 | uL |

*supplemented with Sulforhodamine 101 and 11 mM MgCl₂

PCR was performed in the ABI Prism 7700 instrument using the following amplification parameters: 10 min at 95° C., followed by 40 cycles of 15 s at 95° C. and 1 min at 60° C. The PCR may be run on an ABI 7900HT, a GeneAmp PCR system 9700, or any other appropriate system. The data was analyzed using the SDS software on the ABI 7900HT. Results validated the above methodology, verifying it successfully identified the MZIR098 event in a biological sample derived from corn tissue, including seed, grain, and other plant material samples.

### Example 3. Elite Event MZIR098 Detection by Southern Blot

An extensive genetic characterization of the T-DNA insert of elite event MZIR098 was performed by means of Southern blot analyses and nucleotide sequencing. The genetic stability of the insert was assessed both by Southern blot analyses and by examining the inheritance patterns of the transgenes over at least three generations of MZIR098 maize. Sequencing results confirmed the expected copy number of each of the functional elements in the T-DNA. In addition, the corn genomic sequences flanking the MZIR098 insert were identified and characterized. It was also determined that the MZIR098 insert did not disrupt the function of any known corn gene. These data collectively demonstrate that no deleterious changes occurred in the MZIR098 genome as a result of the T-DNA insertion, further demonstrating that MZIR098 is an elite event.

Southern blot analyses were performed to characterize the transgenic insert of MZIR098 maize by determining the number of vector 17629 T-DNA integration sites, the presence or absence of vector 17629 backbone sequence, and the addition of extraneous fragments of T-DNA. The MZIR098 corn generations used in Southern blot analysis included T2 (two samples, from ear 4 and ear 35), T3, T4, T5, and F1. The T2 through T5 generations were in the genetic background NP2222. The F1 generation was in the background NP2391/NP2222 and was representative of a commercial corn hybrid. Five generations of MZIR098 corn were included to demonstrate stability of the T-DNA insert over multiple generations. The control substances were nontransgenic, near-isogenic NP2222, NP2391, and NP2222/NP2391 corn. All material was grown in a greenhouse. Leaf tissue from seven plants were sampled, pooled, and subjected to DNA extraction. The genomic DNA used for Southern blot analyses was isolated from leaf tissue by a method modified from that described by Murray and Thompson (1980, Nucleic Acids Research, 8: 4321-4325).

The elements of the vector necessary for its replication and selection in different bacterial hosts are categorized as “vector backbone” (the region outside of the T-DNA). In the Southern blot analyses, the presence or absence of vector backbone was determined through the use of two backbone-specific probes that together covered every base pair of vector 17629 outside of the T-DNA. These elements were not expected to be transferred to the plant cell or integrate into the plant genome during T-DNA transfer.

Each Southern blot analysis was performed with genomic DNA extracted from MZIR098 corn and from nontransgenic, near-isogenic corn, which was used as a negative control to identify any endogenous corn DNA sequences that hybridized with the probes. To demonstrate the sensitivity of the analyses, each analysis also included two positive assay controls representing 1 copy and 1/7 copy per genome of a DNA fragment of known size in the corn genome. The positive assay controls were PCR-amplified fragments that corresponded to the two backbone-specific probes or to the T-DNA-specific probe used in characterization of the MZIR098 corn insert.

The positive assay controls for the T-DNA-specific probe and backbone-specific probes 1 and 2 were loaded in a well together with 7.5 μg of digested DNA from nontransgenic, near-isogenic NP2222/NP2391 corn, in order to more accurately reflect their migration speeds in the corn genome matrix. The positive assay control for T-DNA-specific probe was analyzed in the absence of nontransgenic corn genomic DNA, so that endogenous bands would not obscure the positive assay control.

Corn genomic DNA was analyzed via two restriction enzyme digestion strategies. In the first strategy, the genomic DNA was digested with an enzyme that cut within the MZIR098 insert and in the corn genome flanking the MZIR098 insert. This first strategy was used twice, with two different enzymes, to determine the number of vector 17629 T-DNA inserts within the MZIR098 corn genome and the presence or absence of extraneous DNA fragments of the insert in other regions of the MZIR098 corn genome. The enzymes used were HindIII and XcmI. In the second strategy, the genomic DNA was digested with restriction enzymes that cut within the insert to release DNA fragments of predictable size. This strategy was used to determine the number of copies of the T-DNA at each location within the MZIR098 corn genome, the intactness of the insert, and the presence or absence of any closely linked extraneous T-DNA fragments. The enzymes used were BmtI, HindIII, andXcmI. BmtI cleaves flanking sequences outside of the T-DNA insert. The probe used to detect the T-DNA insert comprised the full-length T-DNA (SEQ ID NO: 7).

Genomic DNA samples, at about 7.5 g/lane, were cut with restriction enzymes and run overnight on an agarose gel in 1X TBE buffer at about 32 volts. Gels were photographed, washed, and blotted onto nylon membrane with 10X SSC as the transfer solution. They were linked to the membrane with UV light and pre-hybridized with calf thymus DNA at 65° C. The probes were labeled with radioactive Phosphorus 32. Probes were added and hybridized at 65° C., 3 hrs to overnight. Blots were washed several times and exposed in a phosphorimager cassette. Images were developed and scored.

No unexpected bands were detected by blots performed using either strategy, further supporting that the MZIR098 maize event is an elite event, comprising a fully-intact, single copy T-DNA, with no extraneous DNA fragments, either from the vector 17629 backbone or from partial secondary T-DNA insertions, detected. Additionally, the Southern blot analyses demonstrated that the hybridization bands specific to the MZIR098 insert were identical in all lanes containing genomic DNA extracted from MZIR098 corn plants of generation T2 (ear 4), T2 (ear 35), T3, T4, T5, or F1. These results support the conclusion that the MZIR098 insert is stably inherited from one generation to the next and that MZIR098 corn contains a single T-DNA insert.

### Example 4: Mendelian Inheritance of the T-DNA Insert of Elite Event MZIR098

The purpose of this study was to confirm Mendelian inheritance ratios of eCry3.1Ab, mCry3A, and PAT-08 by determining their segregation ratios in three generations of MZIR098 maize backcrossed (BC) to a maize inbred. Prior to this study, hemizygous MZIR098 maize plants of the F2 generation were crossed with nontransgenic maize line NP2391. The resulting F1 generation was backcrossed to the nontransgenic recurrent parent (NP2391) to yield the BC1F1 generation. MZIR098 maize plants from the BClF1 generation were backcrossed three more times to the nontransgenic recurrent parent (NP2391) to yield the BC2F1, BC3F1, and BC4F1 generations. Positive hemizygous segregants, as determined by glufosinate herbicide resistance and real-time PCR analysis, were utilized in each backcross. Individual plants from three generations of MZIR098 maize backcrossed (BC) to a maize inbred (the BC2F1, BC3F1, and BC4F1 generations) were tested for the presence of eCry3.1Ab, mCry3A, and PAT-08 by real-time polymerase chain reaction (PCR) analysis. The results from real-time PCR analysis were used to determine the segregation ratios of eCry3.1Ab, mCry3A, and PAT-08. The expected segregation ratio for each gene was 1:1 in each generation (i.e., 50% of the plants in each generation were expected to carry the gene). Chi-square analysis of this segregation data was performed to test the hypothesis that the MZIR098 event is inherited according to Mendelian principles, which is consistent with insertion into a chromosome within the maize nuclear genome.

The expected and observed segregation ratios are shown in Table 4. The genes eCry3.1Ab, mCry3A, and PAT-09 co-segregated (i.e., when one gene was present, the other two genes were also present). The critical value for rejection of the hypothesis of segregation according to Mendelian inheritance at a=0.05 was 3.84. All of the chi-square values were less than 3.84 for each generation tested, indicating that eCry3.1Ab, mCry3A, and PAT-08 were inherited in a predictable manner, according to Mendelian principles. These results support the conclusion that the MZIR098 T-DNA insert integrated into a chromosome within the corn nuclear genome.

TABLE 4
Mendelian Inheritance of transgene of event MZIR098

| | BC₂F₁ | | BC₃F₁ | | BC₄F₁ | |
|---|---|---|---|---|---|---|
| | Ob- | Ex- | Ob- | Ex- | Ob- | Ex- |
| Trait^{a} | served | pected | served | pected | served | pected |
| Positive | 85 | 93 | 69 | 70 | 75 | 74.5 |
| Negative | 101 | 93 | 71 | 70 | 74 | 74.5 |
| Total | 186 | 186 | 140 | 140 | 149 | 149 |

| | | | |
|---|---|---|---|
| x² | 1.38* | 0.03* | 0.01* |

^{a}The observed frequencies of eCry3.1Ab, mCry3A, and PAT-08 were identical; the three genes segregated as one locus.
*P < 0.05 (x² < 3.84).

### Example 5: Analysis of Flanking DNA Sequence

Adaptor PCR was used for flanking sequence recovery, using a gene specific primer with homology to the target TDNA border sequences (as exemplified below) of vector 17629 combined with another primer with homology to the adaptor which was ligated to digested genomic DNA from a corn plant comprising event MZIR098. A primary adaptor PCR was followed by a secondary adaptor PCR (nested PCR), using a nested gene specific primer with homology to the target TDNA border sequence of vector 17629 combined with another nested primer with homology to the same adaptor.

The GenomeWalker™ Universal Kit (Clontech, Cat No.638904) was used to recover the genome sequence flanking transgene insert of the event MZIR098. Restriction digestion was completed by combing 8 μl genomic DNA (20 to 100 ng/μl), 1 μl blunt restriction enzyme (PvuII, EcoRV or StuI in separate reactions) and 1 μl digestion buffer specific to selected enzyme, followed by incubating at 37° C. overnight. Ligation of digested genomic DNA to GenomeWalker Adaptor was completed by combining the restriction digestion product with 0.475 μl GenomeWalker Adaptor, 1.1 μl 10X T4 DNA ligase buffer and 0.125ul T4 DNA ligase, followed by incubating at 16° C. overnight. The ligated products were heat-treated to inactivate the enzymes, and were used in primary adaptor PCR and secondary adaptor PCR for flanking sequence recovery. Primary PCR and secondary (nested) PCR were carried out with Sigma high fidelity enzyme (Sigma, Cat. No.D1313), using PCR parameters recommended by manufacturer for the GenomeWalker™ Universal Kit.

The 5′ and 3′ flanking sequences and junction sequences were confirmed using standard PCR procedures. The 5′ flanking and junction sequence was confirmed using a first polynucleotide primer set forth in SEQ ID NO: 14 through SEQ ID NO: 16 combined with a second polynucleotide primer of SEQ ID NO: 17 (see Table 6 below). The 3′ flanking and junction sequence were confirmed using a first polynucleotide primer set forth in SEQ ID NO: 19 through SEQ ID NO: 21 combined with a second polynucleotide primer of SEQ ID NO: 18 (see table below). It will be recognized by the skilled person that other primer sequences can be used to confirm the flanking and junction sequences.

TABLE 5
Primers used to confirm flanking and junction
sequences

| Target | | |
|---|---|---|
| Region | Primer Sequence | SEQ ID NO. |
| 5′ FS | TCCGGACGGTAGCTAGAGG | 14 |
| 5′ FS | CGTTTATTTCTCGGTCGGCG | 15 |
| 5′ FS | TTACGTCGCGGAGAGATGGAT | 16 |
| TDNA_RB | GTGAGTGGACATTTCCCAAACTACCCT | 17 |
| TDNA_LB | CAAGGCCAGTTAGGCCAGTTA | 18 |
| 3′ FS | GACATGGACATGCATGGGT | 19 |
| 3′ FS | CCACACACACACACACAAAGAGAGT | 20 |
| 3′ FS | GTGGCATCGTCTAGCGATCAAC | 21 |

Both 5′ flanking sequence and 3′ flanking sequence of the event MZIR098 were used to search maize genome databases. Identical matches to both flanking sequences were found on Chromosome 10 (MAIZE_REF_3_GENOME), and on a BAC clone, NCBI Accession No. AC204437.3. Using this information, it was determined that the heterologous DNA insertion of event MZIR098 displaced 39 nucleotides of maize genomic DNA, which lies between the 5′ flanking sequence (upstream of the deleted sequence) and the 3′ flanking sequence (downstream of the deleted sequence).

The event MZIR098 insert was found to be flanked on the right border (5′ flanking sequence) by the corn genomic sequence shown in SEQ ID NO: 8 and flanked on the left border (3′ flanking sequence) by the corn genomic sequence shown in SEQ ID NO: 9. The 5′ junction sequence is set forth in SEQ ID NO: 1 and SEQ ID NO: 3. The 3′ junction sequence is set forth in SEQ ID NO: 2 and SEQ ID NO: 4. The genomic integration site of the 17629 vector transgene in event MZIR098 is comprised within SEQ ID NO: 70 and its complement SEQ ID NO: 71. More specifically, the genomic integration site of event MZIR098 is between 18,206-18,246 of NCBI Accession No. AC204437.3 (SEQ ID NO: 70).

### Example 6: T-DNA Insert Sequencing

The nucleotide sequence of the entire transgene DNA insert present in event MZIR098 was determined to demonstrate overall integrity of the insert, contiguousness of the functional elements and to detect any individual basepair changes. The event MZIR098 insert was PCR amplified from genomic DNA derived from individual plants as overlapping fragments to cover the entire TDNA insert which is linked to its 5′ flanking sequence and 3′ flanking sequence on each side.

TABLE 6
Primer sequence combinations used for overlapping
PCR

| PCR | SEQ ID | |
|---|---|---|
| Amplicon | NO. | Sequence |
| Amplicon-A | 14 | TCCGGACGGTAGCTAGAGG |
| | 17 | GTGAGTGGACATTTCCCAAACTACCCT |
| Amplicon-B | 15 | CGTTTATTTCTCGGTCGGCG |
| | 17 | GTGAGTGGACATTTCCCAAACTACCCT |
| Amplicon-C | 16 | TTACGTCGCGGAGAGATGGAT |
| | 17 | GTGAGTGGACATTTCCCAAACTACCCT |
| Amplicon-D | 22 | GTAGGCCGCTTCCCTAATTAGC |
| | 23 | CGCTGATGCCCTTCTGGATCAC |
| Amplicon-E | 24 | GTAGTTTGGGAAATGTCCACTCACCCGT |
| | 25 | ACCGGCAACAGGATTCAATCTTAAG |
| Amplicon-F | 26 | ACCAGATCGGCCTGAAGACC |
| | 27 | CAGAAGTAGAACTACCGGGCCCTAAC |
| Amplicon-G | 28 | GGATTCCTTTCCCACCGCT |
| | 29 | GCTGGGCCAGATGGTGTTCAG |
| Amplicon-H | 30 | GCCCTGCCTTCATACGCTATTTATT |
| | 31 | ACCGGCAACAGGATTCAATCTTAAG |
| Amplicon-I | 32 | CAGCACCAGCCAGATCACCTTCA |
| | 33 | CCACAACACCCTCAACCTCAGCA |
| Amplicon-J | 34 | ACAGTGAACTTTAGGACAGAGCCACAA |
| | 35 | CACATTGCGGATACGGCC |
| Amplicon-K | 36 | GACATGGACATGCATGGGT |
| | 18 | CAAGGCCAGTTAGGCCAGTTA |
| Amplicon-L | 20 | CCACACACACACACACAAAGAGAGT |
| | 18 | CAAGGCCAGTTAGGCCAGTTA |
| Amplicon-M | 21 | GTGGCATCGTCTAGCGATCAAC |
| | 18 | CAAGGCCAGTTAGGCCAGTTA |
| Amplicon-N | 37 | GACGTAAGGGATGACGCACAATCCCA |
| | 31 | ACCGGCAACAGGATTCAATCTTAAG |

PCR amplification was carried out using high fidelity enzyme (Sigma, Cat. No.D1313) with PCR parameters adjusted for different target regions. In one example, PCR was carried out using the following parameters: 30 see at 96° C. for 1 cycle, followed by 35 cycles of 30s at 94° C., 30s at 60° C. and 3 min at 68C, followed by 1 cycle of 7 min at 68° C. In another example, PCR was carried out using the following parameters: 5 min at 95° C. for 1 cycle, followed by 35 cycles of 30s at 94° C., 30s at 59° C. and 1 to 7 min at 68° C., followed by 1 cycle of 10 min at 68° C.

PCR product obtained from the overlapping PCR amplification was treated with EXO-SAP before sequencing using the following protocol: EXO-SAP master mix was prepared by combining Exonuclease I (USB, Cat No.72073), Shrimp Alkaline Phosphatase (USB, Cat No.70092Z) and EX-SAP buffer (20 mM Tris-HCl (pH8.0), 10 mM MgCl2) at 1:1:2. To each PCR product, 1/10 volume of EXO-SAP master mix was added. The reaction was carried out for 30 min at 37° C. followed by 20 min at 80° C. to inactivate the enzymes.

Sequencing was carried out using the ABI3730XL DNA Analyzer with ABI BigDye® chemistry. The final consensus sequence was determined by combining the sequence data from different PCR amplicons to generate consensus sequence of the event MZIR098 insert (SEQ ID NO: 7) linked to its 5′ genome sequence and 3′ genome sequence at each side (SEQ ID NO: 8 and SEQ ID NO: 9). SEQ ID NO: 6 comprises the full-length T-DNA insertion with 100 bp of 5′ and 3′ flanking genomic sequences. c

The consensus sequence data for the event MZIR098 insert demonstrates that the overall integrity of the insert and contiguousness of the functional elements within the insert as intended in pSYN17629 have been maintained. The nucleotide sequence analysis thus demonstrated that the MZIR098 insert contains a single copy of each of the functional elements (eCry3.1Ab, mCry3A, PAT-08, the eNOS-02 enhancer, the prCMP-04 promoter, the prUbi1-18 promoter, the pr35S-04 promoter, and the tNOS-05-01 and tNOS-20 terminators).

### Example 7: Detection of Elite Event MZIR098 eCrv3.1Ab, mCrv3A, and PAT Proteins Via ELISA

The concentrations of eCry3.1Ab, mCry3A, and PAT in various MZIR098 corn tissues were quantified by enzyme-linked immunosorbent assay (ELISA) to establish an expression profile for these proteins as produced in MZIR098 corn. The tissues analyzed were leaves and roots at four growth stages (V6, RI, R6, and senescence), whole plants at three stages (V6, RI, and R6), kernels at two stages (R6 and senescence), and pollen (stage RI). The tissues were collected from MZIR098 corn and a nontransgenic, near-isogenic control corn grown concurrently according to local agronomic practices at four U.S. locations in 2013. The genotypes of the plants used in these studies were NP2391×NP2222(MZIR098) and NP2391×NP2222.

At each location, one plot was planted with MZIR098 corn, and one plot was planted with nontransgenic corn. Five replicate samples of each tissue type were collected from each plot for MZIR908; two replicate samples were collected for the nontransgenic control. For leaves, all the true leaves from one plant were collected per sample. For roots, the entire root ball excluding brace roots were collected per sample. For pollen, a pooled sample was collected from 10 to 15 tassels per plot. For whole plants, the entire plant including the root ball was collected per sample. For kernels, all the kernels from the primary ear of a single plant were collected per sample. All tissue samples except pollen were ground to a powder, and all samples were then lyophilized. The percent dry weight (DW) of each sample was determined from the sample fresh weight before and the sample dry weight after lyophilization.

Protein was extracted from representative aliquots of the lyophilized tissue samples at a ratio of 3 ml protein extraction buffer (PBST buffer) to approximately 30 mg of lyophilized tissue. The samples were homogenized, centrifuged, and the supernatant was collected. For pollen samples, ˜25 mg lyophilized pollen was homogenized using a KLECO homogenizer. Borate extraction buffer was then added, the samples were incubated on ice for at least 20 minutes, centrifuged, and the supernatant was collected. Samples were diluted in ELISA diluent (PBS containing 1% BSA, 0.05% Tween-20).

eCry3.1Ab was quantified using rabbit anti-G6-Cry1Ab and an alkaline phosphatase-conjugated donkey anti-rabbit immunoglobin G following standard techniques. mCry3A was quantified using the QualiPlate™ ELISA kit for Modified Cry3A using standard methods. PAT was quantified using the QualiPlate™ ELISA Kit for LibertyLink® PAT/pat. The sample extracts were analyzed for each trait protein in duplicate or triplicate, and a standard curve was generated for each ELISA plate with known amounts of the corresponding reference protein. Concurrent analysis of tissues from the nontransgenic corn confirmed the absence of plant-matrix effects on the analysis methods. All protein concentrations were adjusted for extraction efficiency.

For glufosinate-treated MZIR098 maize, the eCry3.1Ab protein concentrations ranged from below limit of detection in pollen to 45.94 μg/g fresh weight in leaves (V6 stage). The arithmetic mean expression value for eCry3.1Ab protein in kernels (senescence) is 1.42±0.56 g/g fresh weight. The mCry3A protein concentrations ranged from 0.72 μg/g fresh weight in roots (senescence) to 236.71 μg/g fresh weight in pollen. The arithmetic mean expression value for mCry3A protein in kernels (senescence) is 7.55±1.58 μg/g fresh weight. The PAT protein concentrations ranged from below limit of detection in pollen, roots and kernels (R6 and senescence), leaves (senescence), and whole plants (R6) to 1.75 g/g fresh weight in leaves (V6).

For untreated MZIR098 maize, the eCry3.1Ab protein concentrations ranged from below limit of detection in pollen to 46.68 g/g fresh weight in leaves (V6 stage). The arithmetic mean expression value for eCry3.1Ab protein in kernels (senescence) is 1.50±0.79 g/g fresh weight. The mCry3A protein concentrations ranged from 0.53 g/g fresh weight in roots (senescence) to 246.96 μg/g fresh weight in pollen. The arithmetic mean expression value for mCry3A protein in kernels (senescence) is 8.30±2.02 μg/g fresh weight. The PAT protein concentrations ranged from below limit of detection in pollen, leaves and roots and kernels (R6 and senescence), and whole plants (R6) to 1.85 g/g fresh weight in leaves (V6).

### Example 8: Insect Control Field Efficacy of Elite Event MZIR098

Field trials of 12 different transgenic events with positive and negative controls were planted in three locations. There were three replications of each event per location and each replication was planted in three 20 foot long rows, of which the center row was harvested for root expression studies and root damage ratings. For each replication, 10 plants were sampled. Field trials relied on natural populations of corn rootworm. When 80% of the larvae in the plots reach pupation, roots were dug at the V10 stage, washed to remove soil, and rated using the node injury scale of 0-3 (Oleson et al., 2005, J. Econ. Entomol. 98: 1-8). Following that analysis, about 50% of each root ball was cut into large pieces, frozen, ground and lyophilized, and 100 mg of the lyophilized root tissue was used for ELISA assays. The amounts of mCry3A and eCry3.1Ab protein present in the root samples from V6 and V8 plants from each of the 12 events were determined by ELISA as described in Example 7. Root samples from transgenic MIR604 plants (expressing mCry3A) and from transgenic 5323 (expressing eCry3.1Ab) were also analyzed by ELISA as positive controls.

The expression data and root damage ratings (RDR) were analyzed by one-way analysis of variance (ANOVA) using the SAS JMP statistical package (JMPSAS Institute 2010). The Student's pairwise t test was used for mean separation to distinguish treatment differences. Results from all the tests were considered statistically significant at P<0.05. Results for both the mCry3A and eCry3.1Ab protein levels in V6 and V8 roots and the CRW RDR are shown in Table 7 below.

TABLE 7
Average root protein levels and RDR in field trials

| | | mCry3A | | eCry3.1Ab | | |
|---|---|---|---|---|---|---|
| Event | n | V6 | V8 | V6 | V8 | RDR* |
| 5323 | 30 | n/a | 85.0 H-K | 516.8 C-E | 296.8 B-D | 0.04 B |
| 5323 × | 30 | 294.5 D-F | 195.5 F-H | 470.2 D-F | 257.8 C-E | 0.04 B |
| MIR604 | | | | | | |
| MIR604 | 30 | 278.1 EF | 197.6 FG | n/a | 25.0 H | 0.10 B |
| MZIR098 | 30 | 352.5 C | 253.4 DE | 473.1 D-F | 294.8 B-D | 0.03 B |
| MZIR0A6 | 30 | 312.2 C-E | 310.5 BC | 539.9 CD | 364.1 A | 0.02 B |
| MZIR071 | 30 | 277.9 EF | 194.3 F-I | 679.4 A | 313.1 A-C | 0.03 B |
| MZIR07N | 30 | 258.1 F | 114.6 J | 661.1 AB | 163.7 G | 0.02 B |
| MIZR08A | 30 | 271.4 EF | 168.9 F-J | 555.3 CD | 299.7 B-D | 0.03 B |
| MZIR08G | 30 | 114.2 G | 42.4 K | 330.3 G | 53.1 H | 0.03 B |
| MZIR092 | 30 | 249.9 F | 152.7 G-J | 735.4 A | 295.6 B-D | 0.10 B |
| MZIR09W | 30 | 294.9 D-F | 203.7 F | 490.2 C-F | 231.2 E-G | 0.03 B |
| MZIR0AN | 30 | 259.9 F | 220.2 EF | 491.9 C-F | 238.8 D-F | 0.02 B |
| MZIR0B5 | 30 | 330.2 CD | 276.5 CD | 411.6 FG | 353.8 AB | 0.03 B |
| MZIR0AD | n/a | 279.0 EF | 146.0 IJ | 554.0 CD | 195.2 FG | n/a |
| MZIR0BC | 30 | 292.2 D-F | 203.3 F | 571.8 BC | 301.3 B-D | 0.03 B |
| Non Bt | 30 | 0.0 H | 0.0 K | 0.0 H | 0.0 H | 1.74 A |
| Control | | | | | | |

*Average root damage ratings (RDR)
Means followed by the same letter within the same column are not statistically different among events (P < 0.05). Means were separated using Student's t test

### Example 9: Zygosity Determination of Elite Event MZIR098 by End-Point TaqMan PCR

This protocol describes a procedure for determination of zygosity status of event MZIR098 present in individual plants comprising elite event MZIR098. This method uses duplex end-point TaqMan PCR, where one reaction is specific for the event MZIR098 insertion and other is specific for the corresponding wild type allele sequence where the event MZIR098 transgenic DNA integrated. This wild type allele sequence may be referred to as the native insertion site. Because event MZIR098′ is introgressed into a large number of corn germplasms, a primer/probe set for the native insertion site which is suitable for as many varieties as possible needed to be identified.

DNA samples from biological samples are prepared using methods known in the art. Primers/probe master mixes are prepared as 50× stocks, with primers at a concentration of 15 μM and the probe at a concentration of 5 μM. The following table (Table 8) indicates each reagent and amount for a 10 μL reaction:

TABLE 8
Zygosity Determination by End-Point TaqMan PCR

| Component | Volume Per Reaction | |
|---|---|---|
| DNA sample (10-20 ng DNA/μL) | 3.0 | μL |
| JumpStart ™ ReadyMix ™ (2×)¹ | 5.0 | μL |
| 50× primers/probe master mix for MZIR098² | 0.2 | μL |
| 50× primers/probe master mix for wild type allele² | 0.2 | μL |
| Nuclease-free water | 1.6 | μL |
| Total Volume | 10.0 | μL |

¹with MgCl₂ and Sulforhodamine 101 added
²Final concentration 300 nM (each primer) and 100 nM (each probe)

For Table 8, the sequences of the primers used for detection of event MZIR098 were 5′-ATCTCAGACACCAAACCGAGATC-3′ (SEQ ID NO: 10) and 5′-ACACCGTTAGGCTAGTGCCAGT-3′ (SEQ ID NO: 11). The sequence of the MZIR098 probe was 5′-CAAGTGACAGCGAACGGAGCTGGTTT-3′ (SEQ ID NO: 12). For the native insertion site, the primer/probe set is designed against genomic sequences flanking the T-DNA insert. Alternatively, the primer/probe set for the native insertion site may include multiple primers and multiple probes. This design covers more varieties than a single primer-probe assay due to sequence differences amongst different corn varieties. In this example, two forward primers (5′-ACCAAACCGAGATCCAAGTGA-3′; SEQ ID NO: 65 and 5′-GCGCGTCGACCTGCAC-3′, SEQ ID NO: 66), one reverse primer (5′-GCATGGTTCCTTGTCGGC-3′; SEQ ID NO: 67), and two native insertion site probes (5′ CTAGTTGTACCTGCCCCCGCCTG-3′; SEQ ID NO: 68 and 5′ AACGGAGCTGCCCCCGCC-3′; SEQ ID NO: 69) were used.

PCR was performed in the ABI 7900HT instrument using the following amplification parameters: 10 min at 95° C., followed by 40 cycles of 15 s at 95° C. and 1 min at 60° C. The PCR may be run on an ABI Prism 7700, a GeneAmp PCR system 9700, or any other appropriate system. The data was analyzed using the SDS software on the ABI 7900HT. Data analysis includes making calls (Hom, Het, Null) based on cluster positions, as shown in FIG. 2. Results indicate if the biological sample was taken from a corn plant which is “homozygous”, or contains one copy of the MZIR098 event on each Chromosome 10, for a total of two copies of the MZIR098 event; “heterozygous”, where the plant comprises event MZIR098 in one Chromosome 10 but the second Chromosome 10 does not have the event; or “null”, where event MZIR098 is not present on either Chromosome 10 of the corn plant evaluated. Determining how many copies of event MZIR098 are present in a maize plant is useful for breeding purposes, so that the segregation of event MZIR908 in the resulting progeny of a cross may be determined.

### Example 10: Use of Elite Event MZIR098 Insertion Site for Targeted Integration in Maize

The elite event MZIR098 flanking sequences are disclosed in SEQ ID NO: 8 (5′ flanking sequence) and SEQ ID NO: 9 (3′ flanking sequence) and were used to search maize genome databases. Matches to both flanking sequences were found on a BAC clone of chromosome 10, CH201-411G1 (NCBI Accession No. AC204437.3; SEQ ID NO: 70). Using this information, it was determined that the elite event MZIR098 insertion is in the corn genome on chromosome 10 between nucleotides 18,206-18,246 of NCBI Accession No. AC204437.3 (SEQ ID NO: 70). Additionally, the flanking sequences were used to determine the physical position of the MZIR098 insertion site on the publicly available maize reference assembly of Maize B73, using Maize B73 Reference RefGen_v4 (AGPv4, August 2016). This reference assembly was created by the Arizona Genomics Institute. The assembly was for corn_v4 in Chado, and chromosome pseudo-assemblies of component BAC clones were guided by the physical map, also known as the maize accessioned golden path (AGP). SEQ ID NO: 8 (5′ flanking) aligns to physical position 145,988,054-145,988,634 on chromosome 10, and the SEQ ID NO: 9 (3′ flanking) aligns to 145,988,674-145,988,730 and 145,989,526-145,990,048 on chromosome 10. Fragmentation of SEQ ID NO: 9 may be due to genomic differences in corn varieties (the original event MZIR098 was into NP2222, and this alignment compares to variety B73) or to errors in the genomic sequencing or assembly of the current assembly of the B73 genome. Regardless, the MZIR098 insertion is at about 145,988,634-145,988,674 on chromosome 10 of B73, which is a relevant reference point for any corn variety genome. This location can be found on-line at the Maize Genetics and Genomics Database (maizegdb.org), using the Maize B73 RefGen_v4 (AGPv4, August 2016) data source.

Using publicly available data generated using the 50k SNP Illumina Infinium chip (Illumina, San Diego, California), molecular markers flanking the MZIR098 insertion site were identified. The 50k SNP Illumina Infinium chip is an Illumina BeadChip array of 56,110 maize SNPs developed from B73 genes and initially validated on a variety of germplasm (Americas, Europe, and wild relatives; Ganal et al 2011, Plos One 6(12): e28334. doi:10.1371). Approximately 50% (28,156) of these SNP markers have been mapped onto the very high resolution IBM and LHRF mapping panels (Ganal et al 2011). Using the public Illumina Infinium maize 50k SNP chip marker name, the MZIR098 insertion site is between markers SYN23814 and PZE110102022. More broadly, the MZIR098 insertion is between marker group These markers are described with respect to the positions of marker loci in the genome of the maize B73 variety (RefGen_v4, AGPv4, August 2016) at the Maize Genetics and Genomics Database internet resource (maizegdb.org).

Consistent agronomic performance of the corn elite event MZIR098 over several generations under field conditions suggests that these identified genomic regions around the corn elite event MZIR098 insertion site provide good genomic locations for the targeted integration of other transgenic genes of interest. Such targeted integration overcomes the problems with so-called “positions effects,” where a transgene may insert into a transcriptionally silent region of the genome, or may disrupt the function or expression pattern of a native gene by insertion either into or in proximity to a native gene. Further advantages of such targeted integration include, but are not limited to, reducing the extremely large amount of resources required for the screening and testing of thousands of randomly inserted transgenic events before obtaining a transgenic plant that exhibits the desired level of transgene expression without also exhibiting abnormalities resulting from the inadvertent insertion of the transgene into an important locus in the host genome. Moreover, such targeted integration allows for stacking transgenes at a single genomic location, rendering introgression of the stacked traits into desirable germplasm significantly more efficient.

Using the above disclosed teaching, the skilled person is able to use methods well-known in the art to target transgenes to the same genomic insertion site as that of corn elite event MZIR098 or to a site in close proximity to the insertion site of corn elite event MZIR098. Site specific nucleases, including for example Zinc Finger Nucleases (ZFNs), meganucleases, Transcription Activator-Like Effector Nucelases (TALENS) and Clustered Regularly Interspaced Short Palindromic Repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, such as the genomic insertion site of elite event MZIR098, are well-known in the art (see reviews by Bortesi and Fischer, 2015, Biotechnology Advances 33: 41-52; and by Chen and Gao, 2014, Plant Cell Rep 33: 575-583, and references within). Patent publication WO2016106121 (hereby incorporated by reference in its entirety) exemplifies using the insertion site of the corn event MIR604 for targeted insertion. This application is incorporated in its entirety herein. The MIR604 insertion site in untransformed corn is used as a site for targeted insertion for some examples. The MIR604 event is also used as a site for targeted insertion, to add additional transgenes. The disclosure of the present application, as well as the teachings known in the art and taught, for example, in the WO2016106121 publication provide sufficient disclosure for a position of ordinary skill in the art to perform targeted insertion at the genomic insertion site of elite event MZIR098, either the genomic location in the absence of the event, or the genomic location of the event itself with targeted integration into part of the MZIR098 transgene or proximal to the MZIR098 transgene.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the invention.

### DEPOSIT

Applicants have made a deposit of corn seed of elite event MZIR098 disclosed above on May 1, 2017 in accordance with the Budapest Treaty at the American Type Culture Collection (ATCC), 10801 University Boulevard, Manassas, VA 20110-2209 under ATCC Accession No. PTA-124143. The seed was tested on May 12, 2017 and found to be viable. The deposit will be maintained in the depositary for a period of 30 years, or 5 years after the last request, or the effective life of the patent, whichever is longer, and will be replaced as necessary during that period. Applicants impose no restrictions on the availability of the deposited material from the ATCC; however, applicants have no authority to waive any restrictions imposed by law on the transfer of biological material or its transportation in commerce.

All publications and published patent documents cited in this specification are incorporated herein by reference to the same extent as if each individual publication or patent document was specifically and individually indicated to be incorporated by reference.

1-42. (canceled)

43. A method of producing hybrid corn seed comprising corn elite event MZIR098, comprising the following steps:
a) planting seed of a first inbred corn plant, an example of said seed deposited as ATCC accession No. PTA-124143, and seed of a second inbred corn plant having a genotype different than the first inbred corn plant;
b) cultivating said first and second corn plants until the production of flowers;
c) emasculating said flowers produced at the time of flowing of either the first or the second inbred corn line;
d) pollinating the non-emasculated plant with pollen of the emasculated inbred plant results in hybrid corn; and
e) harvesting the hybrid corn seeds produced.

44. Hybrid corn seed produced by the method of claim 43.

45. Hybrid corn plants produced by growing hybrid corn seed of claim 44.

46-92. (canceled)

93. A method of producing hybrid corn seed comprising a nucleic acid molecule comprising the nucleotide sequence of SEQ ID NO: 5 or 6, comprising the following steps:
a) planting seed of a first inbred corn plant, comprising a nucleic acid molecule comprising the nucleotide sequence of SEQ ID NO: 5 or 6, and seed of a second inbred corn plant having a genotype different than the first inbred corn plant;
b) cultivating said first and second corn plants until the production of flowers;
c) emasculating said flowers produced at the time of flowing of either the first or the second inbred corn line;
d) pollinating the non-emasculated plant with pollen of the emasculated inbred plant results in hybrid corn; and
e) harvesting the hybrid corn seeds produced.

94. Hybrid corn seed produced by the method of claim 93.

95. Hybrid corn plants produced by growing hybrid corn seed of claim 94.


SEQUENCE LISTING (71 sequences)

SEQ ID NO: 1 - DNA, 20 bp, synthetic construct
  misc_feature 1..20: note = Zea mays, Agrobacterium tumefaciens
  source 1..20: mol_type = other DNA
aacggagctggtttaaactg

SEQ ID NO: 2 - DNA, 20 bp, synthetic construct
  misc_feature 1..20: note = Zea mays, Agrobacterium tumefaciens
  source 1..20: mol_type = other DNA
caccacaataccaggcccag

SEQ ID NO: 3 - DNA, 120 bp, synthetic construct
  misc_feature 1..120: note = Zea mays, Agrobacterium tumefaciens
  source 1..120: mol_type = other DNA
gcaacaaaatatacgtgtgtgcgtcgagctgcatattctagttgtaccggcacggcagtcgaacgatctcagacaccaaaccgagatccaagtgacagcgaacggagctggtttaaactg

SEQ ID NO: 4 - DNA, 120 bp, synthetic construct
  misc_feature 1..120: note = Zea mays, Agrobacterium tumefaciens
  source 1..120: mol_type = other DNA
caccacaataccaggcccaggcgcgaccggccgcagcgcgccgacaaggaaccatgccggttgcctaactaacggctgactcgctcgatcgagcggagcatgcggacactgcagcaggtt

SEQ ID NO: 5 - DNA, 8487 bp, synthetic construct
  misc_feature 1..8487: note = Derived from Zea mays, Agrobacterium tumefaciens, Cestrum Yellow Leaf Curl Virus, Bacillus thuringiensis, Cauliflower Mosaic Virus, and Streptomyces viridochromogenes
  source 1..8487: mol_type = other DNA
aacggagctggtttaaactggcactagcctaacggtgttgactaagtaggccgcttccctaattagctaagggacccgggtcattgagcggagaattaagggagtcacgttatgacccccgccgatgacgcgggacaagccgttttacgtttggaactgacagaaccgcaacgaatattggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggacccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgaggggtaccagctcttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattgacggacccggcgcgccatttaaatggtaccggtccggcatgcatgcagggatccacatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctgaatagtgatatcggcgcctgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgtagccctgcaggaaatttaccggtgcccgggcggccagcatggccgtatccgcaatgtgttattaagttgtctaagcgtcaatttgtttacaccacaataccaggcccag

SEQ ID NO: 6 - DNA, 8687 bp, synthetic construct
  misc_feature 1..8687: note = Derived from Zea mays, Agrobacterium tumefaciens, Cestrum Yellow Leaf Curl Virus, Bacillus thuringiensis, Cauliflower Mosaic Virus, and Streptomyces viridochromogenes
  source 1..8687: mol_type = other DNA
gcaacaaaatatacgtgtgtgcgtcgagctgcatattctagttgtaccggcacggcagtcgaacgatctcagacaccaaaccgagatccaagtgacagcgaacggagctggtttaaactggcactagcctaacggtgttgactaagtaggccgcttccctaattagctaagggacccgggtcattgagcggagaattaagggagtcacgttatgacccccgccgatgacgcgggacaagccgttttacgtttggaactgacagaaccgcaacgaatattggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggacccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgaggggtaccagctcttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattgacggacccggcgcgccatttaaatggtaccggtccggcatgcatgcagggatccacatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctgaatagtgatatcggcgcctgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgtagccctgcaggaaatttaccggtgcccgggcggccagcatggccgtatccgcaatgtgttattaagttgtctaagcgtcaatttgtttacaccacaataccaggcccaggcgcgaccggccgcagcgcgccgacaaggaaccatgccggttgcctaactaacggctgactcgctcgatcgagcggagcatgcggacactgcagcaggtt

SEQ ID NO: 7 - DNA, 8467 bp, synthetic construct
  misc_feature 1..8467: note = Derived from Zea mays, Agrobacterium tumefaciens, Cestrum Yellow Leaf Curl Virus, Bacillus thuringiensis, Cauliflower Mosaic Virus, Streptomyces viridochromogenes
  source 1..8467: mol_type = other DNA
gtttaaactggcactagcctaacggtgttgactaagtaggccgcttccctaattagctaagggacccgggtcattgagcggagaattaagggagtcacgttatgacccccgccgatgacgcgggacaagccgttttacgtttggaactgacagaaccgcaacgaatattggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggacccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgaggggtaccagctcttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattgacggacccggcgcgccatttaaatggtaccggtccggcatgcatgcagggatccacatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctgaatagtgatatcggcgcctgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgtagccctgcaggaaatttaccggtgcccgggcggccagcatggccgtatccgcaatgtgttattaagttgtctaagcgtcaatttgtttacaccacaata

SEQ ID NO: 8 - DNA, 1076 bp, Zea mays
  source 1..1076: mol_type = genomic DNA
gtacgtttggtgcacgtaccgatttggccaaccccccacaaccaaccgccgttgcgttgcgtccatcggcctcgctcgcaccggcaccgccgacatgaggtgggcgggcgggcggctccaggtccaggtggcgtcgtcggggtgatgcgcgccgacgtggagacgtggccccttccttcttctacgcaaaatcccccacccacccacccacccccatccatccacacctcctcttcctaatccggacgcgggacgtcaatttgcttgccagcagcgacggcatcggccgcccgccttgtatctaatctaaccccgtaatcattcacgctaacgacgcccgtggcggggttagcgtaggcagcggcccggctggctgtggctgtctctgtctgcttggaagttggaccacggtgcggcgcgtaggggcccggccagctggtgctgtcgtctgcccgctcgcgtggtcgcgtttatttctcggtcggcgcgggcaagcgctcctctaccgggtccggatccggacggtagctagagggcgcagttgcaggtcggtggccgagggcgggcgagtcgtttacgtcgcggagagatggatcggagttgggtgtgcaggtaggtaggtgtgcgcccgccccagcagacttcagatatctgggatgacgtggggagttctgttgggtccgtccggcccgccgtacgtagtacgccagctcgctgcttgtctgtactagagtgaccagccgagctcgcgacgggtactggatgccggtccgtaccggtggtaaacagcgcatgcagtgcagctagctagatccagtactagtatttagctatatattcgtcgtaccagatcagaggcagagctggtccccgcctccccggacaggtaaactgcttccatctgtttccatcggaggcggccctggcgtggcagtggcactggctctggcagcctcgccatgcaacgcaacaaaatatacgtgtgtgcgtcgagctgcatattctagttgtaccggcacggcagtcgaacgatctcagacaccaaaccgagatccaagtgacagcgaacggagctg

SEQ ID NO: 9 - DNA, 1075 bp, Zea mays
  source 1..1075: mol_type = genomic DNA
ccaggcccaggcgcgaccggccgcagcgcgccgacaaggaaccatgccggttgcctaactaacggctgactcgctcgatcgagcggagcatgcggacactgcagcaggttgatagttccaaggagcaatagctgagctagccaagcctagctgtacgatgtgttgacacccatgcatgtccatgtccatggggccggcctgtactgcgagtgcgcgacgatgctcctcagctgcttgcccgccctcaggggcacgacgttatcagtagcctcggtccgtgaagcagggaaacgcgagcccttccacggctacgacctcaactccgatccgaataccacacaccgatcgcaacaacacatgttttccgttgatcgctagacgatgccaccgcccaggttatcttcagcttcgattccatgtgtctgcgctgcatgatgaaaaggttggggggcctgcccgcctggatccgtttccgtgcatgattagatgcagtgcagtgcaggccctgaaacttgctgctttcttctcgcaattcgtcacaggcacaggcggccggccggccgcagtccgcttgtcgccttgcccagcagagatgccacgggctgaccggcgcggggatggggaaggggccggacgggggaacgggcccccgatgacaagcctcctgcagtcctgtcgatatgatgtggagcaaaccaggacaggcgccagcagatgagcgcagagtgggcactctctttgtgtgtgtgtgtgtggtcgtggcatcttgccgtcttgcatggatccatcatggtcggtggctggaccgtcggagggcaccgcaccatagatagaggagcattgcattcacggtatcacagaagaggctgcccaggccggtggtaaagctaggtgacgaggaaaatgttgcatggacaagtgctgcagacatagagtcgtcctcttacactcggcactttcggtgcaggtgctgaggtgttgacccggcgagaacgagcgagcgagggccctcccccgacgcgtgcaagcgatcgagaaataaacctgaatcgagaacggttggtgctgctggtcg

SEQ ID NO: 10 - DNA, 23 bp, Zea mays
  source 1..23: mol_type = other DNA
atctcagacaccaaaccgagatc

SEQ ID NO: 11 - DNA, 22 bp, Agrobacterium tumefaciens
  source 1..22: mol_type = other DNA
acaccgttaggctagtgccagt

SEQ ID NO: 12 - DNA, 26 bp, synthetic construct
  misc_feature 1..26: note = Derived from Zea mays, Agrobacterium tumefaciens
  source 1..26: mol_type = other DNA
caagtgacagcgaacggagctggttt

SEQ ID NO: 13 - DNA, 73 bp, synthetic construct
  misc_feature 1..73: note = Derived from Zea mays, Agrobacterium tumefaciens
  source 1..73: mol_type = other DNA
atctcagacaccaaaccgagatccaagtgacagcgaacggagctggtttaaactggcactagcctaacggtgt

SEQ ID NO: 14 - DNA, 19 bp, Zea mays
  source 1..19: mol_type = other DNA
tccggacggtagctagagg

SEQ ID NO: 15 - DNA, 20 bp, Zea mays
  source 1..20: mol_type = other DNA
cgtttatttctcggtcggcg

SEQ ID NO: 16 - DNA, 21 bp, Zea mays
  source 1..21: mol_type = other DNA
ttacgtcgcggagagatggat

SEQ ID NO: 17 - DNA, 27 bp, synthetic construct
  misc_feature 1..27: note = Derived from Cestrum Yellow Leaf Curl Virus
  source 1..27: mol_type = other DNA
gtgagtggacatttcccaaactaccct

SEQ ID NO: 18 - DNA, 21 bp, synthetic construct
  misc_feature 1..21: note = Derived from Streptomyces viridochromogenes
  source 1..21: mol_type = other DNA
caaggccagttaggccagtta

SEQ ID NO: 19 - DNA, 19 bp, Zea mays
  source 1..19: mol_type = other DNA
gacatggacatgcatgggt

SEQ ID NO: 20 - DNA, 25 bp, Zea mays
  source 1..25: mol_type = other DNA
ccacacacacacacacaaagagagt

SEQ ID NO: 21 - DNA, 22 bp, Zea mays
  source 1..22: mol_type = other DNA
gtggcatcgtctagcgatcaac

SEQ ID NO: 22 - DNA, 22 bp, synthetic construct
  misc_feature 1..22: note = Derived from Agrobacterium tumefaciens
  source 1..22: mol_type = other DNA
gtaggccgcttccctaattagc

SEQ ID NO: 23 - DNA, 22 bp, synthetic construct
  misc_feature 1..22: note = Derived from Bacillus thuringiensis
  source 1..22: mol_type = other DNA
cgctgatgcccttctggatcac

SEQ ID NO: 24 - DNA, 28 bp, synthetic construct
  misc_feature 1..28: note = Derived from Cestrum Yellow Leaf Curl Virus
  source 1..28: mol_type = other DNA
gtagtttgggaaatgtccactcacccgt

SEQ ID NO: 25 - DNA, 25 bp, synthetic construct
  misc_feature 1..25: note = Derived from Agrobacterium tumefaciens
  source 1..25: mol_type = other DNA
accggcaacaggattcaatcttaag

SEQ ID NO: 26 - DNA, 20 bp, synthetic construct
  misc_feature 1..20: note = Derived from Bacillus thuringiensis
  source 1..20: mol_type = other DNA
accagatcggcctgaagacc

SEQ ID NO: 27 - DNA, 26 bp, synthetic construct
  misc_feature 1..26: note = Derived from Zea mays
  source 1..26: mol_type = other DNA
cagaagtagaactaccgggccctaac

SEQ ID NO: 28 - DNA, 19 bp, synthetic construct
  misc_feature 1..19: note = Derived from Zea mays
  source 1..19: mol_type = other DNA
ggattcctttcccaccgct

SEQ ID NO: 29 - DNA, 21 bp, synthetic construct
  misc_feature 1..21: note = Derived from Bacillus thuringiensis
  source 1..21: mol_type = other DNA
gctgggccagatggtgttcag

SEQ ID NO: 30 - DNA, 25 bp, synthetic construct
  misc_feature 1..25: note = Derived from Zea mays
  source 1..25: mol_type = other DNA
gccctgccttcatacgctatttatt

SEQ ID NO: 31 - DNA, 25 bp, synthetic construct
  misc_feature 1..25: note = Derived from Agrobacterium tumefaciens
  source 1..25: mol_type = other DNA
accggcaacaggattcaatcttaag

SEQ ID NO: 32 - DNA, 23 bp, synthetic construct
  misc_feature 1..23: note = Derived from Bacillus thuringiensis
  source 1..23: mol_type = other DNA
cagcaccagccagatcaccttca

SEQ ID NO: 33 - DNA, 23 bp, synthetic construct
  misc_feature 1..23: note = Derived from Streptomyces viridochromogenes
  source 1..23: mol_type = other DNA
ccacaacaccctcaacctcagca

SEQ ID NO: 34 - DNA, 27 bp, synthetic construct
  misc_feature 1..27: note = Derived from Streptomyces viridochromogenes
  source 1..27: mol_type = other DNA
acagtgaactttaggacagagccacaa

SEQ ID NO: 35 - DNA, 18 bp, synthetic construct
  misc_feature 1..18: note = Derived from Agrobacterium tumefaciens
  source 1..18: mol_type = other DNA
cacattgcggatacggcc

SEQ ID NO: 36 - DNA, 19 bp, Zea mays
  source 1..19: mol_type = other DNA
gacatggacatgcatgggt

SEQ ID NO: 37 - DNA, 26 bp, synthetic construct
  misc_feature 1..26: note = Derived from Cauliflower Mosaic Virus
  source 1..26: mol_type = other DNA
gacgtaagggatgacgcacaatccca

SEQ ID NO: 38 - DNA, 20 bp, Zea mays
  source 1..20: mol_type = other DNA
ctggcgtggcagtggcactg

SEQ ID NO: 39 - DNA, 21 bp, synthetic construct
  misc_feature 1..21: note = Derived from Bacillus thuringiensis
  source 1..21: mol_type = other DNA
tgtcggccgtcatggtggatc

SEQ ID NO: 40 - DNA, 23 bp, synthetic construct
  misc_feature 1..23: note = Derived from Zea mays
  source 1..23: mol_type = other DNA
tctttggggaatcctgggatggc

SEQ ID NO: 41 - DNA, 20 bp, Zea mays
  source 1..20: mol_type = other DNA
catggttccttgtcggcgcg

SEQ ID NO: 42 - DNA, 29 bp, synthetic construct
  misc_feature 1..29: note = Derived from Agrobacterium tumefaciens
  source 1..29: mol_type = other DNA
gtacgtttggtgcacgtaccgatttggcc

SEQ ID NO: 43 - DNA, 28 bp, synthetic construct
  misc_feature 1..28: note = Derived from Agrobacterium tumefaciens
  source 1..28: mol_type = other DNA
cgtgactcccttaattctccgctcaatg

SEQ ID NO: 44 - DNA, 30 bp, synthetic construct
  misc_feature 1..30: note = Derived from Agrobacterium tumefaciens
  source 1..30: mol_type = other DNA
tgagatgggtttttatgattagagtcccgc

SEQ ID NO: 45 - DNA, 21 bp, Zea mays
  source 1..21: mol_type = other DNA
cgaccagcagcaccaaccgtt

SEQ ID NO: 46 - DNA, 19 bp, synthetic construct
  misc_feature 1..19: note = Derived from Agrobacterium tumefaciens
  source 1..19: mol_type = other DNA
gggggtcataacgtgactc

SEQ ID NO: 47 - DNA, 24 bp, synthetic construct
  misc_feature 1..24: note = Derived from Agrobacterium tumefaciens
  source 1..24: mol_type = other DNA
gggggtcataacgtgactccctta

SEQ ID NO: 48 - DNA, 20 bp, synthetic construct
  misc_feature 1..20: note = Derived from Agrobacterium tumefaciens
  source 1..20: mol_type = other DNA
ctcccttaattctccgctca

SEQ ID NO: 49 - DNA, 19 bp, synthetic construct
  misc_feature 1..19: note = Derived from Agrobacterium tumefaciens
  source 1..19: mol_type = other DNA
aattctccgctcaatgacc

SEQ ID NO: 50 - DNA, 25 bp, synthetic construct
  misc_feature 1..25: note = Derived from Streptomyces viridochromogenes
  source 1..25: mol_type = other DNA
acccagatctgaatagtgatatcgg

SEQ ID NO: 51 - DNA, 22 bp, synthetic construct
  misc_feature 1..22: note = Derived from Agrobacterium tumefaciens
  source 1..22: mol_type = other DNA
ccgcaattatacatttaatacg

SEQ ID NO: 52 - DNA, 20 bp, synthetic construct
  misc_feature 1..20: note = Derived from Agrobacterium tumefaciens
  source 1..20: mol_type = other DNA
gccctgcaggaaatttaccg

SEQ ID NO: 53 - DNA, 93 bp, synthetic construct
  misc_feature 1..93: note = Derived from Agrobacterium tumefaciens
  source 1..93: mol_type = other DNA
tcattgagcggagaattaagggagtcacgttatgacccccgccgatgacgcgggacaagccgttttacgtttggaactgacagaaccgcaacg

SEQ ID NO: 54 - DNA, 397 bp, synthetic construct
  misc_feature 1..397: note = Derived from Cestrum Yellow Leaf Curl Virus
  source 1..397: mol_type = other DNA
tggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagt

SEQ ID NO: 55 - DNA, 1962 bp, synthetic construct
  misc_feature 1..1962: note = Derived from Bacillus thuringiensis
  source 1..1962: mol_type = other DNA
atgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtag

SEQ ID NO: 56 - DNA, 253 bp, synthetic construct
  misc_feature 1..253: note = Derived from Agrobacterium tumefaciens
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 57 - DNA, 1993 bp, synthetic construct
  misc_feature 1..1993: note = Derived from Zea mays
  source 1..1993: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 58 - DNA, 1797 bp, synthetic construct
  misc_feature 1..1797: note = Derived from Bacillus thuringiensis
  source 1..1797: mol_type = other DNA
atgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactag

SEQ ID NO: 59 - DNA, 277 bp, synthetic construct
  misc_feature 1..277: note = Derived from Agrobacterium tumefaciens
  source 1..277: mol_type = other DNA
ttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattga

SEQ ID NO: 60 - DNA, 521 bp, synthetic construct
  misc_feature 1..521: note = Derived from Cauliflower Mosaic Virus
  source 1..521: mol_type = other DNA
agtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 61 - DNA, 552 bp, synthetic construct
  misc_feature 1..552: note = Derived from Streptomyces viridochromogenes
  source 1..552: mol_type = other DNA
atgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctga

SEQ ID NO: 62 - DNA, 2740 bp, synthetic construct
  misc_feature 1..2740: note = Derived from Cestrum Yellow Leaf Curl Virus, Bacillus thuringiensis, and Agrobacterium tumefaciens
  source 1..2740: mol_type = other DNA
tcattgagcggagaattaagggagtcacgttatgacccccgccgatgacgcgggacaagccgttttacgtttggaactgacagaaccgcaacgaatattggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 63 - DNA, 4095 bp, synthetic construct
  misc_feature 1..4095: note = Derived from Zea mays, Bacillus thuringiensis, and Agrobacterium tumefaciens
  source 1..4095: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgaggggtaccagctcttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattga

SEQ ID NO: 64 - DNA, 1381 bp, synthetic construct
  misc_feature 1..1381: note = Derived from Cauliflower Mosaic Virus, Streptomyces viridochromogenes, and Agrobacterium tumefaciens
  source 1..1381: mol_type = other DNA
agtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctgaatagtgatatcggcgcctgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 65 - DNA, 21 bp, Zea mays
  source 1..21: mol_type = other DNA
accaaaccgagatccaagtga

SEQ ID NO: 66 - DNA, 16 bp, Zea mays
  source 1..16: mol_type = other DNA
gcgcgtcgacctgcac

SEQ ID NO: 67 - DNA, 18 bp, Zea mays
  source 1..18: mol_type = other DNA
gcatggttccttgtcggc

SEQ ID NO: 68 - DNA, 23 bp, Zea mays
  source 1..23: mol_type = other DNA
ctagttgtacctgcccccgcctg

SEQ ID NO: 69 - DNA, 18 bp, Zea mays
  source 1..18: mol_type = other DNA
aacggagctgcccccgcc

SEQ ID NO: 70 - DNA, 172841 bp, Zea mays
  variation 1424..1523: note = n is a, c, g, or t
  variation 8718..8817: note = n is a, c, g, or t
  variation 9815..9914: note = n is a, c, g, or t
  variation 11286..11385: note = n is a, c, g, or t
  variation 35128..35227: note = n is a, c, g, or t
  variation 89878..89977: note = n is a, c, g, or t
  variation 105187..105286: note = n is a, c, g, or t
  variation 108813..108912: note = n is a, c, g, or t
  variation 119229..119328: note = n is a, c, g, or t
  variation 132486..132585: note = n is a, c, g, or t
  variation 134025..134124: note = n is a, c, g, or t
  variation 144520..144619: note = n is a, c, g, or t
  variation 147090..147189: note = n is a, c, g, or t
  variation 151745..151844: note = n is a, c, g, or t
  variation 169545..169644: note = n is a, c, g, or t
  variation 170871..170970: note = n is a, c, g, or t
  source 1..172841: mol_type = genomic DNA
ctctcatgctatggaattggcaggccgttgggcatgcgcttaaagatatggagactttatatcatggggaacaagtgccaagtataacggatcataagagtttatattcactcaggaaggatctcaaccttaggcacgccgttggttggagcttattaagggataatgatttggagattcactatcactgggcaaggcaaatttggttgcagaagcctttgagtcgaaaggagcatgttcattcagctattgttgcccagctacccgatgagattgttgaggatttcaggagacttaacctggggatagatgctcacgcttaaggagtcattattgatgtggaacctaccttggagcaagaaaatccacagaacgacagattggtgatgccaagatacaagagatcaaggatcttattacgaaaggtagaggtccggaatttacggaagatgaacaagacacaatatggttcaagggcaggatatgtgttcctggtattgaaagccttcgtaagactattctaaaggaagcccataacttggattattctattcatcctggtagtaccaagatgtatcaggaattgaagcagaaatactggtggtatggattgaagagagatgtggctgcacatgtggctatgtgcgatgggtgtcaaagagttaaggcctaacaccaaagaccagctggactattacatccactgaagatacccaagtggaaggggaagaaattggtaaggacttcattactggattgcctcgcaccccgtgtcacacccggctttaaggaacaaagccaggtgcatctcatacatgcgccaagaagacaacatatataataacagagtgtatagagataaatgtcgtaaaacatcagagtatttattacatagcggaagacttattaaaaaaataaaagataaaagtaaaacgaactaaggatcgtcggcgccaatgtcaactgagaaacgccacctagatcagatcatactcctcgccttgtggctcctcctgaaccacctgctcttctcctgtggggggggtgtgagacagcaagggtgagctcacacatgatcatcgctcaacaagttgtggggaataatgtgacatgaactcaccaaaggtgagagctcatggagtgtaaggcttatcaaagagaatggttaaagctgagcattgcttttaagtagttggtcaaaattttattagcagttactaagtgtaagtaaataccaaaccttaaataaagtaatagaacaaaattaataataaacccatgcatatgcaaatgacaaaattgaatttaagttccataatttaaacatcagagagtcctgagctgctcatgaccgtgagctcggctagtataccagttttacactaatgcagaggttgtacccttagacgaatctctagattcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttggttacattaatgcacatacaagtaaagccaacatgatgcacttaggttatttaggtgtcttttgttaattatttatttgtttaggtgagatgtccacatgaaatgctacatagggatgacacccatatatgcgaaggaatataatctttactcttaggttttatttttacaaagtgggtgttacaaatcctaccccccttaaaaataatctcgtcctcgagatttaggaagaacgagggaaaaggtgaggaaaatctatgcgaagctcttcttctctttcccaagttgcttcatcttcaccgtggtgactccattggactttgcacatctttaccaccttatttcttgtaactcgagtcaaagtatcctaaatcttgatcgggtattccgtgtaagttaaatcaccctgaacactaagctcttccattggtaactgttcctcagggacacggagccacttcttgagttgagatacgtgaaacacattatgcacatccgatagactagcaggtaactcaagttggtatgccatctctccaactctcctaaagatcaagaatggtccaatatagcgaggggacaatttgcccttaactttgaatctcctcattccacgcagtggtgacaccttgagatacacataatctccttcttcaaattacagtggcctccttctattatcagcataactcttttgcctggtttgagccactctcaaattctctcgaattatacggacttgttcttctgcttcttgaatcaattcaggcccaaagaactgtctctctccagtctgatcccaatacaaaggagtcctgcacttcctcccatgctgaagggcacatgctctcaacatatcttccaaaacttgattagtcctttcagtctgttcatcagtctgagggtgataagccgtactaaaattcaacttcgtgctcatgttctcatgaaagcttctccaaaatcttgaggtaaactgcgaacctcgatcagacacgatcttctttggtactccatgtaaacacacaagccgagccatatacaactctgctagctgagaacctttataagtagtcttgacaggaataaagtgagccactttagtcaatctatccacaatcacccatatagcatcatatcctttctgggtgcgaggcaatccagtaatgaaatccataccaatctcttcccacttccactcgggtatcttcagtgggtgcaatagtccaactggcctctggtgttcagccttaactctttgacatacatcacacatagccacatgtgcagccacatctcttttcaatccataccaccaatacttccgcttcaaatcctgatacatcttagtactaccaggatgaatagaataatttgagtcatgggcctccttcaatatagtctcacgaaggctttcaatatcaggaacacacatcttgtccttgaaccatacagtgccttgctcatcttccgtaaattccagaactcgaccttcagtaatcagatccttaatctctcgtattttagcatcaccaatttgtcctttacggatttcttgctccaaggtaggttccacatcaatagtaactccttcagtgtgagcaactatccccaggttaagtctcctgaaatcctcaacaatctcatcgggtagctgggcaacaacagctgaatgaacatgctcctttcgactcaaggcatctgcaaccaaatttgccttgctcgggtgatagtgaatctccaaatcataatccttaataagctccaaccaacggcgttgcctaaggttgagatccttctgagtgaatatatacttcagactcttatgatctgtgtatacttgacacttgattcccataatgtaatgtctccaaatcttaagtgcatgcacaacggctgccaattccaagtcatgagtggggtagttcaattcatgtctccgtaattgtcgagaagcataagcaatcacatgtccttcctgcatgaccacacatcctaagccttggccacatgcatcgcaatagatatcaaatcctttctgtaggtccggcataaccaatactggtggcgacattaacctcttcttcaatagatcaaagctgtcttgacacttctcgtcccacttaaattcttttcctttctccaaaagcgaagtcataggcttggcaatcttagagaacccttcaataaatctccgataatagcctgctaatcccaagaaactccgaatctcagtcactgtagtgggtactctccactccattatctccttaactttagcaggatccactgatattcctccatcagaaatgatatgtccaagaaatggcacctcgccaatccaaaactcgcatttggtaaacttggtgtagagttgattatctcttagcttttgtagcaccaatctcagatgttcctcatgatcactatcactcttggaataaataagaatatcgtcgatgaatactacgacgagtctgtccaaatactccataaacaccttattcatcaagttcataaaataagctggtgcattggttaatccaaacgacataacagtaaactcatataatccatatcgagtcgagaaagccgtcttgggaatatccgatggtctaatcctcatctgatggtaacccgatcggagatcaatcttcgagaatactctagcacctctcatctgattaaataaatccttaatgcggggtaatggacacttgtccttcacagtgacatcgttaagggtcctataatccacgcacatcccttgcgatccatctttcttctgtacaaaatagaactggtgctccacaaggtgaagaactcggacgaatgtacccatcctcttgtaattccgttaattgcttcttaagttccttttagttcttctacggacatcttgtatggccatttagaaataggggcggttccaggtaagagatcaataacaaactcaacttctctatcgggtggcatccctggtaactcctctggaaagacatccggaaaatctctaaccacacggatgttgtcacccacaacttctcatctactaagaacgccactagtctgatggtggtagttactgcaacttcaacttcaaatctttgtcctttggaactggtgagtgctacggttcccttagcacaatgtacaaagtgcctttgcctttcttaaccatgacataccaaggatcaagcctatactgctctcttctaacagtataggggtagcccatttgggtcaggaacacagggtaagaaagaaaggattatagacaaggcatgtaattacgagtaatgttactacgggggatttattagctaagtagattagtgtgtcacccactaaagctaatacattaccttatggtggtacatgctaagatgcccgtctatactatttcaatcaatcaaagaagcaaatcaggcattgatcatcagaacaatcaaccaacatttttaataaagaaaatagttttaattttgtcttaggcttcctacctcttaaaaatgtcataggggtagggactccaaggtgtgaaatccatcttgtctaggagtagaaaaggtaagaatgataagagtagaacaatagaaggtgagacaagatcaagatgagatgagtatagaatgagtcagagtaaggtaagtaggaaagggtctgtccatttctatctaggtttcgtcctacagtcaacatttcctctgataccacttctgtcacacccggctttaaggaacaaagccaggtgcatctcatacatgcgccaagaagacaacatatataataacagagtgtatagagataaatgtcgtaaaacatcagagtatttattacatagcggaagacttattaaaaaaataaaagataaaagtaaaacgaactaaggatcgtcggcgccaatgtcaactgagaaacgccacctagatcagatcatactcctcgccttgtggctcctcctgaaccacctgctcttctcctgtggggggggggtgtgagacagcaagggtgagctcacacatgatcatcgctcaacaagttgtggggaataatgtgacatgaactcaccaaaggtgagagctcatggagtgtaaggcttatcaaagagaatggttaaagctgagcattgcttttaagtagttggtcaaaattttattagcagttactaagtgtaagtaaataccaaaccttaaataaagtaatagaacaaaattaataataaacccatgcatatgcaaatgacaaaattgaatttaagttccataatttaaacatcagagagtcctgagctgctcatgaccgtgagctcggctagtataccagttttacactctgcagaggttgtaccctttacccacaagtcatgctacccatctgccaaggggtcgcgaatcccatacacctctacctaggaagcgcggcagggcaacactacgaggcctttacaaagttccactagcttccgaaaacccgctacagtttataggaagttccaatgcagggttcctggctgaccgccatcgcagcaaaatcaaccagggacctcccttcattgaccactcccctactgcccttgcccctttcgggtaaggtagtcttccactagctttcctaattaatcagccaagggcgtcccattaaacccttgtggtggcacgtggttctcaagttaagctctatgttccaattaacattaatgatctcaacatgaacataaatagaataacaaaaagaattggaacatagaggtaataaatgattatcccaaaaccatgtaaagcaatagcaaactacccaagtgattcaggggtaaacaaggtaatgagataaacaatctagggtgacctatcgggtcccatcaaaattaacctatgcatggataagtgatattaaagaacattattgggtaaaaagtggtcaagggcacaacttgccttcaatgagctcctgctcagctacttcaacctgctgctcaccagggtcctcggtcacgggctcttctactcgccacaatacaaacaagcacaatgcatatagagaaattaacattacatcaaacatataaacaaaatacacagtaataatctacacattaaaataaaattctaggaacaggaatcataatttttggagttatagaatttaagttatggattttcaaaggttttatgtgctttaaataggattaagtgataaataaatttcttactgttttcatgacataacagaggctctaggtgataaagaataaaattacaaaaatttagaaggtggaatggattattttgaagttcatatgtattttctatgaattatcaaagttctagcaattattttgtattaaaaagtaatttctataattaaataactgaatttaaagaactatggactgggcctcattttctgcaaactacaggggtcttagagtaagattcttaagacacagtgaacaacactgtgcggaccgcgggtgatttccctaaaaacagaggggcctaatcgctaaaccgacccgccgaaggggtatccttcgaacccgagccgcccgatcgtcgaccgagggtcctgattagatcaagaccacacccgaaatggtatgtaatactatgcgctggattggcattcgacggctaccagcgatccccacactcatctaatcctgaccaaccaacccgggatcaacgaccagcacttcatcgttcccaacacactgaccatgcgcggcggcgccgcccagctaatcctacggatgtgagcacggcggcgaactacggatctgtgatttggcgagctattccgaacgcggtgatgagcaatcggtagtggagattacgacgaactcaatgggggcaatcttaccggtggttgatcggtgtagagagccgacgacggggtttggcggcagtagcggcctccgtcgagcaattatggaggtccaggggcccttctcgtccagcgatgtgatgagcggctccagtggtgtcaggcgaaactccctgcactccaattcggccacgggtggcgcggtggtctgggaacaagtacggcggctactttctttcgcaatggcgcacggtggcaatggggaaagggagaggaaaggagggggtcggtccgcagatatagaaggtgaggatcacgacgcgcagagttcggtcaggtcatgggccagcgcagattgtggcatgcgcggttgcggcggcttagctctgaactcggcggagttaggcgacgacgcgcgggttccgcgtgccagagatcgcgctcggggtggtcaacagttctcgcgacgcgcccggggtctttgtacccaggttgagggggcaaatctcgacctgatcctccgatcaacgcggcggctgttacaactacgcgcgcgattcatcggcgagtaaagcggcggcgactttggagacccgtttcacgccagcggcaatcaggggaagatgaccctggcaacacgggtccacttgtctgcgactggtgcgcacgcgcgaagtcttcctggccagtaggcccacgttgtcagcggagccagcagcgcagccatgctcccgcctgggctgcgcggaggggtaatgcgaaactgggccaaaaggggtagccacggcccaggtaggctttatctttttttttctttttttttattttccttttcttttatcttttaaatctctatttgaattcaaatttttttgttgtgaatttgtactcagacaaaagttgtaacttaagagtaccaatttgaaaagttatatttatttttatatagttttctttacaaggtgatccctttcttttccttgttcttttatgttatttccatttttttaaattgtaaatgaggtctttgaatcccaatttgggacacctttgtatttctattgatatgattattgttatcattaaatgcacatacaagtaaaagccaacatgatgcacttaggttatttaggtgtcttttgttaattatttatttgtttaggtgagatgtccacatgaaatgctacatagggatgacacccatatatgcgaaggaatataatctttactcttaggttttatttttacaaagtgggtgttacaaatcctaccccccttaaaaataatctcgtcctcgagatttaggaagaacgagggaaaaggtgaggaaaatctatgcgaagctcctccttctcttcccatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactaatctagagattcgttaccttacccgaaaggggcaagggcagtaggggagtggtcagtgtagggaggtccttggttgattttgctgcgatggcggtcagaccaagaaccctgcattggaacttcttataaactgtagcgggttttcggaagctagtggaactttgtaaaggcctcgtagtgttgccctgccgcgcttcctaggtagaggtgtatgggattcgcgaccccttggcagatgggtagcatgacttgtgggtaaagggtacaacctctgcagagtgtaaaactagtatactagccgtgctcgcggtcatgagcggctcaggactctctgatgtttaaattatggaacttaaattcaattttgtcatttgcattgcatgggtctattattaattttgttcaattactttatctaaggtttggtattcacttgtacttagtaactgctaataaaattttgaccaactacttaaaagcaatgctcagctttaacccctatcattgattagccttacacatcacatgacctcacacctttgtgagtttatgtccaccggttccccacaacttgttgagctatgatcatgtgtgagctcacccttgctgtctcacacccccccacaggagaagagcaggtggttcaggaggagccgcctaacactgaggagttcgatctgatctaggtggcgtttcccagtcgacattggcgccgacgatccttagttcgttttacttttatcttttattttgtaataagtcttccgctatgtaataaatactctgatgttttatgacatttatctctatacactctgttattatatatgttgtcttcttggcgcatgtatgagatgcacccggctttgttccttaaagccgggtgtgacaaagagagaacccttctggcccaactcctaatcctgtgtccccttccccatttctccttccacctcctaagtctgagtataaggtacgaatctctagattcatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagatctaggagattcgtcgtggagatacgacgaactcaatgggggcaatcttaccggggagccatgtaagagccgacgacggggtttggcggcagaagcggcctccgtcgagcaattatggaggtccaggggcccttctcgtccagcgatgtgatgagcggctccagtggtgtcaggcgaaactccctgcactccaattcggccacgggtggcgcggtggtctgggaacaagtacggcggctactttctttcgcaatggcgcacggtggcaatggggaaagggagaggaaaggagggggtcggtccgcagatatagaaggtgaggatcacgacgcgcagagttcggtcaggtcatgggccagcgcagattgtggcatgcgcggttgcggcggcttagctctgaactcggcggagttaggcgacgacgcgcgggttccgcgtgccagagatcgcgctcggggtggtcaacagttctcgcgacgcgcccggggtctttgtacccaggttgagggggcaaatctcgacctgatcctccgatcaacgcggcggctgttacaactacgcgcgcgattcatcggcgagtaaagcggcggcgactttggagacccgtttcacgccagcggcaatcaggggaagatgaccctggcaacacgggtccacttgtctgcgactggtgcgcacgcgcgaagtcttcctggccagtaggcccaccaggcaaatgcgcacagcccaaaagaacggattagaccgtgtgccgaccggtgggccgttggtgccagcctcttccctctgcgccgatggtgggggccacattccagatgcgcgtgctgtggcgacttgcaggcgggcccaggttgtcagttccttccccttcgccgtaacagaactcgcgttctctacgccgcgtgactatcacttccgccgagcccaggaccgctctggattctattgcagagatctcgccgtcaccgccgcgggtatcggatggtcgttacatcgtgtgccgacttatgcggattggcctgggcgcataagtagcgcgccgctcggtatcttggcccaccaaatgaacccctgcctcgcaccacagttgattgtgtagcctcgtcggtgggagaactccgctgccgccgaagaaagctcgacccatagccagtactagagagagaaagacgtgcgccaccgggcggattaacgggtttccgcggtcacctgagtaggggagtgggtgcggcatctgtcgccgttgggcaccgcacccgtgccttccgtccccgcctgcttgcagtgaccccgcgcatttttttttcgcaactcgtgcgtgcccgctcctgtcactcgcggtcgggtggacgaaatctctaggattctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccgattctgatgaatcagcttgattcgtacacgtgccaccacaagggtttaatgggacgcccttggaagacaaattaggaaagctagtggaagactaccttacccgaaaggggcaagggcagtaggggagtggtcagtgtagggaggtccttggttgattttgctgcgatggcggtcagacaagaaccctgcattggaacttcttataaactgtagcgggttttcggaagctagtggaactttgtaaaggcctcgtagtgttgccctgccgcgcttcctaggtagaggtgtatgggattcacgaccccttggcagatgggtagcatgacttgtgggtaaagggtacaacctctgcagagtgtaaaactagtatactagccgtgctcgcggtcatgagcggctcaggactctctgatgtttaaattatggaacttaaattcaattttgtcatttgcattgcatgggtctattattaattttgttcaattactttatctaaggtttggtattcacttatacttagtaactgctaataaaattttgaccaactacttaaaagcaatgctcagctttaacccctatcattgattagccttacacatcacatgacctcacacctttgtgagtttatgtccaccggttccccacaacttgttgagctatgatcatgtgtgagctcacccttgctgtctcacacccccccacaggagaagagcaggtggttcaggaggagccgcctaacactgaggagttcgatctgatctaggtggcgtttcccagtcgacattggcgccgacgatccttagttcgttttacttttatcttttattttgtaataagtcttccgctatgtaataaatactctgatgttttatgacatttatctctatacactctgttattatatatgttgtcttcttggcgcatgtatgagatgcacccggctttgttccttaaagccgggtgtgacaaagagagaacccttctggcccaactcctaatcctgcggcccacttccccattttctcctttccacctcctaagtctgagtataaggtttttaacgctatcatgatatatagtcaatgtggcggtaaacaggagtgttggtgcaggagcgagaagtacgtgggggcgagcgcatgcaatagatattaaacatggctattacaacgtgtacctcagtaccggtagccatggatgcacatgctgactctgcaacaagtgtctgatgcacagaacacgggaggacgaagcatgggctggacctggaccaggaccaggacggctcggcgtgcccctgttgacgctgttcatcacgtactaacaacttttcttctcggtacggatatatattctctcctattcctatccaaagtttacgagttgcgctgcaagcctgcatctgcattgtcagtacgtcctagtcagtagggacccaaccaagacaaggcaacattgtccatcatcacctttatttttgttgacgccggccttgacggcattgcctagtggccagctgtggctacaaataaagagccatctccacccacggcaaatccaaatagtcatgccatgctagtttcgacttacttgctctgtattaaggtcttgttcagttatttcaattccatataaattaaattgtattagaataaattaaaatattttttaatttgctatgtattcaaatcggttcaatatcattcattctacatggtttgtcgtccgggaatactaaagtatcgtgtatatatatagtaataactatcgttgtcacttgtctatcacccgtagctgtactgcaccacgcgccgtggactaggaaagtgggaagcctgatccattccccttgggtcacggacttcataccgtcgtgcatataatttccgaccatttgttctaaaaataaactcactcaatcaattaccaacggaagactatgtgttttctttatctagtacagtatgtgtttttatattaaacaatacagaaaaaatgttttttcctctataactccgactatggggtgtttggttagagggactaaagattagtttctagtttttagtctcatttagtcctttttttgccaaacactaggattaaatataaactaaaatgatttagtctttagtagggactaaaccatattaattccacatttatcactcatttagttcaattgtactaatagtagtagaatgttaaaggtcattttagttttcttataagtcatttaatatattcttactatttttagcctctataaccaaacatgttagagactaaactttagtcttctaattaaactttagtctctagactaaagaaaccaaacatagcctatattttacctatgcgtagccgcgtaggtaacggcgaaagcatgatgacgatacacatacacaccgcccccggccgtggccgccacctagcctgtatgtaccgtgtaccatattcgtaaggctatccgcactcatactactctaaatttctactttaaaaagaatattttatccccgtcagcaagattatctactctataccacaatcctctgcagtcatgtttactctatatctcaactctataccaactaccacatattatattactttttcccacttctcaactaccggttgtccgcacccataccggctgtcactgggccccacaccgacagccccgtatatagagtgagagagtgcaagcgctgaatatagagttttcggtgtcctctatacatgtgcagtcatcggtacagtttaccgctgcagctcccgcgtccactgtaaaatcttaggggagcttttacagctcgcgactgcgaccagcctaaggccgggagcacactagtgctgggaaggccgaaccggacccgcccaagtccattgtgtcttgggccaagaagacataaaagtagagttggacattcgtttttttaaacttgggttcggattttcggttttaaaaaactttgatttttctagacattagaaatcgatcgttttctctaaaataaaaaaatcgagaagttcagtttcgatttcttactttggttttggtttttggtaaaaaccgaataccaaacttatactaagacaacacatacaacaagtttacatgatagattacacataattttcaaaagtaaaaattatataggtacaaatattcagagatacatcatacatcacatataaataagtgaataacaatttaattgattcacacatttagttcacataatcaaatagtcaaattttagaattgataaagtttggtttttggttttttggtatttcggttcggtatacagtaaaaaacgaacttctgatataaaaaaccgaaactgaaccgaactaccgaaatttcgttttggttcggtttaaggtaaaaatgtgcccacccctacctaaaagaaaagtttgagtcgtttcgttgcctgtctaaagagtagaaataatggtcaattccggtcctaaagtttgtagctatgttacaacaatataaattctgtaccttactaaatcaaataaatttaaaaatcgatgtcgggttccgatgggctagaacatagggcccgacctaggcccgcattcgattcatattattttattgtgggatgtgttttacgcttctatttttaggtcatgtgtgctggcccaaacaaaaactaacacatattcctagcactaagccacactgctagtgacaggcaggggagcaaacactagtcgcgtgattggacgtctgcacctgcccattcagcctctgtgtatgtgttgaccatgctgcgactctcgtttggttgcctgcttacacggcgccaaggctgcacccgcggctgcagcttaagcctgaaacatctctcctaattacatgcacatatacgtaaggaacggcctgtttccagtggaccaggctcgcacgagtcagaccacacttacacacccaacccatcagaggatatatcacgctccaagaagcaagaagcagtcctctcggtctcttggccgggcagggcgacaggcttgatgaggccaacagtagacgggaggcatgagccggacaccgcttttccctatcttccgtaggtgccgttcggttgtttgagattagtgcttatctaatttatataaactttaattaactgaaataattttttatgcaatgtggtacaaaagaacaaagacttaggtcttgttcgtttgtgccggattggtgtgggtcgaaaagatttctaattggattgcttctctaatttatataaactttgatttgctggaacgatttcagttacaatccgacacaaacgaagaaggccttagggtgtgttcgtttgttccggaatcgtgacctagaaccaatccaaccaggaatgcttatctaatttgtataggttttgatcacctggaaccattcctgccccaatcctaaacaaacgaacgcagccttaggtcttttcgtttacatcggattacatccggaatcgttccagttaatcaaagtttatataaattagagaagcaatccggttcggaattgttccgacccaccaatccgacgtaaacgaacaagtccttaatcagcacaaaaccaggtcggtcacgcacgggtactggcgtactgccatgcaccaaacgctagtcgtagcgtaggaaaaggaaacagtggtcgtcgaccagcagcaccaaccgttctcgattcaggcttttttctcgatcgcttgcccgcgtcggcgggaggggcctcgctcgctcgttcgccgggtcaacacctcagcacctgcaccgaaagtgccgagtgtaagaggacgactctgtgtgtttgccgccatggcccagcagcctggctgcgctctcaggctgcagttctcgtatttgcgttggcctgacttgatcgggagcctggctgtccgcgtgcaagtttagtctctcagcctggctccacagaaacgagatgaaggtccgttttctgggagcctggctggtcgtggcgcaaggattccacggttaggtagttttagtcttctaacccggctacatactgggtgacaaacaccagctcgtctgagccagtctctgagtctcatatgatacaaatgcgcaaatatagtaagttgcggaagctgggacagactaatcttagcctggaccagttgaacgggccgaactagtcaggcacagggtcgcaaacacgccctctctgtctgcagcacttggccatgcatcattttcctcgtcacctagctttaccaccggcctgggcagcctcttctgtgatatcgtgaatgcaatgctcctctatctatggtgcggtgccctccgacggtccagccaccgaccatgatggatccatgcaagacggcaagatgccaccacacacacacacacacaaagagagtgctgcccactctgcgttcatctgctggcggctgtcctggtttgctccacatcatatcgacaggactgcaggaggcttgtcatggggggcccgttcccccgtccggcccctcccctccccgcgccggccagcccgtggcatggcatctctgcgcaaggcgaccagcggactgcggccggccggccgcctgtgcctgtgacgatttgtcagaagaaaacagcaattttcagggcctgcactgcatctaatcatgcacggaaacggatccaggcgggcgggggcccaaccttttcatcatgcagaagacacatggaatcgaagctgaagatagcctgggcgagtgggcggtggcttctagcgatcaacggaaaacatgtgttgggatcagtgtggtattcggatcggagttcaggtcgtaggtcgtagccgtggaagggctcgcgctccctgcttcacggaccgaggctactgataacgtcgtgcccctgagggcaagcagcgagacagcagctgaggagcatcgtcgcgcagtacaggccccatggacatggacatgcatggcttggctagctcagctattcctcctttgaactatcaacctgcagtgtccgtattataggtgcctaaacggatggcccggcccggcccagcacgggcccgttagatccgacgaataattgggccgtgcccggctggcccgcgtgccgcggcccaggcacggcacgccggcctggtagtcgtgctgagccggcccgttaactggtcacgttctcccattaaattaaggaaaatagcaaaaaactgtgtgtgtgtctagctcagctattgctccttgaaactatcaacctgcattgtcagcattataggtgcccaaacgggtggcacagcccggcccagcacgggcccgttaggcccgatagataatcgggccatgctcggccggcccgcatgccatggtagtcgtgccaggccggcccgttaactggccacgttctccaattaaatcaaggaaaatagcaaaaaaaactgtgtgtgagtgagaatttgaacccgcgacctctctttacaaggctgaaaacattaactaaaagagactaatcaatagaactctagtacatttgttgtaaatagcaaatataaaatgtaaatatatatactatattttataaaatagaaaatatatagtcgtgccgggccgggccgggccgcactgcggaccaaggcagcggcccaagcacagtacaattctcgtgctgaacttggcccagcactattaaacgggtcgtgcttcggaccggccccgttaggcacagtctatttggccatttatggtccgcatgctccgctcgatcgagctagtcagccagcatggttccttgtcggcgcgctgcggccggtcgcgcctgggcctggcggcagctatgcaggccgtctgggctgggcaggcgggggcagctccgttcgccgtcacttggatctcggtttggtgtctgagatcgttcgacacttgaactgcctgccggtacaactagaatatgcagctcgacgcgcacacgtataatcgttgcgttgcatggcgaggcagccagagccagtgccagtgccactgccacgccagggccgcctccgatggaaacagatggaagcagtttacctgtccggggaggcggggaccagctatgcctctgatctggtacgacgaatatatagttaaaactactagtactggatctagctagctgaaatgcactgcatgcgctgtttaccaccggtacggtacggaccggcatccagtacccgtcgcgagctcggctggtcactctagtacagacaagcagcgagctggcgtactacgtacggcgggccggacggacccaacagaactccccacgtcatcccagatatctgaagtctgctggggcgggcgcacacctacctacctgcacacccaactccgatccagctctccgcgacgtatacgactcgcccgccctcggccaccgacctgcagcaactgcgccctctagctaccgtccggatccggacccggtagaggagcgcttgcccgcgccgaccgagaaataaacgcgaccacgcgagcgggcagacgacagcaccagctggccgggcccctacgcgccgcaccgtggtccaacttccaagcagacagagacagccacagccagccgggccgctgcctacgctaaccccgccacgggcgtcgttagcgtgaatgattacggggttagattagatacaaggcgggcggccgatgccgtcgctgctggcaagcaaattgacgtcccgcgtccggattaggaagaggaggtgtggatggatgggggtgggtgggtgggtgggggattttgcgtagaagaaggaaggggccacgtctccacgtcggcgcgcatcaccccgacgacgccacctggacctggagccgcccgcccgcccacctcatgtcggcggtgccggtgcgagcgaggccgatggacgcaacgcaacggcggttggttgtggggggttggccaaatcggtacgtgcaccaaacgtacggcgcctggacatgtgcccgtgccctgtttgtctcgctctcctcctccgccggctgaacctaacaaacgacggccaaagcacccggtgagccgatgaccacctccaatcccggcatccaccatccactacaccatcgctgctgacctgcgccgtaccagttgatcagagtgacggggttttttttttctctttccctgtccactcgtgtaatctccaacagagaagtactaccggcccgtactactttcgtctgcggatacagtgacgtttctttgtcctgcacggctgcacagtgggtggtactgttgactgcgatgaaatcaatatcgttgacatgctgcccatacgtgccagatgccactgttgtgccaagtaaaaaaaaactctgcgtgggacagaaaaatagggaattagctagctagctaggcgcgttgatgtcttgaggaacattagctaggacgtggcgactacagtagtggccggcatcgccgagtgcgaaggagagatgaaacgagcaaggaagaagaagatgagttggacaatcctatttgcactggccggcaccgccgcaccacacaggctatagctcgggggcgcgatcgacgttgaggcaccactaccggcgccctagccgctagctagcctacagctacatacaccgctcgtactataggactacaacgttattgtccacctatcgagctcgagcctcgtgcatgtggtccaggtagggcgaggtgaaggccccgccggccgaccgcgccccagcgacacgtcgtgtttgtccgtcccccgtgctgccctgccgccgctggatccaaaaccccactgcccggtgtacgtacgacctgccgccgacgacgacgagccgcgcgcttgcttgcttgcttgctggcgctgatgattgggtactactggcgatgccctggcgtctcgagagcagagcagcaggtctctcgtaggaaaccgacagctctcggctttagatttgcatgattttttgggtttggagtcgcgggcaggagaggaggaaagccgggccagctattccccccgcagcaaattatgcacgcgagcggcgcgcccgggtcggtggcggcacctatctgccctgcccacctaaaaaagcacagcatggcggcaaggcaattatcaggagcgtcgtcatgctgcgaccacgttatttacttaaaaaaaccacgcagcataaaaaaaaaagtagtcttggctgcggactgcgtagcttcaatccaaaccaaaagtttcaaacggcgaaaccacgcagcatcgaccaggtttttacttgtgtggtgactgagtggagcgacgacggctccgtacggaagcacgtactcctcgtcccagatcattccaagaaacatcgccgggcccagacgccattattccgtcctccaacacacgtagccagaggtacgcaccacgttagggtcaatccgccacgcattctgtcctgggaactagtgaacgaaacgtttagggtcagcagggcaacggtgaggcgacaggtgtgcacatgggttggtgacgagcagggaggagagcgagcgggtggggtgggggtgcgttgtgattgtgacaaccagatgctcgtatcgtatccacgccaatttggttcttgacattattgtcttcacctttctttctagattgtaaattgtcgctgttcgtaaacccatccaatgctgacgcaattaaccagtgtgcccaacaggtaagcttcttcacgggatcgcttccgtggctccgtccacagctaggcctgtgtacatcaattcaccttttatattctacgtggcagaaacctacctaggttcagcgcaaaatttctgtagctcagattctaatgtacacgtacgtccccacccctctttcctactaatctctttttcctctcgccatttctctaccacaaccattacctttccttctttgtctcgttgttatacagtagggtccctaaccaagaaggtatggacaagctgttagagctccttagagtaactctaatagttctctaaacaactctctaaatcgtcttaaatttaataattaaacagaaaaaatggttctctaaatagacttgctaattagttagttgctgtcaactctatataatctctatatttggcaactcgataggagctccttaaatgcacctgacgtcaatttcttcacactggcatgattattttttccattttccacacagtgaggtagcaaggtatagcaagctcgttttccactttctagcacagtgatatagctaggtcaaacacgtttggcataagtttctcctttaccgttggaccttactgtcccataggttgtgtcagcggaccatcactcctccatatgtcatgtcgaccggcctttttctcttctgccctcgacatcctctgcttccaaccctaccgtcagcctgatctgataggtgcagggctgccaaagctagattgaaaatcgaattactccctaaatatgagcagctatcggagactatgttacttttactttggatagctattttgtaacttgctaaacatgattttatcaagctctttttttatagaactattggagttgctttgtggtagtaatttaacaaactaccagatcgttttattactgatatgaaggaagacaagataaaagactataagttagctatgaaaaaaagattgataaagcaacttataaattagaatataaagaatcggtgcacgaggtaggagaggaagatgaggaggaggactagtagtttatagttatattaacgaattgtgtatttttttttggtttaagccatgtacttttaaattttaaatggattatgtaattttatttgcttaattatgtaatcaaatgtatttttatttaaataaaataatatgtttatgtcaactataagtacgtaaaagaaaagttaacgtggtgttgagatgattgtaagatgagattgaagactgtgcgttagataactacgatatataagaggtgacgaaggttatatgactatggtagataattagggtcgatataagagaataatacaacatagccggagatacgtacccgcagtttggaaccagtcatatatagttagcccgttggcagtggatgggcaccggatgcaagttagccggtggacctaacaatttgtgtgccggcggtgagagctcccttccaaacagcacagcacagtacagggggtgtcgtcgacgccttccgcccaactcatccccccttcgacccacaaaatcccaatacaaatactcgcccagcaaacagagagacagggaagggaagccaagaaaaccacgccaccgccaaaagcagctgcagataccactgctgctgcgcctgcctgcacagcccccagcaggagagagagagagagagaaagagggggaggcggcagcggcagccagcaaaggcggcggcttgcggcttgagctgttgatgatgcagggcgagtaccggtcttccatcgaggagtcggctgcggccgctgttgccgcagccgcagccatggctcctctggcggccgcggcggtgaagatggaggcggagcaggcggcgatggcggcgccgcagctgggggcggcgcaccagcagacgcagccgcggcggcagtaccgcggcgtgcgcatgcgcaagtggggcaagtgggtggcggagatccgggagccgcacaagcgcacgcgcatctggctggggtcctacgccacggccgtggcggcggcgcgcgcctacgacacggccgtgttctacctgcgcgggcggtcggcgcggctcaacttccccgaggagatcccctcgttcgggctggcggatggcgtggacgtgggggagcacgcgcgcgacccggccgccgccgccgccggcggcggcggcggctgcacgctgtccgcggcgtccatacggaagaaggccatcgaggtggggtcccgcgtggacgcgctccagaccggcatggtggtcccgccgccgcaccaccgcgagcgccataggcaccacaaccacctgccgcagctgcgggtgcacgcggaggagcagcaggaggaagaggagcagaagccgcagcggcctgcgtggagcgggcgcgtcaagaacccggatctgaaccgtgcgccgagccccgagagctccgacgccgagtgacaagcgagcgagagagctgagcagcaggcaccgcaaggcgaggttcaacgacgacgtccgttatcggttattcccaatcccacgacgcagcatgccgttgtcgtctccgtccgaccgtccccacgtacgtacgtacgtacacgacgatccagctgacccggccgcctgcctatcctatcctatccgtcctcgacgaccggatggtcggcgggcgggagggcggtgcggcgagggttttggccttttgggtacgttgtggataagcacgaaggcagcaggccagcagcagcaggggcggcgtggcgggcgggcaggcaggggcagggcaggcggagacggagggaggcggccacaagcggcggtctttccaaaatgtcaaaaaggacagctgtaacagcgataagaaaaacaagtcatcatcaccctctctcactactagttctactactagcagtatgattattcgaatcggccttgcttgacagcgattaatatctttgccgcctgagttgcaccataaactaatctccccccgccgcccgctttggcatgatccagttgtttcgttccgtcggcatcgccccccgccttggcttggcttaccaacgcagttagctgtcggctggtgcgcgaatccgccgcgttaggtcggctggcccgcttcctcgtcacttcgttcgtgcgccgggcggccggcagccagttagctgaggactgcatgggtgcaatcatggaccagcctcccactgccagctcatcgtggccagtcgctaacgcctaacgaaccgggatagcggtcgccgagggtcagggtgggtgagcagtgcccctgcctgctgccggtgatgctcgcgttgcatttgcatggggcgatttgggtaattgggtgggtgttgttaggccacccaccacatcggaaagggaaaagggaagtgaaggcaaaggcaacgggactacgggagtcttagcgggcgcgcgtcatcatgtttcaacggcgctgtatgcgagcgctgcatatgagcatgacaaagtaccgtctgtgtagctttgttagcagcagctcaacggcgcaggcacttgccccgatgccaaggcgactggcagctgaccagctgtcgtgcgtgcgtgcgagatttccatgggcctgcgagccatgggcggcgcgcgaaggcggaggacggaggctagcgcgcgaacatgccggcagcagtaccgcgaacgacgtggtccggtccggagcaacgacagcgaggcgggtttcagttccagcacataaaatagtttagtacagtgacatgctgtttcagttcgagctacggagctagtaatgatcatcagaatgtccgtacgatatgaatcgttcctccaccttttttttttgaccggagaaacttcatttctattacttgcataacagaaatacattgttcgtaccagctatcacagcaaccaaacatgaaacacccacacaccaacctgattcaggtcaaggacaactatcaggcatagtccaaattcactacatcaagcaggaaagacattaaccaaaaggtggctggcaaaattttcccgaccaaaaaaaaaaaagcgtagcaatacgaatcggcaaatgatctcgcgaaccatcgagtgaacatagaaacatgggggatgtctctgacacgagcctttgttcgggtttcaggtcttcttcaatgcggtgctcttgcagatggggcacacgttcttcaccaccagccattgcttgatgcagccagcgtggaagtcgtgcccgcagtccagcttcccgaggtcgtctccatcagcgtactcctcctggacagggataaaaaggacataaggtaggtatatatattagcctcgttcaactgctaatttcattgctctataaggtgacgctgggacatgttcataggcaaaaaggaattaaattcacagcagataacagatactggtgtacatgatttacatcgacaatggaaatcctgattaaatagacagttctatgctacaggttttgttgccagagttgatgttattttttattagccaccctataaaaaaggttaacagtgttgagtatatttggccgttaggacattgtaatctcgtactgacatatgtatcccgagaggcatcttgccctccaaccctctgaactaaatatttttttccctgaaaggttccgtgcaaattaaacgcacgcattatgcctagtcctctacactatcaccttaagggcttgttcgtttgtgtcggattggtgggtcggaataattccgagccggattgcttctataatttatataaactttgattaatcggaacgattccgggtacaatccgatgtaaacgaacaaggcctaagtagtcttttttagtgatccgatggaagtcatgctagagtcggattgtaattctttcctgagacactaggaaccaagatcaatagcatggaaatctcaagggatgagacaaatattcttagttctaccatttcaaaccgttaatttgtagaaatccagagacctaccagggtatgattctccattggaggcctagatggtataagtagacaagcttagcaacttttagacttaacttgcccttctaatatttgtgggccaatttagttaagtgctgcattgctttgtacacgtgatgatggcctaggtgacctaactgaggtgccgtttggttcacgaaatgtaacataaatggcaacggtaatgatttacactcgattaccatcggtaacaagtttgaataggacggtatcaatttctagtgtggtattcgattacggttggacttaaacaaacatggtttaacgttaccgattaccgttcacgctacaaatatgtgaaccaaaaacacctgaatgcccaattcaatcattcaatgcgattggtgtatctacctacggcaaaagaacaagccatgattctgagatgctcgggagtatatgaaatacctggcagatacaacatggttcagggtccaaagatgcttttagtgtccaagatgaaaatttcctttgtttgagcaaccttatcacagcttcctcgctgaggccagtgccgacatttcctattctttcctcgagtgctaatagctcctgaaagtagggcaaccatcttagattagattttagattcgcaaaggtgcaggtaatgcttcactaaggaacacaaacctcataggacatattgtcaatgtcaaggcgcatgtccctgtgtctatcgtgaatgtcaacaccgccatagaagatagactgttcaagaatacatttggcgtaagaaggaaatatatcaagtactagcaggtaaaaactcaagggcacccaaaacttttgaattcattcagttttattttttcttcgtgctgtacactcatgggattcatgattttctatctacaagtactgaactttcagattactagtttgctacatgttgacccattaattttaagcaaattaaatataaacctcctaccaactaacaaatagtagttttggactacagtagatcacaggaggcaattcattgtcacttatatctgtgtttctgttatatgcttaaacttatgcggtagttgatattagtccttagttttctgctgacagctgtaggtatggtatgtttacgattatagtcatgtttttcttttcttaccatgctattttaaattggaattttaattacagggtaacacaggctggcaggcttaacacaatattatgctgaagtacgattgtactaaccagccaatcataacttaaccacgtccagcaaaaaaaaagaacaatccacaaagaacaattttacttttgtttattttagtggcatgtcgatataatggtagcaaacagagttaaaaaaactcagatggacattgctagtactgacctcaagccttacattctcccctctatggatcatttcaagtgcattccgaatctgttgacaagtaaacttggattgtcataagacaaggaaccatgcacctttacatgatggtcacagtagttaacgcaagcaagaacaagcaaggctagggaacaaaaaggataaaatagtgcagtattttgcataaaaacataaggtgaaacatatcattaaggttcaagtttctctataacaatactagtgaaaatatcttagaaaaaaagtaaaagatacaacaaccggcagtgtgataatatgtggaaataaatgataaaaaaatttcagtatttcaagagttgcaggggcatgaagaagcatgctagatcaaattagggattatgaagtgcacaaacaaaatgaaataaagcatttcgagtagagcagttctaccaatttggtgtgggaatagaatagggtaatttgcttagataggcaaacacaaagatgacaaggataactgaaaacaaggggggcctttgcaagcataaattcataaattagaggatgatttagtgtaatctttgctgccacacaaaaaacacctaataatgatggacttttatgtttccaagtggatgctaaaaaccatttttttgaacagaaccgatcatcttacctccattaaccttcttccttctctaccccttgttgataaaggaacaccccaaacaccatcgccctggcgctccaacagaaaccccggccttaagcgtgggggttgatggccgcgaagtgctccagatagccgaccaatctcctataagaaaataaaagatcaaatcgagaagatgatcaaaccaaattttaagtatggcgtcacttgatattttggtgggaagatgagcaatcaaactgaaaaccaaaatccaaaaatagagaaaaccaaatgcgaaaaatggaggaaaaaaatacctctgataaatttcgaggatggcgccgggtgaggttttggtgactgaatcctgctggcggattaacagttgcactgttattagctcttgatacaggaggaatacttcctgatattgcagtgcctcggacatcagaactccaattagatggttctggcagcaaacgtctcgggtctactacagacgggggaactaaatcagagtagttggcaggcaccacattccttgtaggagcctcttgactaccaagaattctgttctccaaactgccagctctcgatgatgaagctccatggccagtgaaacggtttgcagccggtggcacactagaaacattcactggatgctggtattgcaagttgaggctgctgaccacaggaataacctcctgggtatcatcaaatgcactttgaaaatgaggtggctgatgattccatgaattaggaagtctgatagcatttgaagagggccatacactacagggagcaatctgttgagacgtagtaattcttgcccggaaacttctctgcgaattcccagcattgttgacccctccattaaggtcataggtaccggctgagagccctgcattagttccatatcgtgggagttgatcatcgatagaacttggaaccggcaagcaattgggtgaggacggatttctgataggtgcaggattgtaacttgaagaagcaatgttatggaaagcattgtcgttcctgtgggaaaaactagtgctagcacctgctgaactctggccattgggtccttcaacattttttctcttgcaggctagccggcgtccatccagggaactaccgggtctttggtcaatgctttcttgcatcagtgaaaaatcacacaaagggttttcactagggttcaaagtaaaagaatctgcttgctctgcatctagtaggaaagtcctgtatgggtgtaatctcaggtcagttggctgagaatttacaacactacgatcaccgccaccatagtgctcagggccctgagatgtacagttaacatttgaactttgaccacatagatcgttaacgttggcgttaaggttgatatccacatccagagaaatgccattgtcagctaccagtcgcctttcaccaatcctcagaccaccgccaatactaagatgctctgttttggcattgttagagctgccacttgcacttgcttcgcctgattcccagaaaccaagaaatgtaccatcttgctgtgtctcattcccatatggcatggcagcatcactgtgattcatctgataaccttgaagattctgcgattctactgatccaagaacattgttccaatatgactgctgatccatcacagggttgcctgaactagatgcaatgtcgaatccaaaaacatcagcaaaatgctccacagaattcctctgtccttgcattgtgatgatccaaactagggtagcgagataacatcaattggtctgttgccttagcatctctgatggttcctcgttccagtattttaaaggacctgccagttgtaagtaacacatcaaacattttgggcaacagagctgtgctccaactttattggaacacaagaaaaggactgaaatgaacgggatatagaacggggctacgctttatctaggctattccttcaacatttgccaccaaaatatgtcaactctacatacaaggaactgttgtctgcactccaaaaatattgtaattctactctgggttgttactctatggaagcagctgcacctgttttgctctcacacattactgtagtaggcagcaactaatcagggtattggcaactaatccgactgtaggaatcttggcattaggcgcatttggtgtggcgtacgcgtgcaccaaacttgatggttcgggttgctgttgcgagcaaaaagctgtggtgatgcatgcattttaatgggtgaagcaaagcacggtgaatgtacaatgcatgcgacctaaacagatggaccgcatgcaggatggggcccgttgctcctgtggacgctcgctcacaatgttttttttctcttttgtatttatttcccttccctatcggttttgcaaaaggtgaaaaggacgcagtaaaagaagcaaaaatataaagggaaaaggtgggagtagccatccactatttccaagagaaggatcgattccctctgcgatgacgaaaacgaaatacgggagcagataagaggtttttaactgaaccaagaacacgcccagtacatccatctatctctgtaccgcgagcaaacgaaagaaaaaagtgttttttttcaaacatggaagttgggaattttgcttcggggaagacaagaattattgacagcttaacgagagccgtaccaaggccgctccatgtacaagtactgtggattgaaacgtgaaggaatcagcgaggaaaaaaaacgcatatttgcgaaggaatcatggattgaaacaacaaacaagggattgtttcgctatgcaaaatcaagagatataactagaaatacaaaaaaaggcagtttaacagagagatgcaacaaaccgtaccagaaactggcccagagtttctgaacaactcgcgagaaccgcaatccaggcaaaagaacaggctccgttctgatttctcttctttcctgtctcctgtttctcgatcctctcctctccacggagaggaagagagagagaatcaacccagcagacggaggaaacaagaaccgattggttgcgagagagagaggcaacagacagatggagcggtgggcaagacgagaggaaattcagatagaaagaggtggatcggtggagtcggtggggaagaagaggaggaagagaatgggtggaggaaattttgtcaagagggagagggagaaagacaggggaggaggaggggtgtggtggtggtgacaggaggtgggagagagaggtttggctgaattcgggtttatttatttctccctcttccccacatccaggcactcactctctctctctcttctaggcctttgcaagggtccttttcctccccactaataaaaaccccttaaatgcgcccccattagaattagcaggagtataagatcactagatggacttgtgtaaaaacatataaagcaatgttttatactacaggctatattctgtagactagagtttaaaacgaagggtgggttataaaataactgatgaccttattaataacctgacctcacatgaaggaccaaaggttagaggcctgtatcgtgttgtctttcacatgacctccctgcgcctgacccatttggttttggagccttgcagttgcagcagcagcactacgacagcaaggcctatccttctctctctcatcctttgcatacactgcacttggacagcgcagcatcaggatggatgatggctgctatgctcatgctcatgagctcatcccttgcatcgcattgaatttatctttttcttttcttgtctcggagtagcatccttcgcattgaatacctccgggtgtaacccaagtgccatgagatagtagtagtagtatgtttagttggatcgtgtgcagctagctgctgcacatttgtcagctcatgagaaaaaatcatgaaagcgagttgtttcagaacaagccgcgcgccattcattatatatgcatcaactcgtgagctgtgctggagcctctggtgattttgttttttcacctgctaatcaagcgtcatttcctggcaactaatcaagcgcgctgtatgggctttggttgccccagggcaatggttgtcaggtgcttatattctggcgggcggcgatgcttaaaatgtggaccgcacatcaagtgccaccacaaaataacttgccttgttcaagcagaaaacaggtcaaaaagaaagaagatcacggaggcagtctgaacccaccacgtacgtacggtaccaccaccattctcgctgcgtttcttcagttcctttcgtgttcccaccagcgacgtgtgaggtcgatcatgcgttcaactgcacaaacagcacggaaatgttaggatttatgggcttggcccaattaagttattcaaataaatcccaggaaaatctcaaaagcccatataagtggatggcaaagggataggtggaaccaatagcaccatattgctagctcttgtggagtagagctaacttaaatatggaagccacactcactcaccaagtcatggatgagaggagagagtgtggagagccacacgcgcgcgcgcgctcgctcgcctggccgggccgggccgggccggggcggggcggggcggggcgaagggcgcacgacatgcgcgcgaatggtccgccgaaatccggcccctcgccttgcgggggcgcggctaccttttgccgtttgattttttggtttcttggctgttacgcttatcctaaccgatccctataaaatctcaactgattgcgagattttcgtgggcggataagcactggggtcgcggactcggccctataaaaggagcccggcagccagcctccaaataatcccagatcccagttcgctttcgcctctcttcatagctgagccgccttttagttcccttcgtcccgaccgcagaggtgcatctgcgatcaggagagcaggtctctggaacccttcgtcttctagatcctgcaccgggagagggcgaataaggtttttgggaagcgtcttcacgcgactgctcgtgatcttctgacatcgtcgacccagctgatcctggcgcgcgccaacaatcagtaagtctaatcagtacgcatcatctgatttggcttttatttcagttcttctgatttggtcatgatttatattcggaatttaatttggaatttgtctaattaatcaacaatccaaaaaccttattgtagacaatttcctagtttttctatggctgcttttgccgacgcgctgaagccagaaaagtttaatggtatgcactttaagagatggcaagtcaaggccacgctctggcttactgctatgaatgtcttccatgttagtaaaggcagacctgagggtccactgactcctgaacaggagaaagagtacgaccatgccaatactatgttcacgggagccgttcttagcgcccttgttgaccgtctggttgatgcgaatatgcagtacacagacgggaaacagttgtgggatgcacttactactaagtatggtgcatcagatgctggcagtgacctgtatatcatggagagctttcatgattataagatggttgataatcgctctattgtagagcaagctcatgaaatacagtgtatagccaaggagctcgaccaccttaagatagtccttcctgaccgatttgtggccggatgcattattgcaaagttgccttctacatggaggaacttcgccacagctctgaaacataagagacaggagatatcagttgaaaatctgatagcgtctctggatgttgaggagaaagctcgggctaaggacacgggatctaaaggaggcgagggccactccagcgccaacatggttcagaagaaccacaacaagggcaaaggaaagccaaaatctaacaagcccaacaaaactaccaacttcaagaagaagaagaacaaggctgaattgacatgtttcgcatgtggcgaggcgggtcattttgccaaggattgtcccgatcgagcggatcgccgtggcaaaaagggcaatgtcaacacagtggtcgctagcaatgaggaagacaaagggtatggtaatttacctttcatcttctcagtatttcaatcacctagctggtggcttgatactggtgctaatgttcatgtgtgttctgacatcaacttgttctcttcttatcagggcgcccgggattcttccgtgctaatggggaatgggtcacatgcttctgttcatggcactggcacggtggatctgaagtttacttcgggaaagatcgtgcagctgaagaacgtgcatcatgtcccttctatacacaagaatctcgttagcggaacccttctatgtagagatgggttcaaggtagttttagagtccaataaattagttgtgtccaagtctggacaatttattggtaaaggctatgattgcggaggcttgttccgcttttctttgttagatttcaataataagtctgtgaaccatatttgtgctaatgttgatgatcttgcgagtatttggcattctcgtttgtgtcatattaattttggctctatgtctcggcttgcaaccatgagtttaattccgaatatcaccatagtcaaaggttctaagtgccatagttgtgtgcagtcgaagcaacctcgaaagcctcataaggctgctgaggagagacacctggcaccactagaactcatacattctgatctttgtgagatgaatggtgtgttgacaaaaggtggtaaaagatacttcatgacattgattgatgatgcgtctagattttgctatgtatacttgctaaaaactaaagatgaggctttagactactttaaaatctataaggctgaagttgaaaaccaactagagagaaagatcaaacgtcttagatcagatcgtggtggcgagttctttcccaaagtctttgacgatttctgtgcagaacatggcattattcatgagaggactcctccctattcacccgagtcaaacgggattgctgaaaggaaaaaccgtacgttgactgacctggtgaatgccatgttagacacttgtggtttatctaatgcatggtggggggaggcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgacatgtttcggatgtggcgaggcgggtcattttgccaaggattgtcccgatcgagcggatcgccgtggcaaaaagggcaatgtcaacacagtggtcgctagcaatgaggaagacaaagggtatggtaatttacctttcatcttctcagtatttcaatcacctagctggtggcttgatactggtgctaatgttcatgtgtgttctgacatcaacttgttctcttcttatcagggcgcccgggattcttccgtgctaatggggaatgggtcacatgcttctgttcatggcactggcacggtggatctgaagtttacttcgggaaagatcgtgcagctgaagaacgtgcatcatgtcccttctatacacaagaatctcgttagcggaacccttctatgtagagatgggttcaaggtagttttagagtccaataagttagttgtgtccaagtctggacaatttattggtaaaggctatgattgcggaggcttgttccgcttttctttgttagatttcaataataagtctgtgaaccatatttgtgcaaatgttgatgatcttgcgagtatttggcattctcgtttgtgtcatattaattttggctctatgtctcggcttgcaaccatgagtttaattccgaatatcaccatagtcaaaggttctaagtgccatagttgtgtgcagtcgaagcaacctcgaaagcctcataaggctgctgaggagagacacctggcaccactagaactcatacattctgatctttgtgagatgaatggtgtgttgacaaaaggtggtaagagatacttcatgacattgattgatgatgcgtctagattttgctatgtatacttgctaaaaactaaagatgaggctttagactactttaaaatctataaggctgaggttgaaaaccaactagagagaaagatcaaacgtcttagatcagatcgtggtggcgagttctttcccaaagtctttgatgatttctgtgcagaacatggcattattcatgagaggactcctccctattcacccgagtcaaacgggattgctgaaaggaaaaaccgtacgttgactgacctggtgaatgccatgttagacacttgtggtttatctaaggcatggtggggggaggcagtcctgacttcatgtcatgttctgaatagaattcctatgggcaaagaagagaaaaccccttatgagaagtgggttgggagaaaaccatcactttcatacttgcgcacttgggggtgcatggcgaaagtcaatgtaccaattaataaaaagcgcaagcttggtccaaggacagtggattgtgtctttcttggatatgcttcgtgtagcatagcatatagatttttagtagttaaatctgaagttcctgatgtgtatgttgatactattatggaatcacgtgatgctactttctttgagcatatatttccaatgaaagacattcatagcaattctagatactcttctgagataattcctgaacataatacacctattgagagttttgaacagccacatgaaattgtcctagaggaggatgacaatgatgctcctaaaaggagcaagagacaaagggttgaaaaatcctttggtaatgatttcattgtgtaccttgtggacgatactcctactaccattgcagaagcatttgcatctccagatgcagatgattggaaagaagcagttcataatgagatggactctattctttcaaatggtacgtgggaagtcactgatcgaccctatggatgcaaacccgtgggttgtaagtgggtgtttaaaaagaagctcaagcctgatggtacaattgaaaagtacaaggctaggcttgtggctaaaggctatactcagaaagaaggagaagacttctttgatacttactcacctgttgctagaatgaccactattcgagtactactttctttggctgcctcgtatggtctccttgttcatcagatggatgtaaagacagcttttcttaatggagagctggacgaggaaatctatatggaacagcctgatggatttgtagtaaagggtcaagaaagcaaggtgtgcaagttattgaaatctttgtatggtctgaagcaagcaccaaagcagtggcatgagaagtttgacactactctaacgtctgcaggctttgccattaatgaggcagacaggtgtgtatattatcgctgtggtgggggcgaaggagttatattgtgcttatatgttgatgatatattgatatttggcacaaacattgatgtgatcaatgaagtcaagtcttttctatcaaagagttttgatatgaaagatctgggagaagctgatgtgattctaaacatcaagctgattaaggcagatggtgggattactctctcgcaatctcactatgttgaaaaggttttgaagcgatttggcttctctgagtgcaaaccttctccaactccttatgatcccagtgtgacactgcgaaagaacaagagaattggtttagaccaattgagatactctcagattgtcggttcactcatgtatcttgctggtgcaacaaggcccgatatctcgtttgctgtgagcaaattgagtaggttcatgtcaaaccccgggactgatcattggcatgcacttgagcgggttatgcgctacctgcaaggtacaatgagttatggaattcactattctggtcagcatgcagtacttgaaggatatagtgattcgaactggatatctgatgcagacgagctttatgccaccagtggttatgtctttactattggtggaggtgcggtatcatggaggtcatgcaagcagaccattttgacgaggtcaaccatggaagccgagctagctgcacttgacacagcaaccgttgaggcagaatggttgcgtgaactcttgatggacttgccggtggttgagaaaccaataccagctatccttatgaactgtgacaatcagacagtgattgctaaagtgacgagttctaaggataatggaaagtcatcaagacatgtcaaaagacgattgaagtctgtcagaaagttgagaaactccggagttataagtgtgacttatatttcaacagataaaaatctggcagatccttttaccaagggactaccacgtaatgtgatagaaatcgcatcgagagagatgggtatgagacccgaataaagttgccatggtggaaacccagtctatgtgatcggagatcccgtgaattaggtcctgggaagaacaagccattggtgaactgaggagagtaacttttgaccctctctaagtaaagatgcaatactctcaaatgctgtaaggcaggttggctttgtgccttaatgtgttctgttggcttgtattagcgaagatgttgtcctgcagaacattctttgaaagaacacacctatatgagttagactgtctaacgtcgcagtctatgagatctgggtgatctctagtaaactcatgaagagaccttggagtacgacgtatatgctccacccgagaaggggactactggtagccaagtactagtcatgacttcaagtgaaacccattcacgcaaaacttgcaattcaaggcatagtccattgtccaagttgtgggttggtgtaacttggagttctaggcggaagttcaacttaacagtctctgctgaaaaactagtatattaaacagtagtgaacagtggcgaaaactgcagatgggcatttgagatctggtgggggattgttaggatttatgggcttggcccaattaagttattcaaataaatcccaggaaaatctcaaaagcccatataagtggatggcaaagggataggtggaaccaatagcaccatattgctagctcttgtggagtagagctaacttaaatatggaagccacactcactcaccaagtcatggatgagaggagagagtgtggagagccacacgcgcgcgcgcgctcgctcgcctcgcctggcctggccgggccgggccggggcggggcggggcggggcgaagggcgcgggcgcacgacatgcgcgcgaatggtccgccgaaatccggcccctcgccttgcgggggcgcggctaccttttgccgtttgattttttggtttcttggctgttacgcttatcctaaccgatccctataaaatctcaactgattgcgagattttcgtgggcggataagcactggggtcgcggactcggccctataaaaggagcccggcagccagcctccaaataatcccagatcccagttcgctttcgcctctcttcatagctgagccgccttttagttcccttcgtcccgaccgcagaggtgcatctgcgatcaggagagcaggtctccggaacccttcgtcttctagatcctgcaccgggagagggcgaataaggtttttgggaagcgtcttcacgcgactgctcgtgatcttctgacatcgtcgacccagctgatcctggcgcgcgccaacaatcagtaagtctaatcagtacgcatcatctgatttggcttttatttcagttcttctgatttggtcatgatttatatttggaatttaatttggaatttgtctaattaatcaacaggaaagacgctgctcgtcagccacgccgctcatgaagcaaagaggctgtttttttcgcgcggttttcgggtaaaacgtcccgagcccgaggcctactccgctcccgttgatgcggatgcctgcgctgcgctgcgctgtcgatctggcgcgtgcttcgctgcttttctgaccgggactcgtgctcctcgtccgtccgtcctccctccaccccgggggccggggcgaatatacggccggtggaaggatgcgccggcgatccgccatggtctttaccttttttccccgttgttgccactttgggctaaattacgtaaagaacctcgcctaaatgcggtgccagcttttttctttcacctcacctcgatcgtcacctgctctgctcgctcaccaatcactgaatggatgccttttttttcttcttgattttcttgtttcagtaagttcagacgaaagggtttcctgctctcccatattatccagaatagtgtgtagtagcgccctccaccactgtgttatcctctgccgctctgtgctgtgctagtgtgctgcctgtgcatattaattaccttttgaccagttcttcagagagctggctgagggtttggatttgtctctataattgttgttggtgctggctagtgattactgtaaaagcatatgcaccggatcattcctcctgcgtccactgtgatttggccccacacacacacacacacacgtcgacaacaaactcactctcttattattaatccgtctcaacgagccaacatattctgacagatgcacgctgtaacctgcctggtaaaaaagattgtttttttttctttcaaaagaaaaattgctagctggtggatacgcgctagctatgggctgctgctgctgcgcccatctgccattcccttccctagcgggaacgtaaagaaggtgagagcagtaggcgagcgggcactggctggctagaatgcgatgcaagagcgcagtgttgtgttggtgtgccaccaataccattgaaaggcaagaatcctctcagcatcaacacaacaagtgtataaaacagggcacgcatgcttatcttcttcttctgctttggcttaccggtcgtctcgtctgcttcgtactgctaagactttaggtggtgcatgatgcatggctttacgtgccttgcctttcatccaccaagcatcatccagaaatcctgctggggttttccgtcgaggtagccggctacttctccctccgctcatacaaatagtgacacaggaaggccggcgagaggagacgtcaggctctgcatcagacatggacgcgcgctgcctgcgtaaaagctgcgtaccgtgtgaggcgtggacggacggacaggacagtgtagggcgtgcattttggcgtctcgccgttggacagtttctggttcacccattgactcccgactcccgagccgcatggcaatttcttctacggagtagactattttagtagcactgtagcattgtctgcaaaagtcatgtgtcagcagttattacacaagtagcctgagtgcctgactgctgggacccaccgaccccagtttcaaacttcgaaccgtacccaacccttgtcaaaagagagaacttcacacactgtaacacagcgcctctgccggcgcatggagcagcagggtaaaaagttaatcagatttatatattaaaagatactgatatttcttatactcactgtttgtctaaaacatcgtttagttttagtctatttaacacaccgtttgcacacggcggtacacggtactacacccgggttcttttaatcaaccgttcatcccattaaaataatcgtttcaccctactaagcgactgtttagcgtgaataatggttaggcattattaagttggtatgttttttcaaagaaaaactttggtacaaagtttgtgttcacatgacttcatacaaaattggtatgttttttttttaaaaaaaaatttggtatgttgctgctccctccgcccctcccgtgagccttttggaccaagcaatgcaggcgatggcaagcacactgtaatctactaatctgaaagcgcctgtcacgggaatctcgctttacctgtggctgtgggtgggcccgctcatttccgcggtgcgtgatcccacgcatatgcatgcatgcatacacaacgggtcaactgagcaattactgctgcaactgaccaccgcgggtcgttcgacgacccagccgttcagctttcggccacgcgcccaccgagcatgtcacagcacctgcaggcccctttggaccagcagcctcgctctgctgctctggccaccaaccccgtcgcactcagccactgccgtggagtggattcgcggcggacccgtgccgtcccgtacagtccagacggtgaccgacgatgtaacagtaacagtctgcggacggcgggcggttcgagctgcagcttgctcccccggtccacggccccgtgccgctgctctgctctgctcccgaaagccgcttattgggccgacgttaaagttgcatcacgagcccggtctgctagtagcatgccagtcgaagcgggaaggtcatgcagtgattgcaggctagcgtgcacactcaaggcggcgcccgcagactgcagactgtcgctgccggcgcttccgttgcggcccgactcggccgttgcttgcctccaggctccagcatccacgcacgagccatggcccatgggctacgtgcccacgtctgcttcccgatggctcattacacgtctgggctatatggcctatgggcttcttgcgtgtagacgttttgttttttaaaagtataaaacctctgtttttttttgaaagcgagtaaacagggccagagctacaccaaatctaataatgaattggaatttacttaataaaatagactatttgtcgtgaaaatttacattccaccgctttttcaaaagtttagatataaggctatcttagattaggatgagagatgaaaattaattttatgttgaaccaagatatttatctactctacaatttataacacgttcttcatttgattcttctacagtagaatgttgccgcataaatatctttatcatatggtcaatccatattagattaattgatctatgtctaaatcacgattattcaaatgatctaagcgggaaatgcacttgctttatgactggtttggaaggaattaaggggctttccttccccctcgggccttgtttggttacaaaaggatcgtaggagattgaagaggattaaatccttcttatttaattttaactagaaagggactccctaaccttataagcccttaatctcctttaattctactattactaaaccagaccatattagtgaaaagattcatgataatgtcttgtttggtgacaaggggattggataggattaaagacaaaattagttgattttctcctcaatccgtctcaattcccttgtgaccaaagaagccctaagtgttagttttggagccacaaaactggaatggaattcaattttctttcggatttgtggctcccaaactagccctaagtgcgtcgaatacaatgaaaaattagctcagaatcatcaaccgtctctacatgaatgaaaacgctggtagcaactatctttttttttttacattgccaacttgccatcttgttgagcctctttcaaatgccacaagcaaatctgacctgacccccacggttcttgtaaagaggctgagctgttttggaagacaccagcttttgctaacctgacccccacggttcttgtacactggatggaaacgcttccagatcaaacctgtctctcaagcaacaccacctaaacaacggcaaggagcaacggcgtattgtttccttgtggacacatcacgcataaaaggttatccagtatacaacgagtgtgtcgaggcgtgggcaacaacagtctgaaacgttttctcaaacgtagtttaattataaaactaattatatagataaaaactaaataataattagattacatttaatatttataataaagattaaaactttcaatgtgacaacgtattaaattttggcctctggaaccaaacacctaacagtgttcttgggtttgatgtcaggacccactgagccgatgtcggcctctacgcctcatcacagcacgagcatgacgaggcccttctgccttttgtaccgatgctccggttgctgagagtgcatgcaagaactcagcacacggtgcgctattcaccaactgtttatagtttcctttcttgaaaaaacaccgaccaaaagctctggtttccgcacgcccagacgttagagatggaatgtgcaataatagcagatgatgatgatgatatgactgaacgaggaaaaatggccaaaacgatgatgattgatgaatgggatcgagctgcagagctttactacattcgcgcacggtacaagtgatctcttacttcgacgaccaataagagaaaaagtgggcggacgatgaggaggaggaggcccggttggtgtcgcccaccgttgcctgcggcgacggcccgccatcggcgaagctcagcgccggcggggggtacgcggccaggacgcccaacggcgggagcttcctcatagccgacagcggcggcgagaacccgccggcatccttctcgtcgtacgcgcacgcctcctcctccgtgaccccgtacggcgacgagcccggcgtctcgaacggcggcgtctccccaccgccggcggccgcggtctgccctcggtcgtacaccgggttggagatgcacatgaacgcgtcgccgcacgccgaggtggctcccgacgagcccgagcgcgacggcggcggcggcgaccaccgccacacggcgccctccagcctggggatggcgacgaggaggtccttggtgctgggcgcgtgcaggacgccgtgcgcgtagtagaagggcggcgtgccggcgaacgcctcgcgtgacgacgacgcggcggcgtcgcgcgccggggacggcggcgtcgccagcgccaggccgagcttcgaccaggacgcctcggcgcggagccgccggcgccggaggtgcgcgcagatgaggtccgcgaggaggagcactaggatggtgaggaagatggccaggctgaacgccaccgtgacgctcaccgccaccgccagcctcgacatgccgtcggtgaacatggcccgctggctccgggcgccggcctcacgagaccgtggcgtggcgtggcgagcggagcggtcgcggtgcagagctgagtgtgaggagagagcaagtgtgcaggtgcaactgcaagctcaacaaacacaagtggcgcggcaacgggccaacggtgagcgactgcttgaggtcccatcagacgacaaaatgcccccctcccttgacattgattgactaattcacccggtaattaacatcctcggcacgctttccgaggcgagctagttgttccaaaagctagagcacggcacgattatcgtgtgctttgtctgaatgaaccagcagccacttgtcagtaagggtggtaatggattgtgatccatatggttcttcacaaaatgtcaaggccctaaataaattatagttcaaaaatgactagaaataaaacccgatcctaatccgatttgatccttaaatcttatagtgcaaaatttacagcccattgtcagccctacttgtcagcctctgagccttcgcacagtggcaaccgggtcctggcccgtgtggggtccccaatcatcacgagctcgcgagtgacagccacttgtcagcctctgagccttctcacagtggcaaccggtaattagtgatgagcttggcccgctctctgcttggccctgaggccttgtctgcgtcaactagaaggtcaagtgtagtcaaaccacagtgaaatgagtgaaacacctgtgtatgacagttttgagagtcgagggactatttctacatgactgatgcaatctggctaatgtttgatctgaatcaactgtcgtcattgagctttttcgcgggcaatctggttaatgcaccctcagtgtaaccgcacatcgtgcttctacatgcaggcacaacgcgcctcctggtacggtcgtggacgccgaggttgagggaggccaaggacattcttatactcggttgaagaaggcctgttctttctgaaagctaacactctgcagcgccgccttgtcatcgtgacatagcgtgcaaagtttggttctcagatgtttagcaaatgcgtatagattgacatattgtatagttgtaccagcctactagggtagcgcgtgagcctgatagtatatatgcatgtacgtgatttttttagcgtgaatctcactagtacacagaaaaactatagtgacagtcctaaaactgttttcactggcgtttttcagtgccgtcagtgctaggggctagtgaaaatcgtcattttccattggcggttattcaacaaccaccagtggaaatagcattttcactggcggttttcttaaacaaccaccagtaaaaatagcattttcactggcggttttattaagaagctggagttgttgcgagaggagctaggattctctgctgccaagaggcagtggtggttgagcacttgagctaggaatcgagcgagagtaggtttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtaggtttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtaggtttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtagttttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtagtttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtagttttttgagatatattaccacatgtacatgggtatattcaaagtctgatgcaatgaaatatgcagatacactacaccgagcaaaaaaagctgaagtaaatgcggcctaggatcgggaacaacgggtctcaaaagttgaagcgagaataccgagcgagagtagttttttgagatatattaccacatgtacatgggtatattcaaagtctgatgcaatgaaatatgcagatacactacaccgagcaaaaaaagctgaagtaaatgcggcctaggatcggtaacaacgggtctcaaaagttgaagcgagaataccgagtgataataaaaaaatcgccgttgccggggatcgaacccgggtcgtccgcgtgacaggcggaaatacttaccactatactacaacgacttggatgactgtacaatctacgaccatttctagaagtagcggaatggcacgaagatttcagcatatgtaactataagggcattttacaagttttcggctccgaagacagggaccgattggtcttcgttcggttcagatttgatcatgaccgatctggccggactaaaaaaataatcactaaataatgtacaccctctgcctaaaattaaaattcgttttaggctttaaatagatatacgcagtaattaatgtatgtgttttgtatatctatctagatttatcgtcatacctttaaatatagacgtaaaaatcaagagctaaaacgaatagataatggtaaatatagacacataaattaattattgtgtggacatattaattaactaaaataaattttagttttggacggagagagtatgttattgtaacgctattagaaaaaagggggaacagtcacacatctcaatacgtgacaattggttggtaacatggcatcctatccctgatcccataccgaacatgtagcgttaaggaacgtatctggtgttcaaagggttatgatgttcatgtgcatattttaaatctggtgcatctattttacatttaacagcagcaccaattttgtgaagcaccccttccaccttcctcccatgatggaaggagtttttcgtaaaattagctggtgccgttgaatgcatgatggatgcaccagatttaaaatatgcatatgaacatcgtacccctttgagcaccagatacgttcccatttgagctctcaagttgacctatttgggcctaacggcttaagtcatctgtcatgcttagcagccattaaactgtctaaaaatatataaaaagtgataaaatgcacacaaatcagcaaattcagtgcaggtaacagttggccagatgagtttgtaggtattctggggaacttggtctttgctagatggattttattacagtccccatgtttgaggatatcgggaccatatgaattccatgattagcataatttctctattcttctatgagactgagcatagaaaacagtatgccacctatcgattctttctacctcttgaatagaagtatctaaatttcttctgattattggtaccattgtgatttttttagttggtggcagcatgcatcatcaaacctcgcaagctacgagcaacaggatgctcatgaattctttatttccatccttgaccatatctccatatccatgagaatataaaggatgaccaacaaaaatcgcatgcccaaggcatgcttctctttttgctgttttctgaataaaaggccttcacatatgacatatgttgcagtcaactgaaatctatatcgagtggctatatatattttatattttgtttctagactgcaagttagtttgctaagttatgtttaaattaaggtaagattatttagcatgatccactgaggacagtctcatgcccacgttaagaagatgggtcttctctggaaaccgtggaattcctgttggttagacacctgttggggacttgttctcaaatgctatgaattaagaacaaggcaacataaaatgttgaatattaacatccttcgtccatgaaacaatattcccttgaggataatgaaccatggacgaaggttcatgaagacataattacgaaggttagatctccgtaatcatattctcatataaaataacataagataaaggatatgaaaagataaacgagtcatgcacaaatattaaatttacttaatatattaaattattgaatacaattatacctctgccttggcaaagattggtttccgaaagatgcgattacaatagccagaatgcgtgaacagtaaaggaatactgttcactatttataggcacaggacacagcctgtgaggaattacaattatgcccctcataaaagtttacaacattgacccagacctttatggactaaaaggtcattccatccttaagtcggtttgtaataccgaagcttcatgatgaagaaccttcagccatctcactcaagcaacttcagccaaaagccgcttcatcctagaagaccttcggcgccgaagcatagacccaacagtagcccctttcgcggtgctagatcgtttttcgtaacgagcttgatccgtgaaaaaagtctcttaagcttcggggagccgaaggtccaaaaaacaccttccctgagctcgttgtcgagaaacgatttagtttcccagcgcgtagcggtcccaccttgcagagttactgtttgatctctgtggtccaccttgcagtgaatgcgagcgggtgcccgcctggtgcaaaaatcctggcgcttcgccttcctacctgcaatactatataaacagatgagtaggtgtgaagttaccacaacattcattgctatttgcactgttttgctgccaaaatttttaaccaccgccgaagcttaactctcggaatcgaacgaagctccagtttgaagcctgcttcgtcagaagaaaaaacttgggaggaaaaaagtattagatttttacaaattcagaattaaatggccagagtgcgttctaccgctagggttgagcgtgaggggggcgaaactgaagattcggagtctgttcccatctccgaagcgatgcagaggtccgggctggtaacttcggaaaagattcccagtgatgatgcaaaacaggcagaacaagcagttgccgaagcggaaggagaagatattgaggaaactgatcccgaagacgattatcggattgccatgccaagcaagcccagtcacttggacttcggaaagtctactgtttcgaaggctgatctctccaaaatggtaaagtcgggcttttttaatgagaatcagaagaagctattacgcttcgggggagaggagactaccccaaagccagagaaggatgaaattgtcattttcaagagctttctaaaggctggactaaggttccccctacatgggattattgcagaggtactgaagaggtttggtgtctactttcatcagctgactcccaacgctattgttagacttagcgtttatatctgggccctccgaaaccaagcggtggagccgtttgcggacagcttctgtcgagttcacgaactgcactatcagacaaaggctagaaaagatggattgcatgacaattttggttgctataatttcgcttaccggaaaaccacaaagtttcctgtaatcagctaccgaagcaaatgggcagcgggctggaagtcgaagtggttctatgtcaaggttgatgacgacaaggagaagcttgtgcaaagtccacttgaactaatcttcggagaaacccgcccccattgcaacatgacaccggaaggtcctactcaaaaagcaatggatgaattcagaattatcgcagagcatatcagtacaagagacctggttcaggagtttttggctttcaaggtttttcctagtttaaaagaatgggaaatgccgaagccaaagggggaaaagaaagaaggtgaacttgtgcgtttaccttactatttcaagtttaagaaatactttaaaacaccttgccaagagtggctggatacaattgaagtaatgtgtaatgaaatacttggcaactactccaaaaaagaagatcaattgatgactgcggccttcggcacccgtccgaagcgaagactgaatcgagtgctggatgccttgggttttgaataccctgactatgaaaatctgaacaaaggtgccggaggccagaaaagaaaaaggataactgaagccttaaatgaagatgaaaaagaaccaccaaaaaagaaaattccgaagaagaggaaagcgtcttctccgaagcgaaaaatatccgacgaggaagaaaccctcgcatcaccctctgccactgatgtggaaacaattttgaaggtaatgactgaatccctgcctgcgaagctaagtccattggggcctcaactgacaatgttttttcagaaggaaaaggagtccgaaaaaatggagaaaacgactaaagtaaagagacagaggatcatcggtgtgacagaggtaattgacaagacaccgccaagagcttcagctcagaagacaccagcggctgaggaaaggacagatattgaaatcacaccttcggaggtcgcggctgccgaagctgcctcagctgaagatttgaacctggagagcacaatcgaacatattgacaaaatattgttagacatggctgcagaagaagctaccaccgccgccgaagaggccactgccgcagcgtcgggaaaaaggaaggaaattgtagatgaaacttcagaaaacgaagccttcatgtttcaaaatttagttggagaaaaattgtcagaacccgaaatagaagagcttaaagagtatgccaaatcttgcgggtacaaaccaggagcactcctcttcggaggtattgatgatgagaaattagattgtatccgaaaccaaactggagccaaagttattggtactctgtcaaagagtatcggttttccgaagctagaaatggacattagccgctaccgacgacaacatatcgttggtagtttattttattccaatttcaaggtgaactacttttcccttttattgtctttaataataaaaacatgtctaacaaaggttgttttcgtgcagagtatgctgttgagcaaagccttgaaaatgcagcaagattctgaagacaagaaacacgaagctataattgaaaatttagaaaacaaaataaaggagcaatcagatgctatcgagaaaaagaactttgagctccaggcaaccgaaggtttattggcggaagccgaagcaaaaataacagaactgaacacgaagcttctccgccaatctgagcagtttgaccaagaaaaacaagaacttaatacaaaacttgaagccgaagctcaacaaaattcagatttgaaaaaactattggcgggccttcaagaaaaatgtttggaatttggcaacagatgcattcaacgactaagaaaaatttttcactcagttggagccagcagtgagaaattcaccccctcagctgaagatttatcgaagaccttcgaacatattgagggtgaaattgacgagctcgacgaagttatagctgggcacggtgacttctgtgcctgggtagcttctcggggcactgctgcagccttcctaaaagctggctgcgatcacggaaagattgtcaataggcccaatttcactttatcaccatcaatcctagatgacactcctgatctcgcccgaagtatttccaatagatttgtgaaaatgatatggacaaaaggcgggcgagaaaaggctggagacgaagctcggagtcattttgaaccagtaagaaatcataccttgtgcttacctcttccttcaagctcaattttgacttacaacaacttaattcatataggatgacgaagctgaagccgatgattgaaaagaacgacgccgaagctaaggtcccccgaagattaagtagtagactgtagatgaacttaggaaacttttgagataacttttgtaaatgtaactaaacttgtgcttaatgaattctgttcataccttggattatgctcttacccttccattaatgtatgtgaggtgctttgttgtggacgaaattatcttttttgagccaaaggcgaaaaaacaccttcccttcttttcgtacacagcgaagcatagaaaacaatatttccctttcttccgaagctctcctttttgcacgcgaagaagctcttctcttgtaccgaagcaattacttatgccatgatgatgattatcctacatatgcctggatgaatgtttatgaatgcaaatgctatgatgtaatgtgatgtgcaaatgaatgtccaaacacatatccgaagctatactctcagccattattttcttagaaacaatcacacatcagctctgcattcccttaggaacgactttggagcttcttcgccttttactttcggcggaatcagcgttgactttttcgctgtaagctctgcattccctgaggaacgactttggagcttcttcgccttttacttttggcggaatcagcgttgacttttcgttgtaagctctgcattcccttaggaacgactttggagcttcttcgccttttacttttctgcactcgatggtgcattctcagcttttacatttacatctttgggagattttactcttataaaactaaaaaaggaaattacatgcgatggccccattaaaaacctttctccccctttagaaaggaaaagggtgccatgaaaaagaaaaaacataaaaaagttacatcaagttacacataatatcgccgaagctcatccgcattccaagatctaggaatgtcgttaccgtccatatccttcaatctgtatgaaccgggtcttgacgaagatactactaagaaaggtccctcccatttcaactgcaacttgcccactgtgtctgggttggccactctccgaagcaccaaatgtcctggctcaatattctttagccgaacctttctatctcgccatttgattgtttcggcttgatatttattgatattttccacagcttgaagcctaatcccttctaaagcatctttttccacagaataagcatcttcagaacctgattctgccgaagctactactcttattgatccagttttagcttcctccggagttattgcttcgtcaccgaataataacttgaatggagtaaagcctgtagaccttgatgttgttgtgttgtggctccacaccactttggttaactgatctggccactttcccctaggttgattgaagattaacttcattattcctgtcattataatgtcattgtgaaagttgcctagaggggggggggtgaataggcaagctaaaacgttttcaacaaaaactagaagcaaactgggtaaaactgaattgatctcgaaattcacctagttaactttggaaatgagatgttctaaatgatccacagggttcaaagtagtagatctgagaagggcacttctcaaaatccacacaacaaaaagatatgaacaaatcttccacggaagggtgagagaacgaaaaacacaaacaaacacaatgaataagaacacaagagacacaagatttatcccgaggttcggtcacaccaccaaggtgccctacttcctcgttgaggcgcccacaaagagtcgggtctctttcaaccctaatcctccctttgccgaccacaaaggtcaagcccacacaataatctttgctcaaacgagcgggtaatacaaactttcttgtggtcttccacaagatttggagactcacaagagacacctagtcgtctaggagctagaagctccaagagtaatgaatccacaaagaactcgatgtagtaccaaagctcgaatgaagaagagcaagagagatttagagatggagcacaaaaaccgcagctctcaagctcactcaaagatttctctccaaagatttgaaatgggagaggcaagagatgtgtgagagagagtgggaggtgtttcttgggttagaaatggagttcaaatcgtgctcactgctttggggagagaggtaggaggtagtatatataggtggagctcaaaactagccgttgggcagatttttctgcctgagaccggttgaacctccccccagggcggttgaacctcccctggcaggcctggcagcctgtctgccagtctgactggcagactgacgcgcagactgaccgcagactagtcaaagttgaccaaaaccagttgaaccgcccagggggggcggttgaaccgcccctgacagctcctggcgacctgtttgccagtctgactggcagactgcagatcagaggtcagaggggtcagagaccagctgaactgcccctcaggaccagttcaaccggtcttgaccagagagtgagaaaaccccagctcaactgcccggaggcaagttcagctggtgtatggtcaaagaggttcacagctgaagttgagttcagctgaagttcagcccagctgaaaagctcagctgcagctgaagaagctcaactcagctgaagttcagctcagctgaaaagctcagctgtagctgaagaagctcagctcagctgaagtccagctcagctgaaaagctcagctgcagctgaaggagctcagctcagctgaagtccagctcagctgaaaagctcagctgcagctgaacaagttcagctgcttttcagcaagaacactctaggtttctcaaacctaaccatggtcaaccatagaacgataaagagatttttgcttttcataaatagcttttgaatagagaggtttgagctttggcaaacaccaaccttctttttggatccccctttatagtacgacgattcctatactcaagttaaataaaataaaatgaagtaaactccttgagtcattggtgtctcatgtgtgatttctccatggcattgcttcataaggatcacaaacatctttgtctcaccttttgaagcaaactcaaatcaaaccctgtgacttgtaccatatcaccttatatgagttcaaatcatggcttcaagtcaccttactgatgcatcaacatgttgtaactcttcatagctgattagttcatcgacttagtgcaagtactctcttcttcaccttagccatggtacctcggtctacaagccgtcgcttggccttcaccttcgcttagttcctcgaagccctttccttgctatcttcaccctatcaagccattcttgagtcacatcctattgagcatccattaagagaatcatttcttcaatattgtgaaccttgcttgaatgtcttctagatataactattaagattaatcaagctttagtttgattctcatatattcatatatggactaatagtaatatggtcaagccaattcacgattcctcatatcttattcactttggcttgaccaatcctcttaatcacttcaacctctattatgatcatatttatgcatagtgatttctcaggacttgtccatatattcaaaccaatatagagaccatattatatccatttgcattgtctcactggttatttcaccttgtgttgaaccttgttcactgatcattatacactattcaagtatgttcatcatactgaatttcctgttcaacacttagcaaacttgttagacctttaaatgtgttgttatccaaatcaccaaaactcacaaaagggatgaatgcactttcaatctccccctttttggtgattgatgacaacacatttaaagcttacataagatttgataaataagattttgaatcctatgatatagtttcctccccctaaacatgtgcatttcagaaaataacacttttgtactcaaatgccaaaagcacatatttaggtcaaagtatagacaacttatatcatactattcatagggtgcagtgtgtcataaaataaacgtgatgctcatgacatggaatgcacttatcaactttcttcatcttatgtttttcttatgattcccccttttgttaattatttctccccttttctaaaataaagataagctttaatgcaaaatttctccccctttgtcataaatctccaaaaaggttacaaagaatataaagggtagaaattatgatttaagcaagatgtgaacacactatcatgaaagggatccttagatggcacaaaggtaatggataagtgtcataagtgatgtacctttgcgttttaccttttcaaggggtttccagacttgtgttggatttagaattcatttgcaagctcttgttggcataattgtgacataggtccaagatatcaaatgaagattgtcatactaactgaaagataaatcttgatacctggagtctagatgtcacctagcattttcttatcacaacaagaggcaacatgaaaattgcacaaatatggattatgcaactagtgatcatgtatgaaaaatatcaattgaaaaacaccaattgatacaatttgatagataaatagatcactaatagcatattacactaagttctcctcaagttttagtgcacacatatatatgtattcaattgatacctattgagagtacttatttgcaatttaaagtaccattggcataaaaaagtatcatatgaatataatcatacaacataaggaacatgtgcactatttaatcaattaagttctagaaggagaatctataagttatgctacttcagatatttgtaataaaaaaagatgtgatacatatttaccaattttagatgacgttggagtagcatatacaggagaaactcattctcttatgaaatgtatagaagatacatttattggagcaaagaatctttgatacccatcaaaatttgcagtggaagcgattgtctttgcccgaagattcctttctaattcgagactacacacataatagacttggaaatgaagttagtctcaaagattcaaattatagactattctccccctaaatgtgtgcatacaagtgatgaatacttgtttagcttatgcacttggacttgtgaggccaggggattaagtcctacaatttgaaccttggtacaaataaagtatgaaaatgtgaaattgtaccaatttaaggaattactcataattaaaaggtatattaatagacatgatatgcaatattttaagtcaagattcttgagaagttagcatgtttgactcatacctcactaagatgacttaagactaacttatgatatcaccacaagaggtattaatcaaatatctagagattctttaatcgtcaccacaagtgcaaccttatgatgtgacttaagatattgcatatacatgcacacactagcatgtaagagcctagatacacaaatgtaatacaatagttcatggagtgacacacctttgttctacgccacatggtctccattataatcatgaatttccatcttagcttttgataggattgacatttcaaagagaaactcatcctcttcaaacgatcaagagaaactcattttcttgataggttcaagagaaactcattttcatttctatggaaccattcatcctcatttgaagctctccaaggtgtgagactacacaacatgaacttgaaagaaatcattagtctcacaagatataggctaaactctccctcaatttgtgcatgcaagagtacacaaagtacacttatgcacattaacaatgtgaggttaaaggagatgtttgtactatatcttggcttaacataacatgctttatatagatgatgaaaattaaaccaattgaagatttaagaactgaaagtatatcaattgaaatcctgaagggtaaagcatgctaatatgaacctcaaacctcataatgaaggatatcatataaatatgtcactacatcttcattatttggttgacaaaaaaataactcccttcttgaatcactgtgatctcttttctctttacgatttcatccaaccaagtgatcttgaacttctttcatcttttcttgattcgatcatacttgatatgagatcaattgaaactcaatgattgaaacaaccaagactcttatatccgtagattttaaatccatcttgaaaacacttggaaggaccttattgaaacacttagaaaagagagcattagaaaaacaagggagggtttcacaaatgataatccttatatgtggatggagaatgaatcatcattcttgaaacccaaaaggatagattaaattcctttaaactagtgcacacatatagttgatctcaaagttatatgcactgttgaacattttatattgcaaggaaattaacctatactatggtacatcccactaaggatagaatactaaaacaactgaaggagaggaagttttcataccttggcctcttatcaacttcatcttactttagttgaataatatcctttaagcttgtgatttatccaatttaaaacaagcaccatctcttaacatagattttctatggataaactcagcacaagagatggaattagtagcacaagcactagtttcttatttagcaaccctttatatgtggaaggcaagcgagttgctaaaatagagaaacctcataatatggactaaatttccttgaaccctcatgcacaatatattttgaatgacatggataatatgcatggttgttcaatgaagtctaaaagggctgacttaatccatattatggtaagtgtctaataaagaagaatcaaatccaaattaactagatagagaatacgtcacataattttggattgttcaccatatgataatattcattcatcttccaattagacctttatttcataatcaacaactgatgtatatcctcaagtgcttcttttcccttagtggctacacaagtcacaaaagagataagatttgaaaataatttgcacttgagattcagttgttaccacccttgctactatctccaaatatgatttatacattcattatcgagcatccaacttgatccattgaaggaatgatcctgcaaaacaagtttaagcttcatatcttggtgcccaatttgaattgggtcctttgagattagtaggaagagccttgagtacccacacaatccttattgtgacctttctctttgtgtaggcacccaaatatacttgacaaccatcttacatggtttcaagtcaatatttaatcacacagataaaagctagagttaagaataggagccttttctccttttcttgatacatcattatgttgccttcttgatgatgaacctagcttgaccttgggtgcacctagcttatcaaggttagtcgcacaagcattcatatcataaagacaagttatgcatggaatttacaacttagtgatccaattcaatgtgaagcatatggatatcgattgaagtagatttctaaaatatcaaaatatgggttttcaaaatttagagagtatggatcactaattgtaccttttacagtttaaaaatcatatccatgttagtgcatatgtatgtgatgaatatcaacaaaaagattcttatgcacttttagaattaacgataaggaaattttaaactgcatcaagagtagctatttagaaaacaaagattacaatccatatgaatgagatacaaatttaccattttggattgtcttagtatagcttactcaagagaaacttattctctatgacacaagatatataatgttctcccactagatatgtgcatcaagtatttgaatgacttgccacatgcactttcaattcagttaagagtctcatgaggagtacattacatatcatggtcaaggcatgacaaatttgcaatgaattaacttatccttaagaatacttaccaccaagtagagcgtaccatttgttataccaatttgaaagatcttactatctgtggtggtgtcatcgtagtattcttcttttcatcatttgtggagacttccttctttagcttgttatctcttttccttttaatactagttgaaaaagaactttgaaaagagatattagtagtacaaggaagtatttaatgaatggtgatctttagatatgaattgcacacaaatcatcatttatgaggcataaagacataaattaaattccttttaaattaatgcacatggagcagtgaattcacaatatgcactaatttacaaatttaagccaaaaggaatttaacctttatattgacatttcttttcatagaatatattattaccaattgaaagaatgccacttggaaggaactactattgatcaactaccaattgaagcaatttgttccatacctttgccttgagcattcctaatctttcttttaggtgaagattaaccttttaaagcttgtgattttaccaattgaaagagcacaatctctacttatgaatttccatgaaatatcttaaaagagattgtattagtaacacaagcaatagtttcctatttagcaacctctagtatatgaatgacaagaaggttactaaaacaaggaaacttcatgatatgaactaaattacccttacaagcatcatgcataacatcaattaaaaaaacgaaagtagcatgattatttaattaagtttacatggcaagtttaattcatagcatggtgagtgattaatgtagaagactcaaaaccaatttaaacaagaatgaatacttcacatagtattggttcgatcttctttgaatgttgaatggcacactccatttgggaaacacattctcatgttgtgaaactacataaatcacttagataaaaacattagtctcacaactctagtcatatagagaatttcccttttggtaagtgctttaagaatttgaatttcttacttagcactctattcatttaagaacatgggataatcctctttatgtctaggtcatggcaataatacgataatcaatttgaaatatgccacaagatacatacaaccaatttaaagcatgtagattgtcataatataaaaatatgaacgtgcaatctaccatataattagatcctttccaaagcaaggtaaaatcttttaagttcattctcaagtgcttcatttttcctatgtggctacaaaagacacaaaggaatatactaattaaaagcaatgtgcacttaagaattaattgttaccatcctttggatatcacttggttgtaggccttttgcttgattcccacaaaggttaatccttattccctacaacacaagttcttggtagagatgaatatgaagttagtgatataacaactcaattcatctttgggtcaatcttaaaggatatacaatgtaagattgatagcttgagataagaattgagttaaaccgctcaagcacccccaaagcttcatatcatctgtgggtacctgcaaaacttatcaagtacattttggtacccatctttggatgggtccatcaaggttagccaataaggacttaggcacccaaatagccttggtcctagaatgtggtgaactcatcacctttctagcacaagagtcaaatttgggtctcctaagcatatttgaatgtattgacaagttaggcttaggagttttaccctttggacaaaccttgaatagatgacctttttcacggcaattgtagcaaatgcgatgcttgtccttgcaatgcatttgccttttgctttcatcctttttgatggtcatctttcttgatcgtgcaaggtcttgattcttcatgtgggggcatgaatcaatttcatggcctttctccttgcatccatagcacctcctatcgcttctctttgatctttctttgttgaagcacataggtacattgttagagcaaatcatatgagcattaattttctcaccatggatttttctcatgcccttcttggaaagtttggttttcttttgaaggggttttgtgcatgctacggttgtccccttctcaagctttttcaccatattatcacggttatcttgagaaggttgagcatgacactttcccttcaaacgagttaagctccttcttagcattccaacttcttccttgagctctttattttcttgtgtgatagaaatatcactaattcctgaaatttctagctcaatggaagattggctttcttgagagcaacatttgttagcacatgataatatagtttctacttgagtacatgtgcatatgtgaggttggtatgatttcaaattagttaacacaacctcatgagccatttctaacatgatatgtgaatccataaatttattatgatagcacacaagcatatcatgtttttcttgcaaagctagattttcaatatttagcctttctatcgtgttcttgagcatagtattttctttttctaattgagcaacgcaagataaagcatttgaagtactagtttgctcaattgaaatcttttcatacctttggaccaaatcatcatgagagcactttagcttctcatgctctttggtcaacttctccaagtctttgattttttcgatgaggatatcttcttgcttatgaagcatttccttttgttcttccgctcttttcataagtttgagcaagctcatccggttcttcttacttagttgagcgaagaatttttgaagatcatcctcatcttcactatcactttcatgctcctcctcatcctcactttcgctttcactgtcactctcattagcaacaaagcacatatgagaagtggatttgaaagacgaaccttgtgaagaggtggattcatcgtttggtctccatcgatcattttcttcttcttcaataatgttcttcgcctcatcttccttcattttgaggaacatcctttcggcgattctcatggcaagatctattgccctaggatcaacatctgcaccaaaagataaagtcgaggcaacctcaactttttcaagcttagaagcactttcgtttcttagtccccccgacatgatctttcctcacgcggttaagcgttagaacgaggattaggctctgataccaattgaaagttgcctagagggggggtgaataggcaagttaaaactttttcaacaaaaactagaagcaaactgggtaaaactgaattgatctcgaaattcacccagttaactttggaaatgagatgttctaaatgatccacagggttcaaagtagtagatctgagaagggcacttctcaaaatccacacaacaaaaagatatgaacaaatcttccacggaagggtgagagaacgaaaaacacaaacaaacacaatgaataagaacacaagagacacaagatttatcccgaggttcggtcacaccaccaaggtgccctacttcctcgttgaggcgcccacaaagagccgggtctctttcaaccctaatcctccctttgccgaccacaaaggtcaagcccacacaataatctttgctcaaacgagcgggtaatacaaactttcttgtggtcttccacaagatttggagactcacaagagacacctagtcgtctaggagctagaagctccaagagtaatgaatccacaaagaactcgatgtagtaccaaagctcgaatgaagaagagcaagagagatttagagatggagcacaaaaaccgcagctctcaagctcactcaaagatttctctccaaagatttgaaatgggagaggcaagagatgtgtgagagagagtgggaggtgtttcttgggttagaaatggagttcaaatcgtgctcactgctttggggagagaggtaggaggtagtatatataggtggagctcaaaactagccgttgagcagatttttctgcctgagaccggttgaacctccccccagggcggttgaacctcccctggcaggcctggcagcctgtctgccagtctgactggcagactgacgcgcagactgaccgcagactggtcaaagttgaccaaaaccagttgaaccgcccagggggggtggttgaaccgcccctgacagctcctggcgacctgtttgccagtctgactagcagactgcagatcagaggtcagaggggtcagagaccagctgaactgcccctcaggaccagttcaaccggtcttgaccagagagtttaggagagaaaaccccagctcaactgcccggaggcaagttcagctggtgtatggtcaaagaggttcacagctgaagttgagttcagctgaagttcagcccagctgaaaagctcagctgcagctgaagaagctcaactcagctgaagttcagctcagctgtagctgaagaagctcagctcagctgaagtccagctcagctgaaaagctcagctgcagctgaaggagctcagctcagctgaagtccagctcagctgaaaagctcagctgcagctgaacaagttcagctgcttttcagcaagaacactctaggtttctcaaacctaaccatggtcaaccatagaacgataaagagatttttgcttttcataaatagcttttgaatagagaggtttgagctttggcaaacaccaaccttctttttggatccccctttatagtacgacgattcctatactcaagttaaataaaataaaatgaagtaaactccttgagtcattggtgtctcatgtgtgatttctccatggcattgcttcataaggatcacaaacatctttgtctcaccttttgaagcaaactcaaatcaaaccctgtgacttgtaccatatcaccttatatgagttcaaatcatggcttcaagtcaccttactgatgcatcaacatgttgtaactcttcatagctgattagttcatcgacttagtgcaagtactctcttcttcaccttagccatggtacctcggtctacaagccgtcgcttggccttcaccttcgcttagttcctcgaagccctttcccttgctatcttcaccctatcaagccattcttgaagtcacatcctattgagcatccattaagagaatcatttctttcaatattgtgaacccttgcttgaaatgtcttctagatataaactattaagattaatcaagctttagtttgattctcatatattcatatatggactaaatagtaaatatggtcaagccaattcacgattcctcatatcttattcactttggcttgaccaatcctcttaatcacttcaacctctattatgatcatatttatgcatagtgatttctcaggacttgtccatatattcaaaccaatatagagaccatattatatccatttgcattgtctcactggttatttcaccttgtgttgaaccttgttcactgatcattatacactattcaagtatgttcatcatactgaatttcctgttcaacacttagcaaacttgttagacctttaaatgtgttgttatccaaatcaccaaaactcacaaaagggatgaatgcactttcacattggctctttcaacgagcccatttgactccggatgcctgactgatgcaaaatggatcttcgtaccaatttgatcacagaaatccctaaaagcttcggagtcgaactgtgttccattatccacagtgatggtctttggtaccccgaaacgacaaacaatattctgccagaaaaacttttgaatggtggccgaagttattgtggctaaaggctttgcctcaatccatttagaaaaatattccacagccaccacaacatatcttaagttcccttggaccggtggtaatggacccaacaagtcaaggccccacctttgcaatggccagatgggttgtatgagttgtgttaaagacgaaggttgtttttgatctcttgcacatttctgacaaccttcgcacttttgaactaattccgctgcatccgaagctgccttcggccaataaaacccttgacggaaaacttttccaagtaacggcctagatccaatgtgagatccacacaggcctgcatgtatttctttcatcaactctatgccttcggttctagataaacacttgagcaatggagcacaaactccatgcttgtacaactccccttctatcatgacatatggacgagctcttgcctctatcctcctgttataagtttcgtcatctgaaaggaatttaccctgaagataagagatgatttcagttctccaatcttcgctataaacaggagatatattgaggactgctctttcaaggagttccactgaaggtgcctttattgttcgaagaacacatccgaaggtaaaggcagcccctgtggctgctgacttagctagcaaatcagcatgttcattttgtcctcgagggatattcttgacagaaaatccttcgaaggaagcttcaattcttcgaaccatgtctagatatttttcaagcttcggatctttaaccttgcaactcttgtcgatatgacccgaacaccacctgagaatcagttttaagaatggcccttctgaaaaaaaaaaaacttttagcttccgaagacccaaaagcagggcttcgtactcggtaaatattgtttgtacaactgaaatcgagtcttgccgcataacaagttttaacttgggatggcgaaaccaacacagcagctgctcctgccccgaaggttccccaagacccaccgcaaaacactgtccacacttcggcatctttatttgtttcttcatcctgagcccctggcgtccagtcaacaatgaagtctgctaacgcctgggactggatcgaagatctatgcacataatcaatgcaaaattcattgagctctgcagccccattttccaatccgcccagtagcttctctatttctcataatatccttcaacggctgcgaagaaggaacaacaatattgtatgcttgaaagtagtgccgaagcttcctggatgccatcaaaacagcatataataccttctctaattctgtgtagtttttctttgatatactaagaacttcagatacaaagtacattgggacctgcttcttgacttggccatcaagcttctcctgaacaagtgctgcacttaccgctgagtgcgaagctgccacatataataacaaaggagcccctggcgttggtggagttaatgttgttaaatctatcagatattgcttcagttcctcgaaggctttttgttggcttggtccccattgaaagacttcggctgatttcagcacttcgaagaatggtaaatttctttctgctgatctagctataaatctattaagagatgccaacctccctgttaatctttggacccccttttttgtagttggtggctccattcgaagtatagcttcaattttacttggattagcttcaattccctttgttgataccaagcatccaagaaattttcccttcttcactccgaagacacatttttccgggttcaattttaaaccagcttgtctgaaactggcgaaggtctcctgcaaatcagcaatatgattttcctgttttgtgctttttacaatgatgtcatcaacataagttagcacatttctgccgatctgagagtggagaaccttcgcagtcattctgctgaaacttcctccagcgttttttagcccctcaggcatccgaagatagcaatatgtgccacttggggttatgaagctagtcttcggctcatcttccttcttcatccagatttggtggtagcctgagtaacagtctagcagactcatgagctctgaagaagctgctgcatcaactaacgagtctatccttggcaatgggaattcatccttcggacaagccttgttaagatctgtaaaatcgatacacattcgccacttgccattggccttttttaccataacagtgttagctagccattctgggtactttacttctctgataactcctgcactgaggagccttttgacttcattacgagcaccttcggccttatcatcagacattttccgaagcctctgctttctaggtctgaaggatgggtcaacattgagcgagtgttcaataacatccctatttactccgcagagatcattggctgaccatgcaaaaacatctttgttgttgaacaaaaaccttatcaaggttttctcctgttcttcggataattgagagcccaacagcaccttctgctctgctatgtcttcacataagagcatgggcttcggctgatccgctgaagctgctttctcccttctgaatttgtactgttcacaagcttcagctccatctatgttatggattgcttttgagtcagtccagttcccttcggcccttctggcagcttcctgacttccatgaatagcgatgggtccttgatccgaaggtatcttcatgcaaagataagcaggatgaagaattgcttcgaaagcattgagggtgccacgaccaataattgcattgtaagggtattccatgtcaacaatgtcaaatataacttgctcagttctagtgttgttgatgaacccgaaggtcactgacatggtgatcttgcccagtgctacaatctgccttcctccgaagccacagagaggatgtgtagcatcatgaatcttatcttctggctcttgcatttgtctgaaggccttagcaaatatgatatcagctgcactgcctgtgtcaaccaagacattatggaccagaaatcctttgataacacaagagataaccatggcatcattgtgtgggtaatccttgagttgaaggtcctcttgggagaaggtaatagggatgtgagaccatcttgacttgatgaagggtccttgcaccccaacatgctgtacccttctctgtgcttccttcttctgcttcttgttggctggctctgaacatgaaccgcctgttatcgggagtagcagcttcggagccgaagcagctccagcttgatcgttgaacgaagccatcagctcaaaaaagtggaagtgagttcaccggaggtgggcgccaatgttggggacttgttctcaaatgctatgaattaagaacaagacaacataaaatgttgaatattaacatccttcgtccatgaaacaatattcccttgaggataatgaaccatggacgaaggttcatgaagacataattacgaaggttagatctccgtaatcatattctcatataaaataacataagataaaggatatgaaaagataaacgagtcatgcacaaatatttacttaatatattaaattattgaatacattatacctctgccttggcaaagattggtttccgaaagatgcgattacaatagccagaatgcgtgaacagtaaaggaatactgttcactatttataggcacaggacacagcctgtgaggaattacaattatgcccctcataaaagtttacaacattgacccagacctttatggactaaaaggtcattccatccttaagtcggtttgtaataccgaagcttcatgatgaagaaccttcggccatctcactcaagcaacttcagccaaaagccgcttcttcctagaagaccttcggcgccgaagcatagacccaacaacaccattcattcgttcgttcatgtccatatctcgtcattcatgttcgattggacaagcacattggttgttgcatattgacaagtccgtcgtacctgaatctttgttcggttagggcccccatcacaaagaaccccacatacgtcccgcacggcggcacgagcacgagccccttcaccgacacggcgacaccatcagtccaccggcaccgactactcagaccgagcctccctccactccggcacggcacacgccagcgaaccggtcggggtttccatccaatcccgtcacgcacgccgagccatggcgagaccaaacaaagcttcctcccggcgtccccgcacggcagggggagagtatgcgatgtgaccgagatcgagagcgaccgggcgggagccccatccgcatccgcgggacagcaacgcgcgtccgaacccgagccggcgccaccgctacctgcgtgcgtcgcccgccaccgccagaccgccagagccaatcagccaagtactcccatgtaacacccgaaatcctgtgcttagaattataaatgtttttataggaagttttgtgataaaaggacttctaataaaaatttccgtcctctataaaatataataataagcaagaatctggtattcgaagcatagatctaaaacctaaagctaggagttgaaaccacatataagagagagacttttaaactaactattgtttcttttaatcaaaaccatccctaaaaagataattgaactaaaataattcatgagaaagattacatattaacatagccgaagtaaatataactaaagatgattatttatcttaagtgtgcattcaaagataaaatttccttaaaaaaaataatatgtgtataatccatcattagagcatttatctaaatggatacaaataaataaaagaaataagtaaaaattatgcatcatgcttgacctttgggtgtgaattcttgtattcaaaattgaatctaagattgaattatgaaaattgaaaactaaaatcagaaaagatcaaaagaaaggagaaaataaaaaggaaaaagcaaaagaataatggaagccagcgtctgggcctatttttcgctgcctcggcccattccccactatcttcgcgcggcccattggctcccactgcgccgctcctacaccgacaggtgggacccgcgctatcagacactgtgtcgcacgctggttgcctggtgtctccgactcgtggggccggatcgtcagtcttgaaagggaattaggcttatacctagttcctaaataattttggtggttgaattgcccaacacaaatctttggactaactagtttgctctagtatatagattatacaggtgtaaaaggttcacactcagccaataaaaagaccaagttttggattcaacaaggagcaaaggggcaaccgaaggcacccctggtctggcgcaccggactgtccggtgcgccaccgggacatgtccggtgcaccagggggactcagactcaaactcgccaccttcgggaatttctagaggcgactcggctaaaattcaccggactgtccggtgtacaccggacagtgtccggtgctccaagggaggtcgtcctcaggaactcgccagcctcgggttttcactccagccgctccgctataattcaccggactgtccggtgcatctgcggagcaacggctacttcaggcgccaacggctacctgctgcgcatttattgcgcgccagcgcgcgcagaggtcaggcacgcccatgctggcgcaccggacagtgaacagtgcatgtccggtgcgccaccggacatcaagcggggcccagaagtcagagctccaacggtcagaatccaacggcagagatgacgtggcagggggcaccggactgtccggtgtgcaccggactatccggtgtgccatcgaacagacagcttccaccaacggtcatgtttggtggttggggctataaataccccaaccaccccaccattcattgcatccaagtcttccacttctcaacctcttacaagagctaggcattcaattctagacacacccaaagagatcaaatcctctccaattccatacaaagcctaagtgactagtgagagtgatttgccgtgttcatttgagctcttgcgcttggatcgctttctttctttctcacttgttcttgtgatcaacactcaattgtaatcaaggcaagaggcaccaattgtgtggtggcccttgcggggaagttttgttcccgactttgatttgagaagagaaagctcactcggtccacggaccgtttgagagagggaagggttgaaagagacccggcctttgtggcctcctcaacggggagtaggtttgagagaaccaaacctcggtaaaacaaatcctcgtgtctcacttcattattcgcttgcgatttgttttgcgccctctctcgcggactcttttatatttctaacactaacccggcttgtagttgtgtttatatttgtaaatttcagtttcgccctattcacccccccctctaggcgactatcaattggtatcagagcccggtgcttcattagagcctaaccgctcgaagtgatgtcgggagatcacgccaagaaggagatggagaccggcgacaagcccactacaagtcaagggagcacttcgtcggaagagtcccacaccaagaggaaggagaagaggaaggtctcctccaaagggaaggagaaaagactttcttcgcaccgcgaagaaaagaaggactcctccaaagggaaggagaaatcttcttcgcatcacaaagagaagaaagaaaagtcctcttcccacaagccgcatcggagtggtgacaagaagaagaggatgaggaaggtggtctattacgagaccgacacttcatggacatctacctccggctccgacgcggcgtccgtcacttctaaacgccaagagcgtaagaagtatagtaagattcccctacgctacccccgcatttctaaacatacacctttactttccgttccattaggcaaaccaccaacttttaatggtgaagattatgcaatgtggagtgatttaatgcgatttcatctaacctcactccacaaaagtatatgggatgttgttgagtttggtgtacaggtaccatccataggggatgaagactatgacacagatgaagtggcccaaatcgagcacttcaactctcaagctacaacaatactcctcgcctctataagcaaggaggaatataacaaggtgcaagggttgaaaagcgcaaaagagatttgggacctactcaagaccgcgcacgagggtgatgaactcaccaaaatcaccaagcgcgaaacgatcgagggggagctcggtcgctttcgtcttcgcccaggggaggagccacaagatatgtacaaccggctcaagaccttggtgaatcaagtgcgcaacctcgggagcaaaaagtgggatgaccacgaagtggttaaggttattttaagagctcttattttccttaaccccacacaagttcaattaattcgtggtaatcctagatatccactaatgacccccgaggaagttatcgggaattttgtgagctttgaatgcatgatcaaaggatcaaagaagatcaacgagcttgacgaaccttccacatccgaggcgcaaccggtggccttcaaggcaacggaggagaataaggaggagtctacaccaagtagacaaccaattgacgcctccaagctcgataatgaggagatggctttaatcatcaaaagctttcgccaaatcctcaagcaacggagggggaaggactacaaatcccgctccaagaaggtttgctacaagtgtggtaagcccggtcattttattgcaaaatgtcctatgtctagtgacagtgacaggggcgacgacaagaagggaaagaggagagaaaagaagaagtattacaagaagaagggcggcgatgcccatgtttgtcgggagtgggactccgacgaaagctcaagcgactcctccgacgacgaggacgccgccaacatcgccgtcaccaagggacttctcttccccaacgtcggccacaagtgcctcatggcaaaggacggcaaaaagaagaaggttaactctaaatcctccacaaaatatgaatcctctagtgatgaccatgctagtgatgaggaggataacttgcttaccctttttgccaatcttaacatggaacaaaaagaaaaattaaatgaattgattagtgctattcatgaaaaggatgaccttttggattcccaagaggattttctaattaaagaaaataagaaacatgttaaggttaaaaatgcttacgctctacaagttgaaaaatgtgaaaaattgtctagtgagctaagcacttgccgtgagatgattgacaaccttagaaatgaaaatgctagtttaaatgctaaggttgattctcatgtttgtaatgtttcaattcccaatcctagagataataatgatgatttggttgctaggattgaagaattaaacatttctcttgctagccttagattagagaatgaaaatttgattgctaaggctaaagattttgatgtttgcaaagttacaattgccaatcttagagataagaatgatatacttcgtgctaagattgttgaacttaattcttgcaaaccatctacatctaccattgagcatgtcactatttgtactagatgtagaaatgttgatattgatgctattcatgatcatatggctttaattaaacaacaaaatgatcatatagcaaaactagatgctaaaattgccgagcacaacctagagaatgagaaatttaaatttgctcgtagcatgctttataatgggagacgccctggcatcaaggatggcattggcttccaaaggggagacaatgtcaaacttaatgcccctccaaagaacttatctaactttgttaagggcaaggctctcatgcctcaggataacgagggttacattttataccctgccggctatcttgagagcaaaattaggaaaattcattctaggaagtctcactctggccctaactatgcttttatgtataagggtgagacatctagttctaggcaaccaacccgtgctaagttgcctaagaagaaaatttctaatgcatcaaatgaacatagcatttcatttaagacttttgatgcatcctatgttttgactaacaaatccggcaaggtcgttgccaagtttgttgggggcaaacacaaggggtcaaagacttgtgtttgggtacccaaagttcttgtttctaatgccaaaggacccaaaacagtttgggtacctaaagtcaagaactaaatttgttttataggtttatgcatccgggggctcaagttggatactcgacagcgggtgcacaaaccatatgaccggggagaaaaggatgttctcctcatatgagaaaaaccaagatccccaacgagcgatcacattcagggatggaaatcaaggtttggtcaaaggtttgggtaaaattgctatatctcctgaccattccatttcaaatgttttcttgttgattcattagattacaatttgctttccgtatcccaattgtgtcaaatgggctacaactgtctatttactgatgtaggtgtcactgtctttagaagaagtgatgattcaatagcatttaagggagtgttagagggtcagctatacttggtagattttgataaagctgaactcgacacttgcttagttgctaagactaacttgggttggctctggcaccgccgactagcccatgttggaatgaagaatcttcacaagcttctaaagggagaacacattttgggactaaccaatgttcattttgagaaagacaggatttgtagcgcatgccaagccgggaagcaagttggcacccatcatccacacaagaacattatgacaagtgacaggccactggaactccttcacatggatctattcggcccgatcgcttacataagcatcggcgggagtaagtactgtctagttattgtggatgatttttctcgcttcacttgggtgttctttttgcaggacaaatctcaaacccaagacaccttaaagggattcttgagacgggctcaaaatgagttcggcttaaggatcaagaaaattagaagcgacaacggaacggagttcaagaactctcaaattgaaggctttcttgaggaggagggcatcaagcatgagttttcttctccctacactccacaacaaaatggtgtagtggagaggaagaatcgaactctattggacatggcaagaaccatgcttgatgagtacaagacaccggatcggttttgggccgaggcggtcaacaccgcctgctacgccattaaccggttatatcttcaccgaatcctcaagaagacatcttatgaactcctaacccggtaaaaagccaaatgtttcatattttagagtttttggtagcaaatgctttattcttgttaaaagaggtagaaaatctaaatttgctcctaaaactgtagaaggctttttactaggatatgactcaaacacaagggcatatagagtctttaacaagtcctcaggacttgttgaagtttcttgtgatgttgtgtttgatgagactaacggctctcaagtagagcaagttgatcttgatgagacaggtgaagaacaggctccatgcatagcgctaaggaacatgtccattggggatgtgtgtcctaaggaatccgaagagcctccacacgcacaagatcaaccatcctcctccatgcaagcatctccaccaacccaagatgaggtcgaggctcaagatgttgaacaagaagatcaagaagatgagccacctcaaaatgacggcaacgatcaagggggagatgcaaatgatcaagaaaaggaggatgagcaagaaccaaggccgccacatccaagagtccaccaagcaatccaacgagatcaccccgtcgacaccatcctcggcgacattcataagggggtaaccactcgatctcgggttgcacatttttgtgaacattactcttttgtttcctctattgagccacacagggtagaggaagcactccaagatgcggattgggtgatggcgatgcaagaggagctcaacaacttcacaaggaatgaggtatggcatttagttccatgtcctaatcaaaatgttgtaggaaccaaatgggtcttccgcaacaagcaagacgagcatggtgtggtgacaaggaacaaagctcgactcgtggccaaggggtattcacaagtcgaaggtttggattttggtgaaacctatgcacccgtagctaggcttgaatctattcgcatattattggcctatgatacttaccatggcttcaagctctatcaaatggacgtgaagagtgccttcctcaatggaccaatcaaggaggaggtctatgttgagcaacctcccggctttgaagacagtgagtatcctaatcatgtttataggctctctaaggcgctttatgggctcaagcaagccccaagagcatggtatgaatgccttagagatttcctcattgcaaatggcttcaaagtcggcaaagccgatcctacactctttactaaaactcttgaaaatgacttgtttgtatgccaaatttatgttgatgatattatatttgggtctactaacaagtctacatgtgaagagtttagtagggatcatgacacagaaattcgagatgtctatgatgggggagttgaagtacttcttaggttttcaagtgaagcaactccaagagggcaccttcatcagccaaacgaagtacactcaagacattctaaacaagtttgggatgaaggatgccaagcccatcaagacacccatgggaacaaatgggcatctcgacctcgacacgggaggtaagtccgtggatcaaaaggtataccggtcgatgattggttcattgctctacttatgtgcatctcgaccggacattatgctttccgtatgcatgtgtgcaagattccaagccgaccctaaggaatcacaccttacggccgtgaaacgaatcttgagatatttagcttacactcctaagtttgggctttggtaccctcggggatccacatttgatttgattggttattcggatgccgattgggcggggtgtaagattaataggaagagcacatcggggacttgccagttcttgggaagatccttggtgtcttgggcttcaaagaagcaaaattcggtagctctttctaccgccgaagccgaatatattgccgcaggccattgatgcgcgcaattactttggatgaggcaaaccctgcgggactacggttacaaattaaccaaagtccctttgctatgtgataatgagagtgcaatcaaaatggccgacaatcccgtcgagcacagccgcactaaacacatagccattcggtatcattttcttagggatcaccaacaaaagggggatatcgagatttcatacattaacactaaagatcaattagccgatatctttaccaagcctctcgatgaacaatcttttaccagacttaggcatgagcttaatattcttgattctaggaatttctttttgctaaacttgcacacattgctcataaatatacctttgatcatgtctctcttatatatgctatgaataatgtgctttcaagtgtatttcaaaccaagtcataggtatattgaaagggaattggagtcttcggcgaagacaaaggcttccactccactctactactcatccttcgccgtcactccgagcatctctccaactttggtataatcatcactcctatgtttcatttgccaaaggggacaaaatagtcattaaagggctctaatgattccgtttttggcgatttatgccaaagggggagaaaatatgagcccaaagcaaaaggaccgcaccaccaccctaattttaaacatatgattttcaattggttgaaaatttcaaaatttgtatctctttgtgttctaaaagggagcatgtagtatatgtagtattttcaaaacttgatatctcaaaaccctcttgaacactaagaggagaatttatttgagggggagttttgtttagtcaaaggaatggcatttgaaacagggggagaaaatttcaaatcttgaaaatgctttgcaactcttattcatttacctttgactatttgcaaaagaaccttgaaaagaatttacaaaagaattttgcaaaaacaaaacatgtggtgcaagcgtggtccaaaatgatatataagaaacaatccatgcatatcttgtaagtattaatattggctcaattccaagcaacctttgcacttacattttgcaaactagttcaattatgcacttttgaatttgctttggtttgtgttggcatcaatcaccaaaaagggggagattgaaagggaattaggcttatacctagttcctaaataattttggtggttgaattgcccaacacaaatctttggactaactagtttgctctagtatatagattatacaggtgtaaaaggttcacactcagccaataaaaagaccaagttttggattcaacaaggagcaaaggggcaacccgaaaggcacccctggtctggcgcaccggactgtccggtgcgccaccggacatgtccggtgcaccagggggactcagactcaaactcgccaccttcgggaatttctagaggcgactcggctaaaattcaccggactgtccggtgtacacccggacagtgtccggtgctccaagggaggtcgtcctcaggaactcgccagcctcgggttttcactccagccgctccgctataattcaccgaactgtccgatgtgcaccggagggggcggtgcatctgcggagcaacggctacttcaggcgccaacggctacctgctgcgcatttattgcgcaccagcgcgcgcagaggtcaggcacgcccatgctggcgcaccggacagtgaacagtgcatgtccggtgcgccaccggacatcaagcggggcccagaagtgtaacgccccgaattttgcagttgaattttttcttttctttactcgccaaaattcgggcgttaccttttccttttcttttttcccttcgctaaaccttgaccttttccaaagttctagcgggattcggtttggatttcccgtgtaagaaaaaccctaaatactttatgttgtttgatgcaccatgccgaaccttgcatttcttttgattgctttgaaagtgcaattgcattcatgtagaaagatcggatttcgaaaatgtggagaggatcttttctttcttttttctccctctctttttctctcctctttttctctctctcccgcgccgtgggccgaccccggccggcccaggcccctgcgcgccccccctcttgggcctggaagggccagccgcccccccctctttcccttattccctaaccctctccctctcccccctcattttctccctcgccaccgaatccgccgcccctaccccctacccgccccctgccctagattggcgggccgccccctgccgccgggcgcccctcgccgtcgatccccgacaccggtgagcccccccctcctccctctctccctcaaccttctctcccctttcctccctccccttcccctcgccgcttggccccatggccggtttggccgcccgccccaccccgccgggtcggccgctcggcccccccgcgccctgccccgcgcccctgcgcgcccagcggttcggccgccccgcccccctgctcggccggctcggccgccccctgcgccgccccccccccccccgccagctcggccgcccgccgccggttcaaccggccggctcagcggctcagccgcccggtcagccccggccgcgcccgcgcccgcgccccgcctagggccggttcaaccgccctagggccggttcaacccccccccccttctgttttttttatttttttattttattttatttactttctgtgatcataattactgtattttaagtaggctaatcactgttcatgctatgggaaaataggaagtttaatttaaaattccgttatgttattgattcacgtagctaattgtttaccctgtgcaatgttgatcaactaaaagtgattaggtttccatcagtatatataaatatatataacagaattattttgttaaaaaccaattgtgtcattagtgcataagatttaaccccctgcgagacctttccccgtttctttctaaccataacaaatgcgttatcgaatgtcatacttgatgcatattcgctttatttgttatcttgtatggtgtactgttcttttgtattaaatatgtggatgtatgtatgtatgtttgcgctcgcatagagaacgatccggtcgaagagcccgaggaattcgcaggagaagcccctgagcagcaatcggttggtggaggcaagtgtcctttgacctatctctgtcctattcattctttaattcacctcccgcattacacatttatacctaaggatcgactagcttttgttatccatgtccttgtttacctatttgggtcggattattactacttagtctgatgctattgctcaactctaatcaatgaacatgatgtgattatctatgatacgctgttttcccttctcttattatgatgttgtacttgtggtattcaagggggctcgagcggtttctcgagtgcctctccgtaaggacctgttctatggatgaccgcccgggaaaacagtgcaaccatgagggtggaatggggtgcccttagctgaataattagaggatccgggatgtagttcacttagccgtcgtgccgtcaatggggctcggtgtatgcggctcgctctgccaagtttgggttcgccccttggggaggagtgcggtgcatttaggaaacctaacgggtggctacagccccggggaatctttgtaaaggctacgtagtgaaaccctgcctattcaccttggtagtgtttaagggtttgatcggcccgaggcaagagggaatcacggcttgtgggtaaagtgcacaacctctgcagagtgttatgaaactgatatatcagccgtgctcacggttatgagcggccaagggagctccagagattagtgatacttgatcagagatattttggtacaggtggcaatgagattgatggttctggttatgattatggtattggtaagtggtattctttccgtttggaaaggatacattgggttaataacttgggttaatgttaaaacctggctttctactagtaagtaataacctgaccaactaaaagcaactgcttgactgatccccacataaagctagtccactacagccaaacaggatacttgctgagtatgttgatgtgtactcacccttgctctacacaccaaccccccccccccccaggttgtcagcattgcaaccactgctcaggcgaagatgaagctgtggaaggagacttccagaaattccaagattacgacgagttctaggtgtgggttagcggcaacccccagtcggctgcctgtgaaggccgcgttatctacgtttcttttccgcactttgatttattgtaagaactatatggacgtctcagacgtatgatgtaatagactattttcccttattaatactattttgacttttgtgtgattatgtccatattatgtacctgccgccttctaataactgatcctggcacgtacatgggtgccccctttcctctaaaaccgggtgtgacataagtggtatcagagccgtgctgactgtaggaccgctaacctagagtagaatggtcgttctaaggattatagacctctgtctcttccttgactttgatatcccttcaaaagttggtcctaccgaccaaacctatgttctactatatactataccttgctaaaaatcatgttttattctaatccttcatttacttatgattcattacttgctggtcatattaattctgttctcacccttttgcttgcgatgtcttttgtagatggctcgacttagacacactgcacgaaagtcagtcatccccttcttaccctcccgccttgctgagcgtccgcttcgccgtcccgtggccggacagtccagccacttggagagactacaccaccgcctgcgtgaggagcaggaacgtcgacgacaggagcagcagagctcttccctctcgctccaccaggagatagagtctgtgaggagctgctcccctgtgcttcctctggagccgccccctgcatcaccactgggcgccccagcttctggagtagctgctggaggagacccagacgacggagatggcgatgacagctcgagccacgacaccgacttctctgctaatcttgagccggaaggatgggttgctcgacccatcactcgcgacgctgctcgcgggtgtcacttccatgatgcgctcgacaccctgctacgtcgggcatttgaccggcatacttggtccgtcgagtatcgctgtgtggtctaccagcatagtcgcggggtctacccggaccgctgggaggcaacttgcttggtgcgctgcccggaggacagtctccagggtgcagaggcctgttcagagcactattctatctctgagcgggactcagctgaggcagccatgcaagatgctgcacggcgtgcgctttcgcactactgctcggttttcggtggggcagctgacggtcttgacctgaagtattacccccgccgttcatctggcagcacaggaggcgtgattgtctcacccgtcggtgagggcaatcctaggttgagcagcacagtcaacctagccgccgtgctaaacacggagctggaccatgcattagacgagctgagtagggctcgtgctgagatcgccctgctgcgggctgagcgtgcggaacgtcgttttctggacggtggttcccccgctcccgttgggactcagcacccgtaccgctcacctcagcgtggacaccagtcttatggcaatcccgcctgcaagaccaagataaacctagaaccatagatcgttagagttggatcttgtaattaatacgaaataaatatatacatggaagcttcagtcttagcgttagtctcggtcttagttagttttagttagacagggtagtttgctatatcctgtgcatttatgtttgtcatgatgaactatgtttggtttggatctttgtaatgattgtcaccagagtgtgggtatcccctgcattttggtttacatactatgtcaataaagttagttatatagttgggaaacccattattctcctttcctctttatctgagaagctgtgtggtctgtgttggagatcagtgaagatgctcatctgttcagtgctgttggagaattctatactcttttcttatgctgcaagctttgccagatcagttctgatgtgtggttgcgttctgcagatgtcagagaacaggcgtagaggaggaaggcgtgctcagcaggagcgagccgctccgcaggatgaggtgccccagcagcagcacctgccgcccccgcccccgatgtcgattgagcagatgtttctgatgcagactcaggcagttcaagccatcggtcagactctggccgccattcagcagcagcagcagcagcaacagcagcaagcaccaccccagcctcagatgcctcagatgcctagagacaagcgtgctgaattcatgagaggtcatccaccaacgttcgctcactcttctgaccccatggatgctgaagattggctgcgcactgtggagcgggagttgcataccgctcagtgcgatgaccgggagaaagtcttgtatggtccccgtctgttgagaggagcagcccaatcatggtgggagtcttacctcgccacccatgccaaccccgacaccatcacctgggaagaattcagaggtagctttcgtcagtaccatgtgcctgcaggtctgatgacagtgaagaaggaggagttcctggcccttaagcaagggtcatcgtctgtcagtgagtatcgagacaggtttctgcaattgtctcgctatgctcctgaagatgtcaacactgacgccaagcgacagtaccgttttctgggaggcttggtcgaccctctgcagtatcagctgatgaaccacaccttcccgacattccagcacctgattgacagagcaatcatgacagaaaggaagcgtaaggagatggaagatcgtaagcgcaagatcagtggaccccagcctggaagcagcagccgtccccgtttctcaggcaatcaacctcagcagttcaggcagaaccagcgtccacctcagcagcatcagcagcgtcagcagtatcagcagttccaaaggcagtatcctcagcatcagtaccagaaccgtcagagcaatcagtcaggaggtcagtttcaaaagcagaatcagcaagcacctcgtcttcctgccccagcaaatcagcagaacagtcaggcagcaccagctcaggttggaaacagggcatgtttccactgtggagagcaaggccattgggtgatgcaatgtccgaagaaggcagcccagcagcagtcaggccccaatgccccagcaaagcagaatgtgtctcagcctggagcaggcaatcgctctcagcagcgctataatcatggaagattgaatcacttggaggctgaagcagttcaggagacccccggcatgacagtaggtatgttcccagtcgattcccatattgcagaagtgttatttgatactggagcaacacattctttcattactgcatcatgggtagaagcacataatcttccaattactaccatgtcaacccctattcaaattgactcagccggtggtagaattcgagctgatagcatttgtttaaatgtaagtgtggaaataagggggatagcgtttcccgccaaccttatagtaatgggtactcagggaatagatgtcatcctagggatgaattggttagataagtatcaggcagttatcagttgtgataaaaggacaatcaagttggtgtccccactaggagaggaagtggtgaccgggttagtcccgcctgagccaaagaaaggaagttgttatcagatggctgtcgatagcagtgaagcagacccaattgagagtatcaaggttgtgtctgaattcccagatgtgtttccaaaggatttaccgggtatgccaccagagcggaaagttgaatttgctatagagcttcttcctggaaccgcccctatcttcaagagagcttacagaatatctggaccagagttggatgagcttaagaagcaaattgatgagctgtcagagaaaggttacatccggccaagcacctcgccttgggccgcccctgtcttgttcgtagaaaagaaagatggtactaagaggatgtgcatcgattatcgagctttgaatgaggtcacgatcaagaacaagtatcctttgcccagaatagaagatctgttcgaccagttgagaggagccagtgtgttctccaagatcgatctgaggtcaggttatcatcagctcaggatccgaccttcggacattccgaagacggcattcattaccaagtatggtttatatgagttcacagtgatgtcttttggtttgaccaatgcgccagcattcttcatgaacttgatgaacagtgtattcatggattatcttgataagttcgtggtggtattcattgatgacattctggtttattctcaaagtgaagaagagcatgcggatcatttgaggatggtgttgcagagattgcgagagcaccagttgtattgcaaagttgagcaagtgtgagttctggatcagtgaagtccctgttcttgaggtcacattatcaacaaagaaaggattggctgtggatcccgaagaaagtggcagacatttctaaaactggaaagcgccaacggatgctcgaggaaatcaagagctttcattgggaatggccgggatattatcggcgattcattgaagggttttcgaagattgcgaagccaatgacagcgttgctaggcaacaaggttgagttcaagtggacccagaaatgccaagaggcctttgaagcgctgaaagagaagttgactacagcgcccgtcctagtcttgcccgatgtgcacaagcccttctctgtgtactgtgatgcttgttacacaggtttgggatgtgtgttgatgcaagagggaagagttgtggcttactcgtcccgacagctgaaggttcatgagaagaattacccaatccatgatctagagttggcagcagtggttcacgcactgaagtcatggaggcactatctgtatggacagaaatgtgatgtttacacagaccacaagagtctgaagtacatattcactcagtcagaattgaacatgaggcaacgaagatggttagaattgatcaaagactatgagttggagattcattaccatccaggcaaagcaaacgtagtggcagatgctctgagcagaaagagtcaagtcaatctgatggttgctcgcccgatgccttatgagttggccaaggagtttgacaggttgagtctcggatttctgaacaattcgcgaggagtcacagttgaattggaacctaccttggagcgcgaaaccaaagaagcgcagaaaaatgatgagaagatcagtgagatccggcgactgattctagatggtaaaggcaaagattttcgagaagatgcggaaggcgtgatatggttcaaagaccgcttgtgtgttcccaatgtccagtctattcgggagttgattcttaaggaagctcatgagacagcttattcgattcaccctggcagtgagaagatgtatcaggatctgaagaagaaattctggtggtacggaatgaaaagagaaatcgcagagcatgtggctatgtgcgatagttgtcaaagaattaaggcagaacaccagagaccagctggattgttgcaaccgttgcagatccctcagtggaaatgggatgaaattggtatggatttcatagtcggattgcctcgcacccgagccggctacgattccatttgggtagtagtggaccgcttgaccaagtcagcccacttcatacctgtcaagaccaactacaacagtgcagtattggcagaattgtatatgtctcggatcgtttgtcttcatggtgtgccaaagaagatagtgtcagatagaggaacgcagttcacctctcatttctggcagcagttgcatgaagccttgggcacgcatctgaatttcagttcagcttatcacccgcagacagatggtcagactgaaagaaccaatcaaattcttgaagatatgttgagagcctgtgcgttgcaagatcagtccggatgggacaagcgattgccttatgcagagttttcctataacaacagttatcaggccagtttgaagatgtcaccgtttcaagcgctgtatggaaggagttgcagaactccgttgcaatgggatcagcctggagagaagcaggtgtttgggccagatattttgcttgaagccgaagagaacatcaagatggtccgagagaatctgaagatagcgcaatcaaggcagcgaagctatgcagacacaagaagaagagagctgagtttcgaagtcggagactttgtttatctgaaagtgtcaccgatcagaggagtcagaaggttcggagtgaaaggcaagctagcaccccgctacattggtccgtatcaaattctctcaaagcgtggagaagtggcttatcagctcagtttgccagagagtttgtctgctgtgcataatgtctttcatgtgtctcagttgaagaagtgcttgcgtgtgccggaagagcagttgccagtggaaggtctggaagtccaggaggacttgacctatgttgagaagccagctcagatccttgaggttgcagatagagtcacccgaaggaagaccgtcagaatgtgcaaagtcagatggagtcaccactctgaggaagaagcaacctgggagcgtgaagatgatctgatggccaagtacccggagctctttgctagccagccctgaatctcgagggcgagattcttttaagggggataggtttgtaacgccccgaattttgcagttgaattttttctttctttactcgccaaaattcgggcgttaccttttccttttcttttttcccttcgctaaaccttgaccttttccaaagttctagcgggattcggtttggatttcccgtgtaagaaaaaccctatattctttatgttgtttgatgcaccatgccgaaccttgcattttgttcattctttgaaagtgcaattgcattcatgtagaaagatcggattcacgaatctctagatcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccggtaggaatcctatcttatgtggtggagcggccctgccgacctgcatcttggatgcttgagggcaatgcattcagtagaagattcggatcgaagtggggggagatctttctctctattttccccctccatttctctcctctttttgctcttcacccgcgccgtgggacggaaccccggtcgaccaggtccatgggcgccccccctttagagcctggaaggcccagccgccccccccctctttcccttattccctaacctctccctctccgctcatttctccctcccccacctaaggcggcggccctaccccctaacccgcccccctgtcctaggttggcgcgccgccccctgccgccgggcgccccttgccgttgaatcccgacaacccgggaggccccccctcctccctctcttcctcctccctcttttccctctcctccctcccctttccctcgccgcttggccccaaggccggtttggccgcccgccccaccccgccgggttggccgcttgggcgccccgcgccctgccccgcgcccctgcgcgcccagcggtttggccgccccgcccccctgcttggccgggttggccgccccctgcgccgccccccccccccccccccgccagctcggccgcccgccgccggttcaaccggccggctcagcggctcagccgcccggtcagccccggccgcgcccgcgcccgcgccccgcctagggccggttcaaccgccctagggccggttcaaccgccccccccccttctgttttttttatttttttattttattttatttactttctgtgatcataattactgtattttaagtaggctaatcactgttcatgctatgggaaaataggaagtttaatttaaaatttcgttatgttattgattcacgtagctaattgtttaccctgtgcaatgttgatcaactaaaagtgattaggtttccatcagtatatataaatatatataacagaattattttgttaaaaaccaattgtgtcattagtgcataagatttaaccccctgcgagacctttccccgtttctttctaaccataacaaatgcgttatcgaatgtcatacttgatgcatattcgctttatttgttatcttgtatggtgtactgttcttttgtattaaatatgtggatgtatgtatgtatgtttgcgctcgcatagagaacgatccggtcgaagagcccgaggaattcgcaggagaagcccctgagcagcaatcggttggtggaggcaagtgtcctttgacctatctctgtcctattcattctttaattcacctcccgcattacacatttatacctaaggatcgactagcttttgttatccatgtccttgtttacctatttgggtcggattattactacttagtctgatgctattgctcaactctaatcaatgaacatgatgtgattatctatgatacgctgttttcccttctcttattatgatgttgtacttgtggtattcaagggggctcgagcggtttctcgagtgcctctccgtaaggacctgttctatggatgaccgcccgggaaaacagtgcaaccatgagggtggaatggggtgcccttagctgaataattagaggatctgggatgtagttcacttagccgtcgtgccgtcaatggggctcggtgtatgcggctcgctctgccaagtttgggttcgccccttggggaggagtgcggtgcatttaggaaacctaacgggtggctacagccccggggaatctttgtaaaggctacgtagtgaaaccctgcctattcaccttggtagtgtttaagggtttgatcggcccgaggcaagagggaatcacggcttgtgggtaaagtgcacaacctctgcagagtgttatgaaactgatatatcagccgtgctcacggttatgagcggccaagggagctccagagattagtgatacttgatcagagatattttggtacaggtggcaatgagattgatggttctggttatgattatggtattggtaagtggtattctttccgtttggaaaggatacattgggttaataacttgggttaatgttaaaacctggctttctactagtaagtaataacctgaccaactaaaagcaactgcttgactgatccccacataaagctagtccactacagccaaacaggatacttgctgagtatgttgatgtgtactcacccttgctctacacaccaaaccccccccccccaggttgtcagcattgcaaccactgctcaggcgaagatgaagctgtggaaggagacttccaggagttccaagattacgacgagttctaggtgtgggttagcggcaacccccagtcggctgcctgtgaaggccgcgttatctacgtttcttttccgcactttgatttattgtaagaactatatggacgtctcagacgtatgatgtaatcgactattttcccttattaatactattttgagcactgtgtgatgatgtccatattatgtaactgctgtgtacgtgaataactgatcctggcacgtacatggttcgcattcggtttgccttctaaaaccgggtgtgacaagaagtcagagctccaacggtcagaatccaacggcagagatgacgtggcagggggcaccggactgtctggtgtgcaccggactgtccggtgtgccatcgaacagacaacttccaccaacggtcatgtttggtggttggggctataaataccccaaccaccccaccattcattgcatccaagtcttccacttctcaacctcttacaagagctaggcattcaattctagacacacccaaagagatcaaatcctctccaattccatacaaagcctaagtgactagtgagagtgatttgccgtgttcatttgagctcttgcgcttggatcgctttctttctttctcacttgttcttgtgatcaacactcaattgtaatcaaggcaagaggcaccaattgtgtggtggcccttgcggggaagttttgttcccggctttgatttgagaagagaaagctcattcggtccgcggaccgtttgagagagggaagggttgaaagagacccggcctttgtggcctcctcaacggggagtaggtttgagagaaccgaacctcggtaaaacaaatcctcgtgtctcacttcattattcgcttgcgatttgttttgcgccctctctcgcggactcttttatatttctaacactaacccggcttgtagttgtgtttatatttgtaaatttcagtttcgccctattcaccccccctctaggcgactatcaagtctcgtcactcacctcacgtgaactccaacggaaacaccgcacatatcggcctgaacagaatcccctcccagctcccgatcggggcggacctaactgaatcgaccgatttctgggcacgattacggggagcgggtcaaatccgggcaatactgcatgcgaatctgtccttggcggcactgacttgtgggctccatctgtcgtaatcatcctcaacagagaagaagcgaaacggatggcgcggtggccgggattcctatgccggtcatttcgcgctggctcggactcctctgatccggacccttggccctgtcatctatatatattgagcaccaaagctatcggatggagctgggcgcaatcgagtaggcaggagggcggtgtaggagagcagagagatgagagaggaaccgccgtgggggattctgtaatccatcgcggtcgcaccctcgaagaccctccaggtgattctctggtatctgctgggtgcgtggcgggcttggcgttcgagaatggcggcctgcaccgccgggattgccctatcaccatcggaacaggtcgtcatccgtcgtgcgccgtgaatagctgaatctgctccgcggtcgctggcaagaccctccccaaaggtttgttttctccttcttcatgcgtagctcaagtcgaagccaggtttcatggacttgcgcgttggtgtggtggtgctccggcgcgtcgcggccaggatgggtggggcgccgtcatgtctggtggcctggtggaagaaaacacctctgccgttgatcggatacttaacggccaggattctgagaaacgtaccgctttggtgggttgggatccagaccgtagatctcgtggttggcggctgtgattataaacgttatacccttcgtccaaatcgtacgcgggccgtcgatcctggaacgttcagtcgtgcggctggcatcgttgtttaaaatctccaccctcgatcgcccatctcacgatcaccagtgagtaccggttcggaagggtactcgatctaatctgggccgtgcgagtttgatcggacgactcaggattgaagttaccctttcgggtgcgaaattattcaaaagaacccctcgacttgttctgattcaatccgccatcctcggattgggtgaattcatatgcctgagtccctggtagagatcaaaatagtgtccgaagcccaaatatgatatctggaataataaaacccctttaagaaatgaattttaatattgaaagttaatcctaaaacttcataaattcataacttctccgtttaaaccccgaattgacccgttccagttgcgttaggttcgtataaatattcactacgtagttatagcattggttataccatatctcatgcttttataattagatatttatttaacccatgtttagtagattaacctctctaaatcaaagttaatctgtttaacattatagtttggctaaaatatgatccctaattatattataatttaacataaggttgaactaattatttatttagattattttatcacatgatctattaaatcaacataaggtctagttttaacggcttaaattgcatgggtcttctgaaaaccataccttttcaaccatgaccccgaatttagcggttctcgaacccacgatctcgtagcatcgcgtagaccgttattattcagtttgtacttatggttggtgtgatgttaattttgtctataccatgtttgtatgtattgctacgtttaacagtgaggttacgagtcacctgaagatcatcctggtacctggaatctcgagtcccaggcaagttgtgcccttgatcacttctttttacccagccatgttctgattaatcataatgatctgcataggttaattttgatgggacccaataggttaccctagtttgattatctttataccttgttaccactaaactttctgggtagtacttgctagtgctatatgtggttttgggtatggagatatattattcatgattacacttttgttatcagttgttatttgttgttcatgataagatcattatgttaattggaacatggagaaccacccgggaaaacagtgctaccacaagggaggaatgggaccccttggctgattaattaggaaagctagtggaagactaccttacccgaaaggggcaagggcagtaggggagtggtcagtgtagggaggccctcgggaggattttgctgcgatggcggtcctgcaagggattcctgcattggagcttcctataaactgtagcgggttttctgaagctagtggaactttgtaaaggcctcgtagtgttaccctgcctcacctcctcggtagaggtgtatgggaagtcgcgatcccttggcagatgggtaacatgacttgtgggtaaagatgcgcaacctatgcagagtgtaaaactggtatactagccgtgctcacggtcatgagcagctcggaccctcacatgattaatttatggaacttaaattcaatttgtcatatgcattgcatcgcaggtgatgttgttacttctgttctactatttaattgggttggtatttacttatacttagtaattgctaataaaattttgaccaactttaaaagcaatgctcaactctaaccatcctctttggtaagccttacacttcacatgagctcccacctttggcgagttcatgcacattattccccacaacttgttgagctatgaacgtatgtgagctcactcttgctgtctcacaccccccacaggtcaagaacaggtaccacaggatgaggctcttggaggatgcttcgatgtgttcgtgagaggtctaggccgtcgtctcccagtcaactttgggttgctggaccgttgtctccttataatgtaattatttattttgtatagaactcctgttatatataaagatgtgacattcgatcctgtgccactatacatcatatgtgtgagacttgatcccagcatacctggtgtttatgttcgtgcccgggtcttggtgccctaaaacctgggtgtgacatcccaccgcgcaactgcttccgtcctctgccaccggggcacgccgaaagctcgcctcgcaagcgcgcggccgcgccagcacaccacgcctcgcttcgtttacccgttgcttgccgccgaatcgaccacgctggcgaccaggccagcgccatcgcatcgtccccggaccgctccggtcgcgcccgccaggcctcggggctcgggtagaccaccaccaaaacagaaccttccctccggaccgaaaccgccgcttcccctccccgctctttctgtgaggagggaagcgaggcggatgccgaggcgacggggctcgcgcaccgcactcgtgtgcccgccgcccggggaccaggcgacgtggtgactggtgaggtgaggcaagcgcgcggttttaaattccccgcacgtccttctcgccgtagttttcagtccggaggtacatcaagggaggaacaaacgtgagggctgcctgcctccggacatgggctccagcttcaagtaccgagctggcctctgcctcatcgtcgccgtcgtgctgatttgggtcatatccgcggaggtcacgcaggtgaggtgtggtgtggatcctttgatcttggtgcgtgttcggtgttcctctgttcacttgtttatttttattggagcctcttggatcttggcttggcctttgggacgcatgttggcctattggtacgttttgtgcgtaaatgctctgtgcctatcagattgtatttgttgggacgacctgggggtgttgctctgcatctagatttttaaacgttacgcgcagggttgcctgtgatagaaaatttttcccaggggaaggttatgcatctgccaccactctgccttaaaaagaacatctggcatcattctgaatccagccgaggcttcaccactggactttatttatttacaccataaaaaggcaaagaaaagtcaaattcttttgtcttctactgtatacttttggagctgcctgcatgcatgctcttatggacacccgtcaaatctgctgttaatggtttaacgacttgtacctgcctgtcgtcagtcctcggtgcttagcttgttcatgtaaactatgctaaagcataatcatgcatgtggttgttgtcacagttcttctgtggttgattgagggtggttgcgatatgtttgatcacctacaatacacaaacatgattaaagaagataccgatggtagaatcaggggatactggtagtacaatttcgtcactctcatgactgcaaaacacctcaacttctagttcttgattctttttaaaaaaatatatatttgtagtgtccatattgtgaaatatatcaattttcattttgtaactaaatttcttgtgcaggggatattcacaaagtacaaacatccttttgcagttacttacttgggggcctcccttatggtcatatacctccctttgtcatttctaaaggattctggaaacaccagtgcttcataaattgcaagcaaaacttcctttggtggtggtgcccctttgaagaatgacgaatttcggaagactttagtgctaaattgcactgatgtgagcattcctgtagtgaaagagacaaaaccactcatatatggaatcactgaaatgaatgatggtgttttgaaggacaagcaactttcgactaaggagattgcaacttatggattgtatctttgccccttatggtttgtgacagaggtaattttacaatggctttgcatgttaatttgcaattccaggggacttgaaatgttcatgtgaataacttttgacatttgtttctcaaaaactactgatatatgtttcagaaagcctaaatttcaatcgtaaaagcaatactttgataggtaaaggtaactcatttttctcttacgctgaatgaatgcagtatttatcgaatgcagcgcttgcaagaactagtgttgccagtaccaccgtactgtcttcaacttcagggctcttcactcttttcattggtgttttacttggccaagactccataagtgcttcgaaagttattgctgttttcattagcatggctggcgttgtaatgacaactatgggacagacttgggcatctgatgaatcagaagtaggcaaatctgggtgagttgtttattactaggcctttactcttttatactctgataatcacattggacttaatttaatgttcttgtaatttttttggtcctgcgtacaactagtagttttcagttagttggcttgcctggttttttgggttaaaaaacaactcaaaacttgagtgttttatttctatatttgcatgacttgatagtgatgaacatgaacaaaaacgaatttcaattctgagattggaatttcgttcctattttcaactgcatttccttgctactgatacataagctttacaattgcagggctacacaaaggactcttctaggtgatatgtttggttttatgtcggcaatcgcgtatggtctatttactggtaggttggtttgctttgctttatttgcagtggagctctatgaattcggcccctaattgtttcgatattgtttcagtgctgcttaaaaaattttgtggggaggaaggagaaaaggttgatgtccaaaaactgtttggttatcttggactttttacccttgttgctctctggtggattggtattttttgtttcccttttcgaaattaatataacaggagtatacaatttcgtaatttatgatcgtattctggtatttcttaattgtgcatgcctttcatggttattttatgtaaaaaaaaatatggttattgtgttcatatttcagtctggccattaaccgcactaggcattgagccaaagttttcgatgcctcactctgctaaagtggatgaagtggtggtggcaaatagcctaattggaagtgtactatcagattacttctggtatgtgttaaagaaggctgatatctttttatattaccagaccactctattattacttgcatactcggggtaattgtgacaggctgactgtggaacttatggccagggccctttctgttgtttggactactcccttggtggccacgctaggcatgtctctcacaattccactggcgatggttgcggatatgatcattcacggccgtcactattcagcagtgtatattctttgttctgtccaggtgcgagtatccaacagtttagtatttttttaaaaaaaactagaattatagaaaaatatctactatatcttacggcagtaaccaatatgaatgctacgtgttcctgttcagcattctagctcactgttgagtacatttgtcattctgtttgaccatgtgagctttgttcctttgcaggtattttcaggctttgtcatcgcgagccttgcagatcggctttcacgttctctagggctattatagtttcataaaacgaagcaggttcttgttagagtgtacatctggaagtttaaaggtcgggttggttagacaaaatacttgaggaaacacgggttgggatttgggaaatgccagtaattcgtttcccttttgctcctttaaacataggagcggtgaattttgccgcggcgaatagtaaaggccagcagacgaagaagaaaccgtatattgtagcaaccctttgcaactgtatattctcctactacaaattgagcgacagtgtaaactgccgtcgttctggccttgtcgatccgtgtcgattgcaaatttgcaaatgtcacttttatatccttgtcgatccgtgttcatggcaattgcgcgagttaacaaagaatgaatgggcaaaaaaaaaaaaaaaaaaaaaaaaaacggagctacaaattgcagcgaattgaatgggcaagttgtcatactcctatgcaaagtgtgcgacaatgcaaactgaaaagtgaaaatgacttactgcacaagcaacgtcaaattcaaagtgtacatgctactagcagcttagcgagcaactgaagttctcatagtcgccatgtcggagcgagatgcgatagacggcgaaggcggcactctgtccggggtcgcgcagcagcggcgtggtgaccctgtcgacctcgaccacctcaaaaccatgctcccgcagcacgtccatgcagtccagcacgctgtcccgcacctcgctcgccgcccaccccgcggtgccgccgccgccctcgtccgtccaccacataccccgcagcgtggccgccatcgccggcatgtcctcgggatcgtagaagacgtcggacaagagcacgacgcccaccctcccgtcgccgtcgtcgagcgggagcaggtcgccccagcggagctcgcgcacgcgggcccgcgcggcgctgaggccgttggcgtccgcgttggccctgaggcccggcagcagcgcggccacgtccgtgagcacgcagcgcgcggcgccgaggcaggccacggcggcgatgcccgggagccccgtgccggcgccgagctccacgacggtggcgccacggaggcggcgcctggcgcgggggtccgcggcgaggtgcgcggccaggaccagggacgactcccacagccaggagccggtgaggacgcggcccgtggccgggtcgctcgtgccgtcgcgctccaccaccgccagcgcgcgcccggccacggctacgtgcaggacctgcgggcacagcgcgtccattcccgttcccgcgcgcggctggcttctccctacgacctacgtgccggcctcgtcgtcttaattatctggtcgttgccgccggagcctctgctggaatttctctgtcgttgccggtggcaggttagcgacgggacacttgcggcgtcaatataattgtttgcgcgatggcggtttggtcgtgcattgtagttggttgatgtcgtccatgcttactgaccgactgaatatgttcgttaaccttttcttttggtcagcggccagcgggtggtggttggttcgaacaaaatccttgggccgattgggcgctaaagcttggggccggagccctccgtcagtggccagcccatgggcgacatctcggataaagattgacgaatcacaagaagaaagggaatggtacaaaaaaaaataggaggccgtatgaatgcttgcacttgcacagttgaaagtgcttagattagttatattatcgtttatacgcttgctctaggtttagacaatatttccattgattagaaaatctcataccacttaatgtttgtgtgcaccatatctgtactcgaagagtcttatagtgagatcacaccaaccatatttgtggtgtgaccccacctcacacagagaaaacgaggcacataactgtttggccaaaggctaagacgatggggaacaattcaaaagagaccatctaggataccaacttacatgtggaaaagacctaaaggctatatagttatccgatcggaagcttggcgcttgcaagactccccaacaaggagtatgcaaaagtttgcactcttctcaataccttaaaggtatcagagttctcgacataactctaactcaattaagatttgatactttatttattgcttacctaggttatgtgtttatttcatagtttagcttgctaggctagcgacaatattgaaacctatattgtataattttttgtgatagaaatagcaacacatctgataaaattatagctacatatctagatagattaattactttctggcctaagtttcgactacatggaaattagaggccttagtgtaaaaattgagttttaagtggcataattcgcacctctgtgttaggcgtcacggtctctttaatggtattgcactaaataactcaactgtcatatttttgtgttgccctgatctgttggctcttggactcttggttaattggttggttttaggaattttggttgaagtgcttggttggtttctgttcttgccttctaagttctgatccgtgaagcgtgaatgaatctgtgaaactgtaaactatgcgatcgtgaagaactgaagatgttcatatattgtacagatatgcaatgtgctagatcactgttgatttgttgaatatgtgacgatcaatgaatatattagctcgccacaacaacaaattaaaactaaagcaacacttgcaatcgcgcgcgtgcctggacaagcagcagctcacatcacatgcatgcgtgtgtgacgatatgtgtccaggtgaggcaagcgcgtgcgtgtagttgaccacgtacgagacggtgcgtgtgcgtgcgcgtgaccagcactccagcagccatgtatgcatcgacctcttgccaggcagtagcagcgacgaagagccgagccgagccggccgacacgcacgcgctgcatgcatgcaggtggccgaagacgtcaaacgtcatggcgggcgccagcggtgcgtacgtaccgtgctttccaatcaagcaagagcatgcagcatgcatcgtccactcgtcgtcgccggagctccggcagaacacgggacagctagcggcgcccgcccaggaggacacgggacgcgcgcccgccggccgccgcgtggcgaggctggacggcgtccggggacctagtactagttgagcacgtacggctgcatatatgaggacgaaccgagcgaggctgtctggcgttgttgcaagcggcgcagctgatggcccgaccccggaagcaacaccttcctcttctcctcacgcaagagcgcggcctgccccctttgtggccgtacgacgtggccggccgggcgttaccatccgctagtacgcacaccgagtcgtggaggccatcacgctacgcttgctcagtgttcctctatcgtgttcgtacggggtacgtgcgtacacgcgccatgggcgggccagtgacggaagacacacatgataggtagggctagaaaagatgcgtgatgcagcagcagcacaccacgcgggtcgtcgtcgtctctctcgcgttcgcgttgtagcagccacaccacaccacaccacagtctgcaactctgcatggacgtacggggtgcaaataaggttttgcgagagcagaaaagacccatgtcatcctgtgcagcagtgtgctgagcgactgcgaggcttaggcgcttagccgagcctgcgggatgcgccgatccgtccagaggactccactccaggtccggtacccatccatccgtcgcgggcgggccgtcaacccctttggcttttaatgcatttaatgcagcccttttgtcccttaaaaagctaccaccagccgctcgatcccgcatttggtcttggccacgctcgctagctcccttgcttgcttactttgctcagctagctagctagcgagagggccagagagcttcgccatggcggcgtcccactcgcccaaggtcgcgagggaggtcgccgccgccacctgcgccgtcgacgccaacaccacgttcgtgcaggccgacccggccacgttccgcgcgctcgtgcagaagctcacgggcgccgcgacggacgatgcggcagcggcggcggcggtgaccgtcgcgcacgcgccaccgcccccgccgaggcggcccaagctccaggagaggcggcgcgcggcgccggccaggctggagctggagctggcgcggccgcagccggcgtcgccgtccccgtcgtcgtcgttcttctactaccaccaccaccacgctcacggcgcccacgcccaccaccactgccacgggcccatgcattcgccggtctcgcccatggacgcctacatagcgctggcggtctccgcgtcgccgtcgctgtcgtcgtcctcgtccatgtcgccgcactcgtcgtcgtgcggcggcggcggggccgtggtggtgataagcagggaggaggaggaggagcgggaggagaaggccatcgcgtccaagggcttctacctgcacgcgtcgcccaggggcggcgacgaccagcccaagcttctgccgctgttccccgtacgtccactcaaatcaactccggcgagctgagggctgaggctagctagctagccgtgtgcgttgaccactgcacaaacatgcgtttccggtaattccagtgatgtaaccgcgcgtctatatatttgcgctagaagaactagatgatgatgatccatggatagatgatgcgacgtcgtacgcttgttaattaatttttagctaggcctttactgctttatagttagtagatggctaattaatcatctgaatgtaatgacgcgcgtggcgtgaatgaagtgtgtctcccgtacgtcttatgtgtggtgtggtgtgggtcatgtgttccactagagcaggcaggtcaactcttgatcctgcaatggaacccccatcgaactgcatcaagcaggcaaagctgtttgaaggtttatctataacgcgttccacgcacgtaggctagtactccctccgtttctttttatttgtcgctggatagtgcaaaattacactatccagcgacaaataaaaagaaaacggagggagtacgtagctgcaatgtgggcgggcgacatctgcagaacgttgtcatctacaggagaggaatatgtgtgtgggaagtgggaggtagccaccacagggccgtcacgggctcacgggcgccatgccgcctccgtggctagctgcggcagagcgcagacgatggatcgcagagctcatcatctgctgatggctgcctcacatgatttgtacgcgttggccgggaacgtatgtatgtatgtatccctagcttgagaactgcgctgagacgaacgagatcatccccggttagctaaagcatgattgggagagcccgccgccgcgcacacgtgcgacactggctgcccaagcaaagcgaattgaattcgaagccagccggggcaagcgatacaaaacgaacgccttgcctccgacatctctcaagtccccaacccaacgggatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccttctttggcgatctgaagcaataataaatagataacaaaagtgtaatcatgaataatgtatctccatacccaaaaccacatatagcactagcatgtactacccaaaaatttagtagtaaaatcagtggtgaaacaaggtataaagatagccaaatctagggtaacctattgggtcccatcaaaattaacctatgcagatcattatgattaataagaacatgaatgggtaaaagaagtgatcaagggcacaacttgccttccacgagctcctgctcagcagtctctaccagctgaacctcaggatcctctgtagcttgctcgtctactcgcatcaatacaatacatacatggtatagcaaaaattaacattacaccaaacatgtaaataaaatgcacagtaataatctagaccctaaaatgagattataggaactggaattattaattttggagttatatattttaagttatgaatttccaaaggttttatgcatttaatatagggttaagtgagaaataaattttaacgtgtatttcatgtaaaaacagtggtactatttgatagagaataatattacaaaattataggaactggaatggtccaatttggagttcatatgcattttctacaaattaaacaagtttctgcatttaaatatgtactaaaaccattttctaaaggattttcctggttttattaattatctggactgggcctcaattacagaaatgcgcaggggctaaacaataaaaattcccgagacacagagggtaactcaagtgactgcgggttgattcttaagtttttctagggtctcttctgcgaactggcaaagtgaaggggtatcggccatgatgggccatcggatcagacccgaacgacccagattaggtacgtgcacttaagtgatcagtctgcaacgcgatcccttggatcgggatcctacggcccatagccaatccgtgggttggaacgcagaaccaaatctacccgtttgttcccaaaccaacggttccgatttaaagaaattcatcaccgcgccagcactaatctcaagcgcccaaccagaatccaacggcccagcaccgacaaatccccaccattcatctcccactccacggatcaccggctgtcttcttcctccccacgcccatctaccccttttctctctcttcatggttgtatgctcgcctgaagcccccccatggtgacgagtaccccagagaccggggcccacgattgacaggcaataactaccgcagaccgcagtagctaggcgcgggttatgcgttatgagactcacagggaggttaacgcaccggaacccggatggtgtcggggaagatggccgtcggcggacttgcggtggagcgcggatccttcgcctcccgcccggcgacccacacgatcaacgtcgcacggctctgcttctctgacaccacccgagattctagaaatcgcggaagcctaaacgcgccacgacccttctcctcgcttcactcggctcgtaaaaataccagcaccagggatcctgatttatatcgactacatggagccaatccgaggagggcatgggcgcaacagctgcggccgaatcggccggactgttgacggagtctggcggcgcgcggtaaaaatccgccgtctgttagcgcacgcgcgattcgcgtgaggagaagaacaggagcacgcacctgacgtgtgggcctcaatggtcatctccacgcgataaggaagggcgagcgcgagagaggccgagtggtgggtcccagagaccagtgtcaccagcgaagacgggctgcgcggtgcaaatcaaagatgggccgatttggtagctgtagcccatttaggttcgacttcttttctctttttctctgattttcttcttctcctattttcaattcccaatttgaatttcaagtatgactttgaatttgtactcaaattaaaaatagattttaatcataccagtatggttaagtttgtttatttgtaaattttgttttatattttatattatctctttctttctcctttttcctcaaatcctcttctcattgtcattttatttaattcatattattattgcttttaatgcacaaacaaaaatccaatatgatgcaatggtttatttgtgtcttattaaggatctactctttttttacatgagtggtcacatgtaatgataactagagacacacatacatatataaaggaaacaatttctccttttattcttccttacaaagtgggtattacaagggagaacacgtgatgggtttgactaacgtgcaattcgaaaaagatagaccttgtgcagcttgtcaagcaggtaaacaagtgggaggagcgcatcacagcaagaatgtgatgaccacttcaagacctcttgagctgctacatatggacctcttcggacccgtcgcctatctaagcataggaggaagtaagtacggtctagttattgttgatgacttttcccgcttcacttgggtattctttttgcaggataagactgaaacccaagggaccctcaagcgcttcctaaggagagctcaaaatgagtttgagctcaaggtgaagaagataaggagcgacaacgggtccgaattcaagaaccttcaagtggaggagtctcttgaggaggaaggggtcaagcacgagttctccgctccctacacaccacaacaaaatggtgtggtagagaggaagaataggacgctcatcgatatggcgaggacgatgcttggagagttcaagacccccgagtgtttttggtcggaagccgtgaacacggcttgccacgccatcaacagggtctaccttcatcgcctcctcaagaagacgtcgtacgagctactaaccggtaacaaacccaatgtatcatactttcgagtatttgggagtaaatgctacattctagtgaagaagggtaggaattccaaatttgctcccaaagccgtagaagggtttttgttaggttatgactcaaatacaaaggcgtatagagtcttcaacaaatcatcgggtttggttgaagtctctagcgacgttgtatttgatgagactaatggctctccaagagagcaagttgttgatcttgatgatgtagatgaagaagacgttccaacggccgcaatacgcaccatggcgattggagaggtgcggccacaggaacaaaatgagtgagatcaaccttcttcctcaactatggtgcatcccccaactcaagacgatgaacaggttcatcaacaggaggcgtgtgatcaagggggagcacaagatgatcatgtgatggaggaagaagcacaaccggcacctccaacccaagttcgagcgatgattcaaagggatcatcccgtcgaccaaattctgggtgatattagcaagggagtactactcgatctcgattagtaatttttgggggcatactcttgtctctttctatgaactttcaggtagaaaggcttgctagaatccggactgggtggtggggcatgcagagagagtctcacaacttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctttacttcttatgttcagcttgattcgtaaccttcataaacatctaaagggagaacatgtgataggtaaaacaaatgtaaccttcgaaaaagatagaccttgtgcagcttgtcaagcaggtaaacaggtgggaaggtctcataccaaaaatgtgatgaccacatcaagacctttggagttgcttcatatggaccttttcggacccgtcgcctatctaagcataggaggaagtaagtatggtcttgttatcgttgatgacttttcccgcttcacttgggtattctttttgcaggataaaacagaaacccaagggaccctcaagcgcttcctaaggagagctcaaaatgagtttgagctcaaagtgaagaagataaggagcgacaacgggtcagagttcaagaaccttcaagtagaagagtatcttgaggaggaaggaatcaagcacgagttctccgctccctacacaccacagcaaaatggtgtagtagagaggaagaacaggactctcattgacatggcgaggacgatgcttggagagttcaagacccccgagcggttttgatcggaagccgtgaacacggcttgccacgccataaaccgggtctaccttcatcgcctcctcaagaagacttcatacgaactcctaaccggtaacaaacccaatgtgtcttacttttgtgtatttgggagcaaatgttatattctagtgaagaaaggtagaaattctaagtttgctcccaaagctgtagaagggtttttgttaggttatgactcaaatacaaaggcgtatagggtcttcaacaaatcatcgggtttggttgaagtctctagcgacgttgtatttgatgagactaatggctctccaagagagcaagttgttgatcttgatgatgtagatgaagaagacgttccaacggccgcaatacgcaccatggcgattggagaggtgcggccacaggaacaaaatgagtgagatcaaccttcttcctcaactatggtgcatcccccaactcaagacgatgaacaggttcatcaacaggaggcgtgtgatcaagggggagcacaagatgatcatgtgatggaggaagaagcacaaccggcacctccaacccaagttcgagcgatgattcaaagggatcatcccgtcgaccaaattctgggtgatattagcaagggagtaactactcgatctcgattagttaatttttgtgagcattactcctttgtctcttctattgagcctttcagggtagaagaggccttgctagatccggactgggtgttggccatgcaagaggagctcaacaacttcaagaggaatgaagtttggacactggtgcctcgtcctaagcaaaatgttgtgggaaccaagtgggtgttccgcaacaaacaagacgagcacggggtggtgacaaggaacaaggctcgacttgtggcaaaaggttatgcccaagtcgcaggtttggactttgaggagacttttgctcctgtggctaggctagagtccattcgtatcttgctagcatatgccgctcaccattctttcaggttgttccaaatggatgtgaagagcgctttcctcaacgagccaatcaaggaggaggtgtacgtggagcaaccccctggcttcgaggatgaacggtaccccgaccacgtgtgtaagctctctaaggcgctctatggacttaagcaaaccccaagagcatggtatgaatgccttagagactttctaattgctaatgctttcaaggttgggaaagccgatccaactcttttcactaagacttgtgacggtgatctttttgtgtgccaaatttatgtcgatgacataatatttggttctactaataaaaagtcttgtgaagagtttagcagggtgatgacgcagaaattcgagatgtcgatgatgggcgagttgaactacttccttgggttccaagtgaagcaactcaaggatggcaccttcatctctcaaacgaagtacacgcaagacttgctgaagcggtttgggatgaaggacaccaagcccgcaaagactccgatggggaccgatggacacaccgacctcaacaaaggaggtaagtccgttgatcaaaaggcataccggtcaatgatagggtctttactttatttatgtgctagtagactggatattatgcttagcgtatgcatgtgtgcaaggtttcaatccgatcctaaggagtgtcacttagtggcagtgaagcgaatccttagatatttagtcgctacgccttgcttcgggctctggtatccaaaggggtctacctttgacttgattggatattcagattccgactatgctggatgtaaggttgataggaagagtacatcggggacgtgccaattcttaggaaggtccctgatgtcatggaactctaagaaacaaacttccgttgccctatccaccgctgaggccgagtatgttgccgcaggacagtgttgcgcgcaactactttggatgaggcaaaccctcagggactttggctacaatctgagcaaagtcccactcctatgtgataatgagagtgctatccgcatggcggaaaatcctgttgaacacaaccgcacaaagcacatagacatccggcatcactttttgagagaccaccagcaaaagggggatatcgaagtgtttcatgttagcactgagaaccagctagccgatatctttaccaagcctctagatgagaagaccttttgcaggctgcgtagtgagctaaatgtcttagattcgcggaacttggattgatttatagcatacatgtgtttatgcctttgatcatattccctatgcattttgttgcttagttatggtgctcaagttgtacaaacactccctggacctcacaagtccattgcaaagtgatgcacatatttagggggagatgtgttacaacttgatcctttgagactaaccatgtgcttgagtttgctgatttaatctcaaagaaggattgaaaggaaaaaggtggacttggaccatgaaagacttccactgcactccgatgagagggtaacttattccaagttcatctcatgtactcttattgcctttgcatttttattgaagattttggtgaggcaatggggttcaagggccaagattaatcccattttggtgcttgatgccaaagggggagaaaataaaggccaaagcgataaatggatcagctaccacttgagagattttgaaaatagtagaatagagcttttggtttgtcaaaactcttttattgtctctcttgtcagaaagttggtttcttgtggggagaagttgttgattatggaaaaaagggagtttttgaaatcttgaatcaatttctcttggaatgactctatttatgtcttaacatgtgtgtttgacttagagatagaaatttgagtttgatttgcaaaaacaaaccaagtggtggcaaagaatgatccatatatgccaaaactgattcaaaacaaatttgagttttatttgaagtgattttgcacttgttctacttgctttatgttatgttggcataaatcaccaaaaagggggagattgaaagggaaatgtgcccttgggccatttctaaatattttggtgattgagtgccaacgcaagtgcttttgagttgatctgtgtaaaatggtggacaaagtacaaatcaaggataaaggtatgtttctcagacttagtacattgttttagagactaatgtattgtgtctaagtgctggaaacaggaaaaatcgaattggaattgtcttgcctcgagcagccaagactctgctcagtctgggagcaccggactgtccggtggtgcaccggacagtgtccggtgtgtcaggctggctcgagcgaactggccgctctcgggaattcaccggcgacgtacggctataattcaccggactgtccggtgtgcaccggactgtccggtgagccaacggtcggccgggccaacggtcggccgcgcgatctgcgcgggacacgtggctgagccaacggctagaagggggcatcggactgtccggtgtgcaccggacatgtccggtgcgccaacggctctctggctgccaacggtcggctgcgccatttaaggaaagaaatcgggcaccggacagtgtccggtgtgcaccggactgtccggtgtgccagtcgacagaaggcaagatcagccttcatagaatgctctcaacggctactagctgccttggggctataaaagggacccctaggcgcatggaggagtacatcaagtatactcaaagcaatcctaagcatcgagacttcgattccacgcatttgtttctttgtgatagcatctagagctctagttgagttgtaaactcattgggttgtgttgcgagctcttgttgcgacttgtgtgcgtgttgttgctctgatttctgagtcttgtgtgcgttgctcatccatcccttactccgtgtttctttgtgatcatctaagtgtaagggcgagaggctccaaagtgtggagattcctcgcaaacgggatatagtaaagcaagcaaaacaccgtggtattcaagtgggtctttggaccgcttgagaggggttgattgcaaccctcgtccgttgggacgccacaacgtggagtaggcaagcgttggtcttggtcgaaccacgggataaaccactgtgtcatctctatgattgttctcttgtggttattgtgtttttgctaagacttctctctagccatttggtgattattgtgctaacacttaaccaagttttgtggcttaagttttaagttttacaggatcacctattcaccccccctctaggtgctctcactcactctttatttgcccgcgatttgttttccaccctctctctcggacttgtttctatttctaacgctaacctggcttgtagttgtgcttaagtttataaatttcagatttgccctattcacccccctctaggtgactttcaattggtattagagccccgtgcttcattagagcctaaccgctcgaagtgatgtcgggagcatccgccaagagggagatcgggaccggcgagaagcccgccacaagccatgggaaggctccatatggagagtccgccaacaagatgaagggctcccattcacacgataagtcgcgtcggagcggtgacaagaagaagaagatgaggaaggtggtctactacgagaccgactcttcgtcaccatctacctccggctccgacacgccgtccatcacttctaagcgccatgagcgcaagaagtttagtaagattcccctatgctatcctcacatttctaagcatactccattactttccgtcccattaggcaaaccaccgacatttgacggtgaagattattctaggtggagtgatatgatgatatatcacctaacctcactccacaaatgcatatgggatgctgttgagtttggtgtacaggtaccattcgtataggtaccatgatgaggacgaggtggccaaatcgtgcacttcaactccaagccaccactatactcctcgcatctctaagtcgagaggagtataacaaggtgcaagggttaaaaagcgccaaggagttttgggacgttctcaagaccgcgcacgaaggagatgagttgaccaaaatcaccaagcgggagacgatcgagggagagctcggtcgcttccggcttcgccaaggggaggagccacaagacatgtacaaccgcttgaagaccttggtaaaccaagtgcgcaacctcgggagcaccaaatgggatgaccatgagatggtcaaggttattctaagatccctcgtttttcttaatcccacacaagttcaattaattcgtggtaatcctagatatacactaatgtctcccgaaaaagtaataggaaactttgtgagctttgagttgatgatcaaaggctcaaagaaaatcatggagctagatggcccctccacgcccgaagcacaaccggacgcattcaatgcaatggaggagaagaaggaggagtctacatcaagtagacaacccattgacgcctctaagcttgacaacgaggaaatggcgctcatcatcaagagcttccgccaaatcctcaaacaaaggaaggggaaagattacaagccctgttccaagaaggtgtgctacaagtgtggtaagcccgttcatttcattgctaaatgtccattatctagtgatagtgacaggagcgacgacaagaagggaaagaggagagaaaagaagagatactataagaagaagggcggcgatgcccatgtgtgccgcgaatgggattccgacgagagctcctccgactcctcctccgacgaggacgctgccaacatcgccgtcaccaagggcctcctcttccccaacgtcggccacaagtgcctcatggcaaaggacgacaaaaggaagaaggtaaaatcaagatcctccactaaatatgaaacctctagtgatgaggataattatagtgatgaagaggataatttgcgcaccctttttgccaacctaaacatgcaacaaagggaaaaactaaatgaattaattagtgctattcatgagaaggatgatctcttggactctcaagtggacttcctaattaaagaaaacaagaagcatgttaaggttaagaatgcttatgctctagaagtagaaaaatgtgaaaaattatctagtgagctaagcacttgtcatgatattattgccaaccttagaaatgaaaatgctaaattaatttgctaaggttgattctaatgttagtaatgtttcaattcccaatcttaaaaatgataatattaatttgcttgctaagattgaagaattgaatgtctctcttgctagccttagaaatgataatgaaaaattaattgctaaggctacagaattagatgtttgcaatgctttaatttccgatcttagagataacaatgatattttgcgtgctaagattgttgaacttaattcttgcaaaccccctacatctaccattgagcatgttactatttgcactaggtgtagagatgttaacattgatgctattcatgatcacatagctttaattaaacaacaaaatgatcatataacaaaattagatgctaaaattgctgagcatgacttagaaaatgaaaattttaaatttgctagaagcatgctctatagtgggagacgccctggcatcaaggatggcattggcttccaaaagggagaaatgtcaaacttagtgcccctcctaaaagattatctaactttgttaaaggcaaaactcccatgcctcaggataacgagggttacattttgtaccctgccggttaacccgagagcaaaattaggagaattcactctaggaagtctcactctggccttaatcatgcttttatgtataagggtgagacatctagctctaggcaatcaacccatgcaaaattgcctaagaagaaaactcctattgcatcaaatgattctaacatttcatttaaaacttttgatgcatcttatgtgctaaccaacaaatcaggcaaggtagttgccaagtatgttggggacaaacacaagagctccaagacttgtgtttgggtacccaaagttcttgtatctaatgtcaaaggacccaaaaccgtttgggtacctaaaatcaagaactaaacttgttttgtaggtttatgcatccgggggctcaagttggatcattgatagcgggtgcacaaaccatatgacaggggagaagaaaatgttctcctcctatgagaaaaccaagatccccaacgagctatcacattcggagatgtaaatcaaggtttggttaaaggattgggtaaaatagctatatcacctgaccattgcatttcaaatgtttttcttgtagattctttagattataacttgctttcagtttcgcaattatgtaaaatgggttacaactgtctttttacggatacaggtgttactgtctttagaagaagtgatgattcaatagcatttaagggagtgttagatggtcagctatacttagtagattttgatagagctgaactcaacacttgcttaattgctaaggctaacaaggactggctctggcatcaccgactagcacatgttggaatgaagaatcttcacaaacttctaaagggagaacacattttgggactaacaaatgttcattttgagaaagacaaagatttgtagcgcatgtcaggcaggaaagcaagttggtgttcatcatccacacaagaacatcatgtcaactgacaggccactcgagctcctacacatggatctattcgacccgattgcttacataagcatcggcgggagtaagtattgtctagttattgtggatgaccattctcgcttcacttgggtgttctttttgcaggaaaatctcatacccaagagatcttaaagagattcttgagacgggctcaaaatgagttcggcttgatgatcaagaaaataagaagcgacaacgggacggagttcaagaactcacaaatcgaaggcttccttgaggaggagggcatcaagcatgagttctcctctccccacacgacacaacaaaatggtgtagtggagaggaagaatcgaactctattggatatggcaagaaccatgcttgatgagtacaagacttcggatcggttttgggccgaggcggtcaacacggcttgctacaccatcaaccggttatatctacactgaatcctcaagaagacatcatacgaactcctaaccggtaaaaagcccaatgtttcatattttagagtctttggtagcaaatgctttattcttgttaaaagaggtagaaaatctaaatttgctcctaagactgtagaaggctttttactaggttatgattcaaacacaagggcatttatagtctttaacaagtcctctggattagttgaagtctcttgtgacattgtgtttgataagagtaacggctctcaagtagagcaagttgatcttgatgagctaggtgatgaagaggcttcgtgcgtcgagctaaggaacatgtccattggggacgtgtgtcctaaggaatccgaagagcctccaaatacacaagatcaaccatcttcctccatacaagcatctccaccaactcaagatgaggatgaggctcaaaatgatgaaggagaagatcaagaagttgagctacctcaagaggaaagcaatgatcaagggggagatgcccatgatcaagttgaggaagatgaacaagttccaaaaccgccacacccaaaagtccaccaagcaattcaacgagatcaccccgtgaacaccatcctcggcgacattcaaaagggggtaactacttgatctcgtgttgctcatttctgtgaacattactcttttgttttctctattgagccacacagggtagaggaagcacttcaagattcggattgggtggtggcaatgcaagaggagctaaacaatttcacaaggaatgaggcatgacatttagttccacgtcctaaccaaaatgttgtaggaaccaagtgggttttccgcaacaagcaagacgagcatggtgtggtgacaaggaacaaagcccgacttatggccaaggggtattcacaagtcgaaggtttggatttcggtgaaacctatgcacccgtagctacgcttgaatcaattcgcattttacttgcctatgctacttaccatggcttcaagctttatcaaatggacgtgaaaagtgtcttcctcaatggaccaatcaaggaggaagtctatgttgagcaacctcccgactttgaagatagtgagtaccctaatcacgtatataaactctctaaggcgctttatgggctcaagcaagccccaagagcatggtatgaatgcctaagagattttcttatcactaatggcttcaaagtcggaaaggcaaatcctactttatttactaaaactcttgacaatgatttgtttgtatgccaaaatttatgttgatgatattatatttgggtctactaacgaatctacatgtgaggaatttagtaggatcatgacacagaaattcgagatgtcgatgatgggggagttgaagtacttcttaggatttcaagtgaagcaactccaagagggcaccttcattagccaaacgaagtatattcaagacattctaaacaagtttggaatgaaggatgccaaacccatcaagacacccatgggaacaaatgggcatctcgacctcgacacgggaggtaaatccgtagttcaaaaggtataccggtcgatgataagttctttactctatttatgtgcttcacgaccggatattatgctttccgtatgcatgtgtgcaagattccaagccgaccctaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcaaaatgctttgtttgtgttgcatcatcacaaaagggagatgaaagggatagcctacacttttctatgatttgtgctgaatgcccacacaaataatgactactagctgctctagtctatgagttctacagtgccaaagtcacaataagccaataaaaagaccaagaaagggttcaaacaaagagagcaaaagacatcccaaatggtaccctggtctggcgcaccggactgtccggtgtgccaccggacagtgtctggtgcaccaccggacagtgtccggtgcaccagggaactcgaagctgaacttgccaccttcgggaaaatgggagaccgctccgctataatttaacggactgtccggtgagtcaccggacagtgtccggtgcacaccggactgtccggtgtgccagcggagcaacgactacttcgcgcgcatcggtcgactgcaaccgcattaaatgcgctacaatgcgcgccagagtcagagcacgcgcaggagacgcaccagacagtctacatgacctatccggtgcaccaccagacagcccagaggccccaccaatcagagctccaacggtcgaaccctaacgaccggatgacgtggctggcacaccggacagtgtccggtggcgcaccggactgtccggtggagcaccggactgtccggtgcgccatgcgacagcagccttccaacgaccactttgtggtggttggggttataaatacccccaaccacccccacattcaatggcatctaagttttccaccttcaacacattacaagagctatagcattcaattctagacacaactaaagagatcaaatcctctcccaagtccggaatcactccaaatcaaatagtgactagagagagcgacatttgtgttcatttgagctcttgcgcttggattgcttcttttctttctcattctttcttgtgataaactcaattgtaaccgatgcaagagacaccaattgtgtggtggtccttgcggggactttgcgtcccgtttgattgagaagagaagctcactcggtctaagtgaccgtttgagagagggaaaagggttgaaagaaatccagtctttgtgaccacctcaacggggagtaggtttgcaagaaccgaacctcggtaaaacaaatcatcgtgtctcactctttatttgcccgcgatttgttttccaccctctctctcggactcgtttctatttataacgctaacccggcttgtagttgtgcttaagtttataaatttcagattcgccctattcaccccctctatgcgacttttaattttaatctatgtttttaccgtgaaaggcttgctttgatttttctttttcatagacctagcttatgtgggaattttaatctatgtttttatggttaaaggcttacattgatcaatcctttcgtagacctcactcacgtggaattttttagcactgaatctgtgttttttaagatatgtaatcattattctatataatgataaacaatttcctacaatacagttttgctatatcaaagccagtgatgtcaggcgccgctgtagtgccaactgagcagtagtattgctgtagtatccaaccaattacatgccatatgaaattgttccatttgaaaattttgtcttcaaatacagtgacatggtactctcgcattttggtcggagcaaaactagtagtggagctgaactaaaaacacatatttacaaatccaaaaataaacgatacaccacaaaaaacctcacagcagccaacgattctacttactatcactcagtctcattgaacaatatttaaccgacgaaaatccaacaatagcactccatataggcagcaaataacctagaggtaacccagttttaggattatttacctaacaccgggtcaattaagattgctactgatcatcctgttgtggatgcttctaaaatctgaatctgcgggttccactgtcttcagaaagtgcagaacaaagtccacaccgagcaacctgctggtccaacagccagccatgtaagggcttgttcgatttaaggtagattgagagaaatttgaggggttctctaaggtgtcactgaatgcgtactaggctgaagaacagcattaacagttcagtagtttaacaatacggcatattgcaggtattgctgctcacaaaactgatgaagtgctagttgaaccaaaatcctatgatgaggcaaggcagttcagtgaacgggaagcacaagccatggattcggagttttctgaactctttggaaaatcttggacacttgtgcctccagataacggttaaaccacgtaacccatgactctatatttaaattacccatcaatattaggaagaaatacactatagctaaagtacgtaggccttaatttgtatctaattaccaatgacctcctattgtaataaataatagacataactcagttttcattctattattgtagaaaatcgaataccactcagatctacatctaaatcgagtattaacatcttctactacaataaaattgtttagatcactatagaccatatatatgtttctacattgcctacttatatcaacattaatacattaaaatttaattaaagtaaatgtgcacatcgtgccaatataaatgggcaataaatatatatgttatgttgcataagataaaattgtttccataacaactatttctatatataggttgttagtttagatttacttagatattttaagtgagtttttgacacatctatattgatagtatagccataattgcatatatgattccctaattaagtggaagtgagaactgtgggtgatcattgatggatctatttttcaagctctttaatattactttaaaggaaaaggtcagccattttaaggcttgactattaaaaaaatcaaggttagttgtgctaccacattgatcctagatctcacctcaagctctatgacttatcttgaatgatattatgcgtgttactaacaatttcttatgctattgtagtatttatgaatgtactgacgacttaaaatccttgttctagagaagtgtaaaaacaagagttaactaaacattcaataaacacacgagtttttaacttaacttgagataataagaataaattttataaaatcaaaaattgattcatgactatatgatgcgacaagagccaccttagttagcgataggattcaaattaattgcatgtttcaaactatagagagatataatgcaaaaggaaaaaatgaatattgatgagaagacataaaatccagtcctttgcaaaaaaagattaataattacatgtacatatagactctaaagtatctattatgtgtattacgttagatgataaataaatactatattttgggaatgttccaaacctcccaagataattattagttagataacaagttgttagctagttttaactggactggagcagctaacaaataatagcttttgagaggtataactaacaattagatggtccattacctaatcgatccgaacctctaagctaatttttgttgataactatatattagttttagaggtctgtaatagagtcttaatataaattatccaaacaataactagtgcttatatcctacaattttaaaccgcgtgagagtacatgttgatgaactagtgatattaattaatgtatcataaatattaatagttttatatatatttttaggcaaaattaaatatgggtgacttctcgagaaaaataaggacaacagttaaaaagaagagacgaaggagtatttgacatgtgtgtgtacgtagcaggggtagcatatgaaccgaccaatttcagccaacgtttaagtacgcatcgctcaccttgtctccttccgttgcccttgatgacttggagcatctctagccagctagcatggtactcaaacagtgccggtacgcataatgtatagtactatgtagacactgacagacacacggcaggtactacatgtacaagcgtatgctatacgcttgggtgtgtgcatgaaaaactgctagcatatactctctccgtctttttttatttattttttatttatcgcagtttagtttaaaattaaattagagaaggacaaatatctaaaaggagagttattacttattagtgtgtgtatatatatatatatatatatatatatatatatatatatatatataccagtaaaaacattcggccgacaagatccacccatattatattcatgctcacacctacagcacacggcggagcgacacgagcaattcatctcctgccgccacgccacgccgtgaggaacagtgaagtagatagacgagcaaattaagcgcgcgggcattatctagcaacttttcttcctgcgcgcgatggagcattatctagcaacttttcttcctgcgcgcgagctgtgcgcacagtggccgcacttgccggcaatgtagccatccatccacatagtatgaagatgacggcgtggggtacggtctcgtcccatccggcgaggaaggtaaccgccggtcggcctcccccgggtcgtcttctcttctcccacagttattgcagtgcccgaattagaaacaaggtttacacgagcgcgagacccacacgtatcgaagacagtagcagctcagcagacgacagtgcagcaggccatcatcgtgcgtgcagcagcgagtgatgcaacatgagaccaaacaaaatgagctgcgatgttgacaccccatgccaccattcatcggtgcttgccaatggtgattggtgagtgaatgcagaagggggggagagagaacaaaaagcggcaaaaggacgcactgcctgacgcgacgcctagtcgcctaccaaagcactcgtcttcttcccggaaactcctcatttacagcaggggggcagcaggcccatccatcctcggatccatgtctcacccaatcaagaacacagagaatggaaaagggaaagaaagggggagggggtgtaaatcggtggatcgacgagccgtttgcccttgggtcaaatgatggaccttcgccgtggaatacgatcgaaggacgaacgcccagtcctggttcttggtctacgtgggcctggccagcgaatggaacccattcttctggacccagccggccgggctttgcgtcgtactcgtaacaacgtagggatggaatcgtgatgatgctgcagctgcggtcgcggcggcggccggtactggcggcgggcacgggtgcggcggcggcggccagctcgtgccgccgccgttgttcctggcgccgtggagcctctgctgctgctgctgatgctgcagctgcggaggccagtgcccgcctccgccgccgatcgccagggaaaggcctcccgccgcggtccccggatcgccgttgctcaggagctgccgcgatttgcattgcacagaacaaacaaagcgtcagccagttagctcggtacgtagtcggtacccacttgcggatgcctggggctgtacggcggcaggtggggttgcatcatgcatggcggcgctcgcgtcgagctggagctgccatgccacccggtggttgaaacgtgacaagcttgcttttattaagcaagattttaaacgcacacctcgtttgattcagagaacgctgaacgcgggcgggaccaaaccaaaacggatacctttgccttggtgctccgacgtgcggccgtctgccagtcgatgtcgaacgccatggggacgcgctggtccgaggacttgtcgtcgccgccgccgccgccgccgccgtcaaggctgccccttttgttccccgccgagcttcccagcgacaggtccaggttgtgctcgtcgccggcggcgccgcccgtcgccgctgcatgcacggcgaaacagcggaccgtcgtcagagattgcttcctgtccatataggttgtagcaaaccgccgcacagcgacgaggggggtgatcgttgcagagcgcgggcggggatagacgaacccgccggcgcgacctcctcctcggcgtaaatgctgggatcgaagtttgtcaccgcgtccttgccgttgcacttgatggccgcgcggtcgtacgccctgcgcgcgcgcgcacagagagagagaaagggagagagacacggggcatccatcagtcagtttcgcggcccagggggtagcggggctccgagaaccgccgccaggcgagcacacagtgcggtccaattattttacctggcggcttcctcctcggtgtcgaacaggcccaagtagacgtacctgcagtgcagcgacacagaccggaagtgacagccatgaatgaataatgtgaggcgcaacgacaaagaaaggatgggctgatgccgatgcagatacagggaccagggatggggagggattgggaagaggaagacatgaagtggttgggcaagaagggaaggaaggcgaggagaagggagatggacggacgaccggcatacttcttgccgaggaactggcccatgcgcgcctcccaccgcccgcacttgtggagcgtcacgcccctgtacttggagctgccccgggggaaccccgtgctctgccgccggagcacgtgaacgaactcctccttgctcaggtggcacatctgctggtagaccaagacgtggattagatagacgagaaaaaattactatatatattgttgctatatgctacctagtatagcgcagttgtataataaataaatcacctgcttcatgtcgtcttggtaatcctccaagctgaaattgatgtcggcctccacgccgcggaacttgatcgccgctcgatcgtacgccctgaggttggcgggagaaccagacaggcagagattacaatttggcggcgaacccccgctgttattggcggagaacttggcatgggagtgaggagagccaatacgaatcgaatgaaacagacagtaggcttttattaccgagcagccgcatgggcggtgtcaaatccgcctgcaagtcgccacaagccatttttgtgaggagtcccgagtccggaaccaaaaacgcctcggattgggcggtatctatcgtcttccaagcatggatgcgtatggaatggatggactcatggacgtgcaagaaacttacccagatagacctgcttgccgcaatccctgcattgccacaagcacaagcagagagcatggggatgtgagagagagagagaggcaggggacggggacacacagagggagaggaggaggaagagagagagagagagagagcatggcctgcctattggcatcgtaagaataacttggataagatgtggctacgaaaggcaaaagcaggagaggcgagaaatgccaaaacaagaggaatccaattccaatccaacagcgaacgccctccccccctccctcgcgagctgagctgctgcctgctccgtctctctccctcgagtgaggaaagggcggcggcaacggcaacggagtcgggagtacgggactactcaccatatgtgcgactcccaccgccccgtccggcggtagaacgtgacgccgcggtactgcgagctgcgcgaccgcggcccgcgccggctcttcttgccagccgccgcctgcaccgcagcggggccgccggccggcgcggcggcccggcggagccagcccgcgcgcgcgttcgacgagccgggcgccgcgccgatggtggccgccggggccaggacgaagaactggcgcgtgacgacggcggcggcggcggagtcgtcgtcgggtatgtcgactagcgcggccgccgacgagaaggacgcgccggagtcgtcggcagatggggacggcggggcagcctcggcggccggcgagtcgttaaggtcccacatggctaggaggctaggcgctggtcttcctctcttgctggtacgacgagtacgatgcttgtgtgtgcctgtgcacaccctcctctgctcggtctccttcttgcctcgcactacaactcgggtgatgtgaagagggagaggaccccagccaaaagcaggcgctgttgccttgttggggcatgcaagtactttctcttttgctatccccttggcaaagactcttccaacctaggagagcagaggaggccaaaagcacccgcccgcccgcccgcggataggctcagctgcttgcgctgtgtgccaaggttgccgcggctgcaagttgccactagttccgggcaggacagagtggaatgagcgacgccgagggggcggcgtcgggcgtcggccaacaggcaccagtcatcacaaatccatcagcacttagcagcacctcatcacaaatcctacgtactcttttttatcgcgggcgggcatagcaacaacctataacgctcgttcctcgtcaaagcgaccagaccggcctgccgccgttgtagttttcgtttggataattggactggatccgatacgcagagtcgcagacacaacggccccgttggccgttgggagtgctggatccgcgccttctccttttggggtgcggcacagggtgggtaggggacggtttgctcagcagagtgtcagagtccagagtcagcctatttttactgttactgcaccgtcgtatcgtctctctgccatggcggcagccggagccacatgtggatgaggtcacctgctgctgttggtggcggctatcctctgcattatccaacgtatgcgactgctctatttttcttccctcgccgtcctcggcaggaggatcgagcgcgcgacaatctcttttttgctctgatcgggaccagtgatcttttcccggtcccaggtaattaggccaggctatttccggacacagtctttacccgaacgtatcggaaataatagcaatttagagattaataataatacaaaaaacagtagcatgtttcatatatgtataactaaggttgacaacatactctaaattttacgctataaatttcacaaatcgtattaggatcgggctttcacatattagattaggattaggatcgatctctatttttatttatttttaaactataaattatttagagctcttacaaattatgaaagaacatttggatcacgatctattatcatatctacgtataattcaacagcaaatcaaatcatatactagtgctagtgacaaaataaatatatgcatgcacttttttatagtgcgggttgtcgacactaaaaacaacaacgcaagagtagacgtagaagaccggtgatgatgacgatgtcggcatagagaaagcaaaccaacaacgagccgttcgtaatgacagttcctaaaaacctgatgtgatccctacccgtgcagttaggagacgtaggcaagtcaaagcggccaccagacgctttggtgcgtggggtaccttggcttgcattgcataccggcaggcacatagtttccaacccgtcctcatcggaagcaggacgcggcggccaccaacactagtacaacagtggctcgtagccgtagtcctatcttgcacgtacgatgtacaggagccaggaggggtggtaattggtatacatgccgcgcgcgataccatgtgtatcgtcgctcgctgctcttcgctctgtgctgtgaactaaaagtttcagtgaactggttctcgtctcgatcacggcacgaccagcacctgttctatcaaatctatcgagtatctagtatctgtcagctgtctgtctgcctctccctttcattctctgcagtgttgtgaacttgtgatcaagcgagctgggcagtatttcccccccccccctaccccacccgcccctagctctgttcaggacctgcattgtaatttgtaaatgcctgcctccgagctgttgttttcactcaactcgctcaaaaaggttgtaacgcctcaaaatcctatgttggtattaaagtaaaattgtccaagaatgttgaattaaaataactttcaaaattttgtttctgataaattttttaataaaagtatccctatatgattttagctattcctttatgcaatgaatatatattgtctctaaaatattttatacactaggagcgctattgtctctacataaccattttttttgtatgaattgcatattgaataataaaataccttatatatataatgttgcattgcatgctgaatattttgatttgttgcatttaaaattaaatttgaagtttgaactttattggaaattggaaagcaggaaataggaaattaaaacatcaaataaaaagaaaaaaggaaaaagagtgtaatacccactttgtaataaaaatctaaaaggagagattatattcctttatatacatgtgtgtcatctatatctatcatttcatgtgaacacctcacttccacaaataaataattaaataaaaacactcaaataaatcatgcatcatgctaggattttcttttgtgtgcatattgtgacaaaataaaataaaaatgtgacaagaaaaatattaaagtccaaaaagggaatttagaatctaaaagaaatcctagaaagggaaggaaagaaaattttatgtgataaaaaaatgtaaaataaataaataaaatattattgctatactagtgctttaatgtgacacctgactcaggggtagagctcaaaagcagattcaaatccaaatttgaattcaaaatatttatgaataagaggatagaaaataaaagaaaggagaggggactacctgcgctctgggccgaatttcttctgtgatcggcccatcaggcaaatgcgcacagcccaaaagaacggattagaccgtgtgccgaccggtgggccgttggtgccagcctcttccctctgcgccgatggtgggggccacattccagatgcgcgtgctgtggcgacttgcaggcgggcccaggttgtcagttccttccccttcgccgtaacagaactcgcgttctctacgccgcatgactatcacttccgccgagacctcgcacgcgccacatcttgatcccaggaccgctctggattctattgcagagatctcgccgtcaccgccgcgggtatcggatggtcgttacatcgtgtgccgacttatgcggattggcctgggcgcataagtagcgcgccgctcggtatcttggcccaccaaatgaacccctgcctcgcaccacagttgattgtgtagcctcgtcggtgggagaactccgctgccgccgaagaaagctcgacccatagccagtactagagagagaaagacgtgcgccaccgggcggattaacgggtttccgcggtcacctgagtaggggagtgggtgcggcatctgtcgccgttgggcaccgcacccgtgcctccgtccccgcctgcttgcagtgaccccgcgcacctccatgacgcaactcgtgcgtgcccgctcctgtcactcgcggtcgggttgccgcggctccccattgccgtgcagggcggggccaaactcgacggtgccctcgtcgtttgggggcctgcatggttactcgggggcttggtccacggcgggcataaccaatcgccggagttgggcgtcgtcgccggtccgcatcgcgttgcgagcagcgtcgccatcgctgcggtgtaggaagaaggggctgccatcgccatggacaaagcactgggccgcagccgaagaacttcatctgcgctagggcttcacagcaccgtgacccgcgcttcgtcgtcggccatcccctgcctccttcaacggtgagaattccccctcctccgagtcaacggggccgagtcgcttgctgttggtggagtcttgcgtattccaggagaaggtagcggcataaggtagaagagggagttgtggctgtcagattgatgacgggcggcctagattaccccttcattccttagtcgtgggccgttagatcgtgatccaacacgcgtgattagttgccagctcatgcctttttggttgccgttggatctggatccgaggctccacatggcgtaccccttcgttgtgttaagttggcggccgtgggatgaaaatctaacggctcagggtgcatgcgcatactgctgccgacccatcttacaaaagagccctaatgtcgttaataaataacccgcagtccacgtagttgggctctgagtcttaggaaaacttacaccgagacccctgaacttctctgtattagtgcgcccagtccagagagccagaaaaatcagaaaacaaatatagaattggatttttaatgtagaaataaatgctagaattggtttaattcatagaaaattcatgtgtagtccaaattaatccatttcagtttctataattttgtaataatgttgtttatcaccctgtgtctctgttttaagatgaaaatggtattaaaatttatttcttaattaacctcgtatcaaatacataaaaccttaggaaattcatatatcctccgttttaacttcaatttgatccgttcaagttgcgttagtctcgtagcaaggcgtatatcattattacgcagtatattcttatgtttggtgtgatgttaattttgtctataccatgtttgtttgtattgctacgactagcagtgaggtcacgaggatttgaagaatcaccctggtaactggaatctcaagtgtcaggcaagttgtgcccttgaccactttttacccaataatgttctctaattatcattcactcgtgcataggttaattttgatgggacccaataggacaccctagatttgtttatctcattaccttgtttacccctgaatcacttgggtagtttgctattgctttacatggttttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattaggaaagctagtggaagactaccttacccgaaaggggcaagggcagtaggggagtggtcagtgtagggaggtccttggttgattttgctgcgatggcggtcagacaagaaccttgcattggaacttcttataaactgtagcgggttttcggaagctagtggaactttgtaaaggcctcgtagtgttgccctgccgcgcttcctaggtagaggtgtatgggattcgcgactcctggcagatggttagcatgacttgtgggtaaagggtacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaataggaccccctagatttgtttgcttcattacctttgtttacccctgaatcacttgggtagtttgctattgctttacatggtttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattaggaaagctagtggaagactaccttacccgaaaggggcaagggcagtaggggagtggtcagtgtagggaggtccttggttgattttgctgcgatggcggtcagacaagaaccttgcattggaacttcttataaactgtagcgggttttcggaagctagtggaactttgtaaaggcctcgtagtgttgccctgccgcgcttcctaggtagaggtgtatgggattcgcgaccccttggcagatgggtagcatgacttgtgggtaaagggtacaacctctgcagagtgtaaaactagtatactagccgtgctcgcggtcatgagcggctcaggactctctgatgtttaaattatggaacttaaattcaattttgtcatttgcattgcatgggtctattattaattttgttcaattactttatctaaggtttggtattcacttatacttagtaactgctaataaaattttgaccaactacttaaaagcaatgctcagctttaacccctatcattgattagccttacacatcacatgacctcacacctttgtgagtttatgtccaccggttccccacaacttgttgagctatgatcatgtgtgagctcacccttctgtctcacacccccccacaggagaagagcaggtggttcaggaggagccgcctaacactgaggagttcgatctgatctaggtggcgtttcccagtcgacattggcgccgacgatccttagttcgttttacttttatcttttattttgtaataagtcttccgctatgtaataaatactctgatgttttatgacatttatctctatacactctgttattatatatgttgtcttcttggcgcatgtatgagatgcacccggctttgttccttaaagccgggtgtgacagaagtggtatcagaggaaatgttgactgtaggacgaaacctagatagaaatggacaaaacccttcactacttaccttactctgattctttctacacttatcttgattctgtctcaccttctactgttctactctgatcattcttaccttttctactcttagtcaagatggatttcacaccttggaatccctacccctatgacatttttaagaggtaggaagcctaagacaaaattaaactatttctttataaaatgttggttgattgttctgatgatcaatgcctgatttgcttctttgattgatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaatctagagatttcgtcttttcacatctggaggtaaactgtgatcctctatccaaaatgatcttcttcggtacatcgtgtggagacacaatccgagccatatataactctaccaattgagataccttataagtagtcttgaccagaatggaatgagtcactttggtaaatttatccataataacccatatagaatcatatcctctcggggtgcgaggcaatccagtaatgaagtccttaccaatttcttccccttccacttgggtatcttcagtggatgtaatagtccagctggtctttggtgttaggccttaactctttgacacccatcgcacatagccacatgtgcagccacatctctcttcaatccataccaccagtatttctgcttcaattcctgatacatcttggtactaccaggatgaatagaataatccaagttatgggcttcctttagaatagtcttacgaaggctttcaataccaggaacacatatcctgcccttgaaccatattgtgtcttgttcatcttccgtaaattccggacctctacctttcgtaataagatccttgatctcttgtatcttagcatcaccaatctgtcgttctgtggatttcttgctccaaggtaggttccacatcaataatgactccttaagcgtgagcatctatccccaggttaagtctcctgaaatcctcaacaatctcatcgggtagctgggcaacaatagctgaatgaacatgctcctttcgactcaaggcatctgcaaccaaatttgccttgcccaggtgatagtgaatctccaaatcataatccttaataagctccaaccaacggcgttgcctaaggttgagatccttctgagtgaatataaactcttatgatccgtatatacttggcacttggtccccatgatataatgtctccatatcttaagcgcatgcacaacggctgccaattccaagtcgtgagtggggtagttcaattcatgtctccgtaactgatgagacgcataggcaatcacatgtccttcctgcatgagcacacatcctaagccttggccacatgcatcacaatagatatcaaatcctttctgtaggtctggcataaccaatactggtggtgacatcaatctcttcttcaattgatcaaagctgtcttggcacttctcgtcccacttaaattctctgtccttctccagaagcgaggtcgtaggcttagcaatcttagaaaatccttcaataaatctccgataatatcctactagtctcaagaaactctgaatctcagtaactgtagtgggtactctccactccattatctccttaaccttagtaggatccattgatattcctccattagaaataatatgaccaataaatggcacctcgccaatccaaaactcgcacttgctaaacttggcatatagctggttatctcgtagcttctgtagcaccatcctcagatgttcctcgtgatcactatcactcttggaatagataagaatatcgtcgatgaaaaccacgacgaatctatccaaatcatgaacacattatcctttagatccataaagtaggctagtgcattagttagtccagacgacataacggtgaactcatataaaccatatcgggtcaaggaattcgtcttgggaatatccgatggcctaatattcatttgatggtaacccgatcgaagatcaatcttcgagaatactcttgcacctctcacctgattaaataaatcttcaatgtggggtaatggatacttgttcttcatagtgacaccattaagggacctttaatccacacacatcccttgcaatccatctttcttctgtacaaacaaaaccgatgctctccaaggtgaagactcggacgaatgtacccagcctcttgtaattccgttaattgcttcttaagttcctttagttcttctacggacatcctgtatggccgtttagaaataggagtcgtcccaggtaagagatcaatgaccaactcaacatccatatctggtggcacccctaataactcctctggaaagacatccggaagatctctaactgcacggatgttgtcacccacaaacttctcatcttctaagaatgttgttagactaatggtggtagttactgcaattccaacttcaaatatttctcctttggaactggtgagttctatggttcccttagcacagtgtataaactgtctttgcctttcttaaccatgaaataccaaggatcacgcctatagtgctctcttctaacactataggggtagcccatctgcgttagaaacatagggtgcgaaaggaagaattgtagacaaggcgagtaattaagaggaatattactacgggggatttattagctaagtagattagtgtgtcacctactaacgctaatacattaccttatggtggtacatgctaagatgcccgtctatactatttcaatcaatcaaagaagcaaatcaggcattgatcatcagaacaatcaaccaacatttttaataaagaaaatagttttaattttgtcttaggcttcctacctcttaaaaatgtcataggggtagggattccaaggtgtgaaatccatcttgactaagagtagaaaaggtaagaatgatcagagtagaacagtagaaggtgagacagaatcaagataagtgtagaaagaatcagagtaaggtaagtagtgaagggttttgtccatttctatctaggtttcgtcctacagtcaacatttcctctgataccacttctgtcacacccggctttaaggaacaaagccgggtgcatctcatacatgcgccaagaagacaacatatataataacagagtgtatagagataaatgtcataaaacatcagagtatttattacatagcggaagacttattacaaaataaaagataaaagtaaaacgaactaaggatcgtcggcgccaatgtcgactgggaaacgccacctagatcagatcgaactcctcagtgttaggcggctcctcctgaaccacctgctcttctcctgtgggggggtgtgagacagcaagggtgagctcacacatgatcatagctcaacaagttgtggggaaccggtggacataaactcacaaaggtgtgaggtcatgtgatgtgtaaggctaatcaatgataggggttaaagctgagcattgcttttaagtagttggtcaaaattttattagcagttactaagtataagtgaataccaaaccttagataaagtaattgaacaaaattaataatagacccatgcaatgcaaatgacaaaattgaatttaagttccataatttaaacatcagagagtcctgagccgctcatgaccgcgagcacggctagtatactagttttacactctgcagaggttgtaccctttacccacaagtcatgctacccatctgccaaggggtcacgaatcccatacacctctacctaggaagcgcggcagggcaacactacgaggcctttacaaagttccactagcttccgaaaacccgctacagtttataagaagttccaatgcagggttcttgtctgaccgccatcgcagcaaaatcaaccaaggacctccctacactgaccactcccctactgcccttgcccctttcgggtaaggtagtcttccactagctttcctaattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaaccatgtaaagcaatagcaaactacccaagtgattcaggggtaaacaaggtaatgagataaacaaatctagggtgtcctattgggtcccatcaaaattaacctatgcacgagtgaatgataattagagaacattattgggtaaaaagtggtcaagggcacaacttgcctggcacttgagattccagttaccagggtgattcttcaaatcctcgtgacctcactgctagtcgtagcaatacaaacaaacatggtatagacaaaattaacatcacaccaaacataagaatatactgcgtaataatgatatacgcgttgctacgagactaacgcaacttgaacggatcaaattgaagttaaaacggaggatatatgaatttcctaaggttttatgtatttgatacgaggttaattaagaaataaattttaataccattttcatgttaaaacagagacacagggtgataaacaacattattacaaaattatagaaactgaaatggattaatttggactacacatgaattttctatgaattaaaccaattctagcatttatttctacattaaaaatccaattctatatttgttttctgatttttctggctctctggactgggcgcactaatacagagaagttcaggggtctcggtgtaagttttcctaagactcagagcccaactacgtggactgcgggttatttattaacgacattagggctcttttgtaagatgggtcggcagcagtatgcgcatgcaccctgagccgttagattttcatcccacggccgcaacttaacacaacgaaggggtacgccatgtggagcctcggatccagatccaacggcaaccaaaaaggcatgagctggcaactaatcacgcgtgttggatcacgatctaacggcccacgactaaggaatgaaggggtaatctaggccgcccgtcatcaatctgacagccacaaccccctcttctaccttatgccgctaccttctcctggaatacgcaagactccaccaacagcaagcgactcggccccgttgactcggaggagggggaattctcaccgttgaaggaggcaggggatggccgacgacgaagcgcgggtcacggtgctgtgaagccctagcgcagatgaagttcttcggctgcggcccagtgctttgtccatggcgatggcagccccttcttcctacaccgcagcgatggcgacgctgctcgcaacgcgatgcggaccggcgacgacgcccaactccggcgattggttatgcccgccgtggaccaagcccccgagtaaccatgcaggcccccaaacgacgagggcaccgtcgagtttggccccgccctgcacggcaatggggagccgcggcaacccgaccgcgagtgacaggagcgggcacgcacgagttgcgtcatggaggtgcgcggggtcactgcaagcaggtggggacggaggcacgggtgcggtgcccaacggcgacagatgccgcacccactcccctactcaggtgaccgcggaaacccgttaatccgcccggtggcgcacgtctttctctctctagtactggctatgggtcgagctttcttcggcggcagcggagttctcccaccgacgaggctacacaatcaactgtggtgcgaggcaggggttcatttggtgggccaagataccgagcggcgcgctacttatgcgcccaggccaatccgcataagtcggcacacgatgtaacgaccatccgatacccgcggcggtgacggcgagatctctgcaatagaatccagagcggtcctgggatcaagacgtggcgcgtgcgaggtctcggcggaagtgatagtcatgcggcgtagagaacgcgagttctgttacggtgaaggggaaggaactgacaacctgggcccgcctgcaagtcgccacagcacgcgcatctggaatgtggcccccaccatcggcgcagagggaagaggctggcaccaacggcccaccggtcggcacacggtctaatccgttcttttgggctgtgcgcatttgcctgatgggccgatcacagaagaaattcggcccagagcgcaggtagtccccctctcctttcttttattttctatcctcttattcataaatattttgaattcaaatttggatttgaatctgcttttgagctctacccctgagtcaggtgtcacattaaagcactagtatagcaataatattttatttatttattttacattttttatcacataaaattttctttccttccctttctaggatttcttttagattctaaattccctttttggactttaatatttttcttgtcacatttttattttattttgtcacaatatgcacacaaaagaaaatcctagcatgatgcatgatttatttgagtgtttttatttaattatttatttgtggaagtgaggtgttcacatgaaatgatagatatagatgacacacatgtatataaaggaatataatctctccttttagatttttattacaaagtgggtattacaaatcctaccccccttaacaagaatctcgtcctcgagatttaggaaggactaggaaaaagatggggaaaaatctatgtgaaactcttcttctctttcccatgatgcttcatcttctccatggtgactccattgcactttgcacatttttatcaccttattccttgtaagtcgagtaaaagtgtcaagaatcttgatcggataccccgtgtaagtcaaatcatcctgaacactgagctcttccattggtaactgttcctcaaggacacggagacacttcttaagttgagacacgtaaattacattatacacatcagatatattatcaggtagctcgagttgataggccatctctccaactcgcctaaaaactcagaatggtccaataaagtgagggaacaatttgcccttaactttaaatctcctcattccacgaagtggtgacactttgaggtacacatgatcaccttcctcaaactccagtggtcttcttctattatcagcgtagctcttttgcctggtctgagctaccctcaaattctcccgaattatacggacttgttcctctgcttcttgaataagttcaggcccaaagaaatgtcttcctctagtctggtccaaaaatagaggtgtcctgcatttcctgccatacagagcctcgtacggtgacatcttcagactagcctgatagctattattatatgaaaactcagcataaggtagactcttgtcacaactactaccatgctgaagggcgcaagctctcaacatgtcttccaatacttgattagcccttttagtttgtccatcagtctgaggttggtaagccaaactagaattcaacttcgcatccatattctcatgaaaacttttccaaaatctggaggtaaactgtgatcctctatccaaaatgatcttcttcggtacatcgtgtggagacacaatccgagccatatataactctaccaattgagataccttataagtagtcttgaccagaatggaatgagtcactttggtaaatttatccataataacccatatagaatcatatcctctcggggtgcgaggcaatccagtaatgaagtccttaccaatttcttccccttccacttgggtatcttcagtggatgtaatagtccagctggtctttggtgttaggccttaactctttgacacccatcgcacatagccacatgtgcagccacatctctcttcaatccataccaccagtatttctgcttcaattcctgatacatcttggtactaccaggatgaatagaataatccaagttatgggcttcctttagaatagtcttacgaaggctttcaataccaggaacacatatcctgcccttgaaccatattgtgtcttgttcatcttccgtaaattccggacctctacctttcgtaataagatccttgatctcttgtatcttagcatcaccaatctgtcgttctgtggatttcttgctccaaggtaggttccacatcaataatgactccttaagcgtgagcatctatccccaggttaagtctcctgaaatcctcaacaatctcatcgggtagctgggcaacaatagctgaatgaacatgctcctttcgactcaaggcatctgcaaccaaatttgccttgcccgggtgatagtgaatctccaaatcataatccttaataagctccaaccaacggtgttgcctaaggttgagatccttctgagtgaatataaactcttatgatccgtatatacttggcacttggtccccatgatataatgtctccatatcttaagcgcatgcacacggctggcaattccaagtcgtgagtggggtagttcaattcatgtctccgtaactgatgagacgcataggcaatcacatgtccttcctgcatgagcacacatcctaagccttggccacatgcatcacaatagatatcaaatcctttctgtaggtctggcataaccaatactggtggtgacatcaatctcttcttcaattgatcaaagctgtcttggcacttctcgtcccacttaaattctctgtccttctccagaagcgaggtcgtaggcttagcaatcttagaaaatccttcaataaatctccgataatatcctactagtctcaagaaactctgaatctcagtaactgtagtgggtactctccactccattatctccttaaccttagtaggatccattgatattcctccattagaaataatatgaccaataaatggcacctcgccaatccaaaactcgcacttgctaaacttggcatatagctggttatctcgtagcttctgtagcaccatcctcagatgttcctcgtgatcactatcactcttggaatagataagaatatcgtcgatgaaaaccacgacgaatctatccaaatcatgaacacattatcctttagatccataaagtaggctagtgcattagttagtccaaacgacataacggtgaactcatataaaccatatcgggtcaaggaattcgtcttgggaatatccgatggcctaatattcatttgatggtaacccgatcgaagatcaatcttcgagaatactcttgcacctctcacctgattaaataaatcttcaatgtggggtaatggatacttgttcttcatagtgacaccattaagggacctttaatccacacacatcccttgcaatccatctttcttctgtacaaacaaaaccgatgctctccaaggtgaagactcggacgaatgtacccagcctcttgtaattccgttaattgcttcttaagttcctttagttcttctacggacatcctgtatggccgtttagaaataggagtcgtcccaggtaagagatcaatgaccaactcaacatccatatctggtggcacccctaataactcctctggaaagacatccggaagatctctaactgcacggatgttgtcacccacaaacttctcatcttctaagaatgttgttagactgatggtggtagttactgcaattccaacttcaaatatttctcctttggaactggtgagttctatggttcccttagcacagtgtataaactgtctttgcctttcttaaccatgaaataccaaggatcacgcctatagtgctctcttctaacactataggggtagcccatctgcgttagaaacatagggtgcgaaaggaagaattgtagacaaggcgagtaattaagaggaatattactacgggggatttattagctaagtagattagtgtgtcacctactaacgctaatacattaccttatggtggtacatgctaagatgcccgtctatactatttcaatcaatcaaagaagcaaatcaggcattgatcatcagaacaatcaaccaacatttttaataaagaaaatagttttaattttgtcttaggcttcctacctcttaaaaatgtcataggggtagggattccaaggtgtgaaatccatcttgactaagagtagaaaaggtaagaatgatcagagtagaacagtagaaggtgagacagaatcaagataagtgtagaaagaatcagagtaaggtaagtagtgaagggttttgtccatttctatctaggtttcgtcctacagtcaacatttcctctgataccacttctgtcacacccggctttaaggaacaaagccgggtgcatctcatacatgcgccaagaagacaacatatataataacagagtgtatagagataaatgtcataaaacattagagtatttattacatagcggaagacttattacaaaataaaagataaaagtaaaacgaactaaggatcgtcggcgccaatgtcgactgggaaacgccacctagatcagatcgaactcctcagtgttaggcggctcctcctgaaccacctgctcttctcctgtggggggggtgtgagacagaagggtgagctcacacatgatcatagctcaacaagttgtggggaaccggtggacataaactcacaaaggtgtgaggtcatgtgatgtgtaggctattcatgataggggttaagctgagcattgcttttaagtagtggtcaaattttattatgcaggtactaagtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtttgggagttgaagaatcagcttgattcgttctctacctaggatacgcggcagggcaacactactcgttctttacaagttccactagcttccgaaaacccgctacgttaagaagttccaatgcagggttcttgtctgaccgccatcgcagcaaaatcaaccaaggacctccctacactgaccactcccctactgcccttgcccctttcgggtaaggtagtcttccactagctttcctaattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaaccatgtaaagcaatagcaaactacccaagtgattcaggggtaaacaaggtaatgagataaacaaatctagggtgtcctattgggtcccatcaaaattaacctatgcacgagtgaatgataattagagaacattattgggtaaaaagtggtcaagggcacaacttgcctgacacttgagattccagttaccagggtgattcttcaaatcctcgtgacctcactgctagtcgtagcaatataaacaaacatggtatagacaaaattaacatcacaccaaacataagaatatactgcgtaataatgatatacgcgttgctacgagactaacgcaacttgaacggatcaaattgaagttaaaacggaggatatatgaatttcctaaggttttatgtatttgatacgaggttaattaagaaataaattttaataccattttcatgttaaaacagagacacagggtgataaacaacattattacaaaattatagaaactgaaatggattaatttggactacacatgaattttctatgaattaaaccaattctagcatttatttctacattaaaaatccaattctatatttgttttctgatttttctggctctctggactgggcgcactaatacagagaagttcaggggtctcggtgtaagttttcctaagactcagagcccaactacgtggactgcgggttatttattaacgacattagggctcttttgtaagatgggtcggcagcagtatgcgcatgcaccctgagccgttagattttcatcccacggccgcaacttaacacaacgaaggggtacgccatgtggagcctcggatcgagatccaacggcaaccaaaaaggcatgagctggcaactaatcacgcgtgttggatcacgatctaacggcccacgactaaggaatgaaggggtaatctaggccgcccgtcatcaatctgacagccacaaccccctcttctaccttatgccgctaccttctcctggaatacgcaagactccaccaacagcaagcgactcggccccgttgactcggaggagggggaattctcaccgttgaaggaggcaggggatggccgacgacgaagcgcgggtcacggtgctgtgaagccctagcgcagatgaagttcttcggctgcggcccagtgctttgtccatggcgatggcagccccttctccctacaccgcagcgatggcgacgctgctcgcaacgcgatgcggaccggcgacgacgcccaactccggcgattggttatgcccgccgtggaccaagcccccgagtaaccatgcaggcccccaaacgacgagggcaccgtcgagtttggccccgccctgcacggcaatggggagccgcggcaacccgaccgcgagtgacaggagcgggcacgcacgagttgcgtcatggaggtgcgcggggtcactgcaagcaggcggggacggaggcacgggtgcggtgcccaacggcgacagatgccgcacccactcccctactcaggtgaccgcggaaacccgttaatccgcccggtggcgcacgtctttctctctctagtactggctatgggtcgagctttcttcggcggcagcggagttctcccaccgacgaggctacacaatcaactgtggtgcgaggcaggggttcatttggtgggccaagataccgagcggcgcgctacttatgcgcctaggccaatccgcataagtcggcacacgatgtaacgaccatccgatacccgcggcggtgacggcgagatctctgcaatagaatccagagcggtcctgggatcaagacgtggcgcgtgcgaggtctcggcggaagtgatagtcatgcggcgtagagaacatgagttctgttacggcgaaggggaaggaactgacaacctgggcccgcctgcaagtcgccacagcacgcgcatctggaatgtggcccccaccatcggcgcagagggaagaggctggcaccaacggcccaccggtcggcacacggtgtaatccgtttttttgggctgtgcgcatttgcctgatgggccgatcacaaacgaatcaagctgattcatcagacgtccaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattcccaaccttaggaaagtaattgacaaaattattaatagacccatgcaatgcaaatgacaaaatgaattaagttccataattaaacatcagagagtcctgagccgctcatgaccgcgagcacggctagtatactagttttacactctgcagaggttgtaccctttacccacaaagtcatgctacccatctgccaaggggtcgcgaatcccatacacctcacctaggaagcgcggcagggcaacactacgaggcctttacaaagttccactagcttccgaaaacccgctacagtttataagaagttccaatgcaaggttcttgtctgaccgccatcgcagcaaaaacaaccaaggacctccctacactgaccactcccctactgcccttgcccctttcgggtaaggtagtcttccactagctaaaaataattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaaccatgtaaagcaatagcaaactacccaagtgattcaggggtaaacaaggtaatgagataaacaaatctagggtgtcctattgggtcccatcaaaattaacctatgcacgagtgaatgataattagagaacattattgggtaaaaagtggtcaagggcacaacttgcctgacacttgagattccagttaccagggtgattcttcaaatcctcgtgacctcactgctagtcgtagcaatacaaacaaacatggtatagacaaaattaacatcacaccaaacataagaatatactgcgtaataatgatatacgcgttgctacgagactaacgcaacttgaacggatcaaattgaagttaaaacggaggatatatgaatttcctaaggttttatgtatttgatacgaggttaattaagaaataaattttaataccattttcatgttaaaacagagacacagggtgataaacaacattattacaaaattatagaaactgaaatggattaatttggactacacatgaattttctatgaattaaaccaattctagcatttatttctacattaaaaatccaattctatatttgttttctgatttttctggctctctggactgggcgcactaatacagagaagttcaggggtctcggtgtaagttttcctaagactcagagcccaactacgtggactgcgggttatttattaacgacattagggctcttttgtaagatgggtcggcagcagtatgcgcatgcaccctgagccgttagattttcatcccacggccgcaacttaacacaacgaaggggtacgccatgtggagcctcggatccagatccaacggcaaccaaaaaggcatgagctggcaactaatcacgtgtgttggatcacgatctaacggcccacgactaaggaatgaaggggtaatctaggccgcccgtcatcaatctgacagccacaaccccctcttctaccttatgccgctaccttctcctggaatacgcaagactccaccaacagcaagcgactcggccctgttgactcggaggaggggaattctcaccgttgaaggaggcaggggatggccgacgacgaagcgcgggtcacggtgctgtgaagccctagcgcagatgaagttcttcggctgcggcccagtgctttgtccatggcgatggcagccccttcttcctacaccgcagcgatggcgacgctgctcgcaacgcgatgcggaccggcgacgatgcccaactccggcgattggttatgcccgccgtggaccaagcccccgagtaaccatgcaggcccccaaacgacgagggcaccgtcgagtttggccccgccctgcacggcaatggggagccgcggcaacccgaccgcgagtgacaggagcgggcacgcacgagttgcgtcatggaggtgcgcggggtcactgcaagcaggcggggacggaggcacgggtgcggtgcccaacggcgacagatgccgcacccactcccctactcaggtgaccgcggaaacccgttaatccgcccggtggcgcacgtctttctctctctagtactggctatgggtcgagctttcttcggcggcagcggagttctcccaccgacgaggctacacaatcaactgtggtgcgaggcaggggttcatttggtgggccaagataccgagcggcgcgctacttatgcgcccaggccaatccgcataagtcggcacacgatgtaacgaccatccgatacccgcggcggtgacggcgagatctctgcaatagaatccagagcggtcctgggatcaagacgtggcgcgtgcgaggtctcggcggaagtgatagtcatgcggcgtagagaacgcgagttctgttacggcgaaggggaaggaactgacaacctgggcccgcctgcaagtcgccacagcacgcgcatctggaatgtggcccccaccatcggcgcagagggaagaggctggcaccaacggcccaccggtcggcacacggtctaatccgttcttttgggctgtgcgcatttgcctgatgggccgatcacagaagaaattcggcccagagcgcaggtagtccccctctcctttcttttattttctatcctcttattcataaatattttgaattcaaatttggatttgaatatgcttttgagctctacccctgagtcaggtgtcacattaaagcactagtatagcaataatattttatttatttattttacatttttttatcacataaaattttctttccttccctttctaggatttcttttagattctaaattccctttttggactttaatatttttcttgtcacatttttattttattttgtcacaatatgcacacaaaagaaaatcctagcatgatgcatgatttatttgagtgtttttatttaattatttatttgtggaagtgaggtgttcacatgaaatgatagatatagatgacacacatgtatataaaggaatataatctctccttttagatttttattacaaagtgggtattacaaatcctaccccccttaacaagaatctcgtcctcgagatttaggaaggactaggaaaaagatgggggaaaatctatgtgaaactcttcttctctttcccatgatgcttcatcttctccatggtgactccattgcactttgcacatttttatcaccttattccttgtaagtcgagtaaaagtgtcaagaatcttgatcggataccccgtgtaagtcaaatcatcctgaacactgagctcttccattggtaactgttcctcaaggacacggagacacttcttaagttgagacacgtaaattacattatacacatcagatatattatcaggtagctcgagttgataggccatctctccaactcgcctaaaaactcataatggtccaataaagtgagggaacaatttgcccttaactttaaatctcctcattccacgaagtggtgacactttgaggtacacatgatcaccttcctcaaactccagtggtcttcttctattatcagcgtagctcttttgcctggtctgagctaccctcaaattctcccgaattatacggacttgttcctctgcttcttgaataagttcaggcccaaagaaatgtcttcctctagtctggtccaaaaatagaggtgtcctgcatttcctgccatacagagcctcgtacggtgacatcttcagactagcctgatagctattattatatgaaaactcagcataaggtagactcttgtcacaactactaccatgctgaagggcgcaagctctcaacatgtcttccaatacttgattagcccttttagtttgtccatcagtctgaggttggtaagccaaactagaattcaacttcgcatccatattctcatgaaaacttttccaaaatctagaggtaaactgtgatcctctatccaaaatgatcttcttcggtacatcgtgtggagacacaatccgagccatatataactctaccaattgagataccttataagtagtcttgaccagaatggaatgagtcactttggtaaatttatccataataacccatatagaatcatatcctctcggggtgcgaggcaatccagtaatgaagtccttaccaatttcttccccttccacttgggtatcttcagtggatgtaatagtccagctggtctttggtgttaggccttaactctttgacacccatcgcacatagccacatgtgcagccacatctctcttcaatccataccaccagtatttctgcttcaattcctgatacatcttggtactaccaggatgaatagaataatccaagttatgggcttcctttagaatagtcttacgaaagctttcaataccagaaacactatcctgcccttgaaccatattgggtcttggttcatcttccgtaaattccggacctcttcctttttcaataagatccttgatttctcttgtatcttagcatcaccaatctgtcggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccgattctagagaattcgtctcctttctgtaggtctgttcataccaatactggtggtgaacaaacacatcttcaattgatcaaagctgtcttggcacttcccgacccacttaaattctctgtccttctccagaagcgaggtcgtaggcttagcaatcttagaaaatccttcaataaatctccgataatatcctactagtctcaagaaactctgaatctcagtaactgtagtgggtactctccactccattatctccttaaccttagtaggatccattgatattcctccattagaaataatatgaccaataaatggcacctcgccaatccaaaactcgcacttgctaaacttggcatatagctggttatctcgtagcttctgtagcaccatcctcagatgttcctcgtgatcactatcactcttggaatagataagaatatcgtcgatgaaaaccacgacgaatctatccaaatcatgaacacattatcctttagatccataaagtaggctagtgcattagttagtccaaacgacataacggtgaactcatataaaccatatcgggtcaaggaattcgtcttgggaatatccgatggcctaatattcatttgatggtaacccgatcgaagatcaatcttcgagaatactcttgcacctctcacctgattaaataaatcttcaatgtggggtaatggatacttgttcttcatagtgacaccattaagggacctttaatccacacacatcccttgcaatccatctttcttctgtacaaacaaaaccgatgctctccaaggtgaagactcggacaaatgtacccagcctcttgtaattccgttaattgcttcttaagttcctttagttcttctacggacatcctgtatggccgtttagaaataggagtcgtcccaggtaagagatcaatgaccaactcaacatccatatctggtggcacccctaataactcctctggaaagacatccggaagatctctaactgcacggatgttgtcacccacaaacttctcatcttctaagaatgttgttagactgatggtggtagttactgcaattccaacttcaaatatttctcctttggaactggtgagttctatggttcccttagcacagtgtataaactgtctttgcctttcttaaccatgaaataccaaggatcacgcctatagtgctctcttctaacactataggggtagcccatctgcgttagaaacatagggtgcgaaaggaagaattgtagacaaggcgagtaattaagaggaatattactacgggggatttattagctaagtagattagtgtgtcacctactaacgctaatacattaccttatggtggtacatgctaagatgcccgtctatactatttcaatcaatcaaagaagcaaatcaggcattgatcatcagaacaatcaaccaacatttttaataaagaaaatagttttaattttgtcttaggcttcctacctcttaaaaatgtcataggggtagtgtcggtgttttgggtccgaccgcacacccggggttgcccctcaaggtgtttttaggagtaggacggtgtcgacgactgtagcaaaatggttcgtgccgatcgcacgagggacaatggacaagatttacaggttcgggccgcttagaaacgcgtaacaccctacgtcctggtgagtatatgagcgtggttacaaaaggattctctggatagagggcgcagagagtttttgtgggtggctaagtggagcagagtcgatccatctgaaggggtgccccctaggccttatatattcggccgtggagcattacatgtgatacgataacaacaagtagctgaaagatagtgaacttcttgagcttatccttacgtaacctccgccgacttatcctcgcatggctccgttgcatgggggcttcagcgcgggaaagtcgggtctctgtttgtgattcattcgttcgacgttgtgggccgcctgtgtttgcactaatcagtcccacgtcttctttattttaacggctgtctttccctaggcccacgaaagaatgaccttacgaattattgggcccttgggcctttcgtgaggccttttacttctttagtggacccgggggatatctatcccccacaagcccccgatctttgggttgatttggaggtaatcaacttaaagatccaaggtccaaaaatgactccctgctgttttctcttgctctgatcacttgttcgaccaaagtttagttttgtgcctgtgccttagaaatcggcattttggattataagattctggacttccttgggtgcaccgaaggtgcacccaatgggtgtagcccccgacctttgagtagattttagaagataatctactcgaaggtcttctccgaagattttctccattcgcgctcctgcatctcagttgaggattgatctttccaatcttgttctacgccttagaggtcggcactatattgatttagaatgataatccatggggtgcaccgaaggtgcacccaatgggtgtagcccccgaccttcaagtggatttttgaggataatccactcgaaggtcttccttctgtgtccttttgctgaaaattatcctttctgcttgtaaaaaattggcatgatgtcatccgcgctatgtcatcaattttatgcttcagaggtcagcatgtattttgtcttttgcagccgagttcgcaatccatccatatctcgctaggtagtgtaatttatttgctctgtgactgattggacatttgatggacgttctctgtctagtcttcaagtcatttctgtcgaccatattttgcactctactggctatatttggcttccctctaactcttgttgatatctcatttttgtcttgaggtattcactgcatgccctgcacggatgtgaccgtttgaaaaatatactgataattcctgggcgtcccccccccaatgggtgtgggcaagggacggcttggtacgctgagtgttttagcctggttgcctctgagtagtaatgcgatgggacggctagtgtaatcatatcctttgcgctgttgactggagtcccaatgggtgtagagttgcatgaaacggcgtccgctgtctgacgtgtcttcgggtgggtggaagtgcttattgaatgtctttcgactgttcagtgaccgcggttagaagtgagcggttgcctttcccttctataaataacccctttgcttctctggtttcttcacatctcttcgctgctccttactgcctcttccctttcttctctcccaaaactttaaccatgggcaagaacaacaagcgcaagcgcgagtcgactcctccatcggaggagtttggtgactcagaatactcggaggaggagttctcctccgagcctgaggggtctccggctcccgtctctcccccggcgtcgtccgatgactcggacgactcccaggggatagccgcggaggtctggacatatatccgggccgtcgagcgcgccgggctcgaaggctcggatgagtcggagtgctcctcggatgaggaggactcggacggcggcgacgagggtgaagacgacgacgacgacgacgacgacgacgacgacgacgacggcggctggggtggcggcgacggcagcagggacagcaccaagggcagcagcaggggcagcaccaagggcggcggcggcggcagcagggacagcaccaagggcagcagcaggggcagcaccaaaggcggcggcggcggcaggggcaggggcagggccagtggctagacgccactggtcttcttagatattagtatagtttagtatagctagaataatgtagtagtaattagtgtagtttagtagtagtggtaatgtaaagtagaattagggaagtaccaactcataaagagttggtttgtaagttcgtcaacttccctttaatgaagtatatcgttttatcccctctttttgatgacttgccactgcttttgctgaatgctgaactggttctttttgatatagtagaaacagcgccgagcgatgtgaagattgacatcagcgttttagaagtagcactgagcaatcgaacacaagaccagcgttttagaggcaatgccgaatgatagaaggtcggcatcggcattttagaagtaatgccaagcgacaaaatacggggtcggctttttagaaataacgccgagtgatagaatatcggcgtcagcattttagaagtaatgctgagcgtttgaacacgtggtcggcgttttagaggcagcgccaagtgatttgaaggtcggcgtcggcattttagaagtaatgccgagcgattgaacacggggtcggtgttttagaggcagcgccgagtgattgaaggtcggcgtcggcattttagaagtaatgccgagcgattgaacacgtggtcggcgttttagaggcagcgccgagtgattgaaggtcggcgtcggcattttagaagtaatgccgagcgattgaacacgtggtcggcgttttagaggcagcgccgagtgatttgaaggtcggcgtcggcattttagaagtaatgccgagcgattgaacacgtggtcggcgttttagaggcagcgccgagtgatttgaaggtcggcgtcggcattttagaagtaatgccgagcgattgaacacgtggtcggcgttttagaggcagcgccgagtgatagccagcttctcaagttactttgaggaaaatggccgagtgatgaggtggcacatcggctttcttggaaataactgttagatatccacgtagcatgctgggatttcggagatgaccgccgggtgatgactgggcacttttgaaaaggtatctggctcaagaatatcccttaagagtcgcgctttcagccgcctttgctttccgtgccctagttcttgcatttccgcatctgagtcttctctgccactcgcctcctgctctgtttcgccagcgagaagcaagatggcgcccaagaggaagaccgcgaactcgtctgctgcagtgatccccgccatcgatcccaacagtcagttgcccttcgcaggtaaccatatgtctgtgatttctgaagttgagcttctccgccttgtttccatcggggttctccctccacgggaactctgttcttggcgctcttgccatgggactactgtcccaaccgaagatacccacgagtcagtggtttacactcctttccttcttcgcggcctcggccttcccatatctccttttttccgtggcatccttgatttttatcacatcaacctgactcacttgaaccctaactccattctccaaatttctattttcgttcacctttgcgaagcttatcttggcgtgctgccgcattttggcttatggaagtatctgtatcactgccgtcctgggatggccggggggcaacatcaattggtcggaggtgccagcttagagatgcgtcgggggcggaagactgagtatctcgagataccccttaaagacagcattaaaggttggcgtctggagtggtttataatggataattatggaaattctctcccttcccgctcgggaagacaggcagacgttcgtactccgagttggactgaatctcccacggaccaagaagtagccgaagcgggcgtgttgcttactgaagttggattactgaaagagagaggtctgactgccgaagccgtggtcacagattttgtgttcaaaaacattcagccgctgaaagacagggcctatccggcatatctttaccgagggctggccgactcaacccgagttactaataggagaattccttctgtggatttggtgagccggcttgaaatgatcctcaggggtaaagtttcgaacgttggagctccagtggcatactcggcttggaacctaccttcttctcaagccttcactctttttgtgtccaatccacccgtgatagatagcaatttgggtcttagagtgcgaccctctgctgaagaggtccgttctttagttgcttcgctcggggatatacctgatgatgaacggcagatttattttgaagtgcctctgaaccctagtgatgcagacataaatgacatgctcgatctgctagctgtggattcatctgatactgctcctgatggtacattggcagtggtgccccttccagaggttgatgcgaccttggactcctcgaaacctgtcagtactcgcccgaggcgccctagccgaaccagtcagcccgagccttctgctgatgagcagaagaagaaaaggagacgcctccggcgagtgtccagttttgacgaggataccagcaccttagctcctgctatcgaagaaatgtctgcaactggcctagctgacattgatcccaatgggtgtgctccgcctgctgctgaccccaatgagggtgttgtttgtgctgttgctgtggaagacgaggaggaagagaatgaaactccattgactcggaaaaacagtcggcagttcgtcgctagcggcggtagtagtggggttccttctcctgccttgtctgctcttattggtctgcaagaactgtctatggccaattttgatcaagctctagaggatatggtccctgagaacttgttgttggaacctgcagacgttgatgcaacagaaacttgtgcgggcgtgccagatgctggattgaggtcgtcccgtgcctcgtcgaccttagagcacgatctcgagggccggaatgatgacttggatcgtcctgatcctgcggaggtagctgaaggcccgtcgaccttagaggtggtcacggcagagagcttggatcctttgaatagtgctgacatgtgcccagcccccgagggtgtcgccggggaggactcagctcaggtgaggagcgtaggccactacccagcccccgagggtgttgccgggggtgatccggctcaagtgggcagtgccaaccttgacccagcccccgagggtgcccgagcagggtctccttcctgcacttctatggacgttcacgtgggttcacctccacactctggcggcatggtggtggctcaaactccagatcagggggtcgctttagagggcagcatccccactggtctggtgttaggctctgctgaatgtactgagctcgttcctgctggtctactgcaaactgcttcgggtggtggtctgacacctgattatcagctgatttctcctgacttggggatcccttcgttcttttccaacctccaggtgctgtgccgtgctttaatttgatctctatgttgcatgaattgctgtcattatgctcatctgttcttctcgtcaggctttggtgaatggattggccagtcaactgaaatcacagggtgcttctaccccagaagtggcgtcatctctcgagcgttggaatccagtgttgcttcatcgtcgtgtatctgagttggaggccaccaatgctggtcagtgtcctttcttcttcttctttattcttgcccgtaggttgttttaccttctgacctcattttgtttgaatacttcttgatagggatgactcggcagatcaaagttcttgaagaaaaacattctcaagatcaagctgaaatgactcgacgatgcgctgacttcgaggagaagtattctcaaagtcagatcgagttgagtcaggtctctgcggctttggatgatgctaacgctctgagttcttctcttcatgtccagcttaactctgagaaggtagcttacgaacctgcgcttcgccttatcatgctcttggattctgaatgagtttgatttttgatttttgcttgtaggaagaaaaacgtatccttgctgcttctcgcgacaacttggacagattgtaccgagattccagtagctcgctgaccatcttggagaggagccaccgctttacgatggaggagcttgacaatcagcgtcgtcaactgcaagaatcttcggacgaagtggcccgccttacacagttgctatcagcaaaggatgccaccatcaaggaactccgagcttccaagaaaaccattgcccgggagttagaaactgctcagcaggttgttaaagttgctgaagaagctgctgtcactttcaaagctcagcgcgataaggccctggacaaggccattcgggcgggacgaatcttgatgagaagacctggcgtggttgtccccgaggatataagagccgacgtggttgctgcccctgattcctcgaatcgcccttcttcgtcagtcgttcctgagagagacattggaaaataaagaaacagttcgcgtgctctgagcgcgcagttagcatgagcaagcattcatttagaggacatcagcttatcattcgaatgttatgatttctttcttatcgtatcagaatttattagtttgcaaatttgtggtaatgctacatgataattatgaagtttgtttgtgggatatggaagtcatcccctgaacttcataagcgcgagtatgctataccggtttaagccgtcaatgacttttagccggtaacttccgttagtagggtttagtgatcgccctaggtaaagtaagcgtaagcatgttgcaccgccttaagtcgttgccgacttaagtcgattgttccgtttgtagggcatagtggtcaccccgagttaagtaagcgtgagcatgttgcaccgccttaagtcgttgccgacttaagtcgattgctccgtttgtagggcatagtggtcaccccgagttaagtaagcgtgagcatgttgcaccgccttaagtcgttgccgacttaagtcgattgctccgtttgtagggcatagtggtcaccccgagttaagtaagcgtgagcatgttgcaccgccttaagtcgttgccgacttaagtcgattgctccgtttgtagggcatagtggtcaccccgagttaagtaagcgtgagcatgttgcaccgccttaagtcgttgccgacttaagtcgattgctccgtttgtagggcatagtggtcaccccgagttaagtaagcgtgagcatgttgcaccgccttaagtcgttgccgacttaagtcgattgctccgtttgtagggcatagtggtcaccccgagttaagtaagaatgcaagtcaatcataaacgaaccgagtgtccttgaaatctatctttattgatgacatatttccactttacagagtacatggccgccccggctgttttgaagtacagctaggggtaaaactttctgaggtgctctatgttccaagagttcccaacttccgtgccatccatttgggtgaggcgatatgatccgggacgagtgacttctgctactatgaaaggtccttcccataagggtgataacttgtgccgtccctcccccgttagaattcggcggaggacgaggtctcccttcgaaaagaaacgttgccgcacagctttgtcgtggtagcgccttagagtctgctggtatcgtgccgattgaatcactgcattcagtcgttcttcctcaagtacgtcaatatcctccagcctagtggcttcggcttctgctatgctttcgaaaatcaatcttggcgccccaaacttgagatcagcaggtagcactgcctccgacccatagaccatgaagaaaggagtgtttccatgcagggctcggctaggttgggttctcaagctccaaacaacataaggcaattctcttatccattttcctgcgaatttttcatttttatcaaagaccctttttctaagtgcctccaatatcattccgttggctcgctcaacctgcccgttggctctcggatgggctacagatgcatacttgatctgaatgcttttttgctcgcagaagtcaaagaattctgaactggtaaagttggatcccaagtcagtgatgatactgtttggtatcccaaatctgaatattatgctttgtatgaattccacggctttagcagaggtcaaagaggcaatgggctggaactctatccacttagtgaatttgtcaattgccaccaatacatgggtgtatcctccttgagccttcttgaaaggtccaatcatatccaatccccagcatgcgaaaggccaagttactggtatggtttgtagctgctgcgctggcatgtgctgttgcttcgacaggtattggcaagcttcgcatctctgaactaactcggctgcatcattcttcgccgtcggccaatagaatcctgacctgaaaaccttcccgactaatgttctggatgctgcatgtattccgcactgccctgcatggacttcctccagaagttgcttcccggtggacgggagaacacacttcatgaggacgcctgacgcgcccctccggtataatacctccccaatgagtgtatagtgagctgattgtctggcaatgcgctctgctgagttcttgtcatctggctcttcttcattctttatatacttaataatcggttgcctccagtcatcagagtctgactcgggttggtctatgatgttgcactcttctatttgatccgtcgagatgctcggctgaaggatttcttgcacgaagaccccgggcgggacttgagtccgaccggatccgagcttggacaatacatcagccgccgtgttccgatctctttctatatgatggaactccaggccttcgaatttatcttctagctttcggacggtggcgcagtatcttcccattgaatcacttgaacaatcccattccttgtttatctgactgatgactaccagagaatccccgtacaccatcagcctcttgacacccagcgatatggcgatgtttaatccgtgtatcaaagcttcatattcggctgcgttgttagaggccgggaatagcaattggagagcgtacttaaggtgttcgcctccaggtgcagtgaagagaattcctgctcctgctccctgcagcttcagcgagccatcaaaatacattcgccatacttctgcggtttctggattatccggcacttgctgttctgtccactctgatacgaagtcgaccagtgcttgagttttgatggcagtgcgaggtcggaactcgatatcatgggaccccaactcgcaagcccacttggctattcggccaatggcttccttgttgtgaagaatgtcccctattggaaaaccagtaactactatgactttgtggtcgtcaaagtagtggcgcagcttgcgggcagttagaagtactgcatataatagcttctaaacttgaggatactttttcttcgatgggcctagaacctcactgatgaaatagacgggatgttgtaccgggtaggcatgcccttcttccactcgctcgactaccaacgcagtgcttaccacgtgagtcgtgcaagagatatataacagcagatcttcagccggctgagtcggcgtagctcgccggggcggtttcaatactggcggcgttgtgaggaatttcttcagtgcgtctagagcttcctgtgcctctgaagtccattgaaacttatccaccttcttcagcagtttataaaatggcagacctttttctcccagcctggatatgaatctgctcagggctgccatacatccagtgagtcgctgaaccttcttttgtgatcgaggagcttccatcttcatgatagcttcaatcttatctggattagcctcaattccccggtggctgacgataaatccgagcaactttcctgctggtaccccgaaaacgcatttttcaggattgagcttccatctatatcttctcaagctgttgaaaaccagctgtaaatcttcgatgaagttttctggattctcagttttgatcaccacgtcgtctaagtaagcctccacacgcttgccccagtgatcggctaagcatgtttgaatagccctctgataagtcgctccagcgtttttgaggccgaacggcatggaggtataacagaaagcaccaaacggggtgatgaacgccgttttttcctcgtcttcttttgccaaactaatctgatgatacccggaatagcaatctaagaaagacagcatcgaacatccagcggtggaatccaccacctgatctatcctcgggagcccgaagggatccttcggacagtgtttgttgagatcagtatagtcgacgcacatgcgccaatccactttattctttttgagtacaagaacagggttggctaaccactcggggtgcaatacttctctaataaatccggccgcgaccaagcgggctaactcggcacgaatggcttctctcttgtcgggcgtgaaacgacgcagcttttgccggatcggcctcgcctggggatagaccttcaatttgtgctcggccagttctcttgggactcccggcatatccgcaggttgccatgcgaatacgtctcggttatcttgcaggaactggacgagcgcgcttttctatttgtcactcaaactggagctgatgatggccgtcttgcgctcatcagcgaaccccaggttgatccgcttggtgtcttcagtcggccgcatagaggtcacggcctgagcttcgtttgccggcacggcgaggtcttcatcaggcttagagttcgccggtgtagaaggaatcgctgatggcttggtggtgagggctgcttggatggccactcggaaacattctgcggcgccttggaagtcggcgcgcacagttatgattcctcgtggtcctggcatcttcagtatcatgtacgtgtagtgtgggatggccatgaacttggccagccctggcctcccgatgatggcgttgtacccgcagtcgaagttcgccacctcgaacctcaggaactcggttctgtaattatccggagtcccgaaggtgaccggcatgtagatgtggcccagcggatactccccttccgtcggcacgatgccgaagaaaggagtgtctgactcgtggagctctttgaggtgaactcccaagccttgaagcgtacgggggaaggtgacgttgatgctgctccccccgtccactagcaccttcttcactcggctctctcggatcaccggatcgacgaggagcgggtatttgcctgggtggtcgaagttgagccattgatcctcccgagtgaatgtgattgggtgctccgaccaccggtacggggcgggagggccggtggtcgccaccagtatctggcggtcgttgagcttttgttgtctcttgttctcctgcgacccatgtccgccgaagatgacgttgacctccctgtcaacgcgcgggaaagctcctcctcccccctcctccggctgatggggctgtcgtggttcatctggtcctcctctcggcggaggaggtggtagaggttggaagggtcggccatgcccgacggagtgcttgaagtcccggcagttgcggagagtgtggcgcatatccttgtggtacgggcactgggcgtcgaggatgtcgtccagcgtgcgttcgcctccacgaggtcctcctcgggcgcgagaggcgggtggtccgacggcgtgtacctcttcgcgaggcctcttctcccagcgcttgtcgggcggctggttcgcgtcgcgtcgtggtaccgccggtgcgggctttgctcctccgatgaggtcctgggcccgctcgtcagcggtgatgtagaggtcggcttcccggaacagctcctcggaggtagtcggcgccttttgcagtatggctcggacgaaagctgagtcgttagatcctctgtagaagtcctcgattacggccgcctccgtgacctcggggatgcggtttctcatggtctggaaccttttaaggtatgacctgagagtctcatccccccggcgcttgatggatttgaggtcccatggttgcgctggcttgtcggagagagactggaagttggcggtgaaacgtcgactgaagtctccccagtcgtcgatgcagtgtcggggtagatgtcgtagccactgcagcgcgtcttgcccgaggacgatgggcagatacgcagtcatgacgtcctcggatgccccagcggcccgagcagcggtggtgtagacggctaaccagccccccggatcctgcttaggttcatatttgtcgacattggataccttgaagttgggaggccattggatggccctaaggcgcggagtaagggcggatactccgcacgtgtcttcctgtcgtcgaggatgtcgtctgtcccggggaggggagtggcggtggtggctcctcgaccgtccccgggtggttccaccagttgaagctgttgccgactcagttcgggtggcctggctttgggtcgggaagccatgatctcgatcatactcctctcgacggcgaatttcgttctcgtgccgccgttcgcgcgaagcattgatagagcttcgcgcgtctcggcgactgttgatggcgtgtcgcagatcgttcggcggatgagcgagcggtagaagatggttggctgcctgagtgaacaggcgccggtatccctcggcgtcgggggtccgaggaagtccgtcagctatccgggccagaacgcctccgacttcgctcggcgtattcatggctcgggcgaagtcggggttcaggttgcgcccgaagagcggattctcgcgtcgttgccttgcatcttgctcggcttgctcctgagttcgacggcgatctcgtcgtcgggagttccttcgttccctgaagacgcgttcttccggagtttctcctatctctgagacctcgtctcgcgagacaggttgctccggatcctgttctccagctgcgtgatgtcgtcgaacgttcctgcgccgattccttcgtcggcgggattctcgctgaggcggttcttctccaggcgcggtcggttcatcgtcggagacggcgggtgattctgctctcccgatgaagaggatgtcggggtagaacggtattgctgccgcggcgactttctttccattctcctttgaagtcggaggaacgggaagaacgaccggcctctccctgatctccttccttctcatgatctgtgtccattctttcgtcggggaagtagacagcggagtcttccgggtcagcgagcttctcgctccagcctccccggagacgatcttttcccgaagaaccggaagtagcgtctttccagcattttcggagtttgttggtggagacttctgttccggcagatccgcgaggcggtgtagaattccttcttcatccgccacggaggagattgtcccgaagcagaatgtggatccgggacgcatggtgatcttgctgtggaaggtgacggccattgagctagcttggatcgtcgacacaccccctacctggcgcgccagctatcggtgttttgggtccgaccgcacacccggggttgcccctcaaggtgtttttaggagtaggacggtgtcgacgactgtagcaaaatggttcgtgccgatcgcacgagggacaatggacaagatttacaggttcgggccgcttagaaacgcgtaacaccctacgtcctggtgagtatatgagcgtggttacaaaaggattctctggatagagggcgcagagagtttttgtgggtggctaagtggagcagagtcgatccatctgaaggggtgccccctaggccttatatactcggccgtggagcattacatgtgatacgataacaacaagtagctgaaagatagtgaacttcttgagcttatccttacgtaacctccgccgacttatcctcgcatggctccgttgcatgggggcttcagcgcgggaaagtcgggtctctgtttgtgattcattcgttcgacgttgtgggccgcctgtgtttgcactaatcagtcccacgtcttctttattttaacggctgtctttccctaggcccacgaaagaatgaccttacgaattattgggcccttgggcctttcgtgaggccttttacttctttagtggacccgggggatatctatcccccacaggtagggattccaaggtgtgaaatccatcttgactaagagtagaaaaggtaagaatgatcagagtagaacagtagaaggtgagacagaatcaagataagtgtagaaagaatcagagtaaggtaagtagtgaagggttttgtccatttctatctaggtttcgtcctacagtcaacatttcctctgataccacttctgtcacacccggctttaaggaacaaagccgggtgcatctcatacatgcgccaagaagacaacatatataataacagagtgtatagagataaatgtcataaaacatcagagtatttattacatagcggaagacttattacaaaataaaagataaaagtaaaacgaactaaggatcgtcggcgccaatgtcgactgggaaatgccacctagatcagatcgaactcctcagtgttaggcggctcctcctgaaccacctgctcttctcctgtgggggggtgtgagacagcaagggtgagctcacacatgatcatagctcaacaagttgtggggaaccggtggacataaactcacaaaggtgtgaggtcatgtgatgtgtaaggctaatcaatgataggggttaaagctgagcattgcttttaagtagttggtcaaaattttatttagcagttactaaagtataagtgaataccaaaaccttagataaagtaattgaacaaaaattaataatagaccccatgcaatgcaaatgacaaaattgaatttaagttccataatttaaaacatcagagagtcctgagccgctcatgaccgcgagcacggctagtatactagttttacactctgcagaggttgtaccctttacccacaagtcatgctacccatctgccaaggggtcacgaatcccatacacctctacctaggaagcgcggcagggcaacactacgaggcctttacaaagttccactagcttccgaaaacccgctacagtttataagaagttccaatgcagggttcttgtctgaccgccatcgcagcaaaatcaaccaaggacctccctacactgaccactcccctactgcccttgcccctttcgggtaaggtagtcttccactagctttcctaattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaaccatgtaaagcaatagcaaactacccaagtgattcaggggtaaacaaggtaatgagataaacaaatctagggtgtcctattgggtcccatcaaaattaacctatgcacgagtgaatgataattagagaacattattgggtaaaaagtggtcaagggcacaacttgcctggcactagagattccagttaccagggtgattcttcaaatcctcgtgacctcactgctagtcgtagcaatacaaacaaacatggtatagacaaaattaacatcacaccaaacataagaatatactgcgtaataatgatatacgcgttgctacgagactaacgcaacttgaacggatcaaattgaagttaaaacggaggatatatgaatttcctaaggttttatgtatttgatacgaggttaattaagaaataaattttaataccattttcatgttaaaacagagacacagggtgataaacaacattattacaaaattatagaaactaaaatggattaatttggactacacatgaattttttttttttttaaccaattctagcatttatttctacattaaaaatccaacgaatcaagctgattcatcaaaggtaccaaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaacttaatgaacttgacatgaacataaaaaagaataccaagaataattgaaacatagatataataattgattatcccaaaaccatgtaaagcaatagcaaactacccaagtgattcaggggtaaacaaaggtaatgagataaacaaatctagggtgtcctattggtcccatcaaaattaacctatgcacgagtgaatgataattagagaacattattgggtaaaaaagtggtcaagggcacaacttgcctggcacttgagattccagttaccagggtgattcttcaaatcctcgtgacctcactgctagtcgtagcaatacaaacaaacatggtatagacaaaattaacatcacaccaaacataagaatatactgcgtaataatgatatacgcgttgctacgagactaacgcaacttgaacggatcaaattgaagttaaaacggaggatatatgaatttcctaaggttttatgtatttgatacgaggttaattaagaaataaattttaataccattttcatgttaaaacagagacacagggtgataaacaacattattacaaaattatagaaactgaaatggattaatttggactacacatgaattttctatgaattaaaccaattctagcatttatttctacattaaaaatccaattctatatttgttttctgatttttctggctctctggactgggcacactaatacagagaagttcaggggtctcggtgtaagttttcctaagactcagagcccaactacgtggactgcgggttatttattaacgacattagggctcttttgtaagatgggtcggcagcagtatgcgcatgcaccctgagccgttagattttcatcccacggccgcaacttaacacaacgaaggggtacgccatgtggagcctcggatccagatccaacggcaaccaaaaaggcatgagctggcaactaatcacgcgtgttggatcacgatctaacggcccacgactaaggaatgaaggggtaatctaggccgcccgtcatcaatctgacagccacaaccccctcttctaccttatgccgctaccttctcctggaatacgcaagactccaccaacagcaagcgactcggccctgttgactcggaggaggggaattctcaccgttgaagagcagggatgccgacgacgaagcgcgggtcacggtgctgtgaagccctagcgcagaatgaagttccttcgggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccacataaggtcggctccaagaggtaacgaccatacgataacccgcggcgggaacggcgagatctttagcaaatagaattccaggagcggatcctgggatcaaagacgtgagcttgattcgttctcggcggaagtgatagtcatgcggcgtagagaacgcgagttctgttacggcgaaggggaaggaactgacaacctgggcccgcctgcaagtcgccacagcacgcgcatctggaatgtggcccccaccatcggcgcagagggaagaggctggcaccaacggcccaccggtcggcacacggtctaatccgttcttttgggctgtgcgcatttgcctgatgggccgatcacagaagaaattcggcccagagcgcaggtagtccccctctcctttcttttattttctatcctcttattcataaatattttgaattcaaatttggatttgaatctgcttttgagctctacccctgagtcaggtgtcacattaaagcactagtatagcaataatattttatttatttattttacatttttttatcacataaaattttctttccttccctttctaggatttcttttagattctaaattccctttttggactttaatatttttcttgtcacatttttattttattttgtcacaatatgcacacaaaagaaaatcctagcatgatgcatgatttatttgagtgtttttatttaattatttatttgtggaagtgaggtgttcacatgaaatgatagatatagatgacacacatgtatataaaggaatataatctctccttttagatttttattacaaagtgggtattacaaatcctaccccccttaacaagaatctcgtcctcgagatttaggaaggactaggaaaaagatggggaaaaatctatgtgaaactcttcttctctttcccatgatgcttcatcttctccatggtgactccattgcactttgcacatttttatcaccttattccttgtaagtcgagtaaaagtgtcaagaatcttgatcggataccccgtgtaagtcaaatcatcctgaacactgagctcttccattggtaactgttcctcaaggacacggagacacttcttaagttgagacacgtaaattacattatacacatcagatatattatcaggtagctcgagttgataggccatctctccaactcgcctaaaaactcataatggtccaataaagtgagggaacaatttgcccttaactttaaatctcctcattccacgaagtggtgacactttgaggtacacatgatcaccttcctcaaactccagtggtcttcttctattatcagcgtagctcttttgcctggtctgagctaccctcaaattctcccgaattatacggacttgttcctctgcttcttgaataagttcaggcccaaagaaatgtcttcctctagtctggtccaaaaatagaggtgtcctgcatttcctgccatacagagcctcgtacggtgacatcttcagactagcctgatagctattattatatgaaaactcagcataaggtagactcttgtcacaactactaccatgctgaagggcgcaagctctcaacatgtcttccaatacttgattagcccttttagtttgtccatcagtctgaggttggtaagccaaaactagaattcaacttcgcatccatattctcatgaaaactttttccaaaatctggaggtaaactgtgattcctctatccaaaatgatcttcttcgggtacatcgtgtgggagacacaatccgagccatatattaactctaccaatttgagatactttatagtagtcttgaccagaaatggaatgaagtcactttggtaattttatccataataacccaa

SEQ ID NO: 71 - DNA, 172841 bp, Zea mays
  variation 1872..1971: note = n is a, c, g, or t
  variation 3198..3297: note = n is a, c, g, or t
  variation 20998..21097: note = n is a, c, g, or t
  variation 25653..25752: note = n is a, c, g, or t
  variation 28223..28322: note = n is a, c, g, or t
  variation 38718..38817: note = n is a, c, g, or t
  variation 40257..40356: note = n is a, c, g, or t
  variation 53514..53613: note = n is a, c, g, or t
  variation 63930..64029: note = n is a, c, g, or t
  variation 67556..67655: note = n is a, c, g, or t
  variation 82865..82964: note = n is a, c, g, or t
  variation 137615..137714: note = n is a, c, g, or t
  variation 161457..161556: note = n is a, c, g, or t
  variation 162928..163027: note = n is a, c, g, or t
  variation 164025..164124: note = n is a, c, g, or t
  variation 171319..171418: note = n is a, c, g, or t
  source 1..172841: mol_type = genomic DNA
ttgggttattatggataaaattaccaaagtgacttcattccatttctggtcaagactactataaagtatctcaaattggtagagttaatatatggctcggattgtgtctcccacacgatgtacccgaagaagatcattttggatagaggaatcacagtttacctccagattttggaaaaagttttcatgagaatatggatgcgaagttgaattctagttttggcttaccaacctcagactgatggacaaactaaaagggctaatcaagtattggaagacatgttgagagcttgcgcccttcagcatggtagtagttgtgacaagagtctaccttatgctgagttttcatataataatagctatcaggctagtctgaagatgtcaccgtacgaggctctgtatggcaggaaatgcaggacacctctatttttggaccagactagaggaagacatttctttgggcctgaacttattcaagaagcagaggaacaagtccgtataattcgggagaatttgagggtagctcagaccaggcaaaagagctacgctgataatagaagaagaccactggagtttgaggaaggtgatcatgtgtacctcaaagtgtcaccacttcgtggaatgaggagatttaaagttaagggcaaattgttccctcactttattggaccattatgagtttttaggcgagttggagagatggcctatcaactcgagctacctgataatatatctgatgtgtataatgtaatttacgtgtctcaacttaagaagtgtctccgtgtccttgaggaacagttaccaatggaagagctcagtgttcaggatgatttgacttacacggggtatccgatcaagattcttgacacttttactcgacttacaaggaataaggtgataaaaatgtgcaaagtgcaatggagtcaccatggagaagatgaagcatcatgggaaagagaagaagagtttcacatagatttttccccatctttttcctagtccttcctaaatctcgaggacgagattcttgttaaggggggtaggatttgtaatacccactttgtaataaaaatctaaaaggagagattatattcctttatatacatgtgtgtcatctatatctatcatttcatgtgaacacctcacttccacaaataaataattaaataaaaacactcaaataaatcatgcatcatgctaggattttcttttgtgtgcatattgtgacaaaataaaataaaaatgtgacaagaaaaatattaaagtccaaaaagggaatttagaatctaaaagaaatcctagaaagggaaggaaagaaaattttatgtgataaaaaaatgtaaaataaataaataaaatattattgctatactagtgctttaatgtgacacctgactcaggggtagagctcaaaagcagattcaaatccaaatttgaattcaaaatatttatgaataagaggatagaaaataaaagaaaggagagggggactacctgcgctctgggccgaatttcttctgtgatcggcccatcaggcaaatgcgcacagcccaaaagaacggattagaccgtgtgccgaccggtgggccgttggtgccagcctcttccctctgcgccgatggtgggggccacattccagatgcgcgtgctgtggcgacttgcaggcgggcccaggttgtcagttccttccccttcgccgtaacagaactcgcgttctctacgccgcatgactatcacttccgccgagaacgaatcaagctcacgtctttgatcccaggatccgctcctggaattctatttgctaaagatctcgccgttcccgccgcgggttatcgtatggtcgttacctcttggagccgaccttatgtggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcccgaaggaacttcattctgcgctagggcttcacagcaccgtgacccgcgcttcgtcgtcggcatccctgctcttcaacggtgagaattcccctcctccgagtcaacagggccgagtcgcttgctgttggtggagtcttgcgtattccaggagaaggtagcggcataaggtagaagagggggttgtggctgtcagattgatgacgggcggcctagattaccccttcattccttagtcgtgggccgttagatcgtgatccaacacgcgtgattagttgccagctcatgcctttttggttgccgttggatctggatccgaggctccacatggcgtaccccttcgttgtgttaagttgcggccgtgggatgaaaatctaacggctcagggtgcatgcgcatactgctgccgacccatcttacaaaagagccctaatgtcgttaataaataacccgcagtccacgtagttgggctctgagtcttaggaaaacttacaccgagacccctgaacttctctgtattagtgtgcccagtccagagagccagaaaaatcagaaaacaaatatagaattggatttttaatgtagaaataaatgctagaattggtttaattcatagaaaattcatgtgtagtccaaattaatccatttcagtttctataattttgtaataatgttgtttatcaccctgtgtctctgttttaacatgaaaatggtattaaaatttatttcttaattaacctcgtatcaaatacataaaaccttaggaaattcatatatcctccgttttaacttcaatttgatccgttcaagttgcgttagtctcgtagcaacgcgtatatcattattacgcagtatattcttatgtttggtgtgatgttaattttgtctataccatgtttgtttgtattgctacgactagcagtgaggtcacgaggatttgaagaatcaccctggtaactggaatctcaagtgccaggcaagttgtgcccttgaccacttttttacccaataatgttctctaattatcattcactcgtgcataggttaattttgatgggaccaataggacaccctagatttgtttatctcattacctttgtttacccctgaatcacttgggtagtttgctattgctttacatggttttgggataatcaattattatatctatgtttcaattattcttggtattcttttttatgttcatgtcaagttcattaagttannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatttggtacctttgatgaatcagcttgattcgttggatttttaatgtagaaataaatgctagaattggttaaaaaaaaaaaaaattcatgtgtagtccaaattaatccattttagtttctataattttgtaataatgttgtttatcaccctgtgtctctgttttaacatgaaaatggtattaaaatttatttcttaattaacctcgtatcaaatacataaaaccttaggaaattcatatatcctccgttttaacttcaatttgatccgttcaagttgcgttagtctcgtagcaacgcgtatatcattattacgcagtatattcttatgtttggtgtgatgttaattttgtctataccatgtttgtttgtattgctacgactagcagtgaggtcacgaggatttgaagaatcaccctggtaactggaatctctagtgccaggcaagttgtgcccttgaccactttttacccaataatgttctctaattatcattcactcgtgcataggttaattttgatgggacccaataggacaccctagatttgtttatctcattaccttgtttacccctgaatcacttgggtagtttgctattgctttacatggttttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattaggaaagctagtggaagactaccttacccgaaaggggcaagggcagtaggggagtggtcagtgtagggaggtccttggttgattttgctgcgatggcggtcagacaagaaccctgcattggaacttcttataaactgtagcgggttttcggaagctagtggaactttgtaaaggcctcgtagtgttgccctgccgcgcttcctaggtagaggtgtatgggattcgtgaccccttggcagatgggtagcatgacttgtgggtaaagggtacaacctctgcagagtgtaaaactagtatactagccgtgctcgcggtcatgagcggctcaggactctctgatgttttaaattatggaacttaaattcaattttgtcatttgcattgcatggggtctattattaatttttgttcaattactttatctaaggttttggtattcacttatactttagtaactgctaaataaaattttgaccaactacttaaaagcaatgctcagctttaacccctatcattgattagccttacacatcacatgacctcacacctttgtgagtttatgtccaccggttccccacaacttgttgagctatgatcatgtgtgagctcacccttgctgtctcacacccccccacaggagaagagcaggtggttcaggaggagccgcctaacactgaggagttcgatctgatctaggtggcatttcccagtcgacattggcgccgacgatccttagttcgttttacttttatcttttattttgtaataagtcttccgctatgtaataaatactctgatgttttatgacatttatctctatacactctgttattatatatgttgtcttcttggcgcatgtatgagatgcacccggctttgttccttaaagccgggtgtgacagaagtggtatcagaggaaatgttgactgtaggacgaaacctagatagaaatggacaaaacccttcactacttaccttactctgattctttctacacttatcttgattctgtctcaccttctactgttctactctgatcattcttaccttttctactcttagtcaagatggatttcacaccttggaatccctacctgtgggggatagatatcccccgggtccactaaagaagtaaaaggcctcacgaaaggcccaagggcccaataattcgtaaggtcattctttcgtgggcctagggaaagacagccgttaaaataaagaagacgtgggactgattagtgcaaacacaggcggcccacaacgtcgaacgaatgaatcacaaacagagacccgactttcccgcgctgaagcccccatgcaacggagccatgcgaggataagtcggcggaggttacgtaaggataagctcaagaagttcactatctttcagctacttgttgttatcgtatcacatgtaatgctccacggccgagtatataaggcctagggggcaccccttcagatggatcgactctgctccacttagccacccacaaaaactctctgcgccctctatccagagaatccttttgtaaccacgctcatatactcaccaggacgtagggtgttacgcgtttctaagcggcccgaacctgtaaatcttgtccattgtccctcgtgcgatcggcacgaaccattttgctacagtcgtcgacaccgtcctactcctaaaaacaccttgaggggcaaccccgggtgtgcggtcggacccaaaacaccgatagctggcgcgccaggtagggggtgtgtcgacgatccaagctagctcaatggccgtcaccttccacagcaagatcaccatgcgtcccggatccacattctgcttcgggacaatctcctccgtggcggatgaagaaggaattctacaccgcctcgcggatctgccggaacagaagtctccaccaacaaactccgaaaatgctggaaagacgctacttccggttcttcgggaaaagatcgtctccggggaggctggagcgagaagctcgctgacccggaagactccgctgtctacttccccgacgaaagaatggacacagatcatgagaaggaaggagatcagggagaggccggtcgttcttcccgttcctccgacttcaaaggagaatggaaagaaagtcgccgcggcagcaataccgttctaccccgacatcctcttcatcgggagagcagaatcacccgccgtctccgacgatgaaccgaccgcgcctggagaagaaccgcctcagcgagaatcccgccgacgaaggaatcggcgcaggaacgttcgacgacatcacgcagctggagaacaggatccggagcaacctgtctcgcgagacgaggtctcagagataggagaaactccggaagaacgcgtcttcagggaacgaaggaactcccgacgacgagatcgccgtcgaactcaggagcaagccgagcaagatgcaaggcaacgacgcgagaatccgctcttcgggcgcaacctgaaccccgacttcgcccgagccatgaatacgccgagcgaagtcggaggcgttctggcccggatagctgacggacttcctcggacccccgacgccgagggataccggcgcctgttcactcaggcagccaaccatcttctaccgctcgctcatccgccgaacgatctgcgacacgccatcaacagtcgccgagacgcgcgaagctctatcaatgcttcgcgcgaacggcggcacgagaacgaaattcgccgtcgagaggagtatgatcgagatcatggcttcccgacccaaagccaggccacccgaactgagtcggcaacagcttcaactggtggaaccacccggggacggtcgaggagccaccaccgccactcccctccccgggacagacgacatcctcgacgacaggaagacacgtgcggagtatccgcccttactccgcgccttagggccatccaatggcctcccaacttcaaggtatccaatgtcgacaaatatgaacctaagcaggatccggggggctggttagccgtctacaccaccgctgctcgggccgctggggcatccgaggacgtcatgactgcgtatctgcccatcgtcctcgggcaagacgcgctgcagtggctacgacatctaccccgacactgcatcgacgactggggagacttcagtcgacgtttcaccgccaacttccagtctctctccgacaagccagcgcaaccatgggacctcaaatccatcaagcgccggggggatgagactctcaggtcataccttaaaaggttccagaccatgagaaaccgcatccccgaggtcacggaggcggccgtaatcgaggacttctacagaggatctaacgactcagctttcgtccgagccatactgcaaaaggcgccgactacctccgaggagctgttccgggaagccgacctctacatcaccgctgacgagcgggcccaggacctcatcggaggagcaaagcccgcaccggcggtaccacgacgcgacgcgaaccagccgcccgacaagcgctgggagaagaggcctcgcgaagaggtacacgccgtcggaccacccgcctctcgcgcccgaggaggacctcgtggaggcgaacgcacgctggacgacatcctcgacgcccagtgcccgtaccacaaggatatgcgccacactctccgcaactgccgggacttcaagcactccgtcgggcatggccgacccttccaacctctaccacctcctccgccgagaggaggaccagatgaaccacgacagccccatcagccggaggaggggggaggaggagctttcccgcgcgttgacagggaggtcaacgtcatcttcggcggacatgggtcgcaggagaacaagagacaacaaaagctcaacgaccgccagatactggtggcgaccaccggccctcccgccccgtaccggtggtcggagcacccaatcacattcactcgggaggatcaatggctcaacttcgaccacccaggcaaatacccgctcctcgtcgatccggtgatccgagagagccgagtgaagaaggtgctagtggacggggggagcagcatcaacgtcaccttcccccgtacgcttcaaggcttgggagttcacctcaaagagctccacgagtcagacactcctttcttcggcatcgtgccgacggaaggggagtatccgctgggccacatctacatgccggtcaccttcgggactccggataattacagaaccgagttcctgaggttcgaggtggcgaacttcgactgcgggtacaacgccatcatcgggaggccagggctggccaagttcatggccatcccacactacacgtacatgatactgaagatgccaggaccacgaggaatcataactgtgcgcgccgacttccaaggcgccgcagaatgtttccgagtggccatccaagcagccctcaccaccaagccatcagcgattccttctacaccggcgaactctaagcctgatgaagacctcgccgtgccggcaaacgaagctcaggccgtgacctctatgcggccgactgaagacaccaagcggatcaacctggggttcgctgatgagcgcaagacggccatcatcagctccagtttgagtgacaaatagaaaagcgcgctcgtccagttcctgcaagataaccgagacgtattcgcatggcaacctgcggatatgccgggagtcccaagagaactggccgagcacaaattgaaggtctatccccaggcgaggccgatccggcaaaagctgcgtcgtttcacgcccgacaagagagaagccattcgtgccgagttagcccgcttggtcgcggccggatttattagagaagtattgcaccccgagtggttagccaaccctgttcttgtactcaaaaagaataaagtggattggcgcatgtgcgtcgactatactgatctcaacaaacactgtccgaaggatcccttcgggctcccgaggatagatcaggtggtggattccaccgctggatgttcgatgctgtctttcttagattgctattccgggtatcatcagattagtttggcaaaagaagacgaggaaaaaacggcgttcatcaccccgtttggtgctttctgttatacctccatgccgttcggcctcaaaaacgctggagcgacttatcagagggctattcaaacatgcttagccgatcactggggcaagcgtgtggaggcttacttagacgacgtggtgatcaaaactgagaatccagaaaacttcatcgaagatttacagctggttttcaacagcttgagaagatatagatggaagctcaatcctgaaaaatgcgttttcggggtaccagcaggaaagttgctcggatttatcgtcagccaccggggaattgaggctaatccagataagattgaagctatcatgaagatggaagctcctcgatcacaaaagaaggttcagcgactcactggatgtatggcagccctgagcagattcatatccaggctgggagaaaaaggtctgccattttataaactgctgaagaaggtggataagtttcaatggacttcagaggcacaggaagctctagacgcactgaagaaattcctcacaacgccgccagtattgaaaccgccccggcgagctacgccgactcagccggctgaagatctgctgttatatatctcttgcacgactcacgtggtaagcactgcgttggtagtcgagcgagtggaagaagggcatgcctacccggtacaacatcccgtctatttcatcagtgaggttctaggcccatcgaagaaaaagtatcctcaagtttagaagctattatatgcagtacttctaactgcccgcaagctgcgccactactttgacgaccacaaagtcatagtagttactggttttccaataggggacattcttcacaacaaggaagccattggccgaatagccaagtgggcttgcgagttggggtcccatgatatcgagttccgacctcgcactgccatcaaaactcaagcactggtcgacttcgtatcagagtggacagaacagcaagtgccggataatccagaaaccgcagaagtatggcgaatgtattttgatggctcgctgaagctgcagggagcaggagcaggaattctcttcactgcacctggaggcgaacaccttaagtacgctctccaattgctattcccggcctctaacaacgcagccgaatatgaagctttgatacacggattaaacatcgccatatcgctgggtgtcaagaggctgatggtgtacggggattctctggtagtcatcagtcagataaacaaggaatgggattgttcaagtgattcaatgggaagatactgcgccaccgtccgaaagctagaagataaattcgaaggcctggagttccatcatatagaaagagatcggaacacggcggctgatgtattgtccaagctcggatccggtcggactcaagtcccgcccggggtcttcgtgcaagaaatccttcagccgagcatctcgacggatcaaatagaagagtgcaacatcatagaccaacccgagtcagactctgatgactggaggcaaccgattattaagtatataaagaatgaagaagagccagatgacaagaactcagcagagcgcattgccagacaatcagctcactatacactcattggggaggtattataccggaggggcgcgtcaggcgtcctcatgaagtgtgttctcccgtccaccgggaagcaacttctggaggaagtccatgcagggcagtgcggaatacatgcagcatccagaacattagtcgggaaggttttcaggtcaggattctattggccgacggcgaagaatgatgcagccgagttagttcagagatgcgaagcttgccaatacctgtcgaagcaacagcacatgccagcgcagcagctacaaaccataccagtaacttggcctttcgcatgctggggattggatatgattggacctttcaagaaggctcaaggaggatacacccatgtattggtggcaattgacaaattcactaagtggatagagttccagcccattgcctctttgacctctgctaaagccgtggaattcatacaaagcataatattcagatttgggataccaaacagtatcatcactgacttgggatccaactttaccagttcagaattctttgacttctgcgagcaaaaaagcattcagatcaagtatgcatctgtagcccatccgagagccaacgggcaggttgagcgagccaacggaatgatattggaggcacttagaaaaagggtctttgataaaaatgaaaaattcgcaggaaaatggataagagaattgccttatgttgtttggagcttgagaacccaacctagccgagccctgcatggaaacactcctttcttcatggtctatgggtcggaggcagtgctacctgctgatctcaagtttggggcgccaagattgattttcgaaagcatagcagaagccgaagccactaggctggaggatattgacgtacttgaggaagaacgactgaatgcagtgattcaatcggcacgataccagcagactctaaggcgctaccacgacaaagctgtgcggcaacgtttcttttcgaagggagacctcgtcctccgccgaattctaacgggggagggacggcacaagttatcacccttatgggaaggacctttcatagtagcagaagtcactcgtcccggatcatatcgcctcacccaaatggatggcacggaagttgggaactcttggaacatagagcacctcagaaagttttacccctagctgtacttcaaaacagccggggcggccatgtactctgtaaagtggaaatatgtcatcaataaagatagatttcaaggacactcggttcgtttatgattgacttgcattcttacttaactcggggtgaccactatgccctacaaacggagcaatcgacttaagtcggcaacgacttaaggcggtgcaacatgctcacgcttacttaactcggggtgaccactatgccctacaaacggagcaatcgacttaagtcggcaacgacttaaggcggtgcaacatgctcacgcttacttaactcggggtgaccactatgccctacaaacggagcaatcgacttaagtcggcaacgacttaaggcggtgcaacatgctcacgcttacttaactcggggtgaccactatgccctacaaacggagcaatcgacttaagtcggcaacgacttaaggcggtgcaacatgctcacgcttacttaactcggggtgaccactatgccctacaaacggagcaatcgacttaagtcggcaacgacttaaggcggtgcaacatgctcacgcttacttaactcggggtgaccactatgccctacaaacggaacaatcgacttaagtcggcaacgacttaaggcggtgcaacatgcttacgcttactttacctagggcgatcactaaaccctactaacggaagttaccggctaaaagtcattgacggcttaaaccggtatagcatactcgcgcttatgaagttcaggggatgacttccatatcccacaaacaaacttcataattatcatgtagcattaccacaaatttgcaaactaataaattctgatacgataagaaagaaatcataacattcgaatgataagctgatgtcctctaaatgaatgcttgctcatgctaactgcgcgctcagagcacgcgaactgtttctttattttccaatgtctctctcaggaacgactgacgaagaagggcgattcgaggaatcaggggcagcaaccacgtcggctcttatatcctcggggacaaccacgccaggtcttctcatcaagattcgtcccgcccgaatggccttgtccagggccttatcgcgctgagctttgaaagtgacagcagcttcttcagcaactttaacaacctgctgagcagtttctaactcccgggcaatggttttcttggaagctcggagttccttgatggtggcatcctttgctgatagcaactgtgtaaggcgggccacttcgtccgaagattcttgcagttgacgacgctgattgtcaagctcctccatcgtaaagcggtggctcctctccaagatggtcagcgagctactggaatctcggtacaatctgtccaagttgtcgcgagaagcagcaaggatacgtttttcttcctacaagcaaaaatcaaaaatcaaactcattcagaatccaagagcatgataaggcgaagcgcaggttcgtaagctaccttctcagagttaagctggacatgaagagaagaactcagagcgttagcatcatccaaagccgcagagacctgactcaactcgatctgactttgagaatacttctcctcgaagtcagcgcatcgtcgagtcatttcagcttgatcttgagaatgtttttcttcaagaactttgatctgccgagtcatccctatcaagaagtattcaaacaaaatgaggtcagaaggtaaaacaacctacgggcaagaataaagaagaagaagaaaggacactgaccagcattggtggcctccaactcagatacacgacgatgaagcaacactggattccaacgctcgagagatgacgccacttctggggtagaagcaccctgtgatttcagttgactggccaatccattcaccaaagcctgacgagaagaacagatgagcataatgacagcaattcatgcaacatagagatcaaattaaagcacggcacagcacctggaggttggaaaagaacgaagggatccccaagtcaggagaaatcagctgataatcaggtgtcagaccaccacccgaagcagtttgcagtagaccagcaggaacgagctcagtacattcagcagagcctaacaccagaccagtggggatgctgccctctaaagcgaccccctgatctggagtttgagccaccaccatgccgccagagtgtggaggtgaacccacgtgaacgtccatagaagtgcaggaaggagaccctgctcgggcaccctcgggggctgggtcaaggttggcactgcccacttgagccggatcacccccggcaacaccctcgggggctgggtagtggcctacgctcctcacctgagctgagtcctccccggcgacaccctcgggggctgggcacatgtcagcactattcaaaggatccaagctctctgccgtgaccacctctaaggtcgacgggccttcagctacctccgcaggatcaggacgatccaagtcatcattccggccctcgagatcgtgctctaaggtcgacgaggcacgggacgacctcaatccagcatctggcacgcccgcacaagtttctgttgcatcaacgtctgcaggttccaacaacaagttctcagggaccatatcctctagagcttgatcaaaattggccatagacagttcttgcagaccaataagagcagacaaggcaggagaaggaaccccactactaccgccgctagcgacgaactgccgactgtttttccgagtcaatggagtttcattctcttcctcctcgtcttccacagcaacagcacaaacaacaccctcattggggtcagcagcaggcggagcacacccattgggatcaatgtcagctaggccagttgcagacatttcttcgatagcaggagctaaggtgctggtatcctcgtcaaaactggacactcgccggaggcgtctccttttcttcttctgctcatcagcagaaggctcgggctgactggttcggctagggcgcctcgggcgagtactgacaggtttcgaggagtccaaggtcgcatcaacctctggaaggggcaccactgccaatgtaccatcaggagcagtatcagatgaatccacagctagcagatcgagcatgtcatttatgtctgcatcactagggttcagaggcacttcaaaataaatctgccgttcatcatcaggtatatccccgagcgaagcaactaaagaacggacctcttcagcagagggtcgcactctaagacccaaattgctatctatcacgggtggattggacacaaaaagagtgaaggcttgagaagaaggtaggttccaagccgagtatgccactggagctccaacgttcgaaactttacccctgaggatcatttcaagccggctcaccaaatccacagaaggaattctcctattagtaactcgggttgagtcggccagccctcggtaaagatatgccggataggccctgtctttcagcggctgaatgtttttgaacacaaaatctgtgaccacggcttcggcagtcagacctctctctttcagtaatccaacttcagtaagcaacacgcccgcttcggctacttcttggtccgtgggagattcagtccaactcggagtacgaacgtctgcctgtcttcccgagcgggaagggagagaatttccataattatccattataaaccactccagacgccaacctttaatgctgtctttaaggggtatctcgagatactcagtcttccgcccccgacgcatctctaagctggcacctccgaccaattgatgttgccccccggccatcccaggacggcagtgatacagatacttccataagccaaaatgcggcagcacgccaagataagcttcgcaaaggtgaacgaaaatagaaatttggagaatggagttagggttcaagtgagtcaggttgatgtgataaaaatcaaggatgccacggaaaaaaggagatatgggaaggccgaggccgcgaagaaggaaaggagtgtaaaccactgactcgtgggtatcttcggttgggacagtagtcccatggcaagagcgccaagaacagagttcccgtggagggagaaccccgatggaaacaaggcggagaagctcaacttcagaaatcacagacatatggttacctgcgaagggcaactgactgttgggatcgatggcggggatcactgcagcagacgagttcgcggtcttcctcttgggcgccatcttgcttctcgctggcgaaacagagcaggaggcgagtggcagagaagactcagatgcggaaatgcaagaactagggcacggaaagcaaaggcggctgaaagcgcgactcttaagggatattcttgagccagataccttttcaaaagtgcccagtcatcacccggcggtcatctccgaaatcccagcatgctacgtggatatctaacagttatttccaagaaagccgatgtgccacctcatcactcggccattttcctcaaagtaacttgagaagctggctatcactcggcgctgcctctaaaacgccgaccacgtgttcaatcgctcggcattacttctaaaatgccgacgccgaccttcaaatcactcggcgctgcctctaaaacgccgaccacgtgttcaatcgctcggcattacttctaaaatgccgacgccgaccttcaaatcactcggcgctgcctctaaaacgccgaccacgtgttcaatcgctcggcattacttctaaaatgccgacgccgaccttcaatcactcggcgctgcctctaaaacgccgaccacgtgttcaatcgctcggcattacttctaaaatgccgacgccgaccttcaatcactcggcgctgcctctaaaacaccgaccccgtgttcaatcgctcggcattacttctaaaatgccgacgccgaccttcaaatcacttggcgctgcctctaaaacgccgaccacgtgttcaaacgctcagcattacttctaaaatgctgacgccgatattctatcactcggcgttatttctaaaaagccgaccccgtattttgtcgcttggcattacttctaaaatgccgatgccgaccttctatcattcggcattgcctctaaaacgctggtcttgtgttcgattgctcagtgctacttctaaaacgctgatgtcaatcttcacatcgctcggcgctgtttctactatatcaaaaagaaccagttcagcattcagcaaaagcagtggcaagtcatcaaaaagaggggataaaacgatatacttcattaaagggaagttgacgaacttacaaaccaactctttatgagttggtacttccctaattctactttacattaccactactactaaactacactaattactactacattattctagctatactaaactatactaatatctaagaagaccagtggcgtctagccactggccctgcccctgcccctgccgccgccgccgcctttggtgctgcccctgctgctgcccttggtgctgtccctgctgccgccgccgccgcccttggtgctgcccctgctgctgcccttggtgctgtccctgctgccgtcgccgccaccccagccgccgtcgtcgtcgtcgtcgtcgtcgtcgtcgtcgtcgtcgtcttcaccctcgtcgccgccgtccgagtcctcctcatccgaggagcactccgactcatccgagccttcgagcccggcgcgctcgacggcccggatatatgtccagacctccgcggctatcccctgggagtcgtccgagtcatcggacgacgccgggggagagacgggagccggagacccctcaggctcggaggagaactcctcctccgagtattctgagtcaccaaactcctccgatggaggagtcgactcgcgcttgcgcttgttgttcttgcccatggttaaagttttgggagagaagaaagggaagaggcagtaaggagcagcgaagagatgtgaagaaaccagagaagcaaaggggttatttatagaagggaaaggcaaccgctcacttctaaccgcggtcactgaacagtcgaaagacattcaataagcacttccacccacccgaagacacgtcagacagcggacgccgtttcatgcaactctacacccattgggactccagtcaacagcgcaaaggatatgattacactagccgtcccatcgcattactactcagaggcaaccaggctaaaacactcagcgtaccaagccgtcccttgcccacacccattgggggggggacgcccaggaattatcagtatatttttcaaacggtcacatccgtgcagggcatgcagtgaatacctcaagacaaaaatgagatatcaacaagagttagagggaagccaaatatagccagtagagtgcaaaatatggtcgacagaaatgacttgaagactagacagagaacgtccatcaaatgtccaatcagtcacagagcaaataaattacactacctagcgagatatggatggattgcgaactcggctgcaaaagacaaaatacatgctgacctctgaagcataaaattgatgacatagcgcggatgacatcatgccaattttttacaagcagaaaggataattttcagcaaaaggacacagaaggaagaccttcgagtggattatcctcaaaaatccacttgaaggtcgggggctacacccattgggtgcaccttcggtgcaccccatggattatcattctaaatcaatatagtgccgacctctaaggcgtagaacaagattggaaagatcaatcctcaactgagatgcaggagcgcgaatggagaaaatcttcggagaagaccttcgagtagattatcttctaaaatctactcaaaggtcgggggctacacccattgggtgcaccttcggtgcacccaaggaagtccagaatcttataatccaaaatgccgatttctaaggcacaggcacaaaactaaactttggtcgaacaagtgatcagagcaagagaaaacagcagggagtcatttttggaccttggatctttaagttgattacctccaaatcaacccaaagatcgggggcttgtgggggatagatatcccccgggtccactaaagaagtaaaaggcctcacgaaaggcccaagggcccaataattcgtaaggtcattctttcgtgggcctagggaaagacagccgttaaaataaagaagacgtgggactgattagtgcaaacacaggcggcccacaacgtcgaacgaatgaatcacaaacagagacccgactttcccgcgctgaagcccccatgcaacggagccatgcgaggataagtcggcggaggttacgtaaggataagctcaagaagttcactatctttcagctacttgttgttatcgtatcacatgtaatgctccacggccgaatatataaggcctagggggcaccccttcagatggatcgactctgctccacttagccacccacaaaaactctctgcgccctctatccagagaatccttttgtaaccacgctcatatactcaccaggacgtagggtgttacgcgtttctaagcggcccgaacctgtaaatcttgtccattgtccctcgtgcgatcggcacgaaccattttgctacagtcgtcgacaccgtcctactcctaaaaacaccttgaggggcaaccccgggtgtgcggtcggacccaaaacaccgacactacccctatgacatttttaagaggtaggaagcctaagacaaaattaaaactattttctttattaaaaatgttggttgattgttctgatgatcaatgcctgatttgcttctttgattgattgaaatagtatagacgggcatcttagcatgtaccaccataaggtaatgtattagcgttagtaggtgacacactaatctacttagctaataaatcccccgtagtaatattcctcttaattactcgccttgtctacaattcttcctttcgcaccctatgtttctaacgcagatgggctacccctatagtgttagaagagagcactataggcgtgatccttggtatttcatggttaagaaaggcaaagacagtttatacactgtgctaagggaaccatagaactcaccagttccaaaggagaaatatttgaagttggaattgcagtaactaccaccatcagtctaacaacattcttagaagatgagaagtttgtgggtgacaacatccgtgcagttagagatcttccggatgtctttccagaggagttattaggggtgccaccagatatggatgttgagttggtcattgatctcttacctgggacgactcctatttctaaacggccatacaggatgtccgtagaagaactaaaggaacttaagaagcaattaacggaattacaagaggctgggtacatttgtccgagtcttcaccttggagagcatcggttttgtttgtacagaagaaagatggattgcaagggatgtgtgtggattaaaggtcccttaatggtgtcactatgaagaacaagtatccattaccccacattgaagatttatttaatcaggtgagaggtgcaagagtattctcgaagattgatcttcgatcgggttaccatcaaatgaatattaggccatcggatattcccaagacgaattccttgacccgatatggtttatatgagttcaccgttatgtcgtttggactaactaatgcactagcctactttatggatctaaaggataatgtgttcatgatttggatagattcgtcgtggttttcatcgacgatattcttatctattccaagagtgatagtgatcacgaggaacatctgaggatggtgctacagaagctacgagataaccagctatatgccaagtttagcaagtgcgagttttggattggcgaggtgccatttattggtcatattatttctaatggaggaatatcaatggatcctactaaggttaaggagataatggagtggagagtacccactacagttactgagattcagagtttcttgagactagtaggatattatcggagatttattgaaggattttctaagattgctaagcctacgacctcgcttctggagaaggacagagaatttaagtgggtcgggaagtgccaagacagctttgatcaattgaagatgtgtttgttcaccaccagtattggtatgaacagacctacagaaaggagacgaattctctagaatcggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccgacagattggtgatgctaagatacaagagaaatcaaggatcttattgaaaaaggaagaggtccggaatttacggaagatgaaccaagacccaatatggttcaagggcaggatagtgtttctggtattgaaagctttcgtaagactattctaaaggaagcccataacttggattattctattcatcctggtagtaccaagatgtatcaggaattgaagcagaaatactggtggtatggattgaagagagatgtggctgcacatgtggctatgtgcgatgggtgtcaaagagttaaggcctaacaccaaagaccagctggactattacatccactgaagatacccaagtggaaggggaagaaattggtaaggacttcattactggattgcctcgcaccccgagaggatatgattctatatgggttattatggataaatttaccaaagtgactcattccattctggtcaagactacttataaggtatctcaattggtagagttatatatggctcggattgtgtctccacacgatgtaccgaagaagatcattttggatagaggatcacagtttacctctagattttggaaaagttttcatgagaatatggatgcgaagttgaattctagtttggcttaccaacctcagactgatggacaaactaaaagggctaatcaagtattggaagacatgttgagagcttgcgcccttcagcatggtagtagttgtgacaagagtctaccttatgctgagttttcatataataatagctatcaggctagtctgaagatgtcaccgtacgaggctctgtatggcaggaaatgcaggacacctctatttttggaccagactagaggaagacatttctttgggcctgaacttattcaagaagcagaggaacaagtccgtataattcgggagaatttgagggtagctcagaccaggcaaaagagctacgctgataatagaagaagaccactggagtttgaggaaggtgatcatgtgtacctcaaagtgtcaccacttcgtggaatgaggagatttaaagttaagggcaaattgttccctcactttattggaccattatgagtttttaggcgagttggagagatggcctatcaactcgagctacctgataatatatctgatgtgtataatgtaatttacgtgtctcaacttaagaagtgtctccgtgtccttgaggaacagttaccaatggaagagctcagtgttcaggatgatttgacttacacggggtatccgatcaagattcttgacacttttactcgacttacaaggaataaggtgataaaaatgtgcaaagtgcaatggagtcaccatggagaagatgaagcatcatgggaaagagaagaagagtttcacatagattttcccccatctttttcctagtccttcctaaatctcgaggacgagattcttgttaaggggggtaggatttgtaatacccactttgtaataaaaatctaaaaggagagattatattcctttatatacatgtgtgtcatctatatctatcatttcatgtgaacacctcacttccacaaataaataattaaataaaaacactcaaataaatcatgcatcatgctaggattttcttttgtgtgcatattgtgacaaaataaaataaaaatgtgacaagaaaaatattaaagtccaaaaagggaatttagaatctaaaagaaatcctagaaagggaaggaaagaaaattttatgtgataaaaaaatgtaaaataaataaataaaatattattgctatactagtgctttaatgtgacacctgactcaggggtagagctcaaaagcatattcaaatccaaatttgaattcaaaatatttatgaataagaggatagaaaataaaagaaaggagagggggactacctgcgctctgggccgaatttcttctgtgatcggcccatcaggcaaatgcgcacagcccaaaagaacggattagaccgtgtgccgaccggtgggccgttggtgccagcctcttccctctgcgccgatggtgggggccacattccagatgcgcgtgctgtggcgacttgcaggcgggcccaggttgtcagttccttccccttcgccgtaacagaactcgcgttctctacgccgcatgactatcacttccgccgagacctcgcacgcgccacgtcttgatcccaggaccgctctggattctattgcagagatctcgccgtcaccgccgcgggtatcggatggtcgttacatcgtgtgccgacttatgcggattggcctgggcgcataagtagcgcgccgctcggtatcttggcccaccaaatgaacccctgcctcgcaccacagttgattgtgtagcctcgtcggtgggagaactccgctgccgccgaagaaagctcgacccatagccagtactagagagagaaagacgtgcgccaccgggcggattaacgggtttccgcggtcacctgagtaggggagtgggtgcggcatctgtcgccgttgggcaccgcacccgtgcctccgtccccgcctgcttgcagtgaccccgcgcacctccatgacgcaactcgtgcgtgcccgctcctgtcactcgcggtcgggttgccgcggctccccattgccgtgcagggcggggccaaactcgacggtgccctcgtcgtttgggggcctgcatggttactcgggggcttggtccacggcgggcataaccaatcgccggagttgggcatcgtcgccggtccgcatcgcgttgcgagcagcgtcgccatcgctgcggtgtaggaagaaggggctgccatcgccatggacaaagcactgggccgcagccgaagaacttcatctgcgctagggcttcacagcaccgtgacccgcgcttcgtcgtcggccatcccctgcctccttcaacggtgagaattcccctcctccgagtcaacagggccgagtcgcttgctgttggtggagtcttgcgtattccaggagaaggtagcggcataaggtagaagagggggttgtggctgtcagattgatgacgggcggcctagattaccccttcattccttagtcgtgggccgttagatcgtgatccaacacacgtgattagttgccagctcatgcctttttggttgccgttggatctggatccgaggctccacatggcgtaccccttcgttgtgttaagttgcggccgtgggatgaaaatctaacggctcagggtgcatgcgcatactgctgccgacccatcttacaaaagagccctaatgtcgttaataaataacccgcagtccacgtagttgggctctgagtcttaggaaaacttacaccgagacccctgaacttctctgtattagtgcgcccagtccagagagccagaaaaatcagaaaacaaatatagaattggatttttaatgtagaaataaatgctagaattggtttaattcatagaaaattcatgtgtagtccaaattaatccatttcagtttctataattttgtaataatgttgtttatcaccctgtgtctctgttttaacatgaaaatggtattaaaatttatttcttaattaacctcgtatcaaatacataaaaccttaggaaattcatatatcctccgttttaacttcaatttgatccgttcaagttgcgttagtctcgtagcaacgcgtatatcattattacgcagtatattcttatgtttggtgtgatgttaattttgtctataccatgtttgtttgtattgctacgactagcagtgaggtcacgaggatttgaagaatcaccctggtaactggaatctcaagtgtcaggcaagttgtgcccttgaccactttttacccaataatgttctctaattatcattcactcgtgcataggttaattttgatgggacccaataggacaccctagatttgtttatctcattaccttgtttacccctgaatcacttgggtagtttgctattgctttacatggttttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattatttttagctagtggaagactaccttacccgaaaggggcaagggcagtaggggagtggtcagtgtagggaggtccttggttgtttttgctgcgatggcggtcagacaagaaccttgcattggaacttcttataaactgtagcgggttttcggaagctagtggaactttgtaaaggcctcgtagtgttgccctgccgcgcttcctaggtgaggtgtatgggattcgcgaccccttggcagatgggtagcatgactttgtgggtaaagggtacaacctctgcagagtgtaaaactagtatactagccgtgctcgcggtcatgagcggctcaggactctctgatgtttaattatggaacttaattcattttgtcatttgcattgcatgggtctattaataattttgtcaattactttcctaaggttgggaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttggacgtctgatgaatcagcttgattcgtttgtgatcggcccatcaggcaaatgcgcacagcccaaaaaaacggattacaccgtgtgccgaccggtgggccgttggtgccagcctcttccctctgcgccgatggtgggggccacattccagatgcgcgtgctgtggcgacttgcaggcgggcccaggttgtcagttccttccccttcgccgtaacagaactcatgttctctacgccgcatgactatcacttccgccgagacctcgcacgcgccacgtcttgatcccaggaccgctctggattctattgcagagatctcgccgtcaccgccgcgggtatcggatggtcgttacatcgtgtgccgacttatgcggattggcctaggcgcataagtagcgcgccgctcggtatcttggcccaccaaatgaacccctgcctcgcaccacagttgattgtgtagcctcgtcggtgggagaactccgctgccgccgaagaaagctcgacccatagccagtactagagagagaaagacgtgcgccaccgggcggattaacgggtttccgcggtcacctgagtaggggagtgggtgcggcatctgtcgccgttgggcaccgcacccgtgcctccgtccccgcctgcttgcagtgaccccgcgcacctccatgacgcaactcgtgcgtgcccgctcctgtcactcgcggtcgggttgccgcggctccccattgccgtgcagggcggggccaaactcgacggtgccctcgtcgtttgggggcctgcatggttactcgggggcttggtccacggcgggcataaccaatcgccggagttgggcgtcgtcgccggtccgcatcgcgttgcgagcagcgtcgccatcgctgcggtgtagggagaaggggctgccatcgccatggacaaagcactgggccgcagccgaagaacttcatctgcgctagggcttcacagcaccgtgacccgcgcttcgtcgtcggccatcccctgcctccttcaacggtgagaattccccctcctccgagtcaacggggccgagtcgcttgctgttggtggagtcttgcgtattccaggagaaggtagcggcataaggtagaagagggggttgtggctgtcagattgatgacgggcggcctagattaccccttcattccttagtcgtgggccgttagatcgtgatccaacacgcgtgattagttgccagctcatgcctttttggttgccgttggatctcgatccgaggctccacatggcgtaccccttcgttgtgttaagttgcggccgtgggatgaaaatctaacggctcagggtgcatgcgcatactgctgccgacccatcttacaaaagagccctaatgtcgttaataaataacccgcagtccacgtagttgggctctgagtcttaggaaaacttacaccgagacccctgaacttctctgtattagtgcgcccagtccagagagccagaaaaatcagaaaacaaatatagaattggatttttaatgtagaaataaatgctagaattggtttaattcatagaaaattcatgtgtagtccaaattaatccatttcagtttctataattttgtaataatgttgtttatcaccctgtgtctctgttttaacatgaaaatggtattaaaatttatttcttaattaacctcgtatcaaatacataaaaccttaggaaattcatatatcctccgttttaacttcaatttgatccgttcaagttgcgttagtctcgtagcaacgcgtatatcattattacgcagtatattcttatgtttggtgtgatgttaattttgtctataccatgtttgtttatattgctacgactagcagtgaggtcacgaggatttgaagaatcaccctggtaactggaatctcaagtgtcaggcaagttgtgcccttgaccactttttacccaataatgttctctaattatcattcactcgtgcataggttaattttgatgggacccaataggacaccctagatttgtttatctcattaccttgtttacccctgaatcacttgggtagtttgctattgctttacatggttttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattaggaaagctagtggaagactaccttacccgaaaggggcaagggcagtaggggagtggtcagtgtagggaggtccttggttgattttgctgcgatggcggtcagacaagaaccctgcattggaacttcttaacgtagcgggttttcggaagctagtggaacttgtaaagaacgagtagtgttgccctgccgcgtatcctaggtagagaacgaatcaagctgattcttcaactcccaaaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntacttagtacctgcataataaaatttgaccactacttaaaagcaatgctcagcttaacccctatcatgaatagcctacacatcacatgacctcacacctttgtgagtttatgtccaccggttccccacaacttgttgagctatgatcatgtgtgagctcacccttctgtctcacaccccccccacaggagaagagcaggtggttcaggaggagccgcctaacactgaggagttcgatctgatctaggtggcgtttcccagtcgacattggcgccgacgatccttagttcgttttacttttatcttttattttgtaataagtcttccgctatgtaataaatactctaatgttttatgacatttatctctatacactctgttattatatatgttgtcttcttggcgcatgtatgagatgcacccggctttgttccttaaagccgggtgtgacagaagtggtatcagaggaaatgttgactgtaggacgaaacctagatagaaatggacaaaacccttcactacttaccttactctgattctttctacacttatcttgattctgtctcaccttctactgttctactctgatcattcttaccttttctactcttagtcaagatggatttcacaccttggaatccctacccctatgacatttttaagaggtaggaagcctaagacaaaattaaaactattttctttattaaaaatgttggttgattgttctgatgatcaatgcctgatttgcttctttgattgattgaaatagtatagacgggcatcttagcatgtaccaccataaggtaatgtattagcgttagtaggtgacacactaatctacttagctaataaatcccccgtagtaatattcctcttaattactcgccttgtctacaattcttcctttcgcaccctatgtttctaacgcagatgggctacccctatagtgttagaagagagcactataggcgtgatccttggtatttcatggttaagaaaggcaaagacagtttatacactgtgctaagggaaccatagaactcaccagttccaaaggagaaatatttgaagttggaattgcagtaactaccaccatcagtctaacaacattcttagaagatgagaagtttgtgggtgacaacatccgtgcagttagagatcttccggatgtctttccagaggagttattaggggtgccaccagatatggatgttgagttggtcattgatctcttacctgggacgactcctatttctaaacggccatacaggatgtccgtagaagaactaaaggaacttaagaagcaattaacggaattacaagaggctgggtacattcgtccgagtcttcaccttggagagcatcggttttgtttgtacagaagaaagatggattgcaagggatgtgtgtggattaaaggtcccttaatggtgtcactatgaagaacaagtatccattaccccacattgaagatttatttaatcaggtgagaggtgcaagagtattctcgaagattgatcttcgatcgggttaccatcaaatgaatattaggccatcggatattcccaagacgaattccttgacccgatatggtttatatgagttcaccgttatgtcgtttggactaactaatgcactagcctactttatggatctaaaggataatgtgttcatgatttggatagattcgtcgtggttttcatcgacgatattcttatctattccaagagtgatagtgatcacgaggaacatctgaggatggtgctacagaagctacgagataaccagctatatgccaagtttagcaagtgcgagttttggattggcgaggtgccatttattggtcatattatttctaatggaggaatatcaatggatcctactaaggttaaggagataatggagtggagagtacccactacagttactgagattcagagtttcttgagactagtaggatattatcggagatttattgaaggattttctaagattgctaagcctacgacctcgcttctggagaaggacagagaatttaagtgggacgagaagtgccaagacagctttgatcaattgaagaagagattgatgtcaccaccagtattggttatgccagacctacagaaaggatttgatatctattgtgatgcatgtggccaaggcttaggatgtgtgctcatgcaggaaggacatgtgattgcctatgcgtctcatcagttacggagacatgaattgaactaccccactcacgacttggaattgccagccgtgtgcatgcgcttaagatatggagacattatatcatggggaccaagtgccaagtatatacggatcataagagtttatattcactcagaaggatctcaaccttaggcaacaccgttggttggagcttattaaggattatgatttggagattcactatcacccgggcaaggcaaatttggttgcagatgccttgagtcgaaaggagcatgttcattcagctattgttgcccagctacccgatgagattgttgaggatttcaggagacttaacctggggatagatgctcacgcttaaggagtcattattgatgtggaacctaccttggagcaagaaatccacagaacgacagattggtgatgctaagatacaagagatcaaggatcttattacgaaaggtagaggtccggaatttacggaagatgaacaagacacaatatggttcaagggcaggatatgtgttcctggtattgaaagccttcgtaagactattctaaaggaagcccataacttggattattctattcatcctggtagtaccaagatgtatcaggaattgaagcagaaatactggtggtatggattgaagagagatgtggctgcacatgtggctatgtgcgatgggtgtcaaagagttaaggcctaacaccaaagaccagctggactattacatccactgaagatacccaagtggaaggggaagaaattggtaaggacttcattactggattgcctcgcaccccgagaggatatgattctatatgggttattatggataaatttaccaaagtgactcattccattctggtcaagactacttataaggtatctcaattggtagagttatatatggctcggattgtgtctccacacgatgtaccgaagaagatcattttggatagaggatcacagtttacctccagattttggaaaagttttcatgagaatatggatgcgaagttgaattctagtttggcttaccaacctcagactgatggacaaactaaaagggctaatcaagtattggaagacatgttgagagcttgcgcccttcagcatggtagtagttgtgacaagagtctaccttatgctgagttttcatataataatagctatcaggctagtctgaagatgtcaccgtacgaggctctgtatggcaggaaatgcaggacacctctatttttggaccagactagaggaagacatttctttgggcctgaacttattcaagaagcagaggaacaagtccgtataattcgggagaatttgagggtagctcagaccaggcaaaagagctacgctgataatagaagaagaccactggagtttgaggaaggtgatcatgtgtacctcaaagtgtcaccacttcgtggaatgaggagatttaaagttaagggcaaattgttccctcactttattggaccattctgagtttttaggcgagttggagagatggcctatcaactcgagctacctgataatatatctgatgtgtataatgtaatttacgtgtctcaacttaagaagtgtctccgtgtccttgaggaacagttaccaatggaagagctcagtgttcaggatgatttgacttacacggggtatccgatcaagattcttgacacttttactcgacttacaaggaataaggtgataaaaatgtgcaaagtgcaatggagtcaccatggagaagatgaagcatcatgggaaagagaagaagagtttcacatagatttttccccatctttttcctagtccttcctaaatctcgaggacgagattcttgttaaggggggtaggatttgtaatacccactttgtaataaaaatctaaaaggagagattatattcctttatatacatgtgtgtcatctatatctatcatttcatgtgaacacctcacttccacaaataaataattaaataaaaacactcaaataaatcatgcatcatgctaggattttcttttgtgtgcatattgtgacaaaataaaataaaaatgtgacaagaaaaatattaaagtccaaaaagggaatttagaatctaaaagaaatcctagaaagggaaggaaagaaaattttatgtgataaaaaatgtaaaataaataaataaaatattattgctatactagtgctttaatgtgacacctgactcaggggtagagctcaaaagcagattcaaatccaaatttgaattcaaaatatttatgaataagaggatagaaaataaaagaaaggagagggggactacctgcgctctgggccgaatttcttctgtgatcggcccatcaggcaaatgcgcacagcccaaaagaacggattagaccgtgtgccgaccggtgggccgttggtgccagcctcttccctctgcgccgatggtgggggccacattccagatgcgcgtgctgtggcgacttgcaggcgggcccaggttgtcagttccttccccttcaccgtaacagaactcgcgttctctacgccgcatgactatcacttccgccgagacctcgcacgcgccacgtcttgatcccaggaccgctctggattctattgcagagatctcgccgtcaccgccgcgggtatcggatggtcgttacatcgtgtgccgacttatgcggattggcctgggcgcataagtagcgcgccgctcggtatcttggcccaccaaatgaacccctgcctcgcaccacagttgattgtgtagcctcgtcggtgggagaactccgctgccgccgaagaaagctcgacccatagccagtactagagagagaaagacgtgcgccaccgggcggattaacgggtttccgcggtcacctgagtaggggagtgggtgcggcatctgtcgccgttgggcaccgcacccgtgcctccgtccccacctgcttgcagtgaccccgcgcacctccatgacgcaactcgtgcgtgcccgctcctgtcactcgcggtcgggttgccgcggctccccattgccgtgcagggcggggccaaactcgacggtgccctcgtcgtttgggggcctgcatggttactcgggggcttggtccacggcgggcataaccaatcgccggagttgggcgtcgtcgccggtccgcatcgcgttgcgagcagcgtcgccatcgctgcggtgtaggaagaaggggctgccatcgccatggacaaagcactgggccgcagccgaagaacttcatctgcgctagggcttcacagcaccgtgacccgcgcttcgtcgtcggccatcccctgcctccttcaacggtgagaattccccctcctccgagtcaacggggccgagtcgcttgctgttggtggagtcttgcgtattccaggagaaggtagcggcataaggtagaagagggggttgtggctgtcagattgatgacgggcggcctagattaccccttcattccttagtcgtgggccgttagatcgtgatccaacacgcgtgattagttgccagctcatgcctttttggttgccgttggatctggatccgaggctccacatggcgtaccccttcgttgtgttaagttgcggccgtgggatgaaaatctaacggctcagggtgcatgcgcatactgctgccgacccatcttacaaaagagccctaatgtcgttaataaataacccgcagtccacgtagttgggctctgagtcttaggaaaacttacaccgagacccctgaacttctctgtattagtgcgcccagtccagagagccagaaaaatcagaaaacaaatatagaattggatttttaatgtagaaataaatgctagaattggtttaattcatagaaaattcatgtgtagtccaaattaatccatttcagtttctataattttgtaataatgttgtttatcaccctgtgtctctgttttaacatgaaaatggtattaaaatttatttcttaattaacctcgtatcaaatacataaaaccttaggaaattcatatatcctccgttttaacttcaatttgatccgttcaagttgcgttagtctcgtagcaacgcgtatatcattattacgcagtatattcttatgtttggtgtgatgttaattttgtctataccatgtttgtttgtattgctacgactagcagtgaggtcacgaggatttgaagaatcaccctggtaactggaatctcaagtgccaggcaagttgtgcccttgaccactttttacccaataatgttctctaattatcattcactcgtgcataggttaattttgatgggacccaataggacaccctagatttgtttatctcattaccttgtttacccctgaatcacttgggtagtttgctattgctttacatggttttgggataatcaattattatatctatgttccaattattcttgttattctatttatgttcatgtcaagttcattaatgttaattggaacatagagcttaacttgagaaacacgtgccaccacaagggtttaatgggacgcccttggctgactaattaggaaagctagtggaagactaccttacccgaaaggggcaagggcagtaggggagtggtcagtgtagggaggtccttggttgattttgctgcgatggcggtcagacaagaaccctgcattggaacttcttataaactgtagcgggttttcggaagctagtggaactttgtaaaggcctcgtagtgttgccctgccgcgcttcctaggtagaggtgtatgggattcgtgaccccttggcagatgggtagcatgacttgtgggtaaagggtacaacctctgcagagtgtaaaactagtatactagccgtgctcgcggtcatgagcggctcaggactctctgatgtttaaattatggaacttaaattcaattttgtcatttgcattgcatgggtctattattaattttgttcaattactttatctaaggtttggtattcacttatacttagtaactgctaataaaattttgaccaactacttaaaagcaatgctcagctttaacccctatcattgattagccttacacatcacatgacctcacacctttgtgagtttatgtccaccggttccccacaacttgttgagctatgatcatgtgtgagctcacccttgctgtctcacacccccccacaggagaagagcaggtggttcaggaggagccgcctaacactgaggagttcgatctgatctaggtggcgtttcccagtcgacattggcgccgacgatccttagttcgttttacttttatcttttattttgtaataagtcttccgctatgtaataaatactctgatgttttatgacatttatctctatacactctgttattatatatgttgtcttcttggcgcatgtatgagatgcacccggctttgttccttaaagccgggtgtgacagaagtggtatcagaggaaatgttgactgtaggacgaaacctagatagaaatggacaaaacccttcactacttaccttactctgattctttctacacttatcttgattctgtctcaccttctactgttctactctgatcattcttaccttttctactcttagtcaagatggatttcacaccttggaatccctacccctatgacatttttaagaggtaggaagcctaagacaaaattaaaactattttctttattaaaaatgttggttgattgttctgatgatcaatgcctgatttgcttctttgattgattgaaatagtatagacgggcatcttagcatgtaccaccataaggtaatgtattagcgttagtaggtgacacactaatctacttagctaataaatcccccgtagtaatattcctcttaattactcgccttgtctacaattcttcctttcgcaccctatgtttctaacgcagatgggctacccctatagtgttagaagagagcactataggcgtgatccttggtatttcatggttaagaaaggcaaagacagtttatacactgtgctaagggaaccatagaactcaccagttccaaaggagaaatatttgaagttggaattgcagtaactaccaccattagtctaacaacattcttagaagatgagaagtttgtgggtgacaacatccgtgcagttagagatcttccggatgtctttccagaggagttattaggggtgccaccagatatggatgttgagttggtcattgatctcttacctgggacgactcctatttctaaacggccatacaggatgtccgtagaagaactaaaggaacttaagaagcaattaacggaattacaagaggctgggtacattcgtccgagtcttcaccttggagagcatcggttttgtttgtacagaagaaagatggattgcaagggatgtgtgtggattaaaggtcccttaatggtgtcactatgaagaacaagtatccattaccccacattgaagatttatttaatcaggtgagaggtgcaagagtattctcgaagattgatcttcgatcgggttaccatcaaatgaatattaggccatcggatattcccaagacgaattccttgacccgatatggtttatatgagttcaccgttatgtcgtctggactaactaatgcactagcctactttatggatctaaaggataatgtgttcatgatttggatagattcgtcgtggttttcatcgacgatattcttatctattccaagagtgatagtgatcacgaggaacatctgaggatggtgctacagaagctacgagataaccagctatatgccaagtttagcaagtgcgagttttggattggcgaggtgccatttattggtcatattatttctaatggaggaatatcaatggatcctactaaggttaaggagataatggagtggagagtacccactacagttactgagattcagagtttcttgagactagtaggatattatcggagatttattgaaggattttctaagattgctaagcctacgacctcgcttctggagaaggacagagaatttaagtgggacgagaagtgccaagacagctttgatcaattgaagaagagattgatgtcaccaccagtattggttatgccagacctacagaaaggatttgatatctattgtgatgcatgtggccaaggcttaggatgtgtgctcatgcaggaaggacatgtgattgcctatgcgtctcatcagttacggagacatgaattgaactaccccactcacgacttggaattggcagccgttgtgcatgcgcttaagatatggagacattatatcatggggaccaagtgccaagtatatacggatcataagagtttatattcactcagaaggatctcaaccttaggcaacgccgttggttggagcttattaaggattatgatttggagattcactatcacctgggcaaggcaaatttggttgcagatgccttgagtcgaaaggagcatgttcattcagctattgttgcccagctacccgatgagattgttgaggatttcaggagacttaacctggggatagatgctcacgcttaaggagtcattattgatgtggaacctaccttggagcaagaaatccacagaacgacagattggtgatgctaagatacaagagatcaaggatcttattacgaaaggtagaggtccggaatttacggaagatgaacaagacacaatatggttcaagggcaggatatgtgttcctggtattgaaagccttcgtaagactattctaaaggaagcccataacttggattattctattcatcctggtagtaccaagatgtatcaggaattgaagcagaaatactggtggtatggattgaagagagatgtggctgcacatgtggctatgtgcgatgggtgtcaaagagttaaggcctaacaccaaagaccagctggactattacatccactgaagatacccaagtggaaggggaagaaattggtaaggacttcattactggattgcctcgcaccccgagaggatatgattctatatgggttattatggataaatttaccaaagtgactcattccattctggtcaagactacttataaggtatctcaattggtagagttatatatggctcggattgtgtctccacacgatgtaccgaagaagatcattttggatagaggatcacagtttacctccagatgtgaaaagacgaaatctctagattctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatcaatcaaagaagcaaatcaggcattgatcatcagaacaatcaaccaacattttataaagaaatagtttaattttgtcttaggcttcctacctcttaaaaatgtcataggggtagggattccaaggtgtgaaatccatcttgactaagagtagaaaaggtaagaatgatcagagtagaacagtagaaggtgagacagaatcaagataagtgtagaaagaatcagagtaaggtaagtagtgaagggttttgtccatttctatctaggtttcgtcctacagtcaacatttcctctgataccacttctgtcacacccggctttaaggaacaaagccgggtgcatctcatacatgcgccaagaagacaacatatataataacagagtgtatagagataaatgtcataaaacatcagagtatttattacatagcggaagacttattacaaaataaaagataaaagtaaaacgaactaaggatcgtcggcgccaatgtcgactgggaaacgccacctagatcagatcgaactcctcagtgttaggcggctcctcctgaaccacctgctcttctcctgtgggggggtgtgagacagaagggtgagctcacacatgatcatagctcaacaagttgtggggaaccggtggacataaactcacaaaggtgtgaggtcatgtgatgtgtaaggctaatcaatgataggggttaaagctgagcattgcttttaagtagttggtcaaaattttattagcagttactaagtataagtgaataccaaaccttagataaagtaattgaacaaaattaataatagacccatgcaatgcaaatgacaaaattgaatttaagttccataatttaaacatcagagagtcctgagccgctcatgaccgcgagcacggctagtatactagttttacactctgcagaggttgtaccctttacccacaagtcatgctacccatctgccaaggggtcgcgaatcccatacacctctacctaggaagcgcggcagggcaacactacgaggcctttacaaagttccactagcttccgaaaacccgctacagtttataagaagttccaatgcaaggttcttgtctgaccgccatcgcagcaaaatcaaccaaggacctccctacactgaccactcccctactgcccttgcccctttcgggtaaggtagtcttccactagctttcctaattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaccatgtaaagcaatagcaaactacccaagtgattcaggggtaaacaaaggtaatgaagcaaacaaatctagggggtcctattgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtaccctttacccacaagtcatgctaaccatctgccaggagtcgcgaatcccatacacctctacctaggaagcgcggcagggcaacactacgaggcctttacaaagttccactagcttccgaaaacccgctacagtttataagaagttccaatgcaaggttcttgtctgaccgccatcgcagcaaaatcaaccaaggacctccctacactgaccactcccctactgcccttgcccctttcgggtaaggtagtcttccactagctttcctaattagtcagccaagggcgtcccattaaacccttgtggtggcacgtgtttctcaagttaagctctatgttccaattaacattaatgaacttgacatgaacataaatagaataacaagaataattggaacatagatataataattgattatcccaaaaccatgtaaagcaatagcaaactacccaagtgattcaggggtaaacaaggtaatgagataaacaaatctagggtgtcctattgggtcccatcaaaattaacctatgcacgagtgaatgataattagagaacattattgggtaaaaagtggtcaagggcacaacttgcctgacacttgagattccagttaccagggtgattcttcaaatcctcgtgacctcactgctagtcgtagcaatacaaacaaacatggtatagacaaaattaacatcacaccaaacataagaatatactgcgtaataatgatatacgccttgctacgagactaacgcaacttgaacggatcaaattgaagttaaaacggaggatatatgaatttcctaaggttttatgtatttgatacgaggttaattaagaaataaattttaataccattttcatcttaaaacagagacacagggtgataaacaacattattacaaaattatagaaactgaaatggattaatttggactacacatgaattttctatgaattaaaccaattctagcatttatttctacattaaaaatccaattctatatttgttttctgatttttctggctctctggactgggcgcactaatacagagaagttcaggggtctcggtgtaagttttcctaagactcagagcccaactacgtggactgcgggttatttattaacgacattagggctcttttgtaagatgggtcggcagcagtatgcgcatgcaccctgagccgttagattttcatcccacggccgccaacttaacacaacgaaggggtacgccatgtggagcctcggatccagatccaacggcaaccaaaaaggcatgagctggcaactaatcacgcgtgttggatcacgatctaacggcccacgactaaggaatgaaggggtaatctaggccgcccgtcatcaatctgacagccacaactccctcttctaccttatgccgctaccttctcctggaatacgcaagactccaccaacagcaagcgactcggccccgttgactcggaggagggggaattctcaccgttgaaggaggcaggggatggccgacgacgaagcgcgggtcacggtgctgtgaagccctagcgcagatgaagttcttcggctgcggcccagtgctttgtccatggcgatggcagccccttcttcctacaccgcagcgatggcgacgctgctcgcaacgcgatgcggaccggcgacgacgcccaactccggcgattggttatgcccgccgtggaccaagcccccgagtaaccatgcaggcccccaaacgacgagggcaccgtcgagtttggccccgccctgcacggcaatggggagccgcggcaacccgaccgcgagtgacaggagcgggcacgcacgagttgcgtcatggaggtgcgcggggtcactgcaagcaggcggggacggaggcacgggtgcggtgcccaacggcgacagatgccgcacccactcccctactcaggtgaccgcggaaacccgttaatccgcccggtggcgcacgtctttctctctctagtactggctatgggtcgagctttcttcggcggcagcggagttctcccaccgacgaggctacacaatcaactgtggtgcgaggcaggggttcatttggtgggccaagataccgagcggcgcgctacttatgcgcccaggccaatccgcataagtcggcacacgatgtaacgaccatccgatacccgcggcggtgacggcgagatctctgcaatagaatccagagcggtcctgggatcaagatgtggcgcgtgcgaggtctcggcggaagtgatagtcatgcggcgtagagaacgcgagttctgttacggcgaaggggaaggaactgacaacctgggcccgcctgcaagtcgccacagcacgcgcatctggaatgtggcccccaccatcggcgcagagggaagaggctggcaccaacggcccaccggtcggcacacggtctaatccgttcttttgggctgtgcgcatttgcctgatgggccgatcacagaagaaattcggcccagagcgcaggtagtcccctctcctttcttttattttctatcctcttattcataaatattttgaattcaaatttggatttgaatctgcttttgagctctacccctgagtcaggtgtcacattaaagcactagtatagcaataatattttatttatttattttacatttttttatcacataaaattttctttccttccctttctaggatttcttttagattctaaattccctttttggactttaatatttttcttgtcacatttttattttattttgtcacaatatgcacacaaaagaaaatcctagcatgatgcatgatttatttgagtgtttttatttaattatttatttgtggaagtgaggtgttcacatgaaatgatagatatagatgacacacatgtatataaaggaatataatctctccttttagatttttattacaaagtgggtattacactctttttccttttttctttttatttgatgttttaatttcctatttcctgctttccaatttccaataaagttcaaacttcaaatttaattttaaatgcaacaaatcaaaatattcagcatgcaatgcaacattatatatataaggtattttattattcaatatgcaattcatacaaaaaaaatggttatgtagagacaatagcgctcctagtgtataaaatattttagagacaatatatattcattgcataaaggaatagctaaaatcatatagggatacttttattaaaaaatttatcagaaacaaaattttgaaagttattttaattcaacattcttggacaattttactttaataccaacataggattttgaggcgttacaacctttttgagcgagttgagtgaaaacaacagctcggaggcaggcatttacaaattacaatgcaggtcctgaacagagctaggggcgggtggggtaggggggggggggaaatactgcccagctcgcttgatcacaagttcacaacactgcagagaatgaaagggagaggcagacagacagctgacagatactagatactcgatagatttgatagaacaggtgctggtcgtgccgtgatcgagacgagaaccagttcactgaaacttttagttcacagcacagagcgaagagcagcgagcgacgatacacatggtatcgcgcgcggcatgtataccaattaccacccctcctggctcctgtacatcgtacgtgcaagataggactacggctacgagccactgttgtactagtgttggtggccgccgcgtcctgcttccgatgaggacgggttggaaactatgtgcctgccggtatgcaatgcaagccaaggtaccccacgcaccaaagcgtctggtggccgctttgacttgcctacgtctcctaactgcacgggtagggatcacatcaggtttttaggaactgtcattacgaacggctcgttgttggtttgctttctctatgccgacatcgtcatcatcaccggtcttctacgtctactcttgcgttgttgtttttagtgtcgacaacccgcactataaaaaagtgcatgcatatatttattttgtcactagcactagtatatgatttgatttgctgttgaattatacgtagatatgataatagatcgtgatccaaatgttctttcataatttgtaagagctctaaataatttatagtttaaaaataaataaaaatagagatcgatcctaatcctaatctaatatgtgaaagcccgatcctaatacgatttgtgaaatttatagcgtaaaatttagagtatgttgtcaaccttagttatacatatatgaaacatgctactgttttttgtattattattaatctctaaattgctattatttccgatacgttcgggtaaagactgtgtccggaaatagcctggcctaattacctgggaccgggaaaagatcactggtcccgatcagagcaaaaaagagattgtcgcgcgctcgatcctcctgccgaggacggcgagggaagaaaaatagagcagtcgcatacgttggataatgcagaggatagccgccaccaacagcagcaggtgacctcatccacatgtggctccggctgccgccatggcagagagacgatacgacggtgcagtaacagtaaaaataggctgactctggactctgacactctgctgagcaaaccgtcccctacccaccctgtgccgcaccccaaaaggagaaggcgcggatccagcactcccaacggccaacggggccgttgtgtctgcgactctgcgtatcggatccagtccaattatccaaacgaaaactacaacggcggcaggccggtctggtcgctttgacgaggaacgagcgttataggttgttgctatgcccgcccgcgataaaaaagagtacgtaggatttgtgatgaggtgctgctaagtgctgatggatttgtgatgactggtgcctgttggccgacgcccgacgccgccccctcggcgtcgctcattccactctgtcctgcccggaactagtggcaacttgcagccgcggcaaccttggcacacagcgcaagcagctgagcctatccgcgggcgggcgggcgggtgcttttggcctcctctgctctcctaggttggaagagtctttgccaaggggatagcaaaagagaaagtacttgcatgccccaacaaggcaacagcgcctgcttttggctggggtcctctccctcttcacatcacccgagttgtagtgcgaggcaagaaggagaccgagcagaggagggtgtgcacaggcacacacaagcatcgtactcgtcgtaccagcaagagaggaagaccagcgcctagcctcctagccatgtgggaccttaacgactcgccggccgccgaggctgccccgccgtccccatctgccgacgactccggcgcgtccttctcgtcggcggccgcgctagtcgacatacccgacgacgactccgccgccgccgccgtcgtcacgcgccagttcttcgtcctggccccggcggccaccatcggcgcggcgcccggctcgtcgaacgcgcgcgcgggctggctccgccgggccgccgcgccggccggcggccccgctgcggtgcaggcggcggctggcaagaagagccggcgcgggccgcggtcgcgcagctcgcagtaccgcggcgtcacgttctaccgccggacggggcggtgggagtcgcacatatggtgagtagtcccgtactcccgactccgttgccgttgccgccgccctttcctcactcgagggagagagacggagcaggcagcagctcagctcgcgagggagggggggagggcgttcgctgttggattggaattggattcctcttgttttggcatttctcgcctctcctgcttttgcctttcgtagccacatcttatccaagttattcttacgatgccaataggcaggccatgctctctctctctctctctcttcctcctcctctccctctgtgtgtccccgtcccctgcctctctctctctctcacatccccatgctctctgcttgtgcttgtggcaatgcagggattgcggcaagcaggtctatctgggtaagtttcttgcacgtccatgagtccatccattccatacgcatccatgcttggaagacgatagataccgcccaatccgaggcgtttttggttccggactcgggactcctcacaaaaatggcttgtggcgacttgcaggcggatttgacaccgcccatgcggctgctcggtaataaaagcctactgtctgtttcattcgattcgtattggctctcctcactcccatgccaagttctccgccaataacagcgggggttcgccgccaaattgtaatctctgcctgtctggttctcccgccaacctcagggcgtacgatcgagcggcgatcaagttccgcggcgtggaggccgacatcaatttcagcttggaggattaccaagacgacatgaagcaggtgatttatttattatacaactgcgctatactaggtagcatatagcaacaatatatatagtaattttttctcgtctatctaatccacgtcttggtctaccagcagatgtgccacctgagcaaggaggagttcgttcacgtgctccggcggcagagcacggggttcccccggggcagctccaagtacaggggcgtgacgctccacaagtgcgggcggtgggaggcgcgcatgggccagttcctcggcaagaagtatgccggtcgtccgtccatctcccttctcctcgccttccttcccttcttgcccaaccacttcatgtcttcctcttcccaatccctccccatccctggtccctgtatctgcatcggcatcagcccatcctttctttgtcgttgcgcctcacattattcattcatggctgtcacttccggtctgtgtcgctgcactgcaggtacgtctacttgggcctgttcgacaccgaggaggaagccgccaggtaaaataattggaccgcactgtgtgctcgcctggcggcggttctcggagccccgctaccccctgggccgcgaaactgactgatggatgccccgtgtctctctccctttctctctctctgtgcgcgcgcgcgcagggcgtacgaccgcgcggccatcaagtgcaacggcaaggacgcggtgacaaacttcgatcccagcatttacgccgaggaggaggtcgcgccggcgggttcgtctatccccgcccgcgctctgcaacgatcacccccctcgtcgctgtgcggcggtttgctacaacctatatggacaggaagcaatctctgacgacggtccgctgtttcgccgtgcatgcagcggcgacgggcggcgccgccggcgacgagcacaacctggacctgtcgctgggaagctcggcggggaacaaaaggggcagccttgacggcggcggcggcggcggcggcgacgacaagtcctcggaccagcgcgtccccatggcgttcgacatcgactggcagacggccgcacgtcggagcaccaaggcaaaggtatccgttttggtttggtcccgcccgcgttcagcgttctctgaatcaaacgaggtgtgcgtttaaaatcttgcttaataaaagcaagcttgtcacgtttcaaccaccgggtggcatggcagctccagctcgacgcgagcgccgccatgcatgatgcaaccccacctgccgccgtacagccccaggcatccgcaagtgggtaccgactacgtaccgagctaactggctgacgctttgtttgttctgtgcaatgcaaatcgcggcagctcctgagcaacggcgatccggggaccgcggcgggaggcctttccctggcgatcggcggcggaggcgggcactggcctccgcagctgcagcatcagcagcagcagcagaggctccacggcgccaggaacaacggcggcggcacgagctggccgccgccgccgcacccgtgcccgccgccagtaccggccgccgccgcgaccgcagctgcagcatcatcacgattccatccctacgttgttacgagtacgacgcaaagcccggccggctgggtccagaagaatgggttccattcgctggccaggcccacgtagaccaagaaccaggactgggcgttcgtccttcgatcgtattccacggcgaaggtccatcatttgacccaagggcaaacggctcgtcgatccaccgatttacaccccctccccctttctttcccttttccattctctgtgttcttgattgggtgagacatggatccgaggatggatgggcctgctgcccccctgctgtaaatgaggagtttccgggaagaagacgagtgctttggtaggcgactaggcgtcgcgtcaggcagtgcgtccttttgccgctttttgttctctctcccccccttctgcattcactcaccaatcaccattggcaagcaccgatgaatggtggcatggggtgtcaacatcgcagctcattttgtttggtctcatgttgcatcactcgctgctgcacgcacgatgatggcctgctgcactgtcgtctgctgagctgctactgtcttcgatacgtgtgggtctcgcgctcgtgtaaaccttgtttctaattcgggcactgcaataactgtgggagaagagaagacgacccgggggaggccgaccggcggttaccttcctcgccggatgggacgagaccgtaccccacgccgtcatcttcatactatgtggatggatggctacattgccggcaagtgcggccactgtgcgcacagctcgcgcgcaggaagaaaagttgctagataatgctccatcgcgcgcaggaagaaaagttgctagataatgcccgcgcgcttaatttgctcgtctatctacttcactgttcctcacggcgtggcgtggcggcaggagatgaattgctcgtgtcgctccgccgtgtgctgtaggtgtgagcatgaatataatatgggtggatcttgtcggccgaatgtttttactggtatatatatatatatatatatatatatatatatatatatatatatacacacactaataagtaataactctccttttagatatttgtccttctctaatttaattttaaactaaactgcgataaataaaaaataaataaaaaaagacggagagagtatatgctagcagtttttcatgcacacacccaagcgtatagcatacgcttgtacatgtagtacctgccgtgtgtctgtcagtgtctacatagtactatacattatgcgtaccggcactgtttgagtaccatgctagctggctagagatgctccaagtcatcaagggcaacggaaggagacaaggtgagcgatgcgtacttaaacgttggctgaaattggtcggttcatatgctacccctgctacgtacacacacatgtcaaatactccttcgtctcttctttttaactgttgtccttatttttctcgagaagtcacccatatttaattttgcctaaaaatatatataaaactattaatatttatgatacattaattaatatcactagttcatcaacatgtactctcacgcggtttaaaattgtaggatataagcactagttattgtttggataatttatattaagactctattacagacctctaaaactaatatatagttatcaacaaaaattagcttagaggttcggatcgattaggtaatggaccatctaattgttagttatacctctcaaaagctattatttgttagctgctccagtccagttaaaactagctaacaacttgttatctaactaataattatcttgggaggtttggaacattcccaaaatatagtatttatttatcatctaacgtaatacacataatagatactttagagtctatatgtacatgtaattattaatctttttttgcaaaggactggattttatgtcttctcatcaatattcattttttccttttgcattatatctctctatagtttgaaacatgcaattaatttgaatcctatcgctaactaaggtggctcttgtcgcatcatatagtcatgaatcaatttttgattttataaaatttattcttattatctcaagttaagttaaaaactcgtgtgtttattgaatgtttagttaactcttgtttttacacttctctagaacaaggattttaagtcgtcagtacattcataaatactacaatagcataagaaattgttagtaacacgcataatatcattcaagataagtcatagagcttgaggtgagatctaggatcaatgtggtagcacaactaaccttgatttttttaatagtcaagccttaaaatggctgaccttttcctttaaagtaatattaaagagcttgaaaaatagatccatcaatgatcacccacagttctcacttccacttaattagggaatcatatatgcaattatggctatactatcaatatagatgtgtcaaaaactcacttaaaatatctaagtaaatctaaactaacaacctatatatagaaatagttgttatggaaacaattttatcttatgcaacataacatatatatttattgcccatttatattggcacgatgtgcacatttactttaattaaattttaatgtattaatgttgatataagtaggcaatgtagaaacatatatatggtctatagtgatctaaacaattttattgtagtagaagatgttaatactcgatttagatgtagatctgagtggtattcgattttctacaataatagaatgaaaactgagttatgtctattatttattacaataggaggtcattggtaattagatacaaattaaggcctacgtactttagctatagtgtatttcttcctaatattgatgggtaatttaaatatagagtcatgggttacgtggtttaaccgttatctggaggcacaagtgtccaagattttccaaagagttcagaaaactccgaatccatggcttgtgcttcccgttcactgaactgccttgcctcatcataggattttggttcaactagcacttcatcagttttgtgagcagcaatacctgcaatatgccgtattgttaaactactgaactgttaatgctgttcttcagcctagtacgcattcagtgacaccttagagaacccctcaaatttctctcaatctaccttaaatcgaacaagcccttacatggctggctgttggaccagcaggttgctcggtgtggactttgttctgcactttctgaagacagtggaacccgcagattcagattttagaagcatccacaacaggatgatcagtagcaatcttaattgacccggtgttaggtaaataatcctaaaactgggttacctctaggttatttgctgcctatatggagtgctattgttggattttcgtcggttaaatattgttcaatgagactgagtgatagtaagtagaatcgttggctgctgtgaggttttttgtggtgtatcgtttatttttggatttgtaaatatgtgtttttagttcagctccactactagttttgctccgaccaaaatgcgagagtaccatgtcactgtatttgaagacaaaattttcaaatggaacaatttcatatggcatgtaattggttggatactacagcaatactactgctcagttggcactacagcggcgcctgacatcactggctttgatatagcaaaactgtattgtaggaaattgtttatcattatatagaataatgattacatatcttaaaaaacacagattcagtgctaaaaaattccacgtgagtgaggtctacgaaaggattgatcaatgtaagcctttaaccataaaaacatagattaaaattcccacataagctaggtctatgaaaaagaaaaatcaaagcaagcctttcacggtaaaaacatagattaaaattaaaagtcgcatagagggggtgaatagggcgaatctgaaatttataaacttaagcacaactacaagccgggttagcgttataaatagaaacgagtccgagagagagggtggaaaacaaatcgcgggcaaataaagagtgagacacgatgatttgttttaccgaggttcggttcttgcaaacctactccccgttgaggtggtcacaaagactggatttctttcaacccttttccctctctcaaacggtcacttagaccgagtgagcttctcttctcaatcaaacgggacgcaaagtccccgcaaggaccaccacacaattggtgtctcttgcatcggttacaattgagtttatcacaagaaagaatgagaaagaaaagaagcaatccaagcgcaagagctcaaatgaacacaaatgtcgctctctctagtcactatttgatttggagtgattccggacttgggagaggatttgatctctttagttgtgtctagaattgaatgctatagctcttgtaatgtgttgaaggtggaaaacttagatgccattgaatgtgggggtggttgggggtatttataaccccaaccaccacaaagtggtcgttggaaggctgctgtcgcatggcgcaccggacagtccggtgctccaccggacagtccggtgcgccaccggacactgtccggtgtgccagccacgtcatccggtcgttagggttcgaccgttggagctctgattggtggggcctctgggctgtctggtggtgcaccggataggtcatgtagactgtctggtgcgtctcctgcgcgtgctctgactctggcgcgcattgtagcgcatttaatgcggttgcagtcgaccgatgcgcgcgaagtagtcgttgctccgctggcacaccggacagtccggtgtgcaccggacactgtccggtgactcaccggacagtccgttaaattatagcggagcggtctcccattttcccgaaggtggcaagttcagcttcgagttccctggtgcaccggacactgtccggtggtgcaccagacactgtccggtggcacaccggacagtccggtgcgccagaccagggtaccatttgggatgtcttttgctctctttgtttgaaccctttcttggtctttttattggcttattgtgactttggcactgtagaactcatagactagagcagctagtagtcattatttgtgtgggcattcagcacaaatcatagaaaagtgtaggctatccctttcatctcccttttgtgatgatgcaacacaaacaaagcattttgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttagggtcggcttggaatcttgcacacatgcatacggaaagcataatatccggtcgtgaagcacataaatagagtaaagaacttatcatcgaccggtataccttttgaactacggatttacctcccgtgtcgaggtcgagatgcccatttgttcccatgggtgtcttgatgggtttggcatccttcattccaaacttgtttagaatgtcttgaatatacttcgtttggctaatgaaggtgccctcttggagttgcttcacttgaaatcctaagaagtacttcaactcccccatcatcgacatctcgaatttctgtgtcatgatcctactaaattcctcacatgtagattcgttagtagacccaaatataatatcatcaacataaattttggcatacaaacaaatcattgtcaagagttttagtaaataaagtaggatttgcctttccgactttgaagccattagtgataagaaaatctcttaggcattcataccatgctcttggggcttgcttgagcccataaagcgccttagagagtttatatacgtgattagggtactcactatcttcaaagtcgggaggttgctcaacatagacttcctccttgattggtccattgaggaagacacttttcacgtccatttgataaagcttgaagccatggtaagtagcataggcaagtaaaatgcgaattgattcaagcgtagctacgggtgcataggtttcaccgaaatccaaaccttcgacttgtgaataccccttggccataagtcgggctttgttccttgtcaccacaccatgctcgtcttgcttgttgcggaaaacccacttggttcctacaacattttggttaggacgtggaactaaatgtcatgcctcattccttgtgaaattgtttagctcctcttgcattgccaccacccaatccgaatcttgaagtgcttcctctaccctgtgtggctcaatagagaaaacaaaagagtaatgttcacagaaatgagcaacacgagatcaagtagttacccccttttgaatgtcgccgaggatggtgttcacggggtgatctcgttgaattgcttggtggacttttgggtgtggcggttttggaacttgttcatcttcctcaacttgatcatgggcatctcccccttgatcattgctttcctcttgaggtagctcaacttcttgatcttctccttcatcattttgagcctcatcctcatcttgagttggtggagatgcttgtatggaggaagatggttgatcttgtgtatttggaggctcttcggattccttaggacacacgtccccaatggacatgttccttagctcgacgcacgaagcctcttcatcacctagctcatcaagatcaacttgctctacttgagagccgttactcttatcaaacacaatgtcacaagagacttcaactaatccagaggacttgttaaagactataaatgcccttgtgtttgaatcataacctagtaaaaagccttctacagtcttaggagcaaatttagattttctacctcttttaacaagaataaagcatttgctaccaaagactctaaaatatgaaacattgggctttttaccggttaggagttcgtatgatgtcttcttgaggattcagtgtagatataaccggttgatggtgtagcaagccgtgttgaccgcctcggcccaaaaccgatccgaagtcttgtactcatcaagcatggttcttgccatatccaatagagttcgattcttcctctccactacaccattttgttgtgtcgtgtggggagaggagaactcatgcttgatgccctcctcctcaaggaagccttcgatttgtgagttcttgaactccgtcccgttgtcgcttcttattttcttgatcatcaagccgaactcattttgagcccgtctcaagaatctctttaagatctcttgggtatgagattttcctgcaaaaagaacacccaagtgaagcgagaatggtcatccacaataactagacaatacttactcccgccgatgcttatgtaagcaatcgggtcgaatagatccatgtgtaggagctcgagtggcctgtcagttgacatgatgttcttgtgtggatgatgaacaccaacttgctttcctgcctgacatgcgctacaaatctttgtctttctcaaaatgaacatttgttagtcccaaaatgtgttctccctttagaagtttgtgaagattcttcattccaacatgtgctagtcggtgatgccagagccagtccttgttagccttagcaattaagcaagtgttgagttcagctctatcaaaatctactaagtatagctgaccatctaacactcccttaaatgctattgaatcatcacttcttctaaagacagtaacacctgtatccgtaaaaagacagttgtaacccattttacataattgcgaaactgaaagcaagttataatctaaagaatctacaagaaaaacatttgaaatgcaatggtcaggtgatatagctattttacccaatcctttaaccaaaccttgatttacatctccgaatgtgatagctcgttggggatcttggttttctcataggaggagaacattttcttctcccctgtcatatggtttgtgcacccgctatcaatgatccaacttgagcccccggatgcataaacctacaaaacaagtttagttcttgattttaggtacccaaacggttttgggtcctttgacattagatacaagaactttgggtacccaaacacaagtcttggagctcttgtgtttgtccccaacatacttggcaactaccttgcctgatttgttggttagcacataagatgcatcaaaagttttaaatgaaatgttagaatcatttgatgcaataggagttttcttcttaggcaattttgcatgggttgattgcctagagctagatgtctcacccttatacataaaagcatgattaaggccagagtgagacttcctagagtgaattctcctaattttgctctcgggttaaccggcagggtacaaaatgtaaccctcgttatcctgaggcatgggagttttgcctttaacaaagttagataatcttttaggaggggcactaagtttgacatttctcccttttggaagccaatgccatccttgatgccagggcgtctcccactatagagcatgcttctagcaaatttaaaattttcattttctaagtcatgctcagcaattttagcatctaattttgttatatgatcattttgttgtttaattaaagctatgtgatcatgaatagcatcaatgttaacatctctacacctagtgcaaatagtaacatgctcaatggtagatgtagggggtttgcaagaattaagttcaacaatcttagcacgcaaaatatcattgttatctctaagatcggaaattaaagcattgcaaacatctaattctgtagccttagcaattaatttttcattatcatttctaaggctagcaagagagacattcaattcttcaatcttagcaagcaaattaatattatcatttttaagattgggaattgaaacattactaacattagaatcaaccttagcaaattaatttagcattttcatttctaaggttggcaataatatcatgacaagtgcttagctcactagataatttttcacatttttctacttctagagcataagcattcttaaccttaacatgcttcttgttttctttaattaggaagtccacttgagagtccaagagatcatccttctcatgaatagcactaattaattcatttagtttttccctttgttgcatgtttaggttggcaaaaagggtgcgcaaattatcctcttcatcactataattatcctcatcactagaggtttcatatttagtggaggatcttgattttaccttcttccttttgtcgtcctttgccatgaggcacttgtggccgacgttggggaagaggaggcccttggtgacggcgatgttggcagcgtcctcgtcggaggaggagtcggaggagctctcgtcggaatcccattcgcggcacacatgggcatcgccgcccttcttcttatagtatctcttcttttctctcctctttcccttcttgtcgtcgctcctgtcactatcactagataatggacatttagcaatgaaatgaacgggcttaccacacttgtagcacaccttcttggaacagggcttgtaatctttccccttcctttgtttgaggatttggcggaagctcttgatgatgagcgccatttcctcgttgtcaagcttagaggcgtcaatgggttgtctacttgatgtagactcctccttcttctcctccattgcattgaatgcgtccggttgtgcttcgggcgtggaggggccatctagctccatgattttctttgagcctttgatcatcaactcaaagctcacaaagtttcctattactttttcgggagacattagtgtatatctaggattaccacgaattaattgaacttgtgtgggattaagaaaaacgagggatcttagaataaccttgaccatctcatggtcatcccatttggtgctcccgaggttgcgcacttggtttaccaaggtcttcaagcggttgtacatgtcttgtggctcctccccttggcgaagccggaagcgaccgagctctccctcgatcgtctcccgcttggtgattttggtcaactcatctccttcgtgcgcggtcttgagaacgtcccaaaactccttggcgctttttaacccttgcaccttgttatactcctctcgacttagagatgcgaggagtatagtggtggcttggagttgaagtgcacgatttggccacctcgtcctcatcatggtacctatacgaatggtacctgtacaccaaactcaacagcatcccatatgcatttgtggagtgaggttaggtgatatatcatcatatcactccacctagaataatcttcaccgtcaaatgtcggtggtttgcctaatgggacggaaagtaatggagtatgcttagaaatgtgaggatagcataggggaatcttactaaacttcttgcgctcatggcgcttagaagtgatggacggcgtgtcggagccggaggtagatggtgacgaagagtcggtctcgtagtagaccaccttcctcatcttcttcttcttgtcaccgctccgacgcgacttatcgtgtgaatgggagcccttcatcttgttggcggactctccatatggagccttcccatggcttgtggcgggcttctcgccggtcccgatctccctcttggcggatgctcccgacatcacttcgagcggttaggctctaatgaagcacggggctctaataccaattgaaagtcacctagaggggggtgaatagggcaaatctgaaatttataaacttaagcacaactacaagccaggttagcgttagaaatagaaacaagtccgagagagagggtggaaaacaaatcgcgggcaaataaagagtgagtgagagcacctagagggggggtgaataggtgatcctgtaaaacttaaaacttaagccacaaaacttggttaagtgttagcacaataatcaccaaatggctagagagaagtcttagcaaaaacacaataaccacaagagaacaatcatagagatgacacagtggtttatcccgtggttcgaccaagaccaacgcttgcctactccacgttgtggcgtcccaacggacgagggttgcaatcaacccctctcaagcggtccaaagacccacttgaataccacggtgttttgcttgctttactatatcccgtttgcgaggaatctccacactttggagcctctcgcccttacacttagatgatcacaaagaaacacggagtaagggatggatgagcaacgcacacaagactcagaaatcagagcaacaacacgcacacaagtcgcaacaagagctcgcaacacaacccaatgagtttacaactcaactagagctctagatgctatcacaaagaaacaaatgcgtggaatcgaagtctcgatgcttaggattgctttgagtatacttgatgtactcctccatgcgcctaggggtcccttttatagccccaaggcagctagtagccgttgagagcattctatgaaggctgatcttgccttctgtcgactggcacaccggacagtccggtgcacaccggacactgtccggtgcccgatttctttccttaaatggcgcagccgaccgttggcagccagagagccgttggcgcaccggacatgtccggtgcacaccggacagtccgatgcccccttctagccgttggctcagccacgtgtcccgcgcagatcgcgcggccgaccgttggcccggccgaccgttggctcaccggacagtccggtgcacaccggacagtccggtgaattatagccgtacgtcgccggtgaattcccgagagcggccagttcgctcgagccagcctgacacaccggacactgtccggtgcaccaccggacagtccggtgctcccagactgagcagagtcttggctgctcgaggcaagacaattccaattcgatttttcctgtttccagcacttagacacaatacattagtctctaaaacaatgtactaagtctgagaaacatacctttatccttgatttgtactttgtccaccattttacacagatcaactcaaaagcacttgcgttggcactcaatcaccaaaatatttagaaatggcccaagggcacatttccctttcaatctccccctttttggtgatttatgccaacataacataaagcaagtagaacaagtgcaaaatcacttcaaataaaactcaaatttgttttgaatcagttttggcatatatggatcattctttgccaccacttggtttgtttttgcaaatcaaactcaaatttctatctctaagtcaaacacacatgttaagacataaatagagtcattccaagagaaattgattcaagatttcaaaaactcccttttttccataatcaacaacttctccccacaagaaaccaactttctgacaagagagacaataaaagagttttgacaaaccaaaagctctattctactattttcaaaatctctcaagtggtagctgatccatttatcgctttggcctttattttctccccctttggcatcaagcaccaaaatgggattaatcttggcccttgaaccccattgcctcaccaaaatcttcaataaaaatgcaaaggcaataagagtacatgagatgaacttggaataagttaccctctcatcggagtgcagtggaagtctttcatggtccaagtccacctttttcctttcaatccttctttgagattaaatcagcaaactcaagcacatggttagtctcaaaggatcaagttgtaacacatctccccctaaatatgtgcatcactttgcaatggacttgtgaggtccagggagtgtttgtacaacttgagcaccataactaagcaacaaaatgcatagggaatatgatcaaaggcataaacacatgtatgctataaatcaatccaagttccgcgaatctaagacatttagctcactacgcagcctgcaaaaggtcttctcatctagaggcttggtaaagatatcggctagctggttctcagtgctaacatgaaacacttcgatatcccccttttgctggtggtctctcaaaaagtgatgccggatgtctatgtgctttgtgcggttgtgttcaacaggattttccgccatgcggatagcactctcattatcacataggagtgggactttgctcagattgtagccaaagtccctgagggtttgcctcatccaaagtagttgcgcgcaacactgtcctgcggcaacatactcggcctcagcggtggatagggcaacggaagtttgtttcttagagttccatgacatcagggaccttcctaagaattggcacgtccccgatgtactcttcctatcaaccttacatccagcatagtcggaatctgaatatccaatcaagtcaaaggtagacccctttggataccagagcccgaagcaaggcgtagcgactaaatatctaaggattcgcttcactgccactaagtgacactccttaggatcggattgaaaccttgcacacatgcatacgctaagcataatatccagtctactagcacataaataaagtaaagaccctatcattgaccggtatgccttttgatcaacggacttacctcctttgttgaggtcggtgtgtccatcggtccccatcggagtctttgcgggcttggtgtccttcatcccaaaccgcttcagcaagtcttgcgtgtacttcgtttgagagatgaaggtgccatccttgagttgcttcacttggaacccaaggaagtagttcaactcgcccatcatcgacatctcgaatttctgcgtcatcaccctgctaaactcttcacaagactttttattagtagaaccaaatattatgtcatcgacataaatttggcacacaaaaagatcaccgtcacaagtcttagtgaaaagagttggatcggctttcccaaccttgaaagcattagcaattagaaagtctctaaggcattcataccatgctcttggggtttgcttaagtccatagagcgccttagagagcttacacacgtggtcggggtaccgttcatcctcgaagccagggggttgctccacgtacacctcctccttgattggctcgttgaggaaagcgctcttcacatccatttggaacaacctgaaagaatggtgagcggcatatgctagcaagatacgaatggactctagcctagccacaggagcaaaagtctcctcaaagtccaaacctgcgacttgggcataaccttttgccacaagtcgagccttgttccttgtcaccaccccgtgctcgtcttgtttgttgcggaacacccacttggttcccacaacattttgcttaggacgaggcaccagtgtccaaacttcattcctcttgaagttgttgagctcctcttgcatggccaacacccagtccggatctagcaaggcctcttctaccctgaaaggctcaatagaagagacaaaggagtaatgctcacaaaaattaactaatcgagatcgagtagttactcccttgctaatatcacccagaatttggtcgacgggatgatccctttgaatcatcgctcgaacttgggttggaggtgccggttgtgcttcttcctccatcacatgatcatcttgtgctcccccttgatcacacgcctcctgttgatgaacctgttcatcgtcttgagttgggggatgcaccatagttgaggaagaaggttgatctcactcattttgttcctgtggccgcacctctccaatcgccatggtgcgtattgcggccgttggaacgtcttcttcatctacatcatcaagatcaacaacttgctctcttggagagccattagtctcatcaaatacaacgtcgctagagacttcaaccaaacccgatgatttgttgaagaccctatacgcctttgtatttgagtcataacctaacaaaaacccttctacagctttgggagcaaacttagaatttctacctttcttcactagaatataacatttgctcccaaatacacaaaagtaagacacattgggtttgttaccggttaggagttcgtatgaagtcttcttgaggaggcgatgaaggtagacccggtttatggcgtggcaagccgtgttcacggcttccgatcaaaaccgctcgggggtcttgaactctccaagcatcgtcctcgccatgtcaatgagagtcctgttcttcctctctactacaccattttgctgtggtgtgtagggagcggagaactcgtgcttgattccttcctcctcaagatactcttctacttgaaggttcttgaactctgacccgttgtcgctccttatcttcttcactttgagctcaaactcattttgagctctccttaggaagcgcttgagggtcccttgggtttctgttttatcctgcaaaaagaatacccaagtgaagcgggaaaagtcatcaacgataacaagaccatacttacttcctcctatgcttagataggcgacgggtccgaaaaggtccatatgaagcaactccaaaggtcttgatgtggtcatcacatttttggtatgagaccttcccacctgtttacctgcttgacaagctgcacaaggtctatctttttcgaaggttacatttgttttacctatcacatgttctccctttagatgtttatgaaggttacgaatcaagctgaacataagaagtaaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaagttgtgagactctctctgcatgccccaccacccagtccggattctagcaagcctttctacctgaaagttcatagaaagagacaagagtatgcccccaaaaattactaatcgagatcgagtagtactcccttgctaatatcacccagaatttggtcgacgggatgatccctttgaatcatcgctcgaacttgggttggaggtgccggttgtgcttcttcctccatcacatgatcatcttgtgctcccccttgatcacacgcctcctgttgatgaacctgttcatcgtcttgagttgggggatgcaccatagttgaggaagaaggttgatctcactcattttgttcctgtggccgcacctctccaatcgccatggtgcgtattgcggccgttggaacgtcttcttcatctacatcatcaagatcaacaacttgctctcttggagagccattagtctcatcaaatacaacgtcgctagagacttcaaccaaacccgatgatttgttgaagactctatacgcctttgtatttgagtcataacctaacaaaaacccttctacggctttgggagcaaatttggaattcctacccttcttcactagaatgtagcatttactcccaaatactcgaaagtatgatacattgggtttgttaccggttagtagctcgtacgacgtcttcttgaggaggcgatgaaggtagaccctgttgatggcgtggcaagccgtgttcacggcttccgaccaaaaacactcgggggtcttgaactctccaagcatcgtcctcgccatatcgatgagcgtcctattcttcctctctaccacaccattttgttgtggtgtgtagggagcggagaactcgtgcttgaccccttcctcctcaagagactcctccacttgaaggttcttgaattcggacccgttgtcgctccttatcttcttcaccttgagctcaaactcattttgagctctccttaggaagcgcttgagggtcccttgggtttcagtcttatcctgcaaaaagaatacccaagtgaagcgggaaaagtcatcaacaataactagaccgtacttacttcctcctatgcttagataggcgacgggtccgaagaggtccatatgtagcagctcaagaggtcttgaagtggtcatcacattcttgctgtgatgcgctcctcccacttgtttacctgcttgacaagctgcacaaggtctatctttttcgaattgcacgttagtcaaacccatcacgtgttctcccttgtaatacccactttgtaaggaagaataaaaggagaaattgtttcctttatatatgtatgtgtgtctctagttatcattacatgtgaccactcatgtaaaaaaagagtagatccttaataagacacaaataaaccattgcatcatattggatttttgtttgtgcattaaaagcaataataatatgaattaaataaaatgacaatgagaagaggatttgaggaaaaaggagaaagaaagagataatataaaatataaaacaaaatttacaaataaacaaacttaaccatactggtatgattaaaatctatttttaatttgagtacaaattcaaagtcatacttgaaattcaaattgggaattgaaaataggagaagaagaaaatcagagaaaaagagaaaagaagtcgaacctaaatgggctacagctaccaaatcggcccatctttgatttgcaccgcgcagcccgtcttcgctggtgacactggtctctgggacccaccactcggcctctctcgcgctcgcccttccttatcgcgtggagatgaccattgaggcccacacgtcaggtgcgtgctcctgttcttctcctcacgcgaatcgcgcgtgcgctaacagacggcggatttttaccgcgcgccgccagactccgtcaacagtccggccgattcggccgcagctgttgcgcccatgccctcctcggattggctccatgtagtcgatataaatcaggatccctggtgctggtatttttacgagccgagtgaagcgaggagaagggtcgtggcgcgtttaggcttccgcgatttctagaatctcgggtggtgtcagagaagcagagccgtgcgacgttgatcgtgtgggtcgccgggcgggaggcgaaggatccgcgctccaccgcaagtccgccgacggccatcttccccgacaccatccgggttccggtgcgttaacctccctgtgagtctcataacgcataacccgcgcctagctactgcggtctgcggtagttattgcctgtcaatcgtgggccccggtctctggggtactcgtcaccatgggggggcttcaggcgagcatacaaccatgaagagagagaaaaggggtagatgggcgtggggaggaagaagacagccggtgatccgtggagtgggagatgaatggtggggatttgtcggtgctgggccgttggattctggttgggcgcttgagattagtgctggcgcggtgatgaatttctttaaatcggaaccgttggtttgggaacaaacgggtagatttggttctgcgttccaacccacggattggctatgggccgtaggatcccgatccaagggatcgcgttgcagactgatcacttaagtgcacgtacctaatctgggtcgttcgggtctgatccgatggcccatcatggccgataccccttcactttgccagttcgcagaagagaccctagaaaaacttaagaatcaacccgcagtcacttgagttaccctctgtgtctcgggaatttttattgtttagcccctgcgcatttctgtaattgaggcccagtccagataattaataaaaccaggaaaatcctttagaaaatggttttagtacatatttaaatgcagaaacttgtttaatttgtagaaaatgcatatgaactccaaattggaccattccagttcctataattttgtaatattattctctatcaaatagtaccactgtttttacatgaaatacacgttaaaatttatttctcacttaaccctatattaaatgcataaaacctttggaaattcataacttaaaatatataactccaaaattaataattccagttcctataatctcattttagggtctagattattactgtgcattttatttacatgtttggtgtaatgttaatttttgctataccatgtatgtattgtattgatgcgagtagacgagcaagctacagaggatcctgaggttcagctggtagagactgctgagcaggagctcgtggaaggcaagttgtgcccttgatcacttcttttacccattcatgttcttattaatcataatgatctgcataggttaattttgatgggacccaataggttaccctagatttggctatctttataccttgtttcaccactgattttactactaaatttttgggtagtacatgctagtgctatatgtggttttgggtatggagatacattattcatgattacacttttgttatctatttattattgcttcagatcgccaaagaagggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatcccgttgggttggggacttgagagatgtcggaggcaaggcgttcgttttgtatcgcttgccccggctggcttcgaattcaattcgctttgcttgggcagccagtgtcgcacgtgtgcgcggcggcgggctctcccaatcatgctttagctaaccggggatgatctcgttcgtctcagcgcagttctcaagctagggatacatacatacatacgttcccggccaacgcgtacaaatcatgtgaggcagccatcagcagatgatgagctctgcgatccatcgtctgcgctctgccgcagctagccacggaggcggcatggcgcccgtgagcccgtgacggccctgtggtggctacctcccacttcccacacacatattcctctcctgtagatgacaacgttctgcagatgtcgcccgcccacattgcagctacgtactccctccgttttctttttatttgtcgctggatagtgtaattttgcactatccagcgacaaataaaaagaaacggagggagtactagcctacgtgcgtggaacgcgttatagataaaccttcaaacagctttgcctgcttgatgcagttcgatgggggttccattgcaggatcaagagttgacctgcctgctctagtggaacacatgacccacaccacaccacacataagacgtacgggagacacacttcattcacgccacgcgcgtcattacattcagatgattaattagccatctactaactataaagcagtaaaggcctagctaaaaattaattaacaagcgtacgacgtcgcatcatctatccatggatcatcatcatctagttcttctagcgcaaatatatagacgcgcggttacatcactggaattaccggaaacgcatgtttgtgcagtggtcaacgcacacggctagctagctagcctcagccctcagctcgccggagttgatttgagtggacgtacggggaacagcggcagaagcttgggctggtcgtcgccgcccctgggcgacgcgtgcaggtagaagcccttggacgcgatggccttctcctcccgctcctcctcctcctccctgcttatcaccaccacggccccgccgccgccgcacgacgacgagtgcggcgacatggacgaggacgacgacagcgacggcgacgcggagaccgccagcgctatgtaggcgtccatgggcgagaccggcgaatgcatgggcccgtggcagtggtggtgggcgtgggcgccgtgagcgtggtggtggtggtagtagaagaacgacgacgacggggacggcgacgccggctgcggccgcgccagctccagctccagcctggccggcgccgcgcgccgcctctcctggagcttgggccgcctcggcgggggcggtggcgcgtgcgcgacggtcaccgccgccgccgctgccgcatcgtccgtcgcggcgcccgtgagcttctgcacgagcgcgcggaacgtggccgggtcggcctgcacgaacgtggtgttggcgtcgacggcgcaggtggcggcggcgacctccctcgcgaccttgggcgagtgggacgccgccatggcgaagctctctggccctctcgctagctagctagctgagcaaagtaagcaagcaagggagctagcgagcgtggccaagaccaaatgcgggatcgagcggctggtggtagctttttaagggacaaaagggctgcattaaatgcattaaaagccaaaggggttgacggcccgcccgcgacggatggatgggtaccggacctggagtggagtcctctggacggatcggcgcatcccgcaggctcggctaagcgcctaagcctcgcagtcgctcagcacactgctgcacaggatgacatgggtcttttctgctctcgcaaaaccttatttgcaccccgtacgtccatgcagagttgcagactgtggtgtggtgtggtgtggctgctacaacgcgaacgcgagagagacgacgacgacccgcgtggtgtgctgctgctgcatcacgcatcttttctagccctacctatcatgtgtgtcttccgtcactggcccgcccatggcgcgtgtacgcacgtaccccgtacgaacacgatagaggaacactgagcaagcgtagcgtgatggcctccacgactcggtgtgcgtactagcggatggtaacgcccggccggccacgtcgtacggccacaaagggggcaggccgcgctcttgcgtgaggagaagaggaaggtgttgcttccggggtcgggccatcagctgcgccgcttgcaacaacgccagacagcctcgctcggttcgtcctcatatatgcagccgtacgtgctcaactagtactaggtccccggacgccgtccagcctcgccacgcggcggccggcgggcgcgcgtcccgtgtcctcctgggcgggcgccgctagctgtcccgtgttctgccggagctccggcgacgacgagtggacgatgcatgctgcatgctcttgcttgattggaaagcacggtacgtacgcaccgctggcgcccgccatgacgtttgacgtcttcggccacctgcatgcatgcagcgcgtgcgtgtcggccggctcggctcggctcttcgtcgctgctactgcctggcaagaggtcgatgcatacatggctgctggagtgctggtcacgcgcacgcacacgcaccgtctcgtacgtggtcaactacacgcacgcgcttgcctcacctggacacatatcgtcacacacgcatgcatgtgatgtgagctgctgcttgtccaggcacgcgcgcgattgcaagtgttgctttagttttaatttgttgttgtggcgagctaatatattcattgatcgtcacatattcaacaaatcaacagtgatctagcacattgcatatctgtacaatatatgaacatcttcagttcttcacgatcgcatagtttacagtttcacagattcattcacgcttcacggatcagaacttagaaggcaagaacagaaaccaaccaagcacttcaaccaaaattcctaaaaccaaccaattaaccaagagtccaagagccaacagatcagggcaacacaaaaatatgacagttgagttatttagtgcaataccattaaagagaccgtgacgcctaacacagaggtgcgaattatgccacttaaaactcaatttttacactaaggcctctaatttccatgtagtcgaaacttaggccagaaagtaattaatctatctagatatgtagctataattttatcagatgtgttgctatttctatcacaaaaaattatacaatataggtttcaatattgtcgctagcctagcaagctaaactatgaaataaacacataacctaggtaagcaataaataaagtatcaaatcttaattgagttagagttatgtcgagaactctgatacctttaaggtattgagaagagtgcaaacttttgcatactccttgttggggagtcttgcaagcgccaagcttccgatcggataactatatagcctttaggtcttttccacatgtaagttggtatcctagatggtctcttttgaattgttccccatcgtcttagcctttggccaaacagttatgtgcctcgttttctctgtgtgaggtggggtcacaccacaaatatggttggtgtgatctcactataagactcttcgagtacagatatggtgcacacaaacattaagtggtatgagattttctaatcaatggaaatattgtctaaacctagagcaagcgtataaacgataatataactaatctaagcactttcaactgtgcaagtgcaagcattcatacggcctcctatttttttttgtaccattccctttcttcttgtgattcgtcaatctttatccgagatgtcgcccatgggctggccactgacggagggctccggccccaagctttagcgcccaatcggcccaaggattttgttcgaaccaaccaccacccgctggccgctgaccaaaagaaaaggttaacgaacatattcagtcggtcagtaagcatggacgacatcaaccaactacaatgcacgaccaaaccgccatcgcgcaaacaattatattgacgccgcaagtgtcccgtcgctaacctgccaccggcaacgacagagaaattccagcagaggctccggcggcaacgaccagataattaagacgacgaggccggcacgtaggtcgtagggagaagccagccgcgcgcgggaacgggaatggacgcgctgtgcccgcaggtcctgcacgtagccgtggccgggcgcgcgctggcggtggtggagcgcgacggcacgagcgacccggccacgggccgcgtcctcaccggctcctggctgtgggagtcgtccctggtcctggccgcgcacctcgccgcggacccccgcgccaggcgccgcctccgtggcgccaccgtcgtggagctcggcgccggcacggggctcccgggcatcgccgccgtggcctgcctcggcgccgcgcgctgcgtgctcacggacgtggccgcgctgctgccgggcctcagggccaacgcggacgccaacggcctcagcgccgcgcgggcccgcgtgcgcgagctccgctggggcgacctgctcccgctcgacgacggcgacgggagggtgggcgtcgtgctcttgtccgacgtcttctacgatcccgaggacatgccggcgatggcggccacgctgcggggtatgtggtggacggacgagggcggcggcggcaccgcggggtgggcggcgagcgaggtgcgggacagcgtgctggactgcatggacgtgctgcgggagcatggttttgaggtggtcgaggtcgacagggtcaccacgccgctgctgcgcgaccccggacagagtgccgccttcgccgtctatcgcatctcgctccgacatggcgactatgagaacttcagttgctcgctaagctgctagtagcatgtacactttgaatttgacgttgcttgtgcagtaagtcattttcacttttcagtttgcattgtcgcacactttgcataggagtatgacaacttgcccattcaattcgctgcaatttgtagctccgttttttttttttttttttttttttttgcccattcattctttgttaactcgcgcaattgccatgaacacggatcgacaaggatataaaagtgacatttgcaaatttgcaatcgacacggatcgacaaggccagaacgacggcagtttacactgtcgctcaatttgtagtaggagaatatacagttgcaaagggttgctacaatatacggtttcttcttcgtctgctggcctttactattcgccgcggcaaaattcaccgctcctatgtttaaaggagcaaaagggaaacgaattactggcatttcccaaatcccaacccgtgtttcctcaagtattttgtctaaccaacccgacctttaaacttccagatgtacactctaacaagaacctgcttcgttttatgaaactataatagccctagagaacgtgaaagccgatctgcaaggctcgcgatgacaaagcctgaaaatacctgcaaaggaacaaagctcacatggtcaaacagaatgacaaatgtactcaacagtgagctagaatgctgaacaggaacacgtagcattcatattggttactgccgtaagatatagtagatatttttctataattctagttttttttaaaaaaatactaaactgttggatactcgcacctggacagaacaaagaatatacactgctgaatagtgacggccgtgaatgatcatatccgcaaccatcgccagtggaattgtgagagacatgcctagcgtggccaccaagggagtagtccaaacaacagaaagggccctggccataagttccacagtcagcctgtcacaattaccccgagtatgcaagtaataatagagtggtctggtaatataaaaagatatcagccttctttaacacataccagaagtaatctgatagtacacttccaattaggctatttgccaccaccacttcatccactttagcagagtgaggcatcgaaaactttggctcaatgcctagtgcggttaatggccagactgaaatatgaacacaataaccatatttttttttacataaaataaccatgaaaggcatgcacaattaagaaataccagaatacgatcataaattacgaaattgtatactcctgttatattaatttcgaaaagggaaacaaaaaataccaatccaccagagagcaacaagggtaaaaagtccaagataaccaaacagtttttggacatcaaccttttctccttcctccccacaaaattttttaagcagcactgaaacaatatcgaaacaattaggggccgaattcatagagctccactgcaaataaagcaaagcaaaccaacctaccagtaaatagaccatacgcgattgccgacataaaaccaaacatatcacctagaagagtcctttgtgtagccctgcaattgtaaagcttatgtatcagtagcaaggaaatgcagttgaaaataggaacgaaattccaatctcagaattgaaattcgtttttgttcatgttcatcactatcaagtcatgcaaatatagaaataaaacactcaagttttgagttgttttttaacccaaaaaaccaggcaagccaactaactgaaaactactagttgtacgcaggaccaaaaaaattacaagaacattaaattaagtccaatgtgattatcagagtataaaagagtaaaggcctagtaataaacaactcacccagatttgcctacttctgattcatcagatgcccaagtctgtcccatagttgtcattacaacgccagccatgctaatgaaaacagcaataactttcgaagcacttatggagtcttggccaagtaaaacaccaatgaaaagagtgaagagccctgaagttgaagacagtacggtggtactggcaacactagttcttgcaagcgctgcattcgataaatactgcattcattcagcgtaagagaaaaatgagttacctttacctatcaaagtattgcttttacgattgaaatttaggctttctgaaacatatatcagtagtttttgagaaacaaatgtcaaaagttattcacatgaacatttcaagtcccctggaattgcaaattaacatgcaaagccattgtaaaattacctctgtcacaaaccataaggggcaaagatacaatccataagttgcaatctccttagtcgaaagttgcttgtccttcaaaacaccatcattcatttcagtgattccatatatgagtggttttgtctctttcactacaggaatgctcacatcagtgcaatttagcactaaagtcttccgaaattcgtcattcttcaaaggggcaccaccaccaaaggaagttttgcttgcaatttatgaagcactggtgtttccagaatcctttagaaatgacaaagggaggtatatgaccataagggaggcccccaagtaagtaactgcaaaaggatgtttgtactttgtgaatatcccctgcacaagaaatttagttacaaaatgaaaattgatatatttcacaatatggacactacaaatatatatttttttaaaaagaatcaagaactagaagttgaggtgttttgcagtcatgagagtgacgaaattgtactaccagtatcccctgattctaccatcggtatcttctttaatcatgtttgtgtattgtaggtgatcaaacatatcgcaaccaccctcaatcaaccacagaagaactgtgacaacaaccacatgcatgattatgctttagcatagtttacatgaacaagctaagcaccgaggactgacgacaggcaggtacaagtcgttaaaccattaacagcagatttgacgggtgtccataagagcatgcatgcaggcagctccaaaagtatacagtagaagacaaaagaatttgacttttctttgcctttttatggtgtaaataaataaagtccagtggtgaagcctcggctggattcagaatgatgccagatgttctttttaaggcagagtggtggcagatgcataaccttcccctgggaaaaattttctatcacaggcaaccctgcgcgtaacgtttaaaaatctagatgcagagcaacacccccaggtcgtcccaacaaatacaatctgataggcacagagcatttacgcacaaaacgtaccaataggccaacatgcgtcccaaaggccaagccaagatccaagaggctccaataaaaataaacaagtgaacagaggaacaccgaacacgcaccaagatcaaaggatccacaccacacctcacctgcgtgacctccgcggatatgacccaaatcagcacgacggcgacgatgaggcagaggccagctcggtacttgaagctggagcccatgtccggaggcaggcagccctcacgtttgttcctcccttgatgtacctccggactgaaaactacggcgagaaggacgtgcggggaatttaaaaccgcgcgcttgcctcacctcaccagtcaccacgtcgcctggtccccgggcggcgggcacacgagtgcggtgcgcgagccccgtcgcctcggcatccgcctcgcttccctcctcacagaaagagcggggaggggaagcggcggtttcggtccggagggaaggttctgttttggtggtggtctacccgagccccgaggcctggcgggcgcgaccggagcggtccggggacgatgcgatggcgctggcctggtcgccagcgtggtcgattcggcggcaagcaacgggtaaacgaagcgaggcgtggtgtgctggcgcggccgcgcgcttgcgaggcgagctttcggcgtgccccggtggcagaggacggaagcagttgcgcggtgggatgtcacacccaggttttagggcaccaagacccgggcacgaacataaacaccaggtatgctgggatcaagtctcacacatatgatgtatagtggcacaggatcgaatgtcacatctttatatataacaggagttctatacaaaataaataattacattataaggagacaacggtccagcaacccaaagttgactgggagacgacggcctagacctctcacgaacacatcgaagcatcctccaagagcctcatcctgtggtacctgttcttgacctgtggggggtgtgagacagcaagagtgagctcacatacgttcatagctcaacaagttgtggggaataatgtgcatgaactcgccaaaggtgggagctcatgtgaagtgtaaggcttaccaaagaggatggttagagttgagcattgcttttaaagttggtcaaaattttattagcaattactaagtataagtaaataccaacccaattaaatagtagaacagaagtaacaacatcacctgcgatgcaatgcatatgacaaattgaatttaagttccataaattaatcatgtgagggtccgagctgctcatgaccgtgagcacggctagtataccagttttacactctgcataggttgcgcatctttacccacaagtcatgttacccatctgccaagggatcgcgacttcccatacacctctaccgaggaggtgaggcagggtaacactacgaggcctttacaaagttccactagcttcagaaaacccgctacagtttataggaagctccaatgcaggaatcccttgcaggaccgccatcgcagcaaaatcctcccgagggcctccctacactgaccactcccctactgcccttgcccctttcgggtaaggtagtcttccactagctttcctaattaatcagccaaggggtcccattcctcccttgtggtagcactgttttcccgggtggttctccatgttccaattaacataatgatcttatcatgaacaacaaataacaactgataacaaaagtgtaatcatgaataatatatctccatacccaaaaccacatatagcactagcaagtactacccagaaagtttagtggtaacaaggtataaagataatcaaactagggtaacctattgggtcccatcaaaattaacctatgcagatcattatgattaatcagaacatggctgggtaaaaagaagtgatcaagggcacaacttgcctgggactcgagattccaggtaccaggatgatcttcaggtgactcgtaacctcactgttaaacgtagcaatacatacaaacatggtatagacaaaattaacatcacaccaaccataagtacaaactgaataataacggtctacgcgatgctacgagatcgtgggttcgagaaccgctaaattcggggtcatggttgaaaaggtatggttttcagaagacccatgcaatttaagccgttaaaactagaccttatgttgatttaatagatcatgtgataaaataatctaaataaataattagttcaaccttatgttaaattataatataattagggatcatattttagccaaactataatgttaaacagattaactttgatttagagaggttaatctactaaacatgggttaaataaatatctaattataaaagcatgagatatggtataaccaatgctataactacgtagtgaatatttatacgaacctaacgcaactggaacgggtcaattcggggtttaaacggagaagttatgaatttatgaagttttaggattaactttcaatattaaaattcatttcttaaaggggttttattattccagatatcatatttgggcttcggacactattttgatctctaccagggactcaggcatatgaattcacccaatccgaggatggcggattgaatcagaacaagtcgaggggttcttttgaataatttcgcacccgaaagggtaacttcaatcctgagtcgtccgatcaaactcgcacggcccagattagatcgagtacccttccgaaccggtactcactggtgatcgtgagatgggcgatcgagggtggagattttaaacaacgatgccagccgcacgactgaacgttccaggatcgacggcccgcgtacgatttggacgaagggtataacgtttataatcacagccgccaaccacgagatctacggtctggatcccaacccaccaaagcggtacgtttctcagaatcctggccgttaagtatccgatcaacggcagaggtgttttcttccaccaggccaccagacatgacggcgccccacccatcctggccgcgacgcgccggagcaccaccacaccaacgcgcaagtccatgaaacctggcttcgacttgagctacgcatgaagaaggagaaaacaaacctttggggagggtcttgccagcgaccgcggagcagattcagctattcacggcgcacgacggatgacgacctgttccgatggtgatagggcaatcccggcggtgcaggccgccattctcgaacgccaagcccgccacgcacccagcagataccagagaatcacctggagggtcttcgagggtgcgaccgcgatggattacagaatcccccacggcggttcctctctcatctctctgctctcctacaccgccctcctgcctactcgattgcgcccagctccatccgatagctttggtgctcaatatatatagatgacagggccaagggtccggatcagaggagtccgagccagcgcgaaatgaccggcataggaatcccggccaccgcgccatccgtttcgcttcttctctgttgaggatgattacgacagatggagcccacaagtcagtgccgccaaggacagattcgcatgcagtattgcccggatttgacccgctccccgtaatcgtgcccagaaatcggtcgattcagttaggtccgccccgatcgggagctgggaggggattctgttcaggccgatatgtgcggtgtttccgttggagttcacgtgaggtgagtgacgagacttgatagtcgcctagagggggggtgaatagggcgaaactgaaatttacaaatataaacacaactacaagccgggttagtgttagaaatataaaagagtccgcgagagagggcgcaaaacaaatcgcaagcgaataatgaagtgagacacgaggatttgttttaccgaggttcggttctctcaaacctactccccgttgaggaggccacaaaggccgggtctctttcaacccttccctctctcaaacggtccgcggaccgaatgagctttctcttctcaaatcaaagccgggaacaaaacttccccgcaagggccaccacacaattggtgcctcttgccttgattacaattgagtgttgatcacaagaacaagtgagaaagaaagaaagcgatccaagcgcaagagctcaaatgaacacggcaaatcactctcactagtcacttaggctttgtatggaattggagaggatttgatctctttgggtgtgtctagaattgaatgcctagctcttgtaagaggttgagaagtggaagacttggatgcaatgaatggtggggtggttggggtatttatagccccaaccaccaaacatgaccgttggtggaagttgtctgttcgatggcacaccggacagtccggtgcacaccagacagtccggtgccccctgccacgtcatctctgccgttggattctgaccgttggagctctgacttcttgtcacacccggttttagaaggcaaaccgaatgcgaaccatgtacgtgccaggatcagttattcacgtacacagcagttacataatatggacatcatcacacagtgctcaaaatagtattaataagggaaaatagtcgattacatcatacgtctgagacgtccatatagttcttacaataaatcaaagtgcggaaaagaaacgtagataacgcggccttcacaggcagccgactgggggttgccgctaacccacacctagaactcgtcgtaatcttggaactcctggaagtctccttccacagcttcatcttcgcctgagcagtggttgcaatgctgacaacctggggggggggggtttggtgtgtagagcaagggtgagtacacatcaacatactcagcaagtatcctgtttggctgtagtggactagctttatgtggggatcagtcaagcagttgcttttagttggtcaggttattacttactagtagaaagccaggttttaacattaacccaagttattaacccaatgtatcctttccaaacggaaagaataccacttaccaataccataatcataaccagaaccatcaatctcattgccacctgtaccaaaatatctctgatcaagtatcactaatctctggagctcccttggccgctcataaccgtgagcacggctgatatatcagtttcataacactctgcagaggttgtgcactttacccacaagccgtgattccctcttgcctcgggccgatcaaacccttaaacactaccaaggtgaataggcagggtttcactacgtagcctttacaaagattccccggggctgtagccacccgttaggtttcctaaatgcaccgcactcctccccaaggggcgaacccaaacttggcagagcgagccgcatacaccgagccccattgacggcacgacggctaagtgaactacatcccagatcctctaattattcagctaagggcaccccattccaccctcatggttgcactgttttcccgggcggtcatccatagaacaggtccttacggagaggcactcgagaaaccgctcgagcccccttgaataccacaagtacaacatcataataagagaagggaaaacagcgtatcatagataatcacatcatgttcattgattagagttgagcaatagcatcagactaagtagtaataatccgacccaaataggtaaacaaggacatggataacaaaagctagtcgatccttaggtataaatgtgtaatgcgggaggtgaattaaagaatgaataggacagagataggtcaaaggacacttgcctccaccaaccgattgctgctcaggggcttctcctgcgaattcctcgggctcttcgaccggatcgttctctatgcgagcgcaaacatacatacatacatccacatatttaatacaaaagaacagtacaccatacaagataacaaataaagcgaatatgcatcaagtatgacattcgataacgcatttgttatggttagaaagaaacggggaaaggtctcgcagggggttaaatcttatgcactaatgacacaattggtttttaacaaaataattctgttatatatatttatatatactgatggaaacctaatcacttttagttgatcaacattgcacagggtaaacaattagctacgtgaatcaataacataacgaaattttaaattaaacttcctattttcccatagcatgaacagtgattagcctacttaaaatacagtaattatgatcacagaaagtaaataaaataaaataaaaaaataaaaaaaacagaaggggggggggcggttgaaccggccctagggcggttgaaccggccctaggcggggcgcgggcgcgggcgcggccggggctgaccgggcggctgagccgctgagccggccggttgaaccggcggcgggcggccgagctggcggggggggggggggggggcggcgcagggggcggccaacccggccaagcaggggggcggggcggccaaaccgctgggcgcgcaggggcgcggggcagggcgcggggcgcccaagcggccaacccggcggggtggggcgggcggccaaaccggccttggggccaagcggcgagggaaaggggagggaggagagggaaaagagggaggaggaagagagggaggagggggggcctcccgggttgtcgggattcaacggcaaggggcgcccggcggcagggggcggcgcgccaacctaggacaggggggcgggttagggggtagggccgccgccttaggtgggggagggagaaatgagcggagagggagaggttagggaataagggaaagagggggggggcggctgggccttccaggctctaaagggggggcgcccatggacctggtcgaccggggttccgtcccacggcgcgggtgaagagcaaaaagaggagagaaatggagggggaaaatagagagaaagatctccccccacttcgatccgaatcttctactgaatgcattgccctcaagcatccaagatgcaggtcggcagggccgctccaccacataagataggattcctaccggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgatctagagattcgtgaatccgatctttctacatgaatgcaattgcactttcaaagaatgaacaaaatgcaaggttcggcatggtgcatcaaacaacataaagaatatagggtttttcttacacgggaaatccaaaccgaatcccgctagaactttggaaaaggtcaaggtttagcgaagggaaaaaagaaaaggaaaaggtaacgcccgaattttggcgagtaaagaaagaaaaaattcaactgcaaaattcggggcgttacaaacctatcccccttaaaagaatctcgccctcgagattcagggctggctagcaaagagctccgggtacttggccatcagatcatcttcacgctcccaggttgcttcttcctcagagtggtgactccatctgactttgcacattctgacggtcttccttcgggtgactctatctgcaacctcaaggatctgagctggcttctcaacataggtcaagtcctcctggacttccagaccttccactggcaactgctcttccggcacacgcaagcacttcttcaactgagacacatgaaagacattatgcacagcagacaaactctctggcaaactgagctgataagccacttctccacgctttgagagaatttgatacggaccaatgtagcggggtgctagcttgcctttcactccgaaccttctgactcctctgatcggtgacactttcagataaacaaagtctccgacttcgaaactcagctctcttcttcttgtgtctgcatagcttcgctgccttgattgcgctatcttcagattctctcggaccatcttgatgttctcttcggcttcaagcaaaatatctggcccaaacacctgcttctctccaggctgatcccattgcaacggagttctgcaactccttccatacagcgcttgaaacggtgacatcttcaaactggcctgataactgttgttataggaaaactctgcataaggcaatcgcttgtcccatccggactgatcttgcaacgcacaggctctcaacatatcttcaagaatttgattggttctttcagtctgaccatctgtctgcgggtgataagctgaactgaaattcagatgcgtgcccaaggcttcatgcaactgctgccagaaatgagaggtgaactgcgttcctctatctgacactatcttctttggcacaccatgaagacaaacgatccgagacatatacaattctgccaatactgcactgttgtagttggtcttgacaggtatgaagtgggctgacttggtcaagcggtccactactacccaaatggaatcgtagccggctcgggtgcgaggcaatccgactatgaaatccataccaatttcatcccatttccactgagggatctgcaacggttgcaacaatccagctggtctctggtgttctgccttaattctttgacaactatcgcacatagccacatgctctgcgatttctcttttcattccgtaccaccagaatttcttcttcagatcctgatacatcttctcactgccagggtgaatcgaataagctgtctcatgagcttccttaagaatcaactcccgaatagactggacattgggaacacacaagcggtctttgaaccatatcacgccttccgcatcttctcgaaaatctttgcctttaccatctagaatcagtcgccggatctcactgatcttctcatcatttttctgcgcttctttggtttcgcgctccaaggtaggttccaattcaactgtgactcctcgcgaattgttcagaaatccgagactcaacctgtcaaactccttggccaactcataaggcatcgggcgagcaaccatcagattgacttgactctttctgctcagagcatctgccactacgtttgctttgcctggatggtaatgaatctccaactcatagtctttgatcaattctaaccatcttcgttgcctcatgttcaattctgactgagtgaatatgtacttcagactcttgtggtctgtgtaaacatcacatttctgtccatacagatagtgcctccatgacttcagtgcgtgaaccactgctgccaactctagatcatggattgggtaattcttctcatgaaccttcagctgtcgggacgagtaagccacaactcttccctcttgcatcaacacacatcccaaacctgtgtaacaagcatcacagtacacagagaagggcttgtgcacatcgggcaagactaggacgggcgctgtagtcaacttctctttcagcgcttcaaaggcctcttggcatttctgggtccacttgaactcaaccttgttgcctagcaacgctgtcattggcttcgcaatcttcgaaaacccttcaatgaatcgccgataatatcccggccattcccaatgaaagctcttgatttcctcgagcatccgttggcgctttccagttttagaaatgtctgccactttcttcgggatccacagccaatcctttctttgttgataatgtgacctcaagaacagggacttcactgatccagaactcacacttgctcaactttgcaatacaactggtgctctcgcaatctctgcaacaccatcctcaaatgatccgcatgctcttcttcactttgagaataaaccagaatgtcatcaatgaataccaccacgaacttatcaagataatccatgaatacactgttcatcaagttcatgaagaatgctggcgcattggtcaaaccaaaagacatcactgtgaactcatataaaccatacttggtaatgaatgccgtcttcggaatgtccgaaggtcggatcctgagctgatgataacctgacctcagatcgatcttggagaacacactggctcctctcaactggtcgaacagatcttctattctgggcaaaggatacttgttcttgatcgtgacctcattcaaagctcgataatcgatgcacatcctcttagtaccatctttcttttctacgaacaagacaggggcggcccaaggcgaggtgcttggccggatgtaacctttctctgacagctcatcaatttgcttcttaagctcatccaactctggtccagatattctgtaagctctcttgaagataggggcggttccaggaagaagctctatagcaaattcaactttccgctctggtggcatacccggtaaatcctttggaaacacatctgggaattcagacacaaccttgatactctcaattgggtctgcttcactgctatcgacagccatctgataacaacttcctttctttggctcaggcgggactaacccggtcaccacttcctctcctagtggggacaccaacttgattgtccttttatcacaactgataactgcctgatacttatctaaccaattcatccctaggatgacatctattccctgagtacccattactataaggttggcgggaaacgctatcccccttatttccacacttacatttaaacaaatgctatcagctcgaattctaccaccggctgagtcaatttgaataggggttgacatggtagtaattggaagattatgtgcttctacccatgatgcagtaatgaaagaatgtgttgctccagtatcaaataacacttctgcaatatgggaatcgactgggaacatacctactgtcatgccgggggtctcctgaactgcttcagcctccaagtgattcaatcttccatgattatagcgctgctgagagcgattgcctgctccaggctgagacacattctgctttgctggggcattggggcctgactgctgctgggctgccttcttcggacattgcatcacccaatggccttgctctccacagtggaaacatgccctgtttccaacctgagctggtgctgcctgactgttctgctgatttgctggggcaggaagacgaggtgcttgctgattctgcttttgaaactgacctcctgactgattgctctgacggttctggtactgatgctgaggatactgcctttggaactgctgatactgctgacgctgctgatgctgctgaggtggacgctggttctgcctgaactgctgaggttgattgcctgagaaacggggacggctgctgcttccaggctggggtccactgatcttgcgcttacgatcttccatctccttacgcttcctttctgtcatgattgctctgtcaatcaggtgctggaatgtcgggaaggtgtggttcatcagctgatactgcagagggtcgaccaagcctcccagaaaacggtactgtcgcttggcgtcagtgttgacatcttcaggagcatagcgagacaattgcagaaacctgtctcgatactcactgacagacgatgacccttgcttaagggccaggaactcctccttcttcactgtcatcagacctgcaggcacatggtactgacgaaagctacctctgaattcttcccaggtgatggtgtcggggttggcatgggtggcgaggtaagactcccaccatgattgggctgctcctctcaacagacggggaccatacaagactttctcccggtcatcgcactgagcggtatgcaactcccgctccacagtgcgcagccaatcttcagcatccatggggtcagaagagtgagcgaacgttggtggatgacctctcatgaattcagcacgcttgtctctaggcatctgaggcatctgaggctggggtggtgcttgctgctgttgctgctgctgctgctgctgaatggcggccagagtctgaccgatggcttgaactgcctgagtctgcatcagaaacatctgctcaatcgacatcgggggcgggggcggcaggtgctgctgctggggcacctcatcctgcggagcggctcgctcctgctgagcacgccttcctcctctacgcctgttctctgacatctgcagaacgcaaccacacatcagaactgatctggcaaagcttgcagcataagaaaagagtatagaattctccaacagcactgaacagatgagcatcttcactgatctccaacacagaccacacagcttctcagataaagaggaaaggagaataatgggtttcccaactatataactaactttattgacatagtatgtaaaccaaaatgcaggggatacccacactctggtgacaatcattacaaagatccaaaccaaacatagttcatcatgacaaacataaatgcacaggatatagcaaactaccctgtctaactaaaactaactaagaccgagactaacgctaagactgaagcttccatgtatatatttatttcgtattaattacaagatccaactctaacgatctatggttctaggtttatcttggtcttgcaggcgggattgccataagactggtgtccacgctgaggtgagcggtacgggtgctgagtcccaacgggagcgggggaaccaccgtccagaaaacgacgttccgcacgctcagcccgcagcagggcgatctcagcacgagccctactcagctcgtctaatgcatggtccagctccgtgtttagcacggcggctaggttgactgtgctgctcaacctaggattgccctcaccgacgggtgagacaatcacgcctcctgtgctgccagatgaacggcgggggtaatacttcaggtcaagaccgtcagctgccccaccgaaaaccgagcagtagtgcgaaagcgcacgccgtgcagcatcttgcatggctgcctcagctgagtcccgctcagagatagaatagtgctctgaacaggcctctgcaccctggagactgtcctccgggcagcgcaccaagcaagttgcctcccagcggtccgggtagaccccgcgactatgctggtagaccacacagcgatactcgacggaccaagtatgccggtcaaatgcccgacgtagcagggtgtcgagcgcatcatggaagtgacacccgcgagcagcgtcgcgagtgatgggtcgagcaacccatccttccggctcaagattagcagagaagtcggtgtcgtggctcgagctgtcatcgccatctccgtcgtctgggtctcctccagcagctactccagaagctggggcgcccagtggtgatgcagggggcggctccagaggaagcacaggggagcagctcctcacagactctatctcctggtggagcgagagggaagagctctgctgctcctgtcgtcgacgttcctgctcctcacgcaggcggtggtgtagtctctccaagtggctggactgtccggccacgggacggcgaagcggacgctcagcaaggcgggagggtaagaaggggatgactgactttcgtgcagtgtgtctaagtcgagccatctacaaaagacatcgcaagcaaaagggtgagaacagaattaatatgaccagcaagtaatgaatcataagtaaatgaaggattagaataaaacatgatttttagcaaggtatagtatatagtagaacataggtttggtcggtaggaccaacttttgaagggatatcaaagtcaaggaagagacagaggtctataatccttagaacgaccattctactctaggttagcggtcctacagtcagcacggctctgataccacttatgtcacacccggttttagaggaaagggggcacccatgtacgtgccaggatcagttattagaaggcggcaggtacataatatggacataatcacacaaaagtcaaaatagtattaataagggaaaatagtctattacatcatacgtctgagacgtccatatagttcttacaataaatcaaagtgcggaaaagaaacgtagataacgcggccttcacaggcagccgactgggggttgccgctaacccacacctagaactcgtcgtaatcttggaatttctggaagtctccttccacagcttcatcttcgcctgagcagtggttgcaatgctgacaacctggggggggggggggttggtgtgtagagcaagggtgagtacacatcaacatactcagcaagtatcctgtttggctgtagtggactagctttatgtggggatcagtcaagcagttgcttttagttggtcaggttattacttactagtagaaagccaggttttaacattaacccaagttattaacccaatgtatcctttccaaacggaaagaataccacttaccaataccataatcataaccagaaccatcaatctcattgccacctgtaccaaaatatctctgatcaagtatcactaatctctggagctcccttggccgctcataaccgtgagcacggctgatatatcagtttcataacactctgcagaggttgtgcactttacccacaagccgtgattccctcttgcctcgggccgatcaaacccttaaacactaccaaggtgaataggcagggtttcactacgtagcctttacaaagattccccggggctgtagccacccgttaggtttcctaaatgcaccgcactcctccccaaggggcgaacccaaacttggcagagcgagccgcatacaccgagccccattgacggcacgacggctaagtgaactacatcccggatcctctaattattcagctaagggcaccccattccaccctcatggttgcactgttttcccgggcggtcatccatagaacaggtccttacggagaggcactcgagaaaccgctcgagcccccttgaataccacaagtacaacatcataataagagaagggaaaacagcgtatcatagataatcacatcatgttcattgattagagttgagcaatagcatcagactaagtagtaataatccgacccaaataggtaaacaaggacatggataacaaaagctagtcgatccttaggtataaatgtgtaatgcgggaggtgaattaaagaatgaataggacagagataggtcaaaggacacttgcctccaccaaccgattgctgctcaggggcttctcctgcgaattcctcgggctcttcgaccggatcgttctctatgcgagcgcaaacatacatacatacatccacatatttaatacaaaagaacagtacaccatacaagataacaaataaagcgaatatgcatcaagtatgacattcgataacgcatttgttatggttagaaagaaacggggaaaggtctcgcagggggttaaatcttatgcactaatgacacaattggtttttaacaaaataattctgttatatatatttatatatactgatggaaacctaatcacttttagttgatcaacattgcacagggtaaacaattagctacgtgaatcaataacataacggaattttaaattaaacttcctattttcccatagcatgaacagtgattagcctacttaaaatacagtaattatgatcacagaaagtaaataaaataaaataaaaaaataaaaaaaacagaagggggggggggttgaaccggccctagggcggttgaaccggccctaggcggggcgcgggcgcgggcgcggccggggctgaccgggcggctgagccgctgagccggccggttgaaccggcggcgggcggccgagctggcggggggggggggggcggcgcagggggcggccgagccggccgagcaggggggcggggcggccgaaccgctgggcgcgcaggggcgcggggcagggcgcgggggggccgagcggccgacccggcggggtggggcgggcggccaaaccggccatggggccaagcggcgaggggaaggggagggaggaaaggggagagaaggttgagggagagagggaggaggggggggctcaccggtgtcggggatcgacggcgaggggcgcccggcggcagggggcggcccgccaatctagggcagggggcgggtagggggtaggggcggcggattcggtggcgagggagaaaatgaggggggagagggagagggttagggaataagggaaagaggggggggcggctggcccttccaggcccaagagggggggcgcgcaggggcctgggccggccggggtcggcccacggcgcgggagagagagaaaaagaggagagaaaaagagagggagaaaaaagaaagaaaagatcctctccacattttcgaaatccgatctttctacatgaatgcaattgcactttcaaagcaatcaaaagaaatgcaaggttcggcatggtgcatcaaacaacataaagtatttagggtttttcttacacgggaaatccaaaccgaatcccgctagaactttggaaaaggtcaaggtttagcgaagggaaaaaagaaaaggaaaaggtaacgcccgaattttggcgagtaaagaaaagaaaaaattcaactgcaaaattcggggcgttacacttctgggccccgcttgatgtccggtggcgcaccggacatgcactgttcactgtccggtgcgccagcatgggcgtgcctgacctctgcgcgcgctggtgcgcaataaatgcgcagcaggtagccgttggcgcctgaagtagccgttgctccgcagatgcaccgccccctccggtgcacatcggacagttcggtgaattatagcggagcggctggagtgaaaacccgaggctggcgagttcctgaggacgacctcccttggagcaccggacactgtccgggtgtacaccggacagtccggtgaattttagccgagtcgcctctagaaattcccgaaggtggcgagtttgagtctgagtccccctggtgcaccggacatgtccggtggcgcaccggacagtccggtgcgccagaccaggggtgcctttcgggttgcccctttgctccttgttgaatccaaaacttggtctttttattggctgagtgtgaaccttttacacctgtataatctatatactagagcaaactagttagtccaaagatttgtgttgggcaattcaaccaccaaaattatttaggaactaggtataagcctaattccctttcaatctccccctttttggtgattgatgccaacacaaaccaaagcaaattcaaaagtgcataattgaactagtttgcaaaatgtaagtgcaaaggttgcttggaattgagccaatattaatacttacaagatatgcatggattgtttcttatatatcattttggaccacgcttgcaccacatgttttgtttttgcaaaattcttttgtaaattcttttcaaggttcttttgcaaatagtcaaaggtaaatgaataagagttgcaaagcattttcaagatttgaaattttctccccctgtttcaaatgccattcctttgactaaacaaaactccccctcaaataaattctcctcttagtgttcaagagggttttgagatatcaagttttgaaaatactacatatactacatgctcccttttagaacacaaagagatacaaattttgaaattttcaaccaattgaaaatcatatgtttaaaattagggtggtggtgcggtccttttgctttgggctcatattttctccccctttggcataaatcgccaaaaacggaatcattagagccctttaatgactattttgtcccctttggcaaatgaaacataggagtgatgattataccaaagttggagagatgctcggagtgacggcgaaggatgagtagtagagtggagtggaagcctttgtcttcgccgaagactccaattccctttcaatatacctatgacttggtttgaaatacacttgaaagcacattattcatagcatatataagagagacatgatcaaaggtatatttatgagcaatgtgtgcaagtttagcaaaaagaaattcctagaatcaagaatattaagctcatgcctaagtctggtaaaagattgttcatcgagaggcttggtaaagatatcggctaattgatctttagtgttaatgtatgaaatctcgatatcccccttttgttggtgatccctaagaaaatgataccgaatggctatgtgtttagtgcggctgtgctcgacgggattgtcggccattttgattgcactctcattatcacatagcaaagggactttggttaatttgtaaccgtagtcccgcagggtttgcctcatccaaagtaattgcgcgcatcaatggcctgcggcaatatattcggcttcggcggtagaaagagctaccgaattttgcttctttgaagcccaagacaccaaggatcttcccaagaactggcaagtccccgatgtgctcttcctattaatcttacaccccgcccaatcggcatccgaataaccaatcaaatcaaatgtggatccccgagggtaccaaagcccaaacttaggagtgtaagctaaatatctcaagattcgtttcacggccgtaaggtgtgattccttagggtcggcttggaatcttgcacacatgcatacggaaagcataatgtccggtcgagatgcacataagtagagcaatgaaccaatcatcgaccggtataccttttgatccacggacttacctcccgtgtcgaggtcgagatgcccatttgttcccatgggtgtcttgatgggcttggcatccttcatcccaaacttgtttagaatgtcttgagtgtacttcgtttggctgatgaaggtgccctcttggagttgcttcacttgaaaacctaagaagtacttcaactcccccatcatagacatctcgaatttctgtgtcatgatccctactaaactcttcacatgtagacttgttagtagacccaaatataatatcatcaacataaatttggcatacaaacaagtcattttcaagagttttagtaaagagtgtaggatcggctttgccgactttgaagccatttgcaatgaggaaatctctaaggcattcataccatgctcttggggcttgcttgagcccataaagcgccttagagagcctataaacatgattaggatactcactgtcttcaaagccgggaggttgctcaacatagacctcctccttgattggtccattgaggaaggcactcttcacgtccatttgatagagcttgaagccatggtaagtatcataggccaataatatgcgaatagattcaagcctagctacgggtgcataggtttcaccaaaatccaaaccttcgacttgtgaataccccttggccacgagtcgagctttgttccttgtcaccacaccatgctcgtcttgcttgttgcggaagacccatttggttcctacaacattttgattaggacatggaactaaatgccatacctcattccttgtgaagttgttgagctcctcttgcatcgccatcacccaatccgcatcttggagtgcttcctctaccctgtgtggctcaatagaggaaacaaaagagtaatgttcacaaaaatgtgcaacccgagatcgagtggttacccccttatgaatgtcgccgaggatggtgtcgacggggtgatctcgttggattgcttggtggactcttggatgtggcggccttggttcttgctcatcctccttttcttgatcatttgcatctcccccttgatcgttgccgtcattttgaggtggctcatcttcttgatcttcttgttcaacatcttgagcctcgacctcatcttgggttggtggagatgcttgcatggaggaggatggttgatcttgtgcgtgtggaggctcttcggattccttaggacacacatccccaatggacatgttccttagcgctatgcatggagcctgttcttcacctgtctcatcaagatcaacttgctctacttgagagccgttagtctcatcaaacacaacatcacaagaaacttcaacaagtcctgaggacttgttaaagactctatatgcccttgtgtttgagtcatatcctagtaaaaagccttctacagttttaggagcaaatttagattttctacctcttttaacaagaataaagcatttgctaccaaaaactctaaaatatgaaacatttggctttttaccgggttaggagttcataagatgtcttcttgaggattcggtgaagatataaccggttaatggcgtagcaggcggtgttgaccgcctcggcccaaaaccgatccggtgtcttgtactcatcaagcatggttcttgccatgtccaatagagttcgattcttcctctccactacaccattttgttgtggagtgtagggagaagaaaactcatgcttgatgccctcctcctcaagaaagccttcaatttgagagttcttgaactccgttccgttgtcgcttctaattttcttgatccttaagccgaactcattttgagcccgtctcaagaatccctttaaggtgtcttgggtttgagatttgtcctgcaaaaagaacacccaagtgaagcgagaaaaatcatccacaataactagacagtacttactcccgccgatgcttatgtaagcgatcgggccgaatagatccatgtgaaggagttccagtggcctgtcacttgtcataatgttcttgtgtggatgatgggtgccaacttgcttcccggcttggcatgcgctacaaatcctgtctttctcaaaatgaacattggttagtcccaaaatgtgttctccctttagaagcttgtgaagattcttcattccaacatgggctagtcggcggtgccagagccaacccaagttagtcttagcaactaagcaagtgtcgagttcagctttatcaaaatctaccaagtatagctgaccctctaacactcccttaaatgctattgaatcatcacttcttctaaagacagtgacacctacatcagtaaatagacagttgtagcccatttgacacaattgggatacggaaagcaaattgtaatctaatgaatcaacaagaaaacatttgaaatggaatggtcaggagatatagcaattttacccaaacctttgaccaaaccttgatttccatccctgaatgtgatcgctcgttggggatcttggtttttctcatatgaggagaacatccttttctccccggtcatatggtttgtgcacccgctgtcgagtatccaacttgagcccccggatgcataaacctataaaacaaatttagttcttgactttaggtacccaaactgttttgggtcctttggcattagaaacaagaactttgggtacccaaacacaagtctttgaccccttgtgtttgcccccaacaaacttggcaacgaccttgccggatttgttagtcaaaacataggatgcatcaaaagtcttaaatgaaatgctatgttcatttgatgcattagaaattttcttcttaggcaacttagcacgggttggttgcctagaactagatgtctcacccttatacataaaagcatagttagggccagagtgagacttcctagaatgaattttcctaattttgctctcaagatagccggcagggtataaaatgtaaccctcgttatcctgaggcatgagagccttgcccttaacaaagttagataagttctttggaggggcattaagtttgacattgtctcccctttggaagccaatgccatccttgatgccagggcgtctcccattataaagcatgctacgagcaaatttaaatttctcattctctaggttgtgctcggcaattttagcatctagttttgctatatgatcattttgttgtttaattaaagccatatgatcatgaatagcatcaatatcaacatttctacatctagtacaaatagtgacatgctcaatggtagatgtagatggtttgcaagaattaagttcaacaatcttagcacgaagtatatcattcttatctctaagattggcaattgtaactttgcaaacatcaaaatctttagccttagcaatcaaattttcattctctaatctaaggctagcaagagaaatgtttaattcttcaatcctagcaaccaaatcatcattattatctctaggattgggaattgaaacattacaaacatgagaatcaaccttagcatttaaactagcattttcatttctaaggttgtcaatcatctcacggcaagtgcttagctcactagacaatttttcacatttttcaacttgtagagcgtaagcatttttaaccttaacatgtttcttattttctttaattagaaaatcctcttgggaatccaaaaggtcatccttttcatgaatagcactaatcaattcatttaatttttctttttgttccatgttaagattggcaaaaagggtaagcaagttatcctcctcatcactagcatggtcatcactagaggattcatattttgtggaggatttagagttaaccttcttctttttgccgtcctttgccatgaggcacttgtggccgacgttggggaagagaagtcccttggtgacggcgatgttggcggcgtcctcgtcgtcggaggagtcgcttgagctttcgtcggagtcccactcccgacaaacatgggcatcgccgcccttcttcttgtaatacttcttcttttctctcctctttcccttcttgtcgtcgcccctgtcactgtcactagacataggacattttgcaataaaatgaccgggcttaccacacttgtagcaaaccttcttggagcgggatttgtagtccttccccctccgttgcttgaggatttggcgaaagcttttgatgattaaagccatctcctcattatcgagcttggaggcgtcaattggttgtctacttggtgtagactcctccttattctcctccgttgccttgaaggccaccggttgcgcctcggatgtggaaggttcgtcaagctcgttgatcttctttgatcctttgatcatgcattcaaagctcacaaaattcccgataacttcctcgggggtcattagtggatatctaggattaccacgaattaattgaacttgtgtggggttaaggaaaataagagctcttaaaataaccttaaccacttcgtggtcatcccactttttgctcccgaggttgcgcacttgattcaccaaggtcttgagccggttgtacatatcttgtggctcctcccctgggcgaagacgaaagcgaccgagctccccctcgatcgtttcgcgcttggtgattttggtgagttcatcaccctcgtgcgcggtcttgagtaggtcccaaatctcttttgcgcttttcaacccttgcaccttgttatattcctccttgcttatagaggcgaggagtattgttgtagcttgagagttgaagtgctcgatttgggccacttcatctgtgtcatagtcttcatcccctatggatggtacctgtacaccaaactcaacaacatcccatatacttttgtggagtgaggttagatgaaatcgcattaaatcactccacattgcataatcttcaccattaaaagttggtggtttgcctaatggaacggaaagtaaaggtgtatgtttagaaatgcgggggtagcgtaggggaatcttactatacttcttacgctcttggcgtttagaagtgacggacgccgcgtcggagccggaggtagatgtccatgaagtgtcggtctcgtaatagaccaccttcctcatcctcttcttcttgtcaccactccgatgcggcttgtgggaagaggacttttctttcttctctttgtgatgcgaagaagatttctccttccctttggaggagtccttcttttcttcgcggtgcgaagaaagtcttttctccttccctttggaggagaccttcctcttctccttcctcttggtgtgggactcttccgacgaagtgctcccttgacttgtagtgggcttgtcgccggtctccatctccttcttggcgtgatctcccgacatcacttcgagcggttaggctctaatgaagcaccgggctctgataccaattgatagtcgcctagaggggggggtgaatagggcgaaactgaaatttacaaatataaacacaactacaagccgggttagtgttagaaatataaaagagtccgcgagagagggcgcaaaacaaatcgcaagcgaataatgaagtgagacacgaggatttgttttaccgaggtttggttctctcaaacctactccccgttgaggaggccacaaaggccgggtctctttcaacccttccctctctcaaacggtccgtggaccgagtgagctttctcttctcaaatcaaagtcgggaacaaaacttccccgcaagggccaccacacaattggtgcctcttgccttgattacaattgagtgttgatcacaagaacaagtgagaaagaaagaaagcgatccaagcgcaagagctcaaatgaacacggcaaatcactctcactagtcacttaggctttgtatggaattggagaggatttgatctctttgggtgtgtctagaattgaatgcctagctcttgtaagaggttgagaagtggaagacttggatgcaatgaatggtggggtggttggggtatttatagccccaaccaccaaacatgaccgttggtggaagctgtctgttcgatggcacaccggatagtccggtgcacaccggacagtccggtgccccctgccacgtcatctctgccgttggattctgaccgttggagctctgacttctgggccccgcttgatgtccggtggcgcaccggacatgcactgttcactgtccggtgcgccagcatgggcgtgcctgacctctgcgcgcgctggcgcgcaataaatgcgcagcaggtagccgttggcgcctgaagtagccgttgctccgcagatgcaccggacagtccggtgaattatagcggagcggctggagtgaaaacccgaggctggcgagttcctgaggacgacctcccttggagcaccggacactgtccggtgtacaccggacagtccggtgaattttagccgagtcgcctctagaaattcccgaaggtggcgagtttgagtctgagtccccctggtgcaccggacatgtcccggtggcgcaccggacagtccggtgcgccagaccaggggtgccttcggttgcccctttgctccttgttgaatccaaaacttggtctttttattggctgagtgtgaaccttttacacctgtataatctatatactagagcaaactagttagtccaaagatttgtgttgggcaattcaaccaccaaaattatttaggaactaggtataagcctaattccctttcaagactgacgatccggccccacgagtcggagacaccaggcaaccagcgtgcgacacagtgtctgatagcgcgggtcccacctgtcggtgtaggagcggcgcagtgggagccaatgggccgcgcgaagatagtggggaatgggccgaggcagcgaaaaataggcccagacgctggcttccattattcttttgctttttcctttttattttctcctttcttttgatcttttctgattttagttttcaattttcataattcaatcttagattcaattttgaatacaagaattcacacccaaaggtcaagcatgatgcataatttttacttatttcttttatttatttgtatccatttagataaatgctctaatgatggattatacacatattattttttttaaggaaattttatctttgaatgcacacttaagataaataatcatctttagttatatttacttcggctatgttaatatgtaatctttctcatgaattattttagttcaattatctttttagggatggttttgattaaaagaaacaatagttagtttaaaagtctctctcttatatgtggtttcaactcctagctttaggttttagatctatgcttcgaataccagattcttgcttattattatattttatagaggacggaaatttttattagaagtccttttatcacaaaacttcctataaaaacatttataattctaagcacaggatttcgggtgttacatgggagtacttggctgattggctctggcggtctggcggtggcgggcgacgcacgcaggtagcggtggcgccggctcgggttcggacgcgcgttgctgtcccgcggatgcggatggggctcccgcccggtcgctctcgatctcggtcacatcgcatactctccccctgccgtgcggggacgccgggaggaagctttgtttggtctcgccatggctcggcgtgcgtgacgggattggatggaaaccccgaccggttcgctggcgtgtgccgtgccggagtggagggaggctcggtctgagtagtcggtgccggtggactgatggtgtcgccgtgtcggtgaaggggctcgtgctcgtgccgccgtgcgggacgtatgtggggttctttgtgatgggggccctaaccgaacaaagattcaggtacgacggacttgtcaatatgcaacaaccaatgtgcttgtccaatcgaacatgaatgacgagatatggacatgaacgaacgaatgaatggtgttgttgggtctatgcttcggcgccgaaggtcttctaggaagaagcggcttttggctgaagttgcttgagtgagatggccgaaggttcttcatcatgaagcttcggtattacaaaccgacttaaggatggaatgaccttttagtccataaaggtctgggtcaatgttgtaaacttttatgaggggcataattgtaattcctcacaggctgtgtcctgtgcctataaatagtgaacagtattcctttactgttcacgcattctggctattgtaatcgcatctttcggaaaccaatctttgccaaggcagaggtataatgtattcaataatttaatatattaagtaaatatttgtgcatgactcgtttatcttttcatatcctttatcttatgttattttatatgagaatatgattacggagatctaaccttcgtaattatgtcttcatgaaccttcgtccatggttcattatcctcaagggaatattgtttcatggacgaaggatgttaatattcaacattttatgttgtcttgttcttaattcatagcatttgagaacaagtccccaacattggcgcccacctccggtgaactcacttccacttttttgagctgatggcttcgttcaacgatcaagctggagctgcttcggctccgaagctgctactcccgataacaggcggttcatgttcagagccagccaacaagaagcagaagaaggaagcacagagaagggtacagcatgttggggtgcaaggacccttcatcaagtcaagatggtctcacatccctattaccttctcccaagaggaccttcaactcaaggattacccacacaatgatgccatggttatctcttgtgttatcaaaggatttctggtccataatgtcttggttgacacaggcagtgcagctgatatcatatttgctaaggccttcagacaaatgcaagagccagaagataagattcatgatgctacacatcctctctgtggcttcggaggaaggcagattgtagcactgggcaagatcaccatgtcagtgaccttcgggttcatcaacaacactagaactgagcaagttatatttgacattgttgacatggaatacccttacaatgcaattattggtcgtggcaccctcaatgctttcgaagcaattcttcatcctgcttatctttgcatgaagataccttcggatcaaggacccatcgctattcatggaagtcaggaagctgccagaagggccgaagggaactggactgactcaaaagcaatccataacatagatggagctgaagcttgtgaacagtacaaattcagaagggagaaagcagcttcagcggatcagccgaagcccatgctcttatgtgaagacatagcagagcagaaggtgctgttgggctctcaattatccgaagaacaggagaaaaccttgataaggtttttgttcaacaacaaagatgtttttgcatggtcagccaatgatctctgcggagtaaatagggatgttattgaacactcgctcaatgttgacccatccttcagacctagaaagcagaggcttcggaaaatgtctgatgataaggccgaaggtgctcgtaatgaagtcaaaaggctcctcagtgcaggagttatcagagaagtaaagtacccagaatggctagctaacactgttatggtaaaaaaggccaatggcaagtggcgaatgtgtatcgattttacagatcttaacaaggcttgtccgaaggatgaattcccattgccaaggatagactcgttagttgatgcagcagcttcttcagagctcatgagtctgctagactgttactcaggctaccaccaaatctggatgaagaaggaagatgagccgaagactagcttcataaccccaagtggcacatattgctatcttcggatgcctgaggggctaaaaaacgctggaggaagtttcagcagaatgactgcgaaggttctccactctcagatcggcagaaatgtgctaacttatgttgatgacatcattgtaaaaagcacaaaacaggaaaatcatattgctgatttgcaggagaccttcgccagtttcagacaagctggtttaaaattgaacccggaaaaatgtgtcttcggagtgaagaagggaaaatttcttggatgcttggtatcaacaaagggaattgaagctaatccaagtaaaattgaagctatacttcgaatggagccaccaactacaaaaaagggggtccaaagattaacagggaggttggcatctcttaatagatttatagctagatcagcagaaagaaatttaccattcttcgaagtgctgaaatcagccgaagtctttcaatggggaccaagccaacaaaaagccttcgaggaactgaagcaatatctgatagatttaacaacattaactccaccaacgccaggggctcctttgttattatatgtggcagcttcgcactcagcggtaagtgcagcacttgttcaggagaagcttgatggccaagtcaagaagcaggtcccaatgtactttgtatctgaagttcttagtatatcaaagaaaaactacacagaattagagaaggtattatatgctgttttgatggcatccaggaagcttcggcactactttcaagcatacaatattgttgttccttcttcgcagccgttgaaggatattatgagaaatagagaagctactgggcggattggaaaatggggctgcagagctcaatgaattttgcattgattatgtgcatagatcttcgatccagtcccaggcgttagcagacttcattgttgactggacgccaggggctcaggatgaagaaacaaataaagatgccgaagtgtggacagtgttttgcggtgggtcttggggaaccttcggggcaggagcagctgctgtgttggtttcgccatcccaagttaaaacttgttatgcggcaagactcgatttcagttgtacaaacaatatttaccgagtacgaagccctgcttttgggtcttcggaagctaaaagttttttttttttcagaagggccattcttaaaactgattctcaggtggtgttcgggtcatatcgacaagagttgcaaggttaaagatccgaagcttgaaaaatatctagacatggttcgaagaattgaagcttccttcgaaggattttctgtcaagaatatccctcgaggacaaaatgaacatgctgatttgctagctaagtcagcagccacaggggctgcctttaccttcggatgtgttcttcgaacaataaaggcaccttcagtggaactccttgaaagagcagtcctcaatatatctcctgtttatagcgaagattggagaactgaaatcatctcttatcttcagggtaaattcctttcagatgacgaaacttataacaggaggatagaggcaagagctcgtccatatgtcatgatagaaggggagttgtacaagcatggagtttgtgctccattgctcaagtgtttatctagaaccgaaggcatagagttgatgaaagaaatacatgcaggcctgtgtggatctcacattggatctaggccgttacttggaaaagttttccgtcaagggttttattggccgaaggcagcttcggatgcagcggaattagttcaaaagtgcgaaggttgtcagaaatgtgcaagagatcaaaaacaaccttcgtctttaacacaactcatacaacccatctggccattgcaaaggtggggccttgacttgttgggtccattaccaccggtccaagggaacttaagatatgttgtggtggctgtggaatatttttctaaatggattgaggcaaagcctttagccacaataacttcggccaccattcaaaagtttttctggcagaatattgtttgtcgtttcggggtaccaaagaccatcactgtggataatggaacacagttcgactccgaagcttttagggatttctgtgatcaaattggtacgaagatccattttgcatcagtcaggcatccggagtcaaatgggctcgttgaaagagccaatgtgaaagtgcattcatcccttttgtgagttttggtgatttggataacaacacatttaaaggtctaacaagtttgctaagtgttgaacaggaaattcagtatgatgaacatacttgaatagtgtataatgatcagtgaacaaggttcaacacaaggtgaaataaccagtgagacaatgcaaatggatataatatggtctctatattggtttgaatatatggacaagtcctgagaaatcactatgcataaatatgatcataatagaggttgaagtgattaagaggattggtcaagccaaagtgaataagatatgaggaatcgtgaattggcttgaccatatttactatttagtccatatatgaatatatgagaatcaaactaaagcttgattaatcttaatagtttatatctagaagacatttcaagcaagggttcacaatattgaaagaaatgattctcttaatggatgctcaataggatgtgacttcaagaatggcttgatagggtgaagatagcaagggaaagggcttcgaggaactaagcgaaggtgaaggccaagcgacggcttgtagaccgaggtaccatggctaaggtgaagaagagagtacttgcactaagtcgatgaactaatcagctatgaagagttacaacatgttgatgcatcagtaaggtgacttgaagccatgatttgaactcatataaggtgatatggtacaagtcacagggtttgatttgagtttgcttcaaaaggtgagacaaagatgtttgtgatccttatgaagcaatgccatggagaaatcacacatgagacaccaatgactcaaggagtttacttcattttattttatttaacttgagtataggaatcgtcgtactataaagggggatccaaaaagaaggttggtgtttgccaaagctcaaacctctctattcaaaagctatttatgaaaagcaaaaatctctttatcgttctatggttgaccatggttaggtttgagaaacctagagtgttcttgctgaaaagcagctgaacttgttcagctgcagctgagcttttcagctgagctggacttcagctgagctgagctccttcagctgcagctgagcttttcagctgagctggacttcagctgagctgagcttcttcagctacagctgagctgaacttcagctgagttgagcttcttcagctgcagctgagcttttcagctgggctgaacttcagctgaactcaacttcagctgtgaacctctttgaccatacaccagctgaacttgcctccgggcagttgagctggggttttctctcctaaactctctggtcaagaccggttgaactggtcctgaggggcagttcagctggtctctgacccctctgacctctgatctgcagtctgctagtcagactggcaaacaggtcgccaggagctgtcaggggcggttcaaccaccccccctgggcggttcaactggttttggtcaactttgaccagtctgcggtcagtctgcgcgtcagtctgccagtcagactggcagacaggctgccaggcctgccaggggaggttcaaccgccctggggggaggttcaaccggtctcaggcagaaaaatctgctcaacggctagttttgagctccacctatatatactacctcctacctctctccccaaagcagtgagcacgatttgaactccatttctaacccaagaaacacctcccactctctctcacacatctcttgcctctcccatttcaaatctttggagagaaatctttgagtgagcttgagagctgcggtttttgtgctccatctctaaatctctcttgctcttcttcattcgagctttggtactacatcgagttctttgtggattcattactcttggagcttctagctcctagacgactaggtgtctcttgtgagtctccaaatcttgtggaagaccacaagaaagtttgtattacccgctcgtttgagcaaagattattgtgtgggcttgacctttgtggtcggcaaagggaggattagggttgaaagagacccggctctttgtgggcgcctcaacgaggaagtagggcaccttggtggtgtgaccgaacctcgggataaatcttgtgtctcttgtgttcttattcattgtgtttgtttgtgtttttcgttctctcacccttccgtggaagatttgttcatatctttttgttgtgtggattttgagaagtgcccttctcagatctactactttgaaccctgtggatcatttagaacatctcatttccaaagttaactgggtgaatttcgagatcaattcagttttacccagtttgcttctagtttttgttgaaaaagttttaacttgcctattcaccccccctctaggcaactttcaattggtatcagagcctaatcctcgttctaacgcttaaccgcgtgaggaaagatcatgtcggggggactaagaaacgaaagtgcttctaagcttgaaaaagttgaggttgcctcgactttatcttttggtgcagatgttgatcctagggcaatagatcttgccatgagaatcgccgaaaggatgttcctcaaaatgaaggaagatgaggcgaagaacattattgaagaagaagaaaatgatcgatggagaccaaacgatgaatccacctcttcacaaggttcgtctttcaaatccacttctcatatgtgctttgttgctaatgagagtgacagtgaaagcgaaagtgaggatgaggaggagcatgaaagtgatagtgaagatgaggatgatcttcaaaaattcttcgctcaactaagtaagaagaaccggatgagcttgctcaaacttatgaaaagagcggaagaacaaaaggaaatgcttcataagcaagaagatatcctcatcgaaaaaatcaaagacttggagaagttgaccaaagagcatgagaagctaaagtgctctcatgatgatttggtccaaaggtatgaaaagatttcaattgagcaaactagtacttcaaatgctttatcttgcgttgctcaattagaaaaagaaaatactatgctcaagaacacgatagaaaggctaaatattgaaaatctagctttgcaagaaaaacatgatatgcttgtgtgctatcataataaatttatggattcacatatcatgttagaaatggctcatgaggttgtgttaactaatttgaaatcataccaacctcacatatgcacatgtactcaagtagaaactatattatcatgtgctaacaaatgttgctctcaagaaagccaatcttccattgagctagaaatttcaggaattagtgatatttctatcacacaagaaaataaagagctcaaggaagaagttggaatgctaagaaggagcttaactcgtttgaagggaaagtgtcatgctcaaccttctcaagataaccgtgataatatggtgaaaaagcttgagaaggggacaaccgtagcatgcacaaaaccccttcaaaagaaaaccaaactttccaagaagggcatgagaaaaatccatggtgagaaaattaatgctcatatgatttgctctaacaatgtacctatgtgcttcaacaaagaaagatcaaagagaagcgataggaggtgctatggatgcaaggagaaaggccatgaaattgattcatgcccccacatgaagaatcaagaccttgcacgatcaagaaagatgaccatcaaaaaggatgaaagcaaaaggcaaatgcattgcaaggacaagcatcgcatttgctacaattgccgtgaaaaaggtcatctattcaaggtttgtccaaagggtaaaactcctaagcctaacttgtcaatacattcaaatatgcttaggagacccaaatttgactcttgtgctagaaaggtgatgagttcaccacattctaggaccaaggctatttgggtgcctaagtccttattggctaaccttgatggacccatccaaagatgggtaccaaaatgtacttgataagttttgcaggtacccacagatgatatgaagctttgggggtgcttgagcggtttaactcaattcttatctcaagctatcaatcttacattgtatatcctttaagattgacccaaagatgaattgagttgttatatcactaacttcatattcatctctaccaagaacttgtgttgtagggaataaggattaacctttgtgggaatcaagcaaaaggcctacaaccaagtgatatccaaaggatggtaacaattaattcttaagtgcacattgcttttaattagtatattcctttgtgtcttttgtagccacataggaaaaatgaagcacttgagaatgaacttaaaagattttaccttgctttggaaaggatctaattatatggtagattgcacgttcatatttttatattatgacaatctacatgctttaaattggttgtatgtatcttgtggcatatttcaaattgattatcgtattattgccatgacctagacataaagaggattatcccatgttcttaaatgaatagagtgctaagtaagaaattcaaattcttaaagcacttaccaaaagggaaattctctatatgactagagttgtgagactaatgtttttatctaagtgatttatgtagtttcacaacatgagaatgtgtttcccaaatggagtgtgccattcaacattcaaagaagatcgaaccaatactatgtgaagtattcattcttgtttaaattggttttgagtcttctacattaatcactcaccatgctatgaattaaacttgccatgtaaacttaattaaataatcatgctactttcgtttttttaattgatgttatgcatgatgcttgtaagggtaatttagttcatatcatgaagtttccttgttttagtaaccttcttgtcattcatatactagaggttgctaaataggaaactattgcttgtgttactaatacaatctcttttaagatatttcatggaaattcataagtagagattgtgctctttcaattggtaaaatcacaagctttaaaaggttaatcttcacctaaaagaaagattaggaatgctcaaggcaaaggtatggaacaaattgcttcaattggtagttgatcaatagtagttccttccaagtggcattctttcaattggtaataatatattctatgaaaagaaatgtcaatataaaggttaaattccttttggcttaaatttgtaaattagtgcatattgtgaattcactgctccatgtgcattaatttaaaaggaatttaatttatgtctttatgcctcataaatgatgatttgtgtgcaattcatatctaaagatcaccattcattaaatacttccttgtactactaatatctcttttcaaagttctttttcaactagtattaaaaggaaaagagataacaagctaaagaaggaagtctccacaaatgatgaaaagaagaatactacgatgacaccaccacagatagtaagatctttcaaattggtataacaaatggtacgctctacttggtggtaagtattcttaaggataagttaattcattgcaaatttgtcatgccttgaccatgatatgtaatgtactcctcatgagactcttaactgaattgaaagtgcatgtggcaagtcattcaaatacttgatgcacatatctagtgggagaacattatatatcttgtgtcatagagaataagtttctcttgagtaagctatactaagacaatccaaaatggtaaatttgtatctcattcatatggattgtaatctttgttttctaaatagctactcttgatgcagtttaaaatttccttatcgttaattctaaaagtgcataagaatctttttgttgatattcatcacatacatatgcactaacatggatatgatttttaaactgtaaaaggtacaattagtgatccatactctctaaattttgaaaacccatattttgatattttagaaatctacttcaatcgatatccatatgcttcacattgaattggatcactaagttgtaaattccatgcataacttgtctttatgatatgaatgcttgtgcgactaaccttgataagctaggtgcacccaaggtcaagctaggttcatcatcaagaaggcaacataatgatgtatcaagaaaaggagaaaaggctcctattcttaactctagcttttatctgtgtgattaaatattgacttgaaaccatgtaagatggttgtcaagtatatttgggtgcctacacaaagagaaaggtcacaataaggattgtgtgggtactcaaggctcttcctactaatctcaaaggacccaattcaaattgggcaccaagatatgaagcttaaacttgttttgcaggatcattccttcaatggatcaagttggatgctcgataatgaatgtataaatcatatttggagatagtagcaagggtggtaacaactgaatctcaagtgcaaattattttcaaatcttatctcttttgtgacttgtgtagccactaagggaaaagaagcacttgaggatatacatcagttgttgattatgaaataaaggtctaattggaagatgaatgaatattatcatatggtgaacaatccaaaattatgtgacgtattctctatctagttaatttggatttgattcttctttattagacacttaccataatatggattaagtcagcccttttagacttcattgaacaaccatgcatattatccatgtcattcaaaatatattgtgcatgagggttcaaggaaatttagtccatattatgaggtttctctattttagcaactcgcttgccttccacatataaagggttgctaaataagaaactagtgcttgtgctactaattccatctcttgtgctgagtttatccatagaaaatctatgttaagagatggtgcttgttttaaattggataaatcacaagcttaaaggatattattcaactaaagtaagatgaagttgataagaggccaaggtatgaaaacttcctctccttcagttgttttagtattctatccttagtgggatgtaccatagtataggttaatttccttgcaatataaaatgttcaacagtgcatataactttgagatcaactatatgtgtgcactagtttaaaggaatttaatctatccttttgggtttcaagaatgatgattcattctccatccacatataaggattatcatttgtgaaaccctcccttgtttttctaatgctctcttttctaagtgtttcaataaggtccttccaagtgttttcaagatggatttaaaatctacggatataagagtcttggttgtttcaatcattgagtttcaattgatctcatatcaagtatgatcgaatcaagaaaagatgaaagaagttcaagatcacttggttggatgaaatcgtaaagagaaaagagatcacagtgattcaagaagggagttatttttttgtcaaccaaataatgaagatgtagtgacatatttatatgatatccttcattatgaggtttgaggttcatattagcatgctttacccttcaggatttcaattgatatactttcagttcttaaatcttcaattggtttaattttcatcatctatataaagcatgttatgttaagccaagatatagtacaaacatctcctttaacctcacattgttaatgtgcataagtgtactttgtgtactcttgcatgcacaaattgagggagagtttagcctatatcttgtgagactaatgatttctttcaagttcatgttgtgtagtctcacaccttggagagcttcaaatgaggatgaatggttccatagaaatgaaaatgagtttctcttgaacctatcaagaaaatgagtttctcttgatcgtttgaagaggatgagtttctctttgaaatgtcaatcctatcaaaagctaagatggaaattcatgattataatggagaccatgtggcgtagaacaaaggtgtgtcactccatgaactattgtattacatttgtgtatctaggctcttacatgctagtgtgtgcatgtatatgcaatatcttaagtcacatcataaggttgcacttgtggtgacgattaaagaatctctagatatttgattaatacctcttgtggtgatatcataagttagtcttaagtcatcttagtgaggtatgagtcaaacatgctaacttctcaagaatcttgacttaaaatattgcatatcatgtctattaatataccttttaattatgagtaattccttaaattggtacaatttcacattttcatactttatttgtaccaaggttcaaattgtaggacttaatcccctggcctcacaagtccaagtgcataagctaaacaagtattcatcacttgtatgcacacatttagggggagaatagtctataatttgaatctttgagactaacttcatttccaagtctattatgtgtgtagtctcgaattagaaaggaatcttcgggcaaagacaatcgcttccactgcaaattttgatgggtatcaaagattctttgctccaataaatgtatcttctatacatttcataagagaatgagtttctcctgtatatgctactccaacgtcatctaaaattggtaaatatgtatcacatctttttttattacaaatatctgaagtagcataacttatagattctccttctagaacttaattgattaaatagtgcacatgttccttatgttgtatgattatattcatatgatacttttttatgccaatggtactttaaattgcaaataagtactctcaataggtatcaattgaatacatatatatgtgtgcactaaaacttgaggagaacttagtgtaatatgctattagtgatctatttatctatcaaattgtatcaattggtgtttttcaattgatatttttcatacatgatcactagttgcataatccatatttgtgcaattttcatgttgcctcttgttgtgataagaaaatgctaggtgacatctagactccaggtatcaagatttatctttcagttagtatgacaatcttcatttgatatcttggacctatgtcacaattatgccaacaagagcttgcaaatgaattctaaatccaacacaagtctggaaaccccttgaaaaggtaaaacgcaaaggtacatcacttatgacacttatccattacctttgtgccatctaaggatccctttcatgatagtgtgttcacatcttgcttaaatcataatttctaccctttatattctttgtaacctttttggagatttatgacaaagggggagaaattttgcattaaagcttatctttattttagaaaaggggagaaataattaacaaaagggggaatcataagaaaaacataagatgaagaaagttgataagtgcattccatgtcatgagcatcacgtttattttatgacacactgcaccctatgaatagtatgatataagttgtctatactttgacctaaatatgtgcttttggcatttgagtacaaaagtgttattttctgaaatgcacatgtttagggggaggaaactatatcataggattcaaaatcttatttatcaaatcttatgtaagctttaaatgtgttgtcatcaatcaccaaaaagggggagattgaaagtgcattcatcccttttgtgagttttggtgatttggataacaacacatttaaaggtctaacaagtttgctaagtgttgaacaggaaattcagtatgatgaacatacttgaatagtgtataatgatcagtgaacaaggttcaacacaaggtgaaataaccagtgagacaatgcaaatggatataatatggtctctatattggtttgaatatatggacaagtcctgagaaatcactatgcataaatatgatcataatagaggttgaagtgattaagaggattggtcaagccaaagtgaataagatatgaggaatcgtgaattggcttgaccatattactattagtccatatatgaatatatgagaatcaaactaaagcttgattaatcttaatagttatatctagaagacattcaagcaaggttcacaatattgaagaaatgattctcttaatggatgctcaataggatgtgactcaagaatggcttgatagggtgaagatagcaaggaaagggcttcgaggaactaagcgaaggtgaaggccaagcgacggcttgtagaccgaggtaccatggctaaggtgaagaagagagtacttgcactaagtcgatgaactaatcagctatgaagagttacaacatgttgatgcatcagtaaggtgacttgaagccatgatttgaactcatataaggtgatatggtacaagtcacagggtttgatttgagtttgcttcaaaaggtgagacaaagatgtttgtgatccttatgaagcaatgccatggagaaatcacacatgagacaccaatgactcaaggagtttacttcattttattttatttaacttgagtataggaatcgtcgtactataaagggggatccaaaaagaaggttggtgtttgccaaagctcaaacctctctattcaaaagctatttatgaaaagcaaaaatctctttatcgttctatggttgaccatggttaggtttgagaaacctagagtgttcttgctgaaaagcagctgaacttgttcagctgcagctgagcttttcagctgagctggacttcagctgagctgagctccttcagctgcagctgagcttttcagctgagctggacttcagctgagctgagcttcttcagctacagctgagcttttcagctgagctgaacttcagctgagttgagcttcttcagctgcagctgagcttttcagctgggctgaacttcagctgaactcaacttcagctgtgaacctctttgaccatacaccagctgaacttgcctccgggcagttgagctggggttttctcactctctggtcaagaccggttgaactggtcctgaggggcagttcagctggtctctgacccctctgacctctgatctgcagtctgccagtcagactggcaaacaggtcgccaggagctgtcaggggcggttcaaccgccccccctgggcggttcaactggttttggtcaactttgactagtctgcggtcagtctgcgcgtcagtctgccagtcagactggcagacaggctgccaggcctgccaggggaggttcaaccgccctggggggaggttcaaccggtctcaggcagaaaaatctgcccaacggctagttttgagctccacctatatatactacctcctacctctctccccaaagcagtgagcacgatttgaactccatttctaacccaagaaacacctcccactctctctcacacatctcttgcctctcccatttcaaatctttggagagaaatctttgagtgagcttgagagctgcggtttttgtgctccatctctaaatctctcttgctcttcttcattcgagctttggtactacatcgagttctttgtggattcattactcttggagcttctagctcctagacgactaggtgtctcttgtgagtctccaaatcttgtggaagaccacaagaaagtttgtattacccgctcgtttgagcaaagattattgtgtgggcttgacctttgtggtcggcaaagggaggattagggttgaaagagacccgactctttgtgggcgcctcaacgaggaagtagggcaccttggtggtgtgaccgaacctcgggataaatcttgtgtctcttgtgttcttattcattgtgtttgtttgtgtttttcgttctctcacccttccgtggaagatttgttcatatctttttgttgtgtggattttgagaagtgcccttctcagatctactactttgaaccctgtggatcatttagaacatctcatttccaaagttaactaggtgaatttcgagatcaattcagttttacccagtttgcttctagtttttgttgaaaacgttttagcttgcctattcacccccccccctctaggcaactttcacaatgacattataatgacaggaataatgaagttaatcttcaatcaacctaggggaaagtggccagatcagttaaccaaagtggtgtggagccacaacacaacaacatcaaggtctacaggctttactccattcaagttattattcggtgacgaagcaataactccggaggaagctaaaactggatcaataagagtagtagcttcggcagaatcaggttctgaagatgcttattctgtggaaaaagatgctttagaagggattaggcttcaagctgtggaaaatatcaataaatatcaagccgaaacaatcaaatggcgagatagaaaggttcggctaaagaatattgagccaggacatttggtgcttcggagagtggccaacccagacacagtgggcaagttgcagttgaaatgggagggacctttcttagtagtatcttcgtcaagacccggttcatacagattgaaggatatggacggtaacgacattcctagatcttggaatgcggatgagcttcggcgatattatgtgtaacttgatgtaacttttttatgttttttctttttcatggcacccttttcctttctaaagggggagaaaggtttttaatggggccatcgcatgtaatttccttttttagttttataagagtaaaatctcccaaagatgtaaatgtaaaagctgagaatgcaccatcgagtgcagaaaagtaaaaggcgaagaagctccaaagtcgttcctaagggaatgcagagcttacaacgaaaagtcaacgctgattccgccaaaagtaaaaggcgaagaagctccaaagtcgttcctcagggaatgcagagcttacagcgaaaaagtcaacgctgattccgccgaaagtaaaaggcgaagaagctccaaagtcgttcctaagggaatgcagagctgatgtgtgattgtttctaagaaaataatggctgagagtatagcttcggatatgtgtttggacattcatttgcacatcacattacatcatagcatttgcattcataaacattcatccaggcatatgtaggataatcatcatcatggcataagtaattgcttcggtacaagagaagagcttcttcgcgtgcaaaaaggagagcttcggaagaaagggaaatattgttttctatgcttcgctgtgtacgaaaagaagggaaggtgttttttcgcctttggctcaaaaaagataatttcgtccacaacaaagcacctcacatacattaatggaagggtaagagcataatccaaggtatgaacagaattcattaagcacaagtttagttacatttacaaaagttatctcaaaagtttcctaagttcatctacagtctactacttaatcttcgggggaccttagcttcggcgtcgttcttttcaatcatcggcttcagcttcgtcatcctatatgaattaagttgttgtaagtcaaaattgagcttgaaggaagaggtaagcacaaggtatgatttcttactggttcaaaatgactccgagcttcgtctccagccttttctcgcccgccttttgtccatatcattttcacaaatctattggaaatacttcgggcgagatcaggagtgtcatctaggattgatggtgataaagtgaaattgggcctattgacaatctttccgtgatcgcagccagcttttaggaaggctgcagcagtgccccgagaagctacccaggcacagaagtcaccgtgcccagctataacttcgtcgagctcgtcaatttcaccctcaatatgttcgaaggtcttcgataaatcttcagctgagggggtgaatttctcactgctggctccaactgagtgaaaaatttttcttagtcgttgaatgcatctgttgccaaattccaaacatttttcttgaaggcccgccaatagttttttcaaatctgaattttgttgagcttcggcttcaagttttgtattaagttcttgtttttcttggtcaaactgctcagattggcggagaagcttcgtgttcagttctgttatttttgcttcggcttccgccaataaaccttcggttgcctggagctcaaagttctttttctcgatagcatctgattgctcctttattttgttttctaaattttcaattatagcttcgtgtttcttgtcttcagaatcttgctgcattttcaaggctttgctcaacagcatactctgcacgaaaacaacctttgttagacatgtttttattattaaagacaataaaagggaaaagtagttcaccttgaaattggaataaaataaactaccaacgatatgttgtcgtcggtagcggctaatgtccatttctagcttcggaaaaccgatactctttgacagagtaccaataactttggctccagtttggtttcggatacaatctaatttctcatcatcaatacctccgaagaggagtgctcctggtttgtacccgcaagatttggcatactctttaagctcttctatttcgggttctgacaatttttctccaactaaattttgaaacatgaaggcttcgttttctgaagtttcatctacaatttccttcctttttcccgacgctgcggcagtggcctcttcggcggcggtggtagcttcttctgcagccatgtctaacaatattttgtcaatatgttcgattgtgctctccaggttcaaatcttcagctgaggcagcttcggcagccgcgacctccgaaggtgtgatttcaatatctgtcctttcctcagccgctggtgtcttctgagctgaagctcttggcggtgtcttgtcaattacctctgtcacaccgatgatcctctgtctctttactttagtcgttttctccattttttcggactccttttccttctgaaaaaacattgtcagttgaggccccaatggacttagcttcgcaggcagggattcagtcattaccttcaaaattgtttccacatcagtggcagagggtgatgcgagggtttcttcctcgtcggatatttttcgcttcggagaagacgctttcctcttcttcggaattttcttttttggtggttctttttcatcttcatttaaggcttcagttatcctttttcttttctggcctccggcacctttgttcagattttcatagtcagggtattcaaaacccaaggcatccagcactcgattcagtcttcgcttcggacgggtgccgaaggccgcagtcatcaattgatcttcttttttggagtagttgccaagtatttcattacacattacttcaattgtatccagccactcttggcaaggtgttttaaagtatttcttaaacttgaaatagtaaggtaaacgcacaagttcaccttctttcttttccccctttggcttcggcatttcccattcttttaaactaggaaaaaccttgaaagccaaaaactcctgaaccaggtctcttgtactgatatgctctgcgataattctgaattcatccattgctttttgagtaggaccttccggtgtcatgttgcaatgggggcgggtttctccgaagattagttcaagtggactttgcacaagcttctccttgtcgtcatcaaccttgacatagaaccacttcgacttccagcccgctgcccatttgcttcggtagctgattacaggaaactttgtggttttccggtaagcgaaattatagcaaccaaaattgtcatgcaatccatcttttctagcctttgtctgatagtgcagttcgtgaactcgacagaagctgtccgcaaacggctccaccgcttggtttcggagggcccagatataaacgctaagtctaacaatagcgttgggagtcagctgatgaaagtagacaccaaacctcttcagtacctctgcaataatcccatgtagggggaaccttagtccagcctttagaaagctcttgaaaatgacaatttcatccttctctggctttggggtagtctcctctcccccgaagcgtaatagcttcttctgattctcattaaaaaagcccgactttaccattttggagagatcagccttcgaaacagtagactttccgaagtccaagtgactgggcttgcttggcatggcaatccgataatcgtcttcgggatcagtttcctcaatatcttctccttccgcttcggcaactgcttgttctgcctgttttgcatcatcactgggaatcttttccgaagttaccagcccggacctctgcatcgcttcggagatgggaacagactccgaatcttcagtttcgcccccctcacgctcaaccctagcggtagaacgcactctggccatttaattctgaatttgtaaaaatctaatacttttttcctcccaagttttttcttctgacgaagcaggcttcaaactggagcttcgttcgattccgagagttaagcttcggcggtggttaaaaattttggcagcaaaacagtgcaaatagcaatgaatgttgtggtaacttcacacctactcatctgtttatatagtattgcaggtaggaaggcgaagcgccaggatttttgcaccaggcgggcacccgctcgcattcactgcaaggtggaccacagagatcaaacagtaactctgcaaggtgggaccgctacgcgctgggaaactaaatcgtttctcgacaacgagctcagggaaggtgttttttggaccttcggctccccgaagcttaagagacttttttcacggatcaagctcgttacgaaaaacgatctagcaccgcgaaaggggctactgttgggtctatgcttcggcgccgaaggtcttctaggatgaagcggcttttggctgaagttgcttgagtgagatggctgaaggttcttcatcatgaagcttcggtattacaaaccgacttaaggatggaatgaccttttagtccataaaggtctgggtcaatgttgtaaacttttatgaggggcataattgtaattcctcacaggctgtgtcctgtgcctataaatagtgaacagtattcctttactgttcacgcattctggctattgtaatcgcatctttcggaaaccaatctttgccaaggcagaggtataattgtattcaataatttaatatattaagtaaatttaatatttgtgcatgactcgtttatcttttcatatcctttatcttatgttattttatatgagaatatgattacggagatctaaccttcgtaattatgtcttcatgaaccttcgtccatggttcattatcctcaagggaatattgtttcatggacgaaggatgttaatattcaacattttatgttgccttgttcttaattcatagcatttgagaacaagtccccaacaggtgtctaaccaacaggaattccacggtttccagagaagacccatcttcttaacgtgggcatgagactgtcctcagtggatcatgctaaataatcttaccttaatttaaacataacttagcaaactaacttgcagtctagaaacaaaatataaaatatatatagccactcgatatagatttcagttgactgcaacatatgtcatatgtgaaggccttttattcagaaaacagcaaaaagagaagcatgccttgggcatgcgatttttgttggtcatcctttatattctcatggatatggagatatggtcaaggatggaaataaagaattcatgagcatcctgttgctcgtagcttgcgaggtttgatgatgcatgctgccaccaactaaaaaaatcacaatggtaccaataatcagaagaaatttagatacttctattcaagaggtagaaagaatcgataggtggcatactgttttctatgctcagtctcatagaagaatagagaaattatgctaatcatggaattcatatggtcccgatatcctcaaacatggggactgtaataaaatccatctagcaaagaccaagttccccagaatacctacaaactcatctggccaactgttacctgcactgaatttgctgatttgtgtgcattttatcactttttatatatttttagacagtttaatggctgctaagcatgacagatgacttaagccgttaggcccaaataggtcaacttgagagctcaaatgggaacgtatctggtgctcaaaggggtacgatgttcatatgcatattttaaatctggtgcatccatcatgcattcaacggcaccagctaattttacgaaaaactccttccatcatgggaggaaggtggaaggggtgcttcacaaaattggtgctgctgttaaatgtaaaatagatgcaccagatttaaaatatgcacatgaacatcataaccctttgaacaccagatacgttccttaacgctacatgttcggtatgggatcagggataggatgccatgttaccaaccaattgtcacgtattgagatgtgtgactgttcccccttttttctaatagcgttacaataacatactctctccgtccaaaactaaaatttattttagttaattaatatgtccacacaataattaatttatgtgtctatatttaccattatctattcgttttagctcttgatttttacgtctatatttaaaggtatgacgataaatctagatagatatacaaaacacatacattaattactgcgtatatctatttaaagcctaaaacgaattttaattttaggcagagggtgtacattatttagtgattatttttttagtccggccagatcggtcatgatcaaatctgaaccgaacgaagaccaatcggtccctgtcttcggagccgaaaacttgtaaaatgcccttatagttacatatgctgaaatcttcgtgccattccgctacttctagaaatggtcgtagattgtacagtcatccaagtcgttgtagtatagtggtaagtatttccgcctgtcacgcggacgacccgggttcgatccccggcaacggcgatttttttattatcactcggtattctcgcttcaacttttgagacccgttgttaccgatcctaggccgcatttacttcagctttttttgctcggtgtagtgtatctgcatatttcattgcatcagactttgaatatacccatgtacatgtggtaatatatctcaaaaaactactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaaactactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaactactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaaactactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaacctactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaacctactctcgctcggtattctcgcttcaacttttgagacccgttgttcccgatcctaggccgcatttacttcagctttttttgctcggtgtagtgtatctgcatatttcattgcatcagactttgaatatacccatgtacatgtggtaatatatctcaaaaacctactctcgctcgattcctagctcaagtgctcaaccaccactgcctcttggcagcagagaatcctagctcctctcgcaacaactccagcttcttaataaaaccgccagtgaaaatgctatttttactggtggttgtttaagaaaaccgccagtgaaaatgctatttccactggtggttgttgaataaccgccaatggaaaatgacgattttcactagcccctagcactgacggcactgaaaaacgccagtgaaaacagttttaggactgtcactatagtttttctgtgtactagtgagattcacgctaaaaaaatcacgtacatgcatatatactatcaggctcacgcgctaccctagtaggctggtacaactatacaatatgtcaatctatacgcatttgctaaacatctgagaaccaaactttgcacgctatgtcacgatgacaaggcggcgctgcagagtgttagctttcagaaagaacaggccttcttcaaccgagtataagaatgtccttggcctccctcaacctcggcgtccacgaccgtaccaggaggcgcgttgtgcctgcatgtagaagcacgatgtgcggttacactgagggtgcattaaccagattgcccgcgaaaaagctcaatgacgacagttgattcagatcaaacattagccagattgcatcagtcatgtagaaatagtccctcgactctcaaaactgtcatacacaggtgtttcactcatttcactgtggtttgactacacttgaccttctagttgacgcagacaaggcctcagggccaagcagagagcgggccaagctcatcactaattaccggttgccactgtgagaaggctcagaggctgacaagtggctgtcactcgcgagctcgtgatgattggggaccccacacgggccaggacccggttgccactgtgcgaaggctcagaggctgacaagtagggctgacaatgggctgtaaattttgcactataagatttaaggatcaaatcggattaggatcgggttttatttctagtcatttttgaactataatttatttagggccttgacattttgtgaagaaccatatggatcacaatccattaccacccttactgacaagtggctgctggttcattcagacaaagcacacgataatcgtgccgtgctctagcttttggaacaactagctcgcctcggaaagcgtgccgaggatgttaattaccgggtgaattagtcaatcaatgtcaagggaggggggcattttgtcgtctgatgggacctcaagcagtcgctcaccgttggcccgttgccgcgccacttgtgtttgttgagcttgcagttgcacctgcacacttgctctctcctcacactcagctctgcaccgcgaccgctccgctcgccacgccacgccacggtctcgtgaggccggcgcccggagccagcgggccatgttcaccgacggcatgtcgaggctggcggtggcggtgagcgtcacggtggcgttcagcctggccatcttcctcaccatcctagtgctcctcctcgcggacctcatctgcgcgcacctccggcgccggcggctccgcgccgaggcgtcctggtcgaagctcggcctggcgctggcgacgccgccgtccccggcgcgcgacgccgccgcgtcgtcgtcacgcgaggcgttcgccggcacgccgcccttctactacgcgcacggcgtcctgcacgcgcccagcaccaaggacctcctcgtcgccatccccaggctggagggcgccgtgtggcggtggtcgccgccgccgccgtcgcgctcgggctcgtcgggagccacctcggcgtgcggcgacgcgttcatgtgcatctccaacccggtgtacgaccgagggcagaccgcggccgccggcggtggggagacgccgccgttcgagacgccgggctcgtcgccgtacggggtcacggaggaggaggcgtgcgcgtacgacgagaaggatgccggcgggttctcgccgccgctgtcggctatgaggaagctcccgccgttgggcgtcctggccgcgtaccccccgccggcgctgagcttcgccgatggcgggccgtcgccgcaggcaacggtgggcgacaccaaccgggcctcctcctcctcatcgtccgcccactttttctcttattggtcgtcgaagtaagagatcacttgtaccgtgcgcgaatgtagtaaagctctgcagctcgatcccattcatcaatcatcatcgttttggccatttttcctcgttcagtcatatcatcatcatcatctgctattattgcacattccatctctaacgtctgggcgtgcggaaaccagagcttttggtcggtgttttttcaagaaaggaaactataaacagttggtgaatagcgcaccgtgtgctgagttcttgcatgcactctcagcaaccggagcatcggtacaaaaggcagaagggcctcgtcatgctcgtgctgtgatgaggcgtagaggccgacatcggctcagtgggtcctgacatcaaacccaagaacactgttaggtgtttggttccagaggccaaaatttaatacgttgtcacattgaaagttttaatctttattataaatattaaatgtaatctaattattatttagtttttatctatataattagttttataattaaactacgtttgagaaaacgtttcagactgttgttgcccacgcctcgacacactcgttgtatactggataaccttttatgcgtgatgtgtccacaaggaaacaatacgccgttgctccttgccgttgtttaggtggtgttgcttgagagacaggtttgatctggaagcgtttccatccagtgtacaagaaccgtgggggtcaggttagcaaaagctggtgtcttccaaaacagctcagcctctttacaagaaccgtgggggtcaggtcagatttgcttgtggcatttgaaagaggctcaacaagatggcaagttggcaatgtaaaaaaaaaaagatagttgctaccagcgttttcattcatgtagagacggttgatgattctgagctaatttttcattgtattcgacgcacttagggctagtttgggagccacaaatccgaaagaaaattgaattccattccagttttgtggctccaaaactaacacttagggcttctttggtcacaagggaattgagacggattgaggagaaaatcaactaattttgtctttaatcctatccaatccccttgtcaccaaacaagacattatcatgaatcttttcactaatatggtctggtttagtaatagtagaattaaaggagattaagggcttataaggttagggagtccctttctagttaaaattaaataagaaggatttaatcctcttcaatctcctacgatccttttgtaaccaaacaaggcccgagggggaaggaaagccccttaattccttccaaaccagtcataaagcaagtgcatttcccgcttagatcatttgaataatcgtgatttagacatagatcaattaatctaatatggattgaccatatgataaagatatttatgcggcaacattctactgtagaagaatcaaatgaagaacgtgttataaattgtagagtagataaatatcttggttcaacataaaattaattttcatctctcatcctaatctaagatagccttatatctaaacttttgaaaaagcggtggaatgtaaattttcacgacaaatagtctattttattaagtaaattccaattcattattagatttggtgtagctctggccctgtttactcgctttcaaaaaaaaacagaggttttatacttttaaaaaacaaaacgtctacacgcaagaagcccataggccatatagcccagacgtgtaatgagccatcgggaagcagacgtgggcacgtagcccatgggccatggctcgtgcgtggatgctggagcctggaggcaagcaacggccgagtcgggccgcaacggaagcgccggcagcgacagtctgcagtctgcgggcgccgccttgagtgtgcacgctagcctgcaatcactgcatgaccttcccgcttcgactggcatgctactagcagaccgggctcgtgatgcaactttaacgtcggcccaataagcggctttcgggagcagagcagagcagcggcacggggccgtggaccgggggagcaagctgcagctcgaaccgcccgccgtccgcagactgttactgttacatcgtcggtcaccgtctggactgtacgggacggcacgggtccgccgcgaatccactccacggcagtggctgagtgcgacggggttggtggccagagcagcagagcgaggctgctggtccaaaggggcctgcaggtgctgtgacatgctcggtgggcgcgtggccgaaagctgaacggctgggtcgtcgaacgacccgcggtggtcagttgcagcagtaattgctcagttgacccgttgtgtatgcatgcatgcatatgcgtgggatcacgcaccgcggaaatgagcgggcccacccacagccacaggtaaagcgagattcccgtgacaggcgctttcagattagtagattacagtgtgcttgccatcgcctgcattgcttggtccaaaaggctcacgggaggggcggagggagcagcaacataccaaatttttttttaaaaaaaaaacataccaattttgtatgaagtcatgtgaacacaaactttgtaccaaagtttttctttgaaaaaacataccaacttaataatgcctaaccattattcacgctaaacagtcgcttagtagggtgaaacgattattttaatgggatgaacggttgattaaaagaacccgggtgtagtaccgtgtaccgccgtgtgcaaacggtgtgttaaatagactaaaactaaacgatgttttagacaaacagtgagtataagaaatatcagtatcttttaatatataaatctgattaactttttaccctgctgctccatgcgccggcagaggcgctgtgttacagtgtgtgaagttctctcttttgacaagggttgggtacggttcgaagtttgaaactggggtcggtgggtcccagcagtcaggcactcaggctacttgtgtaataactgctgacacatgacttttgcagacaatgctacagtgctactaaaatagtctactccgtagaagaaattgccatgcggctcgggagtcgggagtcaatgggtgaaccagaaactgtccaacggcgagacgccaaaatgcacgccctacactgtcctgtccgtccgtccacgcctcacacggtacgcagcttttacgcaggcagcgcgcgtccatgtctgatgcagagcctgacgtctcctctcgccggccttcctgtgtcactatttgtatgagcggagggagaagtagccggctacctcgacggaaaaccccagcaggatttctggatgatgcttggtggatgaaaggcaaggcacgtaaagccatgcatcatgcaccacctaaagtcttagcagtacgaagcagacgagacgaccggtaagccaaagcagaagaagaagataagcatgcgtgccctgttttatacacttgttgtgttgatgctgagaggattcttgcctttcaatggtattggtggcacaccaacacaacactgcgctcttgcatcgcattctagccagccagtgcccgctcgcctactgctctcaccttctttacgttcccgctagggaagggaatggcagatgggcgcagcagcagcagcccatagctagcgcgtatccaccagctagcaatttttcttttgaaagaaaaaaaaacaatcttttttaccaggcaggttacagcgtgcatctgtcagaatatgttggctcgttgagacggattaataataagagagtgagtttgttgtcgacgtgtgtgtgtgtgtgtgtggggccaaatcacagtggacgcaggaggaatgatccggtgcatatgcttttacagtaatcactagccagcaccaacaacaattatagagacaaatccaaaccctcagccagctctctgaagaactggtcaaaaggtaattaatatgcacaggcagcacactagcacagcacagagcggcagaggataacacagtggtggagggcgctactacacactattctggataatatgggagagcaggaaaccctttcgtctgaacttactgaaacaagaaaatcaagaagaaaaaaaaggcatccattcagtgattggtgagcgagcagagcaggtgacgatcgaggtgaggtgaaagaaaaaagctggcaccgcatttaggcgaggttctttacgtaatttagcccaaagtggcaacaacggggaaaaaaggtaaagaccatggcggatcgccggcgcatccttccaccggccgtatattcgccccggcccccggggtggagggaggacggacggacgaggagcacgagtcccggtcagaaaagcagcgaagcacgcgccagatcgacagcgcagcgcagcgcaggcatccgcatcaacgggagcggagtaggcctcgggctcgggacgttttacccgaaaaccgcgcgaaaaaaacagcctctttgcttcatgagcggcgtggctgacgagcagcgtctttcctgttgattaattagacaaattccaaattaaattccaaatataaatcatgaccaaatcagaagaactgaaataaaagccaaatcagatgatgcgtactgattagacttactgattgttggcgcgcgccaggatcagctgggtcgacgatgtcagaagatcacgagcagtcgcgtgaagacgcttcccaaaaaccttattcgccctctcccggtgcaggatctagaagacgaagggttccggagacctgctctcctgatcgcagatgcacctctgcggtcgggacgaagggaactaaaaggcggctcagctatgaagagaggcgaaagcgaactgggatctgggattatttggaggctggctgccgggctccttttatagggccgagtccgcgaccccagtgcttatccgcccacgaaaatctcgcaatcagttgagattttatagggatcggttaggataagcgtaacagccaagaaaccaaaaaatcaaacggcaaaaggtagccgcgcccccgcaaggcgaggggccggatttcggcggaccattcgcgcgcatgtcgtgcgcccgcgcccttcgccccgccccgccccgccccggcccggcccggccaggccaggcgaggcgagcgagcgcgcgcgcgcgtgtggctctccacactctctcctctcatccatgacttggtgagtgagtgtggcttccatatttaagttagctctactccacaagagctagcaatatggtgctattggttccacctatccctttgccatccacttatatgggcttttgagattttcctgggatttatttgaataacttaattgggccaagcccataaatcctaacaatcccccaccagatctcaaatgcccatctgcagttttcgccactgttcactactgtttaatatactagtttttcagcagagactgttaagttgaacttccgcctagaactccaagttacaccaacccacaacttggacaatggactatgccttgaattgcaagttttgcgtgaatgggtttcacttgaagtcatgactagtacttggctaccagtagtccccttctcgggtggagcatatacgtcgtactccaaggtctcttcatgagtttactagagatcacccagatctcatagactgcgacgttagacagtctaactcatataggtgtgttctttcaaagaatgttctgcaggacaacatcttcgctaatacaagccaacagaacacattaaggcacaaagccaacctgccttacagcatttgagagtattgcatctttacttagagagggtcaaaagttactctcctcagttcaccaatggcttgttcttcccaggacctaattcacgggatctccgatcacatagactgggtttccaccatggcaactttattcgggtctcatacccatctctctcgatgcgatttctatcacattacgtggtagtcccttggtaaaaggatctgccagatttttatctgttgaaatataagtcacacttataactccggagtttctcaactttctgacagacttcaatcgtcttttgacatgtcttgatgactttccattatccttagaactcgtcactttagcaatcactgtctgattgtcacagttcataaggatagctggtattggtttctcaaccaccggcaagtccatcaagagttcacgcaaccattctgcctcaacggttgctgtgtcaagtgcagctagctcggcttccatggttgacctcgtcaaaatggtctgcttgcatgacctccatgataccgcacctccaccaatagtaaagacataaccactggtggcataaagctcgtctgcatcagatatccagttcgaatcactatatccttcaagtactgcatgctgaccagaatagtgaattccataactcattgtaccttgcaggtagcgcataacccgctcaagtgcatgccaatgatcagtcccggggtttgacatgaacctactcaatttgctcacagcaaacgagatatcgggccttgttgcaccagcaagatacatgagtgaaccgacaatctgagagtatctcaattggtctaaaccaattctcttgttctttcgcagtgtcacactgggatcataaggagttggagaaggtttgcactcagagaagccaaatcgcttcaaaaccttttcaacatagtgagattgcgagagagtaatcccaccatctgccttaatcagcttgatgtttagaatcacatcagcttctcccagatctttcatatcaaaactctttgatagaaaagacttgacttcattgatcacatcaatgtttgtgccaaatatcaatatatcatcaacatataagcacaatataactccttcgcccccaccacagcgataatatacacacctgtctgcctcattaatggcaaagcctgcagacgttagagtagtgtcaaacttctcatgccactgctttggtgcttgcttcagaccatacaaagatttcaataacttgcacaccttgctttcttgaccctttactacaaatccatcaggctgttccatatagatttcctcgtccagctctccattaagaaaagctgtctttacatccatctgatgaacaaggagaccatacgaggcagccaaagaaagtagtactcgaatagtggtcattctagcaacaggtgagtaagtatcaaagaagtcttctccttctttctgagtatagcctttagccacaagcctagccttgtacttttcaattgtaccatcaggcttgagcttctttttaaacacccacttacaacccacgggtttgcatccatagggtcgatcagtgacttcccacgtaccatttgaaagaatagagtccatctcattatgaactgcttctttccaatcatctgcatctggagatgcaaatgcttctgcaatggtagtaggagtatcgtccacaaggtacacaatgaaatcattaccaaaggatttttcaaccctttgtctcttgctccttttaggagcatcattgtcatcctcctctaggacaatttcatgtggctgttcaaaactctcaataggtgtattatgttcaggaattatctcagaagagtatctagaattgctatgaatgtctttcattggaaatatatgctcaaagaaagtagcatcacgtgattccataatagtatcaacatacacatcaggaacttcagatttaactactaaaaatctatatgctatgctacacgaagcatatccaagaaagacacaatccactgtccttggaccaagcttgcgctttttattaattggtacattgactttcgccatgcacccccaagtgcgcaagtatgaaagtgatggttttctcccaacccacttctcataaggggttttctcttctttgcccataggaattctattcagaacatgacatgaagtcaggactgcctccccccaccatgccttagataaaccacaagtgtctaacatggcattcaccaggtcagtcaacgtacggtttttcctttcagcaatcccgtttgactcgggtgaatagggaggagtcctctcatgaataatgccatgttctgcacagaaatcatcaaagactttgggaaagaactcgccaccacgatctgatctaagacgtttgatctttctctctagttggttttcaacctcagccttatagattttaaagtagtctaaagcctcatctttagtttttagcaagtatacatagcaaaatctagacgcatcatcaatcaatgtcatgaagtatctcttaccaccttttgtcaacacaccattcatctcacaaagatcagaatgtatgagttctagtggtgccaggtgtctctcctcagcagccttatgaggctttcgaggttgcttcgactgcacacaactatggcacttagaacctttgactatggtgatattcggaattaaactcatggttgcaagccgagacatagagccaaaattaatatgacacaaacgagaatgccaaatactcgcaagatcatcaacatttgcacaaatatggttcacagacttattattgaaatctaacaaagaaaagcggaacaagcctccgcaatcatagcctttaccaataaattgtccagacttggacacaactaacttattggactctaaaactaccttgaacccatctctacatagaagggttccgctaacgagattcttgtgtatagaagggacatgatgcacgttcttcagctgcacgatctttcccgaagtaaacttcagatccaccgtgccagtgccatgaacagaagcatgtgacccattccccattagcacggaagaatcccgggcgccctgataagaagagaacaagttgatgtcagaacacacatgaacattagcaccagtatcaagccaccagctaggtgattgaaatactgagaagatgaaaggtaaattaccataccctttgtcttcctcattgctagcgaccactgtgttgacattgccctttttgccacggcgatccgctcgatcgggacaatccttggcaaaatgacccgcctcgccacatccgaaacatgtcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcctccccccaccatgcattagataaaccacaagtgtctaacatggcattcaccaggtcagtcaacgtacggtttttcctttcagcaatcccgtttgactcgggtgaatagggaggagtcctctcatgaataatgccatgttctgcacagaaatcgtcaaagactttgggaaagaactcgccaccacgatctgatctaagacgtttgatctttctctctagttggttttcaacttcagccttatagattttaaagtagtctaaagcctcatctttagtttttagcaagtatacatagcaaaatctagacgcatcatcaatcaatgtcatgaagtatcttttaccaccttttgtcaacacaccattcatctcacaaagatcagaatgtatgagttctagtggtgccaggtgtctctcctcagcagccttatgaggctttcgaggttgcttcgactgcacacaactatggcacttagaacctttgactatggtgatattcggaattaaactcatggttgcaagccgagacatagagccaaaattaatatgacacaaacgagaatgccaaatactcgcaagatcatcaacattagcacaaatatggttcacagacttattattgaaatctaacaaagaaaagcggaacaagcctccgcaatcatagcctttaccaataaattgtccagacttggacacaactaatttattggactctaaaactaccttgaacccatctctacatagaagggttccgctaacgagattcttgtgtatagaagggacatgatgcacgttcttcagctgcacgatctttcccgaagtaaacttcagatccaccgtgccagtgccatgaacagaagcatgtgacccattccccattagcacggaagaatcccgggcgccctgataagaagagaacaagttgatgtcagaacacacatgaacattagcaccagtatcaagccaccagctaggtgattgaaatactgagaagatgaaaggtaaattaccataccctttgtcttcctcattgctagcgaccactgtgttgacattgccctttttgccacggcgatccgctcgatcgggacaatccttggcaaaatgacccgcctcgccacatgcgaaacatgtcaattcagccttgttcttcttcttcttgaagttggtagttttgttgggcttgttagattttggctttcctttgcccttgttgtggttcttctgaaccatgttggcgctggagtggccctcgcctcctttagatcccgtgtccttagcccgagctttctcctcaacatccagagacgctatcagattttcaactgatatctcctgtctcttatgtttcagagctgtggcgaagttcctccatgtagaaggcaactttgcaataatgcatccggccacaaatcggtcaggaaggactatcttaaggtggtcgagctccttggctatacactgtatttcatgagcttgctctacaatagagcgattatcaaccatcttataatcatgaaagctctccatgatatacaggtcactgccagcatctgatgcaccatacttagtagtaagtgcatcccacaactgtttcccgtctgtgtactgcatattcgcatcaaccagacggtcaacaagggcgctaagaacggctcccgtgaacatagtattggcatggtcgtactctttctcctgttcaggagtcagtggaccctcaggtctgcctttactaacatggaagacattcatagcagtaagccagagcgtggccttgacttgccatctcttaaagtgcataccattaaacttttctggcttcagcgcgtcggcaaaagcagccatagaaaaactaggaaattgtctacaataaggtttttggattgttgattaattagacaaattccaaattaaattccgaatataaatcatgaccaaatcagaagaactgaaataaaagccaaatcagatgatgcgtactgattagacttactgattgttggcgcgcgccaggatcagctgggtcgacgatgtcagaagatcacgagcagtcgcgtgaagacgcttcccaaaaaccttattcgccctctcccggtgcaggatctagaagacgaagggttccagagacctgctctcctgatcgcagatgcacctctgcggtcgggacgaagggaactaaaaggcggctcagctatgaagagaggcgaaagcgaactgggatctgggattatttggaggctggctgccgggctccttttatagggccgagtccgcgaccccagtgcttatccgcccacgaaaatctcgcaatcagttgagattttatagggatcggttaggataagcgtaacagccaagaaaccaaaaaatcaaacggcaaaaggtagccgcgcccccgcaaggcgaggggccggatttcggcggaccattcgcgcgcatgtcgtgcgcccttcgccccgccccgccccgccccggcccggcccggcccggccaggcgagcgagcgcgcgcgcgcgtgtggctctccacactctctcctctcatccatgacttggtgagtgagtgtggcttccatatttaagttagctctactccacaagagctagcaatatggtgctattggttccacctatccctttgccatccacttatatgggcttttgagattttcctgggatttatttgaataacttaattgggccaagcccataaatcctaacatttccgtgctgtttgtgcagttgaacgcatgatcgacctcacacgtcgctggtgggaacacgaaaggaactgaagaaacgcagcgagaatggtggtggtaccgtacgtacgtggtgggttcagactgcctccgtgatcttctttctttttgacctgttttctgcttgaacaaggcaagttattttgtggtggcacttgatgtgcggtccacattttaagcatcgccgcccgccagaatataagcacctgacaaccattgccctggggcaaccaaagcccatacagcgcgcttgattagttgccaggaaatgacgcttgattagcaggtgaaaaaacaaaatcaccagaggctccagcacagctcacgagttgatgcatatataatgaatggcgcgcggcttgttctgaaacaactcgctttcatgattttttctcatgagctgacaaatgtgcagcagctagctgcacacgatccaactaaacatactactactactatctcatggcacttgggttacacccggaggtattcaatgcgaaggatgctactccgagacaagaaaagaaaaagataaattcaatgcgatgcaagggatgagctcatgagcatgagcatagcagccatcatccatcctgatgctgcgctgtccaagtgcagtgtatgcaaaggatgagagagagaaggataggccttgctgtcgtagtgctgctgctgcaactgcaaggctccaaaaccaaatgggtcaggcgcagggaggtcatgtgaaagacaacacgatacaggcctctaacctttggtccttcatgtgaggtcaggttattaataaggtcatcagttattttataacccacccttcgttttaaactctagtctacagaatatagcctgtagtataaaacattgctttatatgtttttacacaagtccatctagtgatcttatactcctgctaattctaatgggggcgcatttaaggggtttttattagtggggaggaaaaggacccttgcaaaggcctagaagagagagagagagtgagtgcctggatgtggggaagagggagaaataaataaacccgaattcagccaaacctctctctcccacctcctgtcaccaccaccacacccctcctcctcccctgtctttctccctctccctcttgacaaaatttcctccacccattctcttcctcctcttcttccccaccgactccaccgatccacctctttctatctgaatttcctctcgtcttgcccaccgctccatctgtctgttgcctctctctctcgcaaccaatcggttcttgtttcctccgtctgctgggttgattctctctctcttcctctccgtggagaggagaggatcgagaaacaggagacaggaaagaagagaaatcagaacggagcctgttcttttgcctggattgcggttctcgcgagttgttcagaaactctgggccagtttctggtacggtttgttgcatctctctgttaaactgcctttttttgtatttctagttatatctcttgattttgcatagcgaaacaatcccttgtttgttgtttcaatccatgattccttcgcaaatatgcgttttttttcctcgctgattccttcacgtttcaatccacagtacttgtacatggagcggccttggtacggctctcgttaagctgtcaataattcttgtcttccccgaagcaaaattcccaacttccatgtttgaaaaaaaacacttttttctttcgtttgctcgcggtacagagatagatggatgtactgggcgtgttcttggttcagttaaaaacctcttatctgctcccgtatttcgttttcgtcatcgcagagggaatcgatccttctcttggaaatagtggatggctactcccaccttttccctttatatttttgcttcttttactgcgtccttttcaccttttgcaaaaccgatagggaagggaaataaatacaaaagagaaaaaaaacattgtgagcgagcgtccacaggagcaacgggccccatcctgcatgcggtccatctgtttaggtcgcatgcattgtacattcaccgtgctttgcttcacccattaaaatgcatgcatcaccacagctttttgctcgcaacagcaacccgaaccatcaagtttggtgcacgcgtacgccacaccaaatgcgcctaatgccaagattcctacagtcggattagttgccaataccctgattagttgctgcctactacagtaatgtgtgagagcaaaacaggtgcagctgcttccatagagtaacaacccagagtagaattacaatatttttggagtgcagacaacagttccttgtatgtagagttgacatattttggtggcaaatgttgaaggaatagcctagataaagcgtagccccgttctatatcccgttcatttcagtccttttcttgtgttccaataaagttggagcacagctctgttgcccaaaatgtttgatgtgttacttacaactggcaggtcctttaaaatactggaacgaggaaccatcagagatgctaaggcaacagaccaattgatgttatctcgctaccctagtttggatcatcacaatgcaaggacagaggaattctgtggagcattttgctgatgtttttggattcgacattgcatctagttcaggcaaccctgtgatggatcagcagtcatattggaacaatgttcttggatcagtagaatcgcagaatcttcaaggttatcagatgaatcacagtgatgctgccatgccatatgggaatgagacacagcaagatggtacatttcttggtttctgggaatcaggcgaagcaagtgcaagtggcagctctaacaatgccaaaacagagcatcttagtattggcggtggtctgaggattggtgaaaggcgactggtagctgacaatggcatttctctggatgtggatatcaaccttaacgccaacgttaacgatctatgtggtcaaagttcaaatgttaactgtacatctcagggccctgagcactatggtggcggtgatcgtagtgttgtaaattctcagccaactgacctgagattacacccatacaggactttcctactagatgcagagcaagcagattcttttactttgaaccctagtgaaaaccctttgtgtgatttttcactgatgcaagaaagcattgaccaaagacccggtagttccctggatggacgccggctagcctgcaagagaaaaaatgttgaaggacccaatggccagagttcagcaggtgctagcactagtttttcccacaggaacgacaatgctttccataacattgcttcttcaagttacaatcctgcacctatcagaaatccgtcctcacccaattgcttgccggttccaagttctatcgatgatcaactcccacgatatggaactaatgcagggctctcagccggtacctatgaccttaatggaggggtcaacaatgctgggaattcgcagagaagtttccgggcaagaattactacgtctcaacagattgctccctgtagtgtatggccctcttcaaatgctatcagacttcctaattcatggaatcatcagccacctcattttcaaagtgcatttgatgatacccaggaggttattcctgtggtcagcagcctcaacttgcaataccagcatccagtgaatgtttctagtgtgccaccggctgcaaaccgtttcactggccatggagcttcatcatcgagagctggcagtttggagaacagaattcttggtagtcaagaggctcctacaaggaatgtggtgcctgccaactactctgatttagttcccccgtctgtagtagacccgagacgtttgctgccagaaccatctaattggagttctgatgtccgaggcactgcaatatcaggaagtattcctcctgtatcaagagctaataacagtgcaactgttaatccgccagcaggattcagtcaccaaaacctcacccggcgccatcctcgaaatttatcagaggtatttttttcctccatttttcgcatttggttttctctatttttggattttggttttcagtttgattgctcatcttcccaccaaaatatcaagtgacgccatacttaaaatttggtttgatcatcttctcgatttgatcttttattttcttataggagattggtcggctatctggagcacttcgcggccatcaacccccacgcttaaggccggggtttctgttggagcgccagggcgatggtgtttggggtgttcctttatcaacaaggggtagagaaggaagaaggttaatggaggtaagatgatcggttctgttcaaaaaaatggtttttagcatccacttggaaacataaaagtccatcattattaggtgttttttgtgtggcagcaaagattacactaaatcatcctctaatttatgaatttatgcttgcaaaggccccccttgttttcagttatccttgtcatctttgtgtttgcctatctaagcaaattaccctattctattcccacaccaaattggtagaactgctctactcgaaatgctttatttcattttgtttgtgcacttcataatccctaatttgatctagcatgcttcttcatgcccctgcaactcttgaaatactgaaatttttttatcatttatttccacatattatcacactgccggttgttgtatcttttactttttttctaagatattttcactagtattgttatagagaaacttgaaccttaatgatatgtttcaccttatgtttttatgcaaaatactgcactattttatcctttttgttccctagccttgcttgttcttgcttgcgttaactactgtgaccatcatgtaaaggtgcatggttccttgtcttatgacaatccaagtttacttgtcaacagattcggaatgcacttgaaatgatccatagaggggagaatgtaaggcttgaggtcagtactagcaatgtccatctgagtttttttaactctgtttgctaccattatatcgacatgccactaaaataaacaaaagtaaaattgttctttgtggattgttctttttttttgctggacgtggttaagttatgattggctggttagtacaatcgtacttcagcataatattgtgttaagcctgccagcctgtgttaccctgtaattaaaattccaatttaaaatagcatggtaagaaaagaaaaacatgactataatcgtaaacataccatacctacagctgtcagcagaaaactaaggactaatatcaactaccgcataagtttaagcatataacagaaacacagatataagtgacaatgaattgcctcctgtgatctactgtagtccaaaactactatttgttagttggtaggaggtttatatttaatttgcttaaaattaatgggtcaacatgtagcaaactagtaatctgaaagttcagtacttgtagatagaaaatcatgaatcccatgagtgtacagcacgaagaaaaaataaaactgaatgaattcaaaagttttgggtgcccttgagtttttacctgctagtacttgatatatttccttcttacgccaaatgtattcttgaacagtctatcttctatggcggtgttgacattcacgatagacacagggacatgcgccttgacattgacaatatgtcctatgaggtttgtgttccttagtgaagcattacctgcacctttgcgaatctaaaatctaatctaagatggttgccctactttcaggagctattagcactcgaggaaagaataggaaatgtcggcactggcctcagcgaggaagctgtgataaggttgctcaaacaaaggaaattttcatcttggacactaaaagcatctttggaccctgaaccatgttgtatctgccaggtatttcatatactcccgagcatctcagaatcatggcttgttcttttgccgtaggtagatacaccaatcgcattgaatgattgaattgggcattcaggtgtttttggttcacatatttgtagcgtgaacggtaatcggtaacgttaaaccatgtttgtttaagtccaaccgtaatcgaataccacactagaaattgataccgtcctattcaaacttgttaccgatggtaatcgagtgtaaatcattaccgttgccatttatgttacatttcgtgaaccaaacggcacctcagttaggtcacctaggccatcatcacgtgtacaaagcaatgcagcacttaactaaattggcccacaaatattagaagggcaagttaagtctaaaagttgctaagcttgtctacttataccatctaggcctccaatggagaatcataccctggtaggtctctggatttctacaaattaacggtttgaaatggtagaactaagaatatttgtctcatcccttgagatttccatgctattgatcttggttcctagtgtctcaggaaagaattacaatccgactctagcatgacttccatcggatcactaaaaaagactacttaggccttgttcgtttacatcggattgtacccggaatcgttccgattaatcaaagtttatataaattatagaagcaatccggctcggaattattccgacccaccaatccgacacaaacgaacaagcccttaaggtgatagtgtagaggactaggcataatgcgtgcgtttaatttgcacggaacctttcagggaaaaaaatatttagttcagagggttggagggcaagatgcctctcgggatacatatgtcagtacgagattacaatgtcctaacggccaaatatactcaacactgttaaccttttttatagggtggctaataaaaaataacatcaactctggcaacaaaacctgtagcatagaactgtctatttaatcaggatttccattgtcgatgtaaatcatgtacaccagtatctgttatctgctgtgaatttaattcctttttgcctatgaacatgtcccagcgtcaccttatagagcaatgaaattagcagttgaacgaggctaatatatatacctaccttatgtcctttttatccctgtccaggaggagtacgctgatggagacgacctcgggaagctggactgcgggcacgacttccacgctggctgcatcaagcaatggctggtggtgaagaacgtgtgccccatctgcaagagcaccgcattgaagaagacctgaaacccgaacaaaggctcgtgtcagagacatcccccatgtttctatgttcactcgatggttcgcgagatcatttgccgattcgtattgctacgcttttttttttttggtcgggaaaattttgccagccaccttttggttaatgtctttcctgcttgatgtagtgaatttggactatgcctgatagttgtccttgacctgaatcaggttggtgtgtgggtgtttcatgtttggttgctgtgatagctggtacgaacaatgtatttctgttatgcaagtaatagaaatgaagtttctccggtcaaaaaaaaaaggtggaggaacgattcatatcgtacggacattctgatgatcattactagctccgtagctcgaactgaaacagcatgtcactgtactaaactattttatgtgctggaactgaaacccgcctcgctgtcgttgctccggaccggaccacgtcgttcgcggtactgctgccggcatgttcgcgcgctagcctccgtcctccgccttcgcgcgccgcccatggctcgcaggcccatggaaatctcgcacgcacgcacgacagctggtcagctgccagtcgccttggcatcggggcaagtgcctgcgccgttgagctgctgctaacaaagctacacagacggtactttgtcatgctcatatgcagcgctcgcatacagcgccgttgaaacatgatgacgcgcgcccgctaagactcccgtagtcccgttgcctttgccttcacttcccttttccctttccgatgtggtgggtggcctaacaacacccacccaattacccaaatcgccccatgcaaatgcaacgcgagcatcaccggcagcaggcaggggcactgctcacccaccctgaccctcggcgaccgctatcccggttcgttaggcgttagcgactggccacgatgagctggcagtgggaggctggtccatgattgcacccatgcagtcctcagctaactggctgccggccgcccggcgcacgaacgaagtgacgaggaagcgggccagccgacctaacgcggcggattcgcgcaccagccgacagctaactgcgttggtaagccaagccaaggcggggggcgatgccgacggaacgaaacaactggatcatgccaaagcgggcggcggggggagattagtttatggtgcaactcaggcggcaaagatattaatcgctgtcaagcaaggccgattcgaataatcatactgctagtagtagaactagtagtgagagagggtgatgatgacttgtttttcttatcgctgttacagctgtcctttttgacattttggaaagaccgccgcttgtggccgcctccctccgtctccgcctgccctgcccctgcctgcccgcccgccacgccgcccctgctgctgctggcctgctgccttcgtgcttatccacaacgtacccaaaaggccaaaaccctcgccgcaccgccctcccgcccgccgaccatccggtcgtcgaggacggataggataggataggcaggcggccgggtcagctggatcgtcgtgtacgtacgtacgtacgtggggacggtcggacggagacgacaacggcatgctgcgtcgtgggattgggaataaccgataacggacgtcgtcgttgaacctcgccttgcggtgcctgctgctcagctctctcgctcgcttgtcactcggcgtcggagctctcggggctcggcgcacggttcagatccgggttcttgacgcgcccgctccacgcaggccgctgcggcttctgctcctcttcctcctgctgctcctccgcgtgcacccgcagctgcggcaggtggttgtggtgcctatggcgctcgcggtggtgcggcggcgggaccaccatgccggtctggagcgcgtccacgcgggaccccacctcgatggccttcttccgtatggacgccgcggacagcgtgcagccgccgccgccgccggcggcggcggcggccgggtcgcgcgcgtgctcccccacgtccacgccatccgccagcccgaacgaggggatctcctcggggaagttgagccgcgccgaccgcccgcgcaggtagaacacggccgtgtcgtaggcgcgcgccgccgccacggccgtggcgtaggaccccagccagatgcgcgtgcgcttgtgcggctcccggatctccgccacccacttgccccacttgcgcatgcgcacgccgcggtactgccgccgcggctgcgtctgctggtgcgccgcccccagctgcggcgccgccatcgccgcctgctccgcctccatcttcaccgccgcggccgccagaggagccatggctgcggctgcggcaacagcggccgcagccgactcctcgatggaagaccggtactcgccctgcatcatcaacagctcaagccgcaagccgccgcctttgctggctgccgctgccgcctccccctctttctctctctctctctctcctgctgggggctgtgcaggcaggcgcagcagcagtggtatctgcagctgcttttggcggtggcgtggttttcttggcttcccttccctgtctctctgtttgctgggcgagtatttgtattgggattttgtgggtcgaaggggggatgagttgggcggaaggcgtcgacgacaccccctgtactgtgctgtgctgtttggaagggagctctcaccgccggcacacaaattgttaggtccaccggctaacttgcatccggtgcccatccactgccaacgggctaactatatatgactggttccaaactgcgggtacgtatctccggctatgttgtattattctcttatatcgaccctaattatctaccatagtcatataaccttcgtcacctcttatatatcgtagttatctaacgcacagtcttcaatctcatcttacaatcatctcaacaccacgttaacttttcttttacgtacttatagttgacataaacatattattttatttaaataaaaatacatttgattacataattaagcaaataaaattacataatccatttaaaatttaaaagtacatggcttaaaccaaaaaaaaatacacaattcgttaatataactataaactactagtcctcctcctcatcttcctctcctacctcgtgcaccgattctttatattctaatttataagttgctttatcaatctttttttcatagctaacttatagtcttttatcttgtcttccttcatatcagtaataaaacgatctggtagtttgttaaattactaccacaaagcaactccaatagttctataaaaaaagagcttgataaaatcatgtttagcaagttacaaaatagctatccaaagtaaaagtaacatagtctccgatagctgctcatatttagggagtaattcgattttcaatctagctttggcagccctgcacctatcagatcaggctgacggtagggttggaagcagaggatgtcgagggcagaagagaaaaaggccggtcgacatgacatatggaggagtgatggtccgctgacacaacctatgggacagtaaggtccaacggtaaaggagaaacttatgccaaacgtgtttgacctagctatatcactgtgctagaaagtggaaaacgagcttgctataccttgctacctcactgtgtggaaaatggaaaaaataatcatgccagtgtgaagaaattgacgtcaggtgcatttaaggagctcctatcgagttgccaaatatagagattatatagagttgacagcaactaactaattagcaagtctatttagagaaccattttttctgtttaattattaaatttaagacgatttagagagttgtttagagaactattagagttactctaaggagctctaacagcttgtccataccttcttggttagggaccctactgtataacaacgagacaaagaaggaaaggtaatggttgtggtagagaaatggcgagaggaaaaagagattagtaggaaagaggggtggggacgtacgtgtacattagaatctgagctacagaaattttgcgctgaacctaggtaggtttctgccacgtagaatataaaaggtgaattgatgtacacaggcctagctgtggacggagccacggaagcgatcccgtgaagaagcttacctgttgggcacactggttaattgcgtcagcattggatgggtttacgaacagcgacaatttacaatctagaaagaaaggtgaagacaataatgtcaagaaccaaattggcgtggatacgatacgagcatctggttgtcacaatcacaacgcacccccaccccacccgctcgctctcctccctgctcgtcaccaacccatgtgcacacctgtcgcctcaccgttgccctgctgaccctaaacgtttcgttcactagttcccaggacagaatgcgtggcggattgaccctaacgtggtgcgtacctctggctacgtgtgttggaggacggaataatggcgtctgggcccggcgatgtttcttggaatgatctgggacgaggagtacgtgcttccgtacggagccgtcgtcgctccactcagtcaccacacaagtaaaaacctggtcgatgctgcgtggtttcgccgtttgaaacttttggtttggattgaagctacgcagtccgcagccaagactacttttttttttatgctgcgtggtttttttaagtaaataacgtggtcgcagcatgacgacgctcctgataattgccttgccgccatgctgtgcttttttaggtgggcagggcagataggtgccgccaccgacccgggcgcgccgctcgcgtgcataatttgctgcggggggaatagctggcccggctttcctcctctcctgcccgcgactccaaacccaaaaaatcatgcaaatctaaagccgagagctgtcggtttcctacgagagacctgctgctctgctctcgagacgccagggcatcgccagtagtacccaatcatcagcgccagcaagcaagcaagcaagcgcgcggctcgtcgtcgtcggcggcaggtcgtacgtacaccgggcagtggggttttggatccagcggcggcagggcagcacgggggacggacaaacacgacgtgtcgctggggcgcggtcggccggcggggccttcacctcgccctacctggaccacatgcacgaggctcgagctcgataggtggacaataacgttgtagtcctatagtacgagcggtgtatgtagctgtaggctagctagcggctagggcgccggtagtggtgcctcaacgtcgatcgcgcccccgagctatagcctgtgtggtgcggcggtgccggccagtgcaaataggattgtccaactcatcttcttcttccttgctcgtttcatctctccttcgcactcggcgatgccggccactactgtagtcgccacgtcctagctaatgttcctcaagacatcaacgcgcctagctagctagctaattccctatttttctgtcccacgcagagtttttttttacttggcacaacagtggcatctggcacgtatgggcagcatgtcaacgatattgatttcatcgcagtcaacagtaccacccactgtgcagccgtgcaggacaaagaaacgtcactgtatccgcagacgaaagtagtacgggccggtagtacttctctgttggagattacacgagtggacagggaaagagaaaaaaaaaaccccgtcactctgatcaactggtacggcgcaggtcagcagcgatggtgtagtggatggtggatgccgggattggaggtggtcatcggctcaccgggtgctttggccgtcgtttgttaggttcagccggcggaggaggagagcgagacaaacagggcacgggcacatgtccaggcgccgtacgtttggtgcacgtaccgatttggccaaccccccacaaccaaccgccgttgcgttgcgtccatcggcctcgctcgcaccggcaccgccgacatgaggtgggcgggcgggcggctccaggtccaggtggcgtcgtcggggtgatgcgcgccgacgtggagacgtggccccttccttcttctacgcaaaatcccccacccacccacccacccccatccatccacacctcctcttcctaatccggacgcgggacgtcaatttgcttgccagcagcgacggcatcggccgcccgccttgtatctaatctaaccccgtaatcattcacgctaacgacgcccgtggcggggttagcgtaggcagcggcccggctggctgtggctgtctctgtctgcttggaagttggaccacggtgcggcgcgtaggggcccggccagctggtgctgtcgtctgcccgctcgcgtggtcgcgtttatttctcggtcggcgcgggcaagcgctcctctaccgggtccggatccggacggtagctagagggcgcagttgctgcaggtcggtggccgagggcgggcgagtcgtatacgtcgcggagagctggatcggagttgggtgtgcaggtaggtaggtgtgcgcccgccccagcagacttcagatatctgggatgacgtggggagttctgttgggtccgtccggcccgccgtacgtagtacgccagctcgctgcttgtctgtactagagtgaccagccgagctcgcgacgggtactggatgccggtccgtaccgtaccggtggtaaacagcgcatgcagtgcatttcagctagctagatccagtactagtagttttaactatatattcgtcgtaccagatcagaggcatagctggtccccgcctccccggacaggtaaactgcttccatctgtttccatcggaggcggccctggcgtggcagtggcactggcactggctctggctgcctcgccatgcaacgcaacgattatacgtgtgcgcgtcgagctgcatattctagttgtaccggcaggcagttcaagtgtcgaacgatctcagacaccaaaccgagatccaagtgacggcgaacggagctgcccccgcctgcccagcccagacggcctgcatagctgccgccaggcccaggcgcgaccggccgcagcgcgccgacaaggaaccatgctggctgactagctcgatcgagcggagcatgcggaccataaatggccaaatagactgtgcctaacggggccggtccgaagcacgacccgtttaatagtgctgggccaagttcagcacgagaattgtactgtgcttgggccgctgccttggtccgcagtgcggcccggcccggcccggcacgactatatattttctattttataaaatatagtatatatatttacattttatatttgctatttacaacaaatgtactagagttctattgattagtctcttttagttaatgttttcagccttgtaaagagaggtcgcgggttcaaattctcactcacacacagttttttttgctattttccttgatttaattggagaacgtggccagttaacgggccggcctggcacgactaccatggcatgcgggccggccgagcatggcccgattatctatcgggcctaacgggcccgtgctgggccgggctgtgccacccgtttgggcacctataatgctgacaatgcaggttgatagtttcaaggagcaatagctgagctagacacacacacagttttttgctattttccttaatttaatgggagaacgtgaccagttaacgggccggctcagcacgactaccaggccggcgtgccgtgcctgggccgcggcacgcgggccagccgggcacggcccaattattcgtcggatctaacgggcccgtgctgggccgggccgggccatccgtttaggcacctataatacggacactgcaggttgatagttcaaaggaggaatagctgagctagccaagccatgcatgtccatgtccatggggcctgtactgcgcgacgatgctcctcagctgctgtctcgctgcttgccctcaggggcacgacgttatcagtagcctcggtccgtgaagcagggagcgcgagcccttccacggctacgacctacgacctgaactccgatccgaataccacactgatcccaacacatgttttccgttgatcgctagaagccaccgcccactcgcccaggctatcttcagcttcgattccatgtgtcttctgcatgatgaaaaggttgggcccccgcccgcctggatccgtttccgtgcatgattagatgcagtgcaggccctgaaaattgctgttttcttctgacaaatcgtcacaggcacaggcggccggccggccgcagtccgctggtcgccttgcgcagagatgccatgccacgggctggccggcgcggggaggggaggggccggacgggggaacgggccccccatgacaagcctcctgcagtcctgtcgatatgatgtggagcaaaccaggacagccgccagcagatgaacgcagagtgggcagcactctctttgtgtgtgtgtgtgtgtggtggcatcttgccgtcttgcatggatccatcatggtcggtggctggaccgtcggagggcaccgcaccatagatagaggagcattgcattcacgatatcacagaagaggctgcccaggccggtggtaaagctaggtgacgaggaaaatgatgcatggccaagtgctgcagacagagagggcgtgtttgcgaccctgtgcctgactagttcggcccgttcaactggtccaggctaagattagtctgtcccagcttccgcaacttactatatttgcgcatttgtatcatatgagactcagagactggctcagacgagctggtgtttgtcacccagtatgtagccgggttagaagactaaaactacctaaccgtggaatccttgcgccacgaccagccaggctcccagaaaacggaccttcatctcgtttctgtggagccaggctgagagactaaacttgcacgcggacagccaggctcccgatcaagtcaggccaacgcaaatacgagaactgcagcctgagagcgcagccaggctgctgggccatggcggcaaacacacagagtcgtcctcttacactcggcactttcggtgcaggtgctgaggtgttgacccggcgaacgagcgagcgaggcccctcccgccgacgcgggcaagcgatcgagaaaaaagcctgaatcgagaacggttggtgctgctggtcgacgaccactgtttccttttcctacgctacgactagcgtttggtgcatggcagtacgccagtacccgtgcgtgaccgacctggttttgtgctgattaaggacttgttcgtttacgtcggattggtgggtcggaacaattccgaaccggattgcttctctaatttatataaactttgattaactggaacgattccggatgtaatccgatgtaaacgaaaagacctaaggctgcgttcgtttgtttaggattggggcaggaatggttccaggtgatcaaaacctatacaaattagataagcattcctggttggattggttctaggtcacgattccggaacaaacgaacacaccctaaggccttcttcgtttgtgtcggattgtaactgaaatcgttccagcaaatcaaagtttatataaattagagaagcaatccaattagaaatcttttcgacccacaccaatccggcacaaacgaacaagacctaagtctttgttcttttgtaccacattgcataaaaaattatttcagttaattaaagtttatataaattagataagcactaatctcaaacaaccgaacggcacctacggaagatagggaaaagcggtgtccggctcatgcctcccgtctactgttggcctcatcaagcctgtcgccctgcccggccaagagaccgagaggactgcttcttgcttcttggagcgtgatatatcctctgatgggttgggtgtgtaagtgtggtctgactcgtgcgagcctggtccactggaaacaggccgttccttacgtatatgtgcatgtaattaggagagatgtttcaggcttaagctgcagccgcgggtgcagccttggcgccgtgtaagcaggcaaccaaacgagagtcgcagcatggtcaacacatacacagaggctgaatgggcaggtgcagacgtccaatcacgcgactagtgtttgctcccctgcctgtcactagcagtgtggcttagtgctaggaatatgtgttagtttttgtttgggccagcacacatgacctaaaaatagaagcgtaaaacacatcccacaataaaataatatgaatcgaatgcgggcctaggtcgggccctatgttctagcccatcggaacccgacatcgatttttaaatttatttgatttagtaaggtacagaatttatattgttgtaacatagctacaaactttaggaccggaattgaccattatttctactctttagacaggcaacgaaacgactcaaacttttcttttaggtaggggtgggcacatttttaccttaaaccgaaccaaaacgaaatttcggtagttcggttcagtttcggttttttatatcagaagttcgttttttactgtataccgaaccgaaataccaaaaaaccaaaaaccaaactttatcaattctaaaatttgactatttgattatgtgaactaaatgtgtgaatcaattaaattgttattcacttatttatatgtgatgtatgatgtatctctgaatatttgtacctatataatttttacttttgaaaattatgtgtaatctatcatgtaaacttgttgtatgtgttgtcttagtataagtttggtattcggtttttaccaaaaaccaaaaccaaagtaagaaatcgaaactgaacttctcgatttttttattttagagaaaacgatcgatttctaatgtctagaaaaatcaaagttttttaaaaccgaaaatccgaacccaagtttaaaaaaacgaatgtccaactctacttttatgtcttcttggcccaagacacaatggacttgggcgggtccggttcggccttcccagcactagtgtgctcccggccttaggctggtcgcagtcgcgagctgtaaaagctcccctaagattttacagtggacgcgggagctgcagcggtaaactgtaccgatgactgcacatgtatagaggacaccgaaaactctatattcagcgcttgcactctctcactctatatacggggctgtcggtgtggggcccagtgacagccggtatgggtgcggacaaccggtagttgagaagtgggaaaaagtaatataatatgtggtagttggtatagagttgagatatagagtaaacatgactgcagaggattgtggtatagagtagataatcttgctgacggggataaaatattctttttaaagtagaaatttagagtagtatgagtgcggatagccttacgaatatggtacacggtacatacaggctaggtggcggccacggccgggggcggtgtgtatgtgtatcgtcatcatgctttcgccgttacctacgcggctacgcataggtaaaatataggctatgtttggtttctttagtctagagactaaagtttaattagaagactaaagtttagtctctaacatgtttggttatagaggctaaaaatagtaagaatatattaaatgacttataagaaaactaaaatgacctttaacattctactactattagtacaattgaactaaatgagtgataaatgtggaattaatatggtttagtccctactaaagactaaatcattttagtttatatttaatcctagtgtttggcaaaaaaaggactaaatgagactaaaaactagaaactaatctttagtccctctaaccaaacaccccatagtcggagttatagaggaaaaaacattttttctgtattgtttaatataaaaacacatactgtactagataaagaaaacacatagtcttccgttggtaattgattgagtgagtttatttttagaacaaatggtcggaaattatatgcacgacggtatgaagtccgtgacccaaggggaatggatcaggcttcccactttcctagtccacggcgcgtggtgcagtacagctacgggtgatagacaagtgacaacgatagttattactatatatatacacgatactttagtattcccggacgacaaaccatgtagaatgaatgatattgaaccgatttgaatacatagcaaattaaaaaatattttaatttattctaatacaatttaatttatatggaattgaaataactgaacaagaccttaatacagagcaagtaagtcgaaactagcatggcatgactatttggatttgccgtgggtggagatggctctttatttgtagccacagctggccactaggcaatgccgtcaaggccggcgtcaacaaaaataaaggtgatgatggacaatgttgccttgtcttggttgggtccctactgactaggacgtactgacaatgcagatgcaggcttgcagcgcaactcgtaaactttggataggaataggagagaatatatatccgtaccgagaagaaaagttgttagtacgtgatgaacagcgtcaacaggggcacgccgagccgtcctggtcctggtccaggtccagcccatgcttcgtcctcccgtgttctgtgcatcagacacttgttgcagagtcagcatgtgcatccatggctaccggtactgaggtacacgttgtaatagccatgtttaatatctattgcatgcgctcgcccccacgtacttctcgctcctgcaccaacactcctgtttaccgccacattgactatatatcatgatagcgttaaaaaccttatactcagacttaggaggtggaaaggagaaaatggggaagtgggccgcaggattaggagttgggccagaagggttctctctttgtcacacccggctttaaggaacaaagccgggtgcatctcatacatgcgccaagaagacaacatatataataacagagtgtatagagataaatgtcataaaacatcagagtatttattacatagcggaagacttattacaaaataaaagataaaagtaaaacgaactaaggatcgtcggcgccaatgtcgactgggaaacgccacctagatcagatcgaactcctcagtgttaggcggctcctcctgaaccacctgctcttctcctgtgggggggtgtgagacagcaagggtgagctcacacatgatcatagctcaacaagttgtggggaaccggtggacataaactcacaaaggtgtgaggtcatgtgatgtgtaaggctaatcaatgataggggttaaagctgagcattgcttttaagtagttggtcaaaattttattagcagttactaagtataagtgaataccaaaccttagataaagtaattgaacaaaattaataatagacccatgcaatgcaaatgacaaaattgaatttaagttccataatttaaacatcagagagtcctgagccgctcatgaccgcgagcacggctagtatactagttttacactctgcagaggttgtaccctttacccacaagtcatgctacccatctgccaaggggtcgtgaatcccatacacctctacctaggaagcgcggcagggcaacactacgaggcctttacaaagttccactagcttccgaaaacccgctacagtttataagaagttccaatgcagggttcttgtctgaccgccatcgcagcaaaatcaaccaaggacctccctacactgaccactcccctactgcccttgcccctttcgggtaaggtagtcttccactagctttcctaatttgtcttccaagggcgtcccattaaacccttgtggtggcacgtgtacgaatcaagctgattcatcagaatcggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaatcctagagatttcgtccacccgaccgcgagtgacaggagcgggcacgcacgagttgcgaaaaaaaaatgcgcggggtcactgcaagcaggcggggacggaaggcacgggtgcggtgcccaacggcgacagatgccgcacccactcccctactcaggtgaccgcggaaacccgttaatccgcccggtggcgcacgtctttctctctctagtactggctatgggtcgagctttcttcggcggcagcggagttctcccaccgacgaggctacacaatcaactgtggtgcgaggcaggggttcatttggtgggccaagataccgagcggcgcgctacttatgcgcccaggccaatccgcataagtcggcacacgatgtaacgaccatccgatacccgcggcggtgacggcgagatctctgcaatagaatccagagcggtcctgggctcggcggaagtgatagtcacgcggcgtagagaacgcgagttctgttacggcgaaggggaaggaactgacaacctgggcccgcctgcaagtcgccacagcacgcgcatctggaatgtggcccccaccatcggcgcagagggaagaggctggcaccaacggcccaccggtcggcacacggtctaatccgttcttttgggctgtgcgcatttgcctggtgggcctactggccaggaagacttcgcgcgtgcgcaccagtcgcagacaagtggacccgtgttgccagggtcatcttcccctgattgccgctggcgtgaaacgggtctccaaagtcgccgccgctttactcgccgatgaatcgcgcgcgtagttgtaacagccgccgcgttgatcggaggatcaggtcgagatttgccccctcaacctgggtacaaagaccccgggcgcgtcgcgagaactgttgaccaccccgagcgcgatctctggcacgcggaacccgcgcgtcgtcgcctaactccgccgagttcagagctaagccgccgcaaccgcgcatgccacaatctgcgctggcccatgacctgaccgaactctgcgcgtcgtgatcctcaccttctatatctgcggaccgaccccctcctttcctctccctttccccattgccaccgtgcgccattgcgaaagaaagtagccgccgtacttgttcccagaccaccgcgccacccgtggccgaattggagtgcagggagtttcgcctgacaccactggagccgctcatcacatcgctggacgagaagggcccctggacctccataattgctcgacggaggccgcttctgccgccaaaccccgtcgtcggctcttacatggctccccggtaagattgcccccattgagttcgtcgtatctccacgacgaatctcctagatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgaatctagagattcgtaccttatactcagacttaggaggtggaaggagaaatggggaaggggacacaggattaggagttgggccagaagggttctctctttgtcacacccggctttaaggaacaaagccgggtgcatctcatacatgcgccaagaagacaacatatataataacagagtgtatagagataaatgtcataaaacatcagagtatttattacatagcggaagacttattacaaaataaaagataaaagtaaaacgaactaaggatcgtcggcgccaatgtcgactgggaaacgccacctagatcagatcgaactcctcagtgttaggcggctcctcctgaaccacctgctcttctcctgtgggggggtgtgagacagcaagggtgagctcacacatgatcatagctcaacaagttgtggggaaccggtggacataaactcacaaaggtgtgaggtcatgtgatgtgtaaggctaatcaatgataggggttaaagctgagcattgcttttaagtagttggtcaaaattttattagcagttactaagtacaagtgaataccaaaccttagataaagtaattgaacaaaattaataatagacccatgcaatgcaaatgacaaaattgaatttaagttccataatttaaacatcagagagtcctgagccgctcatgaccgcgagcacggctagtatactagttttacactctgcagaggttgtaccctttacccacaagtcatgctacccatctgccaaggggtcgcgaatcccatacacctctacctaggaagcgcggcagggcaacactacgaggcctttacaaagttccactagcttccgaaaacccgctacagtttataagaagttccaatgcagggttcttggtctgaccgccatcgcagcaaaatcaaccaaggacctccctacactgaccactcccctactgcccttgcccctttcgggtaaggtaacgaatctctagattagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatgggaagagaaggaggagcttcgcatagattttcctcaccttttccctcgttcttcctaaatctcgaggacgagattatttttaaggggggtaggatttgtaacacccactttgtaaaaataaaacctaagagtaaagattatattccttcgcatatatgggtgtcatccctatgtagcatttcatgtggacatctcacctaaacaaataaataattaacaaaagacacctaaataacctaagtgcatcatgttggcttttacttgtatgtgcatttaatgataacaataatcatatcaatagaaatacaaaggtgtcccaaattgggattcaaagacctcatttacaatttaaaaaaatggaaataacataaaagaacaaggaaaagaaagggatcaccttgtaaagaaaactatataaaaataaatataacttttcaaattggtactcttaagttacaacttttgtctgagtacaaattcacaacaaaaaaatttgaattcaaatagagatttaaaagataaaagaaaaggaaaataaaaaaaaagaaaaaaaaagataaagcctacctgggccgtggctaccccttttggcccagtttcgcattacccctccgcgcagcccaggcgggagcatggctgcgctgctggctccgctgacaacgtgggcctactggccaggaagacttcgcgcgtgcgcaccagtcgcagacaagtggacccgtgttgccagggtcatcttcccctgattgccgctggcgtgaaacgggtctccaaagtcgccgccgctttactcgccgatgaatcgcgcgcgtagttgtaacagccgccgcgttgatcggaggatcaggtcgagatttgccccctcaacctgggtacaaagaccccgggcgcgtcgcgagaactgttgaccaccccgagcgcgatctctggcacgcggaacccgcgcgtcgtcgcctaactccgccgagttcagagctaagccgccgcaaccgcgcatgccacaatctgcgctggcccatgacctgaccgaactctgcgcgtcgtgatcctcaccttctatatctgcggaccgaccccctcctttcctctccctttccccattgccaccgtgcgccattgcgaaagaaagtagccgccgtacttgttcccagaccaccgcgccacccgtggccgaattggagtgcagggagtttcgcctgacaccactggagccgctcatcacatcgctggacgagaagggcccctggacctccataattgctcgacggaggccgctactgccgccaaaccccgtcgtcggctctctacaccgatcaaccaccggtaagattgcccccattgagttcgtcgtaatctccactaccgattgctcatcaccgcgttcggaatagctcgccaaatcacagatccgtagttcgccgccgtgctcacatccgtaggattagctgggcggcgccgccgcgcatggtcagtgtgttgggaacgatgaagtgctggtcgttgatcccgggttggttggtcaggattagatgagtgtggggatcgctggtagccgtcgaatgccaatccagcgcatagtattacataccatttcgggtgtggtcttgatctaatcaggaccctcggtcgacgatcgggcggctcgggttcgaaggataccccttcggcgggtcggtttagcgattaggcccctctgtttttagggaaatcacccgcggtccgcacagtgttgttcactgtgtcttaagaatcttactctaagacccctgtagtttgcagaaaatgaggcccagtccatagttctttaaattcagttatttaattatagaaattactttttaatacaaaataattgctagaactttgataattcatagaaaatacatatgaacttcaaaataatccattccaccttctaaatttttgtaattttattctttatcacctagagcctctgttatgtcatgaaaacagtaagaaatttatttatcacttaatcctatttaaagcacataaaacctttgaaaatccataacttaaattctataactccaaaaattatgattcctgttcctagaattttattttaatgtgtagattattactgtgtattttgtttatatgtttgatgtaatgttaatttctctatatgcattgtgcttgtttgtattgtggcgagtagaagagcccgtgaccgaggaccctggtgagcagcaggttgaagtagctgagcaggagctcattgaaggcaagttgtgcccttgaccactttttacccaataatgttctttaatatcacttatccatgcataggttaattttgatgggacccgataggtcaccctagattgtttatctcattaccttgtttacccctgaatcacttgggtagtttgctattgctttacatggttttgggataatcatttattacctctatgttccaattctttttgttattctatttatgttcatgttgagatcattaatgttaattggaacatagagcttaacttgagaaccacgtgccaccacaagggtttaatgggacgcccttggctgattaattaggaaagctagtggaagactaccttacccgaaaggggcaagggcagtaggggagtggtcaatgaagggaggtccctggttgattttgctgcgatggcggtcagccaggaaccctgcattggaacttcctataaactgtagcgggttttcggaagctagtggaactttgtaaaggcctcgtagtgttgccctgccgcgcttcctaggtagaggtgtatgggattcgcgaccccttggcagatgggtagcatgacttgtgggtaaagggtacaacctctgcagagtgtaaaactggtatactagccgagctcacggtcatgagcagctcaggactctctgatgtttaaattatggaacttaaattcaattttgtcatttgcatatgcatgggtttattattaattttgttctattactttatttaaggtttggtatttacttacacttagtaactgctaataaaattttgaccaactacttaaaagcaatgctcagctttaaccattctctttgataagccttacactccatgagctctcacctttggtgagttcatgtcacattattccccacaacttgttgagcgatgatcatgtgtgagctcacccttgctgtctcacaccccccccccacaggagaagagcaggtggttcaggaggagccacaaggcgaggagtatgatctgatctaggtggcgtttctcagttgacattggcgccgacgatccttagttcgttttacttttatcttttatttttttaataagtcttccgctatgtaataaatactctgatgttttacgacatttatctctatacactctgttattatatatgttgtcttcttggcgcatgtatgagatgcacctggctttgttccttaaagccgggtgtgacagaagtggtatcagaggaaatgttgactgtaggacgaaacctagatagaaatggacagaccctttcctacttaccttactctgactcattctatactcatctcatcttgatcttgtctcaccttctattgttctactcttatcattcttaccttttctactcctagacaagatggatttcacaccttggagtccctacccctatgacatttttaagaggtaggaagcctaagacaaaattaaaactattttctttattaaaaatgttggttgattgttctgatgatcaatgcctgatttgcttctttgattgattgaaatagtatagacgggcatcttagcatgtaccaccataaggtaatgtattagctttagtgggtgacacactaatctacttagctaataaatcccccgtagtaacattactcgtaattacatgccttgtctataatcctttctttcttaccctgtgttcctgacccaaatgggctacccctatactgttagaagagagcagtataggcttgatccttggtatgtcatggttaagaaaggcaaaggcactttgtacattgtgctaagggaaccgtagcactcaccagttccaaaggacaaagatttgaagttgaagttgcagtaactaccaccatcagactagtggcgttcttagtagatgagaagttgtgggtgacaacatccgtgtggttagagattttccggatgtctttccagaggagttaccagggatgccacccgatagagaagttgagtttgttattgatctcttacctggaaccgcccctatttctaaatggccatacaagatgtccgtagaagaactaaaaggaacttaagaagcaattaacggaattacaagaggatgggtacattcgtccgagttcttcaccttgtggagcaccagttctattttgtacagaagaaagatggatcgcaagggatgtgcgtggattataggacccttaacgatgtcactgtgaaggacaagtgtccattaccccgcattaaggatttatttaatcagatgagaggtgctagagtattctcgaagattgatctccgatcgggttaccatcagatgaggattagaccatcggatattcccaagacggctttctcgactcgatatggattatatgagtttactgttatgtcgtttggattaaccaatgcaccagcttattttatgaacttgatgaataaggtgtttatggagtatttggacagactcgtcgtagtattcatcgacgatattcttatttattccaagagtgatagtgatcatgaggaacatctgagattggtgctacaaaagctaagagataatcaactctacaccaagtttaccaaatgcgagttttggattggcgaggtgccatttcttggacatatcatttctgatggaggaatatcagtggatcctgctaaagttaaggagataatggagtggagagtacccactacagtgactgagattcggagtttcttgggattagcaggctattatcggagatttattgaagggttctctaagattgccaagcctatgacttcgcttttggagaaaggaaaagaatttaagtgggacgagaagtgtcaagacagctttgatctattgaagaagaggttaatgtcgccaccagtattggttatgccggacctacagaaaggatttgatatctattgcgatgcatgtggccaaggcttaggatgtgtggtcatgcaggaaggacatgtgattgcttatgcttctcgacaattacggagacatgaattgaactaccccactcatgacttggaattggcagccgttgtgcatgcacttaagatttggagacattacattatgggaatcaagtgtcaagtatacacagatcataagagtctgaagtatatattcactcagaaggatctcaaccttaggcaacgccgttggttggagcttattaaggattatgatttggagattcactatcacccgagcaaggcaaatttggttgcagatgccttgagtcgaaaggagcatgttcattcagctgttgttgcccagctacccgatgagattgttgaggatttcaggagacttaacctggggatagttgctcacactgaaggagttactattgatgtggaacctaccttggagcaagaaatccgtaaaggacaaattggtgatgctaaaatacgagagattaaggatctgattactgaaggtcgagttctggaatttacggaagatgagcaaggcactgtatggttcaaggacaagatgtgtgttcctgatattgaaagccttcgtgagactatattgaaggaggcccatgactcaaattattctattcatcctggtagtactaagatgtatcaggatttgaagcggaagtattggtggtatggattgaaaagagatgtggctgcacatgtggctatgtgtgatgtatgtcaaagagttaaggctgaacaccagaggccagttggactattgcacccactgaagatacccgagtggaagtgggaagagattggtatggatttcattactggattgcctcgcacccagaaaggatatgatgctatatgggtgattgtggatagattgactaaagtggctcactttattcctgtcaagactacttataaaggttctcagctagcagagttgtatatggctcggcttgtgtgtttacatggagtaccaaagaagatcgtgtctgatcgaggttcgcagtttacctcaagattttggagaagctttcatgagaacatgagcacgaagttgaattttagtacggcttatcaccctcagactgatgaacagactgaaaggactaatcaagttttggaagatatgttgagagcatgtgcccttcagcatgggaggaagtgcaggactcctttgtattgggatcagactggagagagacagttctttgggcctgaattgattcaagaagcagaagaacaagtccgtataattcgagagaatttgagagtggctcaaaccaggcaaaagagttatgctgataatagaaggaggccactgtaatttgaagaaggagattatgtgtatctcaaggtgtcaccactgcgtggaatgaggagattcaaagttaagggcaaattgtcccctcgctatattggaccattcttgatctttaggagagttggagagatggcataccaacttgagttacctgctagtctatcggatgtgcataatgtgtttcacgtatctcaactcaagaagtggctccgtgtccctgaggaacagttaccaatggaagagcttagtgttcagggtgatttaacttacacggaatacccgatcaagatttaggatactttgactcgagttacaagaaataaggtggtaaagatgtgcaaagtccaatggagtcaccacggtgaagatgaagcaacttgggaaagagaagaagagcttcgcatagattttcctcaccttttccctcgttcttcctaaatctcgaggacgagattatttttaaggggggtaggatttgtaacacccactttgtaaaaataaaacctaagagtaaagattatattccttcgcatatatgggtgtcatccctatgtagcatttcatgtggacatctcacctaaacaaataaataattaacaaaagacacctaaataacctaagtgcatcatgttggctttacttgtatgtgcattaatgtaaccaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgaatctagagattcgtctaagggtacaacctctgcattagtgtaaaactggtatactagccgagctcacggtcatgagcagctcaggactctctgatgtttaaattatggaacttaaattcaattttgtcatttgcatatgcatgggtttattattaattttgttctattactttatttaaggtttggtatttacttacacttagtaactgctaataaaattttgaccaactacttaaaagcaatgctcagctttaaccattctctttgataagccttacactccatgagctctcacctttggtgagttcatgtcacattattccccacaacttgttgagcgatgatcatgtgtgagctcacccttgctgtctcacaccccccccacaggagaagagcaggtggttcaggaggagccacaaggcgaggagtatgatctgatctaggtggcgtttctcagttgacattggcgccgacgatccttagttcgttttacttttatcttttatttttttaataagtcttccgctatgtaataaatactctgatgttttacgacatttatctctatacactctgttattatatatgttgtcttcttggcgcatgtatgagatgcacctggctttgttccttaaagccgggtgtgacacggggtgcgaggcaatccagtaatgaagtccttaccaatttcttccccttccacttgggtatcttcagtggatgtaatagtccagctggtctttggtgttaggccttaactctttgacacccatcgcacatagccacatgtgcagccacatctctcttcaatccataccaccagtatttctgcttcaattcctgatacatcttggtactaccaggatgaatagaataatccaagttatgggcttcctttagaatagtcttacgaaggctttcaataccaggaacacatatcctgcccttgaaccatattgtgtcttgttcatcttccgtaaattccggacctctacctttcgtaataagatccttgatctcttgtatcttggcatcaccaatctgtcgttctgtggattttcttgctccaaggtaggttccacatcaataatgactccttaagcgtgagcatctatccccaggttaagtctcctgaaatcctcaacaatctcatcgggtagctgggcaacaatagctgaatgaacatgctcctttcgactcaaaggcttctgcaaccaaatttgccttgcccagtgatagtgaatctccaaatcattatcccttaataagctccaaccaacggcgtgcctaaggttgagatccttcctgagtgaatataaactcttatgatccgttatacttggcacttgttccccatgatataaagtctccatatctttaagcgcatgcccaacggcctgccaattccatagcatgagag